(19) United States
(12) Patent Application Publication
(10) Pub. No.: US 2009/0238825 A1
(43) Pub. Date: Sep. 24, 2009

(54) NOVEL RABBIT ANTIBODY HUMANIZATION METHODS AND HUMANIZED RABBIT ANTIBODIES

(76) Inventors: Brian R. Kovacevich, Snohomish, WA (US); John Latham, Seattle, WA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/399,156

(22) Filed: Mar. 6, 2009

Related U.S. Application Data
(63) Continuation-in-part of application No. 12/124,723, filed on May 21, 2008.
Application No. 12/124,723 is a continuation-in-part of application No. 12/153,611, filed on May 21, 2008.
Application No. 12/153,611 is a continuation-in-part of application No. 12/153,612, filed on May 21, 2008.
Application No. 12/153,612 is a continuation-in-part of application No. 12/391,717, filed on Feb. 24, 2009.
Application No. 12/391,717 is a continuation-in-part of application No. 12/323,066, filed on Nov. 25, 2008.
Application No. 12/323,066 is a continuation-in-part of application No. 12/153,612, filed on May 21, 2008.
Application No. 12/153,612 is a continuation-in-part of application No. 12/153,611, filed on May 21, 2008.
Application No. 12/153,611 is a continuation-in-part of application No. 12/124,723, filed on May 21, 2008.
Application No. 12/391,717 is a continuation-in-part of application No. 12/323,147, filed on Nov. 25, 2008.
Application No. 12/323,147 is a continuation-in-part of application No. 12/153,612, filed on May 21, 2008.
Application No. 12/391,717 is a continuation-in-part of application No. 12/153,611, filed on May 21, 2008.
Application No. 12/153,611 is a continuation-in-part of application No. 12/124,723, filed on May 21, 2008.
Application No. 12/391,717 is a continuation-in-part of application No. 12/323,194, filed on Nov. 25, 2008.
Application No. 12/323,194 is a continuation-in-part of application No. 12/153,612, filed on May 21, 2008.
Application No. 12/153,612 is a continuation-in-part of application No. 12/153,611, filed on May 21, 2008.
Application No. 12/153,611 is a continuation-in-part of application No. 12/124,723, filed on May 21, 2008.
Application No. 12/391,717 is a continuation-in-part of application No. 12/366,567, filed on Feb. 5, 2009.
Application No. 12/366,567 is a continuation-in-part of application No. 12/323,066, filed on Nov. 25, 2008.
Application No. 12/323,066 is a continuation-in-part of application No. 12/153,612, filed on May 21, 2008.
Application No. 12/391,717 is a continuation-in-part of application No. 12/153,611, filed on May 21, 2008.
Application No. 12/391,717 is a continuation-in-part of application No. 12/124,723, filed on May 21, 2008.
Application No. 12/124,723 is a continuation-in-part of application No. 12/323,147, filed on Nov. 25, 2008.
Application No. 12/323,147 is a continuation-in-part of application No. 12/153,612, filed on May 21, 2008.
Application No. 12/153,612 is a continuation-in-part of application No. 12/124,723, filed on May 21, 2008.
Application No. 12/124,723 is a continuation-in-part of application No. 12/153,612, filed on May 21, 2008.
Application No. 12/153,612 is a continuation-in-part of application No. 12/124,723, filed on May 21, 2008.
(60) Provisional application No. 60/924,550, filed on May 21, 2007.
(60) Provisional application No. 60/924,551, filed on May 21, 2007.
(60) Provisional application No. 60/924,551, filed on May 21, 2007.
(60) Provisional application No. 60/924,550, filed on May 21, 2007.
(60) Provisional application No. 60/924,550, filed on May 21, 2007.
(60) Provisional application No. 60/924,551, filed on May 21, 2007.
(60) Provisional application No. 60/924,550, filed on May 21, 2007.
(60) Provisional application No. 60/924,551, filed on May 21, 2007.
(60) Provisional application No. 60/924,550, filed on May 21, 2007.
(60) Provisional application No. 60/924,551, filed on May 21, 2007.
(60) Provisional application No. 60/924,550, filed on May 21, 2007.
(60) Provisional application No. 60/924,551, filed on May 21, 2007.
(60) Provisional application No. 60/924,550, filed on May 21, 2007.
(60) Provisional application No. 60/924,551, filed on May 21, 2007.
(60) Provisional application No. 60/924,550, filed on May 21, 2007.
(60) Provisional application No. 60/924,551, filed on May 21, 2007.

(30) Foreign Application Priority Data
May 21, 2008 (US) PCT/US08/64414
May 21, 2008 (US) PCT/US08/64432

(51) Int. Cl. A61K 39/395; C07K 16/18; C07K 16/08; C07K 16/12; C07H 21/04; C12N 15/63; C12N 1/21; C12N 1/19; C12N 5/10; C40B 30/00

(52) U.S. Cl. 424/133.1; 530/387.3; 536/23.53; 435/320.1; 435/252.3; 435/254.2; 435/325; 506/7

(57) ABSTRACT

The present invention is directed to novel and improved methods for humanizing rabbit heavy and light variable regions. The resulting humanized rabbit heavy and light chains and antibodies and antibody fragments containing are well suited for use in immunotherapy and immunodiagnosis as they retain the antigen binding affinity of the parent antibody and based on their very high level of sequence identity to human antibody sequences should be essentially non-immunogenic in humans. The invention exemplifies the protocol for the manufacture of therapeutic humanized anti-human TNF-alpha and anti-human IL-6 antibodies.

### RELATED APPLICATIONS

This application is a continuation-in-part of U.S. Ser. No. 12/124,723, filed May 21, 2008, which relates to and claims priority to provisional application U.S. Ser. No. 60/924,550 and 60/924,551 and utility U.S. patent application Ser. No. 11/802,235 each of which was filed on May 21, 2007. In addition, this application claims priority to and incorporates by reference in its entirety PCT applications filed on May 21, 2008 entitled “Antibodies To IL-6 And Use Thereof” (PCT/US2008/064432) and “Antibodies To TNF Alpha And Use Thereof” (PCT/US2008/064414) and which PCT applications were filed under Attorney Docket Numbers 67858-701802 and 67858-701902, respectively;

this application is also a continuation-in-part of U.S. Ser. No. 12/153,611, filed May 21, 2008, which claims the benefit of Provisional Application 60/924,551, filed May 21, 2007;

this application is also a continuation-in-part of U.S. Ser. No. 12/153,612, filed May 21, 2008, which claims the benefit of Provisional Application 60/924,550, filed May 21, 2007;

this application is also a continuation-in-part of U.S. Ser. No. 12/391,717, filed Feb. 24, 2009, which is a continuation-in-part of U.S. Ser. No. 12/323,066 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008; U.S. Ser. No. 12/391,717, filed Feb. 24, 2009 is also a continuation-in-part of U.S. Ser. No. 12/323,147 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008; U.S. Ser. No. 12/391,717, filed Feb. 24, 2009 is also a continuation-in-part of U.S. Ser. No. 12/323,194 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008; U.S. Ser. No. 12/391,717, filed Feb. 24, 2009 is also a continuation-in-part of U.S. Ser. No. 12/366,567 (Attorney Docket No. 67858.702101) filed Feb. 5, 2009, which is a continuation-in-part of U.S. Ser. No. 12/323,066 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008; and is a continuation-in-part of U.S. Ser. No. 12/323,147 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008; and is a continuation-in-part of U.S. Ser. No. 12/323,194 filed Nov. 25, 2008, which is a continuation-in-part of U.S. Ser. No. 12/153,612 filed May 21, 2008, which claims the benefit of U.S. Ser. No. 60/924,550 filed May 21, 2007, and is a continuation-in-part of U.S. Ser. No. 12/153,611 filed May 21, 2008, which claims the benefit of U.S. Ser. No. 60/924,551 filed May 21, 2007, and is a continuation-in-part of U.S. Ser. No. 12/124,723 filed May 21, 2008;

each of the foregoing is hereby incorporated by reference in its entirety.

The sequence listing file named “67858704101.txt” having a size of 438,735 bytes which was created on Mar. 5, 2009 is hereby incorporated by reference in its entirety.

### BACKGROUND OF THE INVENTION

1. Field of the Invention

This invention provides a novel and improved amino acid sequence- and homology-based method for modifying (humanizing) rabbit antibody amino acid variable heavy and light chain polypeptide sequences or antibodies from closely related species such as other lagomorphs. The resulting modified antibody sequences are less or non-immunogenic in humans relative to the parent antibody, e.g., rabbit antibody and retain the same or substantially the same antigen binding affinity relative to the parent antibody from which the modified (humanized) antibody sequences are derived.

The invention further provides humanized variable light and variable heavy chains derived from rabbit antibodies which are produced by such methods. As shown infra, the methods of the object invention reproducibly yield humanized antibodies that retain the antigenic specificity and affinity of the original rabbit antibodies. The inventive procedure in general relies on transferring specific amino acid residues (“selectivity determining residues”) contained in rabbit antibody complementarity determining regions (CDRs) from rabbit antibodies onto homologous human antibody variable heavy and light chain polypeptide sequences.

This invention in more specific embodiments exemplifies humanized antibodies and humanized antibody fragments and variants thereof having binding specificity to interleukin-6 (IL-6) or tumor necrosis factor alpha (hereinafter “TNF-alpha”) which were produced using the novel humanization protocols provided herein. However, it should be understood that the novel humanization protocols provided herein are applicable to the humanization of rabbit or other lagomorph derived antibodies that specifically bind to any desired antigen. This includes by way of example antibodies specific to antigens from infectious agents (viruses, bacteria, fungi, parasites and the like), allergens, human antigens such as enzymes, hormones, autoantigens, growth factors, cytokines, receptors, receptor ligands, immunoregulatory and immunomodulatory molecules, et al.

The invention also pertains to methods of using humanized antibody and antibody fragments produced according to the invention as therapeutics and for diagnostic purposes such as for in vitro and in vivo screening assays for detecting diseases and disorders associated with such antigens. For example this includes in vivo imaging screening methods using antibodies to IL-6 or TNF-alpha and methods of treating diseases or disorders associated with TNF-alpha or IL-6 by administering said humanized antibodies or fragments thereof.

2. Description of Related Art

Antibodies play a vital role in our immune responses. They can inactivate viruses and bacterial toxins, and are essential in recruiting the complement system and various types of white blood cells to kill invading microorganisms and large parasites. Antibodies are synthesized exclusively by B lymphocytes, and are produced in millions of forms, each with a different amino acid sequence and a different binding site for an antigen. Antibodies, collectively called immunoglobulins (Ig), are among the most abundant protein components in the blood. Alberts et al., Molecular Biology of the Cell, 2nd ed., 1989, Garland Publishing, Inc.

A typical antibody is a Y-shaped molecule with two identical heavy (H) chains (each containing about 440 amino acids) and two identical light (L) chains (each containing about 220 amino acids). The four chains are held together by a combination of noncovalent and covalent (disulfide) bonds. The proteolytic enzymes, such as papain and pepsin, can split an antibody molecule into different characteristic fragments. Papain produces two separate and identical Fab fragments, each with one antigen-binding site, and one Fc fragment. Pepsin produces one F(ab′)₂ fragment. Alberts et al., Molecular Biology of the Cell, 2nd ed., 1989, Garland Publishing, Inc.

Both L and H chains have a variable sequence at their amino-terminal ends but a constant sequence at their carboxyl-terminal ends. The L chains have a constant region about 110 amino acids long and a variable region of the same size. The H chains also have a variable region about 110 amino acids long, but the constant region of the H chains is about 330 or 440 amino acid long, depending on the class of the H chain. Alberts et al., Molecular Biology of the Cell, 2nd ed., 1989, Garland Publishing, Inc. at pp 1019.

Only part of the variable region participates directly in the binding of antigen. Studies have shown that the variability in the variable regions of both L and H chains is for the most part restricted to three small hypervariable regions (also called complementarity-determining regions, or CDRs) in each chain. The remaining parts of the variable region, known as framework regions (FR), are relatively constant. Alberts et al., Molecular Biology of the Cell, 2nd ed., 1989, Garland Publishing, Inc. at pp 1019-1020.

Natural immunoglobulins have been used in assays, diagnosis and, to a more limited extent, therapy. However, such uses, especially in therapy, have been hindered by the polyclonal nature of natural immunoglobulins. The advent of monoclonal antibodies of defined specificity increased the opportunities for therapeutic use. However, most monoclonal antibodies are produced following immunization of a rodent host animal with the target protein, and subsequent fusion of a rodent spleen cell producing the antibody of interest with a rodent myeloma cell. They are, therefore, essentially rodent proteins and as such are naturally immunogenic in humans, frequently giving rise to an undesirable immune response termed the HAMA (Human Anti-Mouse Antibody) response.

Many groups have devised techniques to decrease the immunogenicity of therapeutic antibodies. Traditionally, a human template is selected by the degree of homology to the donor antibody, i.e., the most homologous human antibody to the non-human antibody in the variable region is used as the template for humanization. The rationale is that the framework sequences serve to hold the CDRs in their correct spatial orientation for interaction with an antigen, and that framework residues can sometimes even participate in antigen binding. Thus, if the selected human framework sequences are most similar to the sequences of the donor frameworks, it will maximize the likelihood that affinity will be retained in the humanized antibody. Winter (EP No. 0239400), for instance, proposed generating a humanized antibody by site-directed mutagenesis using long oligonucleotides in order to graft three complementarity determining regions (CDR1, CDR2 and CDR3) from each of the heavy and light chain variable regions. Although this approach has been shown to work, it limits the possibility of selecting the best human template supporting the donor CDRs.

Although a humanized antibody is less immunogenic than its natural or chimeric counterpart in a human, many groups find that a CDR grafted humanized antibody may demonstrate a significantly decreased binding affinity (e.g., Riechmann et al., 1988, Nature 3 32:323-327). For instance, Reichmann and colleagues found that transfer of the CDR regions alone was not sufficient to provide satisfactory antigen binding activity in the CDR-grafted product, and that it was also necessary to convert a serine residue at position 27 of the human sequence to the corresponding rat phenylalanine residue. These results indicated that changes to residues of the human sequence outside the CDR regions may be necessary to obtain effective antigen binding activity. Even so, the binding affinity was still significantly less than that of the original monoclonal antibody.

For example, Queen et al (U.S. Pat. No. 5,530,101) described the preparation of a humanized antibody that binds to the interleukin-2 receptor, by combining the CDRs of a murine monoclonal (anti-Tac MAb) with human immunoglobulin framework and constant regions. The human framework regions were chosen to maximize homology with the anti-Tac MAb sequence. In addition, computer modeling was used to identify framework amino acid residues which were likely to interact with the CDRs or antigen, and mouse amino acids were used at these positions in the humanized antibody. The humanized anti-Tac antibody obtained was reported to have an affinity for the interleukin-2 receptor (p55) of 3×10⁻⁹ M⁻¹, which was still only about one-third of that of the murine MAb.

Other groups identified further positions within the framework of the variable regions (i.e., outside the CDRs and structural loops of the variable regions) at which the amino acid identities of the residues may contribute to obtaining CDR-grafted products with satisfactory binding affinity. See, e.g., U.S. Pat. Nos. 6,054,297 and 5,929,212. Still, it is impossible to know beforehand how effective a particular CDR grafting arrangement will be for any given antibody of interest.

Leung (U.S. patent application Publication No. US 2003/0040606) describes a framework patching approach, in which the variable region of the immunoglobulin is compartmentalized into FR1, CDR1, FR2, CDR2, FR3, CDR3 and FR4, and the individual FR sequence is selected by the best homology between the non-human antibody and the human antibody template. This approach, however, is labor intensive, and the optimal framework regions may not be easily identified.

As more therapeutic antibodies are being developed and are holding more promising results, it is important to be able to reduce or eliminate the body's immune response elicited by the administered antibody. Thus, new approaches allowing efficient and rapid engineering of antibodies to be human-like, and/or allowing a reduction in labor to humanize an antibody provide great benefits and medical value.

Citation or discussion of a reference herein shall not be construed as an admission that such is prior art to the present invention.

### SUMMARY OF THE INVENTION

The invention is based, in part, on a new humanization strategy for producing humanized variable heavy and/or light regions and humanized antibodies or antibody fragments containing such humanized variable heavy and light regions derived from rabbit or other lagomorph antibodies. Preferably these rabbit or other lagomorph derived antibodies which are used for humanization are derived from a clonal B cell population obtained from immunized rabbits.

More specifically, the present invention provides a novel humanization strategy for the humanization of antibody variable light chains derived from rabbit or another lagomorph antibodies that relies on the selection of appropriate homologous human light chain variable sequences and the retention of specific selectivity determining residues contained in the rabbit light chain CDRs as part of the humanization strategy.

“Selectivity determining residues” are defined in more detail infra but essentially correspond to specific amino acid residues which are contained in the rabbit CDR regions which based on their structure and/or chemical properties compared to a corresponding amino acid residue contained in a human germline CDR used for deriving the humanized antibody are believed to have a significant effect on antigen recognition and/or antigen binding.

Also more specifically the present invention provides a novel strategy for humanization of antibody variable heavy chains derived from rabbit or another lagomorph antibodies that relies on the selection of appropriate homologous heavy chain variable sequences and the retention of specific selectivity determining residues as part of the humanization strategy.

Also more specifically, the present invention provides novel humanization strategies for producing humanized antibodies and antibody fragments comprising humanized variable heavy and/or light chains which are derived from rabbit or another lagomorph antibody variable heavy and light chain polypeptides.

Even more specifically, the invention provides a humanization strategy for producing a humanized light chain antibody sequence derived from a lagomorph (rabbit) light chain antibody sequence comprising the following steps:

(i) obtaining a rabbit light chain antibody sequence from a rabbit antibody that specifically binds to a desired antigen and identifying the amino residues spanning the beginning of Framework 1 (FR1) to the end of Framework 3 (FR3) inclusive;

(ii) conducting a homology search using said rabbit light antibody amino acid sequence spanning the beginning of FR1 to the end of FR3 sequence against a library containing human light chain antibody variable sequences and identifying a human light chain antibody sequence that exhibits substantial sequence homology thereto, i.e., which preferably possesses at least 80%-90% identity thereto and/or which exhibits the most sequence identity at the amino acid level relative to other human light chain antibody variable sequences in the library;

(iii) identifying in both the rabbit and human light chain variable sequences the arrangement and the specific residues thereof that correspond to FR1. FR2, FR3, CDR1, CDR2 regions and aligning these discrete regions in the rabbit and selected human antibody light chain;

(iv) constructing a DNA or amino acid sequence wherein at least the amino acid residues in the CDR1 and CDR2 regions of the selected homologous human light chain sequence that differ from the corresponding selectivity determining residues in the rabbit light chain CDR1 and CDR2 are substituted with the corresponding selectivity determining residues in the rabbit CDR1 and CDR2 regions;

(v) further attaching to the DNA or amino acid sequence obtained by step (iv) a DNA sequence encoding or polypeptide containing the corresponding amino acid residues of the rabbit CDR3 light chain antibody sequence;

(vi) further selecting a human light chain framework 4 region (FR4) that is homologous to the FR4 contained in the rabbit light chain and which preferably differs therefrom by at most 2-4 amino acid residues and attaching a DNA sequence encoding said human FR4 or the corresponding amino residues of said human FR4 onto the DNA or amino acid sequence obtained after step (v); and

(vii) synthesizing a DNA or amino acid sequence encoding or containing the humanized rabbit light chain sequence that results from steps (i) through (vi).

Also more specifically, the invention provides a humanization strategy for producing a humanized heavy chain antibody sequence from a rabbit heavy chain antibody sequence comprising the following steps:

(i) obtaining a rabbit heavy chain antibody sequence from an rabbit antibody that specifically binds to a desired antigen and identifying the amino residues spanning the beginning of Framework 1 (FR1) to the end of Framework 3 (FR3) inclusive;

(ii) conducting a homology search (e.g., by BLAST searching of human germline antibody sequence containing libraries) using said rabbit heavy antibody amino acid sequence spanning the beginning of FR1 to the end of FR3 sequence and identifying a human heavy chain antibody sequence that is homologous thereto, i.e. which preferably possesses at least 80%-90% identical thereto at the amino acid level and/or which exhibits the most sequence identity at the amino acid level relative to other human heavy chain antibody variable sequences in the library;

(iii) identifying in both the rabbit and human heavy chain sequences the arrangement of and the specific residues thereof that correspond to FR1, FR2, FR3, CDR1, CDR2 regions and aligning these discrete regions of the rabbit against the corresponding regions of the selected homologous human antibody heavy chain;

(iv) constructing a DNA or amino acid sequence wherein at least the amino acid residues in the CDR1 and CDR2 regions of the selected homologous human heavy chain sequence which differ from the corresponding selectivity determining residues in the rabbit heavy chain CDR1 and CDR2 regions are substituted by the corresponding selectivity determining residues contained in the CDR1 and CDR2 regions of the rabbit heavy chain sequence and further optionally replacing the terminal 1-3 amino acids of the human heavy FR1 region with the corresponding terminal 1-3 amino acids of the rabbit heavy chain FR1; and/or optionally replacing the terminal amino acid of the human heavy chain framework 2 region with the corresponding terminal amino acid residue of the rabbit heavy chain framework 2 and/or optionally replacing the fourth amino acid from the terminus of the rabbit heavy chain CDR2 (typically a tryptophan) with the corresponding human CDR2 residue (typically a serine);

(v) further attaching to the DNA or amino acid sequence obtained by step (iv) a DNA sequence encoding or having the corresponding amino acid residues of the rabbit heavy chain CDR3 which is contained in the same rabbit heavy chain antibody sequence; and which rabbit CDR3 is typically 5-19 amino acids in length (and wherein said CDR3 typically precedes the residues WGXG and further wherein X is typically Q or P);

(vi) further selecting a human heavy chain framework 4 region (FR4) that is homologous thereto (preferably differs from the FR4 contained in the humanized rabbit antibody heavy chain sequence by at most 4 amino acid residues) and attaching a DNA sequence encoding said selected homologous human FR4 or the corresponding amino residues of said human FR4 onto the DNA or amino acid sequence obtained after step (v) (frequently this human FR4 DNA or polypeptide sequence will encode or comprise WGQGTLVTVSS); and

(vii) synthesizing a DNA or amino acid sequence encoding or containing the humanized rabbit heavy chain sequence that results from steps (i) through (vi).

Also more specifically, the invention provides a humanization strategy for producing a humanized antibody or antibody fragment containing at least one humanized light chain antibody sequence derived from a rabbit light chain antibody sequence and/or at least one humanized heavy chain sequence derived from a rabbit antibody heavy chain wherein such humanized light and/or heavy chain sequences are derived from rabbit heavy and light chains according to the following steps:

(i) obtaining a rabbit light chain antibody sequence from an rabbit antibody specific to a desired antigen and identifying the amino residues spanning the beginning of Framework 1 (FR1) to the end of Framework 3 (FR3) inclusive;

(ii) conducting a homology search using said rabbit light antibody amino acid sequence spanning the beginning of FR1 to the end of FR3 sequence against a library containing human light chain antibody sequences and identifying a human light chain antibody sequence that is homologous thereto, i.e., which preferably is at least 80%-90% identical thereto at the amino acid level and/or which exhibits the most sequence identity at the amino acid level relative to other human light chain antibody variable sequences in the library;

(iii) identifying in both the rabbit and human light chain sequences the arrangement of and the specific residues thereof that correspond to FR1. FR2, FR3, CDR1, CDR2 regions and aligning these discrete regions of the rabbit light chain with the corresponding regions of the selected homologous human light chain region;

(iv) constructing a DNA or amino acid sequence wherein at least the amino acid residues in the CDR1 and CDR2 regions of the selected homologous human light chain sequence which differ from the corresponding selectivity determining residues in the rabbit variable light chain CDR1 and CDR2 regions are substituted by the corresponding selectivity amino acid residues in the rabbit CDR1 and CDR2 regions of the rabbit light chain sequence;

(v) further attaching to the DNA or amino acid sequence obtained by step (iv) a DNA sequence encoding or having the corresponding amino acid residues of CDR3 contained in the rabbit light chain antibody sequence;

(vi) further selecting a human light chain framework 4 region (FR4) that is homologous to FR4 contained in said rabbit antibody light chain and which human FR4 preferably differs from the FR4 of the rabbit antibody light chain sequence by at most 2-4 amino acid residues and attaching a DNA sequence encoding said human FR4 or the corresponding amino residues of said human FR4 onto the DNA or amino acid sequence obtained after step (v); and

(vii) synthesizing a DNA or amino acid sequence encoding or containing the humanized rabbit light chain sequence that results from steps (i) through (vi);

and/or further producing a humanized heavy chain antibody sequence from a rabbit heavy chain antibody sequence comprising the following steps:

(i) obtaining a rabbit heavy chain antibody sequence from an rabbit antibody specific to a desired antigen and identifying the amino residues spanning the beginning of Framework 1 (FR1) to the end of Framework 3 (FR3) inclusive;

(ii) conducting a homology search e.g., by BLAST searching of human germline antibody sequence containing libraries using said rabbit heavy antibody amino acid sequence spanning the beginning of FR1 to the end of FR3 sequence and identifying a human heavy chain antibody sequence that is at least 85%-90% identical thereto at the amino acid level and/or which exhibits the most sequence identity at the amino acid level relative to other human heavy chain antibody variable sequences contained in the library;

(iii) identifying in both the rabbit and human heavy chain sequences the arrangement of and the specific residues thereof that correspond to FR1, FR2, FR3, CDR1, CDR2 regions and aligning these discrete regions of the rabbit antibody against the selected homologous human heavy chain;

(iv) constructing a DNA or amino acid sequence wherein at least the amino acid residues contained in the CDR1 and CDR2 regions of the selected homologous human heavy chain sequence which differ from the corresponding selectivity determining residues in the rabbit variable heavy chain CDR1 and CDR2 regions are substituted by the corresponding selectivity determining residues of the CDR1 and CDR2 regions of the rabbit heavy chain sequence and/or optionally replacing the final 1-3 amino acids of the human heavy FR1 region with the terminal 1-3 amino acids of the rabbit heavy chain FR1; and/or optionally replacing the terminal amino acid of the human heavy chain framework 2 region with the terminal amino acid residue of the rabbit heavy chain framework 2; and/or further optionally replacing the fourth amino acid from the terminus of the rabbit heavy chain CDR2 (typically a tryptophan) with the corresponding human CDR2 residue (typically a serine);

(v) further attaching to the DNA or amino acid sequence obtained by step (iv) a DNA sequence encoding or the corresponding amino acid residues of the rabbit heavy chain CDR3 contained in the same rabbit heavy chain antibody sequence; (which CDR3 is typically 5-19 amino acids in length) (and which CDR3 further typically precedes WGXG);

(vi) further selecting a human heavy chain framework 4 region (FR4) that is homologous thereto (i.e., that preferably differs from the FR4 contained in the humanized rabbit antibody heavy chain sequence by at most 2-4 amino acid residues) and attaching a DNA sequence encoding said selected homologous human FR4 or the corresponding amino residues of said human FR4 onto the DNA or amino acid sequence obtained after step (v) (typically the human FR4 DNA or polypeptide sequence will encode or comprise WGQGTLVTVSS); and

(vii) synthesizing a DNA or amino acid sequence encoding or containing the humanized rabbit heavy chain sequence that results from steps (i) through (vi);

and using said synthesized humanized heavy and light chain DNA or amino acid sequences produced as set forth above to produce a humanized antibody or fragment or DNA sequences encoding containing at least one humanized rabbit light chain and/or at least one humanized rabbit heavy chain.

Also the invention provides novel and improved humanized antibody heavy and light chains and antibodies comprising said humanized heavy and light chains produced by the subject humanization methods and use thereof in therapy and diagnostic methods.

In particular the invention provides humanized antibody light chains which contain the following: (i) the amino acid residues spanning the first residue of FR1 through the terminus of FR3 including the CDR1 and CDR2 regions of a human light chain germline sequence that is selected from a library of human germline sequences based on its greater homology (sequence identity) at the amino acid level to the amino acid residues spanning FR1 through FR3 (preferably sequence possessing greatest percent sequence identity at the amino acid level relative to said region in the rabbit variable light chain spanning FR1 through FR3 relative to the other human light chain germline sequences in the library) to the corresponding amino acid residues of the light chain of a parent rabbit antibody having specificity to a desired antigen that is to be humanized and (ii) further wherein the CDR residues in CDR1 and CDR2 corresponding to “selectivity determining residues” in the light chain of the same parent rabbit antibody are replaced with the corresponding rabbit selectivity determining residues; (iii) the amino acid residues encompassing the entire CDR3 region of the same parent rabbit antibody; (iv) the amino acid residues encompassing the entire FR4 region of an antibody light chain derived from a library of human germline sequences based on its greater homology (sequence identity) to the corresponding FR4 region contained in the light chain of the same parent rabbit antibody; and (v) further wherein few or none of the FR residues of the human FR1, FR2, FR3 and FR4 regions in the selected homologous human FR regions are substituted with the corresponding rabbit FR residues (i.e., the residues present at the corresponding site(s) in the parent rabbit light chain antibody sequence being humanized).

In addition the invention provides humanized antibody heavy chain polypeptides which contain at least the following (i) the amino acid residues spanning the first residue of FR1 through the terminus of FR3 including the CDR1 and CDR2 regions of a human germline sequence that is selected from a library of human germline sequences based on its greater homology (percent sequence identity at the amino acid level) of the selected amino acid residues spanning FR1 through FR3 (relative to other human germline sequences in the library) to the corresponding amino acid residues of the heavy chain of a parent rabbit antibody having specificity to a desired antigen that is to be humanized and (ii) further wherein the CDR residues in the CDR1 and CDR2 regions of the human heavy chain corresponding to “selectivity determining residues” in the CDR1 and CDR2 regions of the heavy chain of the same parent rabbit antibody are replaced with the corresponding heavy chain selectivity determining residues contained in the CDR1 and CDR2 regions of the rabbit heavy chain; (iii) the amino acid residues encompassing the entire CDR3 region of the same parent rabbit antibody; (iv) the FR4 region derived from a library of human germline sequences based on its greater homology (sequence identity) to the corresponding FR4 region contained in the heavy chain of the same parent rabbit antibody; and (v) wherein the final 1-3 amino acids of the human heavy FR1 region are optionally replaced with the terminal 1-3 amino acids of the corresponding rabbit heavy chain FR1 residues; and/or the terminal amino acid of the human heavy chain framework 2 region are optionally replaced with the corresponding terminal amino acid residue of the rabbit heavy chain framework 2; and/or the fourth amino acid from the terminus of the rabbit heavy chain CDR2 (typically a tryptophan) is optionally replaced with the corresponding human CDR2 residue (typically a serine); and (vi) wherein few or none of the remaining FR residues of the selected homologous human FR regions are substituted with the corresponding rabbit FR residues (i.e., FR residues present at corresponding site(s) in rabbit antibody heavy chain being humanized).

Further, the invention provides novel and improved humanized antibodies containing the foregoing humanized heavy and light chain polypeptides and nucleic acid sequences encoding said humanized heavy and light chain polypeptides and humanized antibodies containing said humanized heavy and light chains as well as vectors and host cells containing said vectors and nucleic acid sequences and use thereof in therapeutic and diagnostic methods and compositions.

The invention further contemplates attaching said humanized heavy or light chain DNA or polypeptide(s) or to a DNA or polypeptide sequence containing or encoding a desired antibody constant domain, preferably a human antibody constant domain and/or the attachment (direct or indirect) at the carboxy or amino terminus of the antibody polypeptide or nucleic acid sequence to a desired effector moiety e.g., toxins, drugs, radionuclides, fluorophores, enzymes, cytokines, or translocating sequences such as signal peptides, and polypeptides that facilitate affinity isolation.

### BRIEF SUMMARY OF THE INVENTION

As discussed the invention provides novel and improved methods for obtaining humanized variable light and variable heavy chains derived from rabbit antibodies and humanized heavy and/or light chain polypeptides and DNAs encoding produced by such methods. The methods of the subject invention reproducibly yield humanized antibodies which should be substantially non-immunogenic in humans and which retain the antigenic specificity and substantially or entirely the binding affinity of the parent rabbit antibodies. The inventive procedure in general relies on transferring specific amino acid residues from the donor rabbit antibodies (in particular selectivity determining residues that are putatively instrumental in antigen recognition and binding and if necessary a few number of framework residues) onto homologous acceptor human antibody variable heavy and light chain sequences.

More specifically, the present invention is directed to a novel humanization strategy for humanization of antibody variable light chains derived from rabbit antibodies which incorporates a discrete number of rabbit light chain CDR residues referred to herein as “selectivity determining residues” and optionally no or very few framework residues onto homologous human antibody light chain sequences.

Also more specifically the present invention provides a novel strategy for humanization of antibody variable heavy chains derived from rabbit antibodies which incorporates no or very few discrete number of rabbit CDRs onto homologous human heavy chain sequences.

Further more specifically, the present invention is directed to novel and improved humanization strategies for producing humanized antibodies and humanized antibody fragments comprising humanized variable heavy and/or light chains which are derived from rabbit antibody variable heavy and light chain polypeptides and appropriate homologous human antibody variable heavy and light chain polypeptides such that at least specific residues contained in the human heavy and light chain CDRs which differ from the corresponding selectivity determining residues in the rabbit heavy and light chain CDRs (selectivity determining residues) are retained in the humanized heavy and/or light chain regions and wherein very few or no framework residues in the human light chain and very few framework residues in the human heavy chain are substituted with the corresponding rabbit framework residues.

Still more specifically, the invention is directed to a humanization strategy for producing a humanized light chain antibody sequence derived from a donor rabbit light chain antibody sequence and acceptor human light chain antibody sequence comprising the following steps:

(i) obtaining a rabbit light chain antibody sequence from a rabbit antibody that specifically binds to a desired antigen and identifying the amino residues spanning the beginning of Framework 1 (FR1) to the end of Framework 3 (FR3) inclusive;

(ii) conducting a homology search using said rabbit light antibody amino acid sequence spanning the beginning of FR1 to the end of FR3 sequence against a library containing human light chain antibody sequences and identifying a human light chain antibody sequence that exhibits substantial sequence homology thereto, i.e., which preferably possesses at least 80%-90% identity thereto at the amino acid level and/or which preferably possesses greatest percent sequence identity at the amino acid level relative to other sequences in the library containing human light chain antibody sequences;

(iii) identifying in both the rabbit and human light chain sequences the orientation of and the specific residues thereof that correspond to FR1. FR2, FR3, CDR1, CDR2 regions and aligning these discrete regions in the rabbit and selected human antibody light chain;

(iv) constructing a DNA or amino acid sequence wherein at least the residues in the CDR1 and CDR2 regions of the selected homologous human light chain sequence which differ from the corresponding selectivity determining residues contained in the rabbit light chain CDR1 and CDR2 regions are substituted by the corresponding selectivity determining residues contained in the CDR1 and CDR2 regions of the rabbit light chain sequence;

(v) further attaching to the DNA or amino acid sequence obtained by step (iv) a DNA sequence encoding or polypeptide containing the corresponding amino acid residues of the rabbit CDR3 light chain antibody sequence;

(vi) further selecting a human light chain framework 4 region (FR4) that is homologous to the FR4 contained in the rabbit light chain and which preferably differs therefrom by at most 2-4 amino acid residues and attaching a DNA sequence encoding said human FR4 or the corresponding amino residues of said human FR4 onto the DNA or amino acid sequence obtained after step (v); and

(vii) synthesizing a DNA or amino acid sequence encoding or containing the humanized rabbit light chain sequence that results from steps (i) through (vi).

Also more specifically, the invention provides a humanization strategy for producing a humanized heavy chain antibody sequence from a rabbit heavy chain antibody sequence comprising the following steps:

(i) obtaining a rabbit heavy chain antibody sequence from an rabbit antibody that specifically binds to a desired antigen and identifying the amino residues spanning the beginning of Framework 1 (FR1) to the end of Framework 3 (FR3) inclusive;

(ii) conducting a homology search (e.g., by BLAST searching of human germline antibody sequence containing libraries) using said rabbit heavy antibody amino acid sequence spanning the beginning of FR1 to the end of FR3 sequence and identifying a human heavy chain antibody sequence that is homologous thereto, i.e. which preferably possesses at least 85%-90% identical thereto at the amino acid level and/or which preferably possesses greatest percent sequence identity at the amino acid level relative to other sequences in the library containing human heavy chain antibody sequences;

(iii) identifying in both the rabbit and human light chain sequences the residues thereof that correspond to FR1, FR2, FR3, CDR1, CDR2 regions and aligning these discrete regions of the rabbit against the corresponding regions of the selected homologous human antibody heavy chain;

(iv) constructing a DNA or amino acid sequence wherein at least the amino acid residues contained in the CDR1 and CDR2 regions of the selected homologous human heavy chain sequence that differ from the corresponding selectivity determining residues in the CDR1 and CDR2 regions of the rabbit heavy chain sequence are substituted by the corresponding selectivity determining residues contained in the CDR1 and CDR2 regions of the rabbit heavy chain sequence and optionally replacing the terminal 1-3 amino acids of the human heavy FR1 region with the corresponding terminal 1-3 amino acids of the rabbit heavy chain FR1; and/or optionally replacing the terminal amino acid of the human heavy chain framework 2 region with the corresponding terminal amino acid residue of the rabbit heavy chain framework 2 and/or optionally replacing the fourth amino acid from the terminus of the rabbit heavy chain CDR2 (typically a tryptophan) with the corresponding human CDR2 residue (typically a serine);

(v) further attaching to the DNA or amino acid sequence obtained by step (iv) a DNA sequence encoding or having the corresponding amino acid residues of the rabbit heavy chain CDR3 which is contained in the same rabbit heavy chain antibody sequence; and which rabbit CDR3 is typically 5-19 amino acids in length (this CDR3 typically precedes the residues WGXG);

(vi) further selecting a human heavy chain framework 4 region (FR4) that is homologous thereto (preferably differs from the FR4 contained in the humanized rabbit antibody heavy chain sequence by at most 1-4 amino acid residues) and attaching a DNA sequence encoding said selected homologous human FR4 or the corresponding amino residues of said human FR4 onto the DNA or amino acid sequence obtained after step (v) (frequently this human FR4 DNA or polypeptide sequence will encode or comprise WGQGTLVTVSS); and

(vii) synthesizing a DNA or amino acid sequence encoding or containing the humanized rabbit heavy chain sequence that results from steps (i) through (vi).

Also more specifically, the invention provides a humanization strategy for producing a humanized antibody or antibody fragment containing at least one humanized light chain antibody sequence derived from a rabbit light chain antibody sequence and/or at least one humanized heavy chain sequence derived from a rabbit antibody heavy chain wherein such humanized light and heavy chain sequences are derived from rabbit heavy and light chains according to the following steps:

(i) obtaining a rabbit light chain antibody sequence from an rabbit antibody specific to a desired antigen and identifying the amino residues spanning the beginning of Framework 1 (FR1) to the end of Framework 3 (FR3) inclusive;

(ii) conducting a homology search using said rabbit light antibody amino acid sequence spanning the beginning of FR1 to the end of FR3 sequence against a library containing human light chain antibody sequences and identifying a human light chain antibody sequence that is homologous thereto, i.e., which preferably is at least 80%-90% identical thereto at the amino acid level and/or which preferably possesses greatest percent sequence identity at the amino acid level relative to other sequences in the library containing human light chain antibody sequences;

(iii) identifying in both the rabbit and human light chain sequences the orientation of and the specific residues thereof that correspond to FR1. FR2, FR3, CDR1, CDR2 regions and aligning these discrete regions of the rabbit light chain with the corresponding regions of the selected homologous human light chain region;

(iv) constructing a DNA or amino acid sequence wherein at least the residues contained in the CDR1 and CDR2 regions of the selected homologous human light chain sequence which differ from the corresponding selectivity determining residues contained in the CDR1 and CDR2 regions of the rabbit are substituted by the corresponding CDR1 and CDR2 regions of the rabbit light chain sequence;

(v) further attaching to the DNA or amino acid sequence obtained by step (iv) a DNA sequence encoding or having the corresponding amino acid residues of CDR3 contained in the rabbit light chain antibody sequence (this CDR3 typically comprises 9-15 amino acid residues and often precedes FGGG residues);

(vi) further selecting a human light chain framework 4 region (FR4) that is homologous to FR4 contained in said rabbit antibody light chain and which human FR4 preferably differs from the FR4 of the rabbit antibody light chain sequence by at most 2-4 amino acid residues and attaching a DNA sequence encoding said human FR4 or the corresponding amino residues of said human FR4 onto the DNA or amino acid sequence obtained after step (v); and

(vii) synthesizing a DNA or amino acid sequence encoding or containing the humanized rabbit light chain sequence that results from steps (i) through (vi);

and/or further producing a humanized heavy chain antibody sequence from a rabbit heavy chain antibody sequence comprising the following steps:

(i) obtaining a rabbit heavy chain antibody sequence from an rabbit antibody specific to a desired antigen and identifying the amino residues spanning the beginning of Framework 1 (FR1) to the end of Framework 3 (FR3) inclusive;

(ii) conducting a homology search e.g., by BLAST searching of human germline antibody sequence containing libraries using said rabbit heavy antibody amino acid sequence spanning the beginning of FR1 to the end of FR3 sequence and identifying a human heavy chain antibody sequence that is at least 80%-90% identical thereto at the amino acid level and/or which preferably greatest percent sequence identity at the amino acid level relative to other sequences in the library containing human heavy chain antibody sequences;

(iii) identifying in both the rabbit and human heavy chain sequences the residues thereof that correspond to FR1, FR2, FR3, CDR1, CDR2 regions and aligning these discrete regions of the rabbit antibody against the selected homologous human heavy chain;

(iv) constructing a DNA or amino acid sequence wherein at least the residues contained in the CDR1 and CDR2 regions of the selected homologous human heavy chain sequence which differ from the corresponding selectivity determining residues contained in the rabbit heavy chain CDR1 and CDR2 regions are substituted by the corresponding CDR1 and CDR2 regions of the rabbit heavy chain sequence and/or optionally replacing the final 1-3 amino acids of the human heavy FR1 region with the terminal 1-3 amino acids of the rabbit heavy chain FR1; and/or optionally replacing the terminal amino acid of the human heavy chain framework 2 region with the terminal amino acid residue of the rabbit heavy chain framework 2; and/or further optionally replacing the fourth amino acid from the terminus of the rabbit heavy chain CDR2 (typically a tryptophan) with the corresponding human CDR2 residue (typically a serine);

(v) further attaching to the DNA or amino acid sequence obtained by step (iv) a DNA sequence encoding or the corresponding amino acid residues of the rabbit heavy chain CDR3 contained in the same rabbit heavy chain antibody sequence; (which CDR3 is typically 5-19 amino acids in length and typically precedes WGXG)

(vi) further selecting a human heavy chain framework 4 region (FR4) that is homologous thereto (i.e., that preferably differs from the FR4 contained in the humanized rabbit antibody heavy chain sequence by at most 2-4 amino acid residues) and attaching a DNA sequence encoding said selected homologous human FR4 or the corresponding amino residues of said human FR4 onto the DNA or amino acid sequence obtained after step (v) (frequently the human FR4 DNA or polypeptide sequence will encode or comprise WGQGTLVTVSS); and

(vii) synthesizing a DNA or amino acid sequence encoding or containing the humanized rabbit heavy chain sequence that results from steps (i) through (vi); and producing a nucleic acid sequence or polypeptide containing at least one of said humanized light chains and heavy chains; and

synthesizing a DNA encoding a humanized antibody or an antibody fragment or a polypeptide comprising a humanized antibody or an antibody fragment that contains a DNA encoding or polypeptide containing at least humanized light chain sequence and/or at least one humanized heavy chain produced according to the foregoing steps.

The invention further contemplates attaching said humanized antibody DNA or polypeptides to desired constant domains, preferably human constant domains and/or the attachment (direct or indirect) at the carboxy or amino terminus to desired effector moieties e.g., toxins, drugs, radionuclides, fluorophores, enzymes, cytokines, translocating sequences such as signal peptides, and polypeptides that facilitate affinity isolation.

The invention in more specific embodiments is directed to specific humanized antibodies and fragments thereof having binding specificity for TNF-alpha or IL-6 in particular humanized antibodies having specific epitopic specificity and/or functional properties.

One embodiment of the invention encompasses specific humanized antibodies and fragments thereof capable of binding to IL-6 or TNF-alpha and/or the TNF-alpha/TNFR or IL-6/IL-6R complex.

Another embodiment of this invention relates to the humanized antibodies that possess binding affinities (Kds) less than 50 picomolar and/or K_{off} values less than or equal to 10⁻⁴ S⁻¹.

In preferred embodiments of the invention these humanized antibodies and humanized antibody fragments and versions will be derived from rabbit immune cells (B lymphocytes) or less preferably hybridomas secreting rabbit antibodies specific to a desired antigen. In addition the rabbit antibodies used for humanization may be further selected based on their homology (sequence identity) to human germ line antibody sequences. These antibodies may further facilitate retention of functional properties after humanization since lesser amino acids are modified when using the subject humanization methods.

A further embodiment of the invention is directed to humanized antibody fragments produced according to the invention e.g., specific to IL-6 or TNF-alpha containing humanized V_{H}, V_{L} and CDR polypeptides produced according to the invention, e.g., derived from antibodies secreted by rabbit immune cells and the polynucleotides encoding the same, as well as the use of these antibody fragments and the polynucleotides encoding them in the creation of novel antibodies and polypeptide compositions capable of recognizing desired antigens such as IL-6, TNF-alpha and/or TNF-alpha/TNFR or IL-6/IL-6R complexes.

The invention also contemplates conjugates of the subject humanized rabbit antibodies and fragments, e.g., humanized anti-TNF-alpha or anti-IL-6 antibodies and binding fragments thereof conjugated to one or more functional or detectable moieties. The invention also contemplates methods of making said humanized anti-TNF-alpha, IL-6 or anti-TNF-alpha/TNFR or anti-IL-6/IL-6R complex antibodies and binding fragments thereof. In one embodiment, binding fragments include, but are not limited to, humanized Fab, Fab′, F(ab′)₂, Fv and scFv fragments.

Embodiments of the invention further pertain to the use of the subject humanized antibodies specific to a desired antigen, e.g., humanized anti-TNF-alpha or anti-IL-6 antibodies for the diagnosis, assessment and treatment of diseases and disorders associated with the particular antigen. e.g., TNF-alpha, IL-6 or the aberrant expression thereof. The invention also contemplates the use of humanized antibody fragments according to the invention, e.g., humanized anti-TNF-alpha or anti-IL-6 antibodies for the diagnosis, assessment and treatment of diseases and disorders associated with a particular antigen, e.g., IL-6, TNF-alpha or the aberrant expression thereof.

Other embodiments of the invention relate to the production of humanized antibodies and humanized antibody fragments produced according to the novel and improved humanization protocols derived from rabbit antibody sequences in recombinant host cells, preferably diploid yeast such as diploid Pichia and other yeast strains.

### BRIEF DESCRIPTION OF THE FIGURES

FIG. 1 contains a flow chart depicting schematically the inventive rabbit antibody humanization protocol.

FIG. 2 contains alignments of specific exemplary variable light and variable heavy chain polypeptide sequence, i.e., antigen specific rabbit antibody variable light chain polypeptides and variable heavy chain polypeptides sequences and homologous human sequences identified in a library of human germline sequences and the final humanized sequences produced using the inventive humanization protocols Framework regions are identified therein as FR1-FR4. Complementarity determining regions (CDRs) are identified as CDR1-CDR3. Amino acid residues are numbered as shown in the Figure and conform to the Kabat numbering scheme. The initial rabbit sequences are referred to in the Figure and infra as RbtVL and RbtVh for the rabbit variable light and variable heavy chain polypeptide sequences respectively. Three of the most similar human germline antibody sequences spanning from the beginning of FR1 to the end of FR3 identified in a library of human germline sequences are aligned below the rabbit sequences. The human sequence that is considered the most similar to the rabbit sequence is immediately below the rabbit sequence. In this exemplification of the inventive humanization strategy the most similar human germline sequences are L12A for the light chain and 3-64-04 for the heavy chain. Human CDR3 sequences are not shown. The closest human Framework 4 sequence is aligned below the rabbit Framework 4 sequence. The vertical dashes indicate a residue where the rabbit residue is identical with one or more of the human residues at the same position. The bold residues indicate that the human residue at that position is identical to the rabbit residue at the same position. The final humanized sequences are called VLh and VHh for the variable light and variable heavy sequences respectively. In this Figure the underlined residues indicate that the residue is the same as the rabbit residue at that position but different than the human residues at that position in the three aligned human sequences.

FIGS. 3A and 3B similarly each contain an alignment of the same original parent rabbit variable heavy and light chain sequences derived from an IL-6 specific antibody, homologous human germline sequences, and two humanized variable heavy and light chain sequences produced therefrom using the subject humanization strategies. Specifically, FIG. 3A contains the original rabbit light and heavy chain sequences, three homologous human germline sequences and 2 humanized heavy and 2 humanized light chain sequences referred to therein as “aggres” and “consrv”. It can be seen from the alignment that the humanized “aggres” and “consrv” sequences differ from each other in the presence or absence of specific rabbit framework residues. FIG. 3B shows alternative versions of the light and heavy chain sequences that are shown in FIG. 3A, which differ in the sequence of the light chain FR3 region and heavy chain CDR2 region.

FIG. 4 compares the dissociation constants of chimeric versus humanized antibodies derived from rabbit antibodies specific to hIL-6 and TNF-alpha which were produced using the inventive humanization procedures.

FIG. 5 contains an experiment comparing the antagonism of IL-6 dependent T1165 cell proliferation by different humanized antibodies derived from a specific rabbit anti-IL-6 antibody produced using the inventive humanization procedures.

FIG. 6 contains an experiment comparing the antagonism of hIL-6 dependent T1165 cell proliferation by different humanized antibodies derived from a specific rabbit anti-hIL-6 antibody produced using the inventive humanization procedures.

FIG. 7 contains an experiment comparing the antagonism of hTNF-alpha dependent cytotoxicity by a chimeric anti-TNF-alpha antibody derived from a rabbit anti-hTNF-alpha to antibody to a humanized antibody derived therefrom produced according to the inventive humanization procedures.

### DETAILED DESCRIPTION OF PREFERRED EMBODIMENTS

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

As used herein, the terms “acceptor” and “acceptor antibody” or “original” or “parent” antibody refer to the human antibody or nucleic acid sequence providing or encoding sequences used to produce humanized antibody sequences from rabbit antibody variable sequences according to the invention. Typically the acceptor antibody will provide at least 80%, at least 85%, at least 90%, at least 95%, at least 96, 97, 98, 99 or 100% of the amino acid sequences of one or more of the framework regions. In some embodiments, the term “acceptor” refers to the antibody or nucleic acid sequence providing or encoding the constant region(s). In yet another embodiment, the term “acceptor” refers to the antibody or nucleic acid sequence providing or encoding one or more of the framework regions and the constant region(s). In a specific embodiment, the term “acceptor” refers to a human antibody or nucleic acid sequence that provides or encodes at least 80%, preferably, at least 85%, at least 90%, at least 95%, at least 96, 97, 98, 99, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, Fab fragments, Fab′ fragments, F(ab′)₂ fragments, disulfide-linked Fvs (sdFv), anti-idiotypic (anti-Id) antibodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. As noted the invention in general relates to humanized antibodies and humanized antibody fragments produced by combining specific residues rabbit donor antibodies specific to a desired antigen and homologous human (acceptor) antibody sequences.

A typical antibody contains two heavy chains paired with two light chains. A full-length heavy chain is about 50 kD in size (approximately 446 amino acids in length), and is encoded by a heavy chain variable region gene (about 116 amino acids) and a constant region gene. In the present invention essentially two nucleic acid or genetic components encoding a humanized variable light and a humanized variable heavy chain sequence containing specific CDR residues of a rabbit antibody of desired antigen specificity and functional properties and which can be referred to simply as exons are fused together to produce a construct encoding a humanized variable chain which results in the expression of a humanized variable region when this construct is expressed in an appropriate expression system.

The subject humanized antibodies if constant regions are present will contain human constant regions. There are different constant region genes encoding heavy chain constant region of different isotypes such as alpha, gamma (IgG1, IgG2, IgG3, IgG4), delta, epsilon, and mu sequences. A full-length light chain is about 25 Kd in size (approximately 214 amino acids in length), and is encoded by a light chain variable region gene (about 110 amino acids) and a kappa or lambda constant region gene. The variable regions of the light and/or heavy chain are responsible for binding to an antigen, and the constant regions are responsible for the effector functions typical of an antibody.

As used herein, the term “analog” in the context of a proteinaceous agent (e.g., proteins, polypeptides, and peptides, such as antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 96, 97, 98, 99 or 100% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 96, 97, 98, 99 or 100% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. As noted, the present invention in its humanization strategies selects human variable regions that possess high sequence identity or homology to the corresponding variable region of the rabbit light or heavy chain variable region that is used to derive a corresponding “humanized” variant. Typically the selected human variable region will possess at least 80% or greater sequence identity to the corresponding rabbit variable sequence over a specified portion of the variable region containing the CDR1 and CDR2 regions. Ideally the selected human variable region will possess the greatest homology or sequence identity to the rabbit variable region as compared to all other members of a population or library of human germline sequences containing human antibody variable region encoding sequences as determined by appropriate methods such as BLAST searching. In addition a preferred or lead candidate rabbit antibody used in the subject humanization strategies may be selected from a population of rabbit antibodies (of comparable affinities and/or functional characteristics) based on its high homology or sequence identity to a human germline sequence. This is possible as the present invention in preferred embodiments produces its parent antibodies using a B cell immunization protocol that has been found to give rise to a number (e.g., 10 or more) of high affinity antibodies specific to the target recognizing different epitopes on the antigen target such as IL-6. In some instances this identity may be so substantial that the humanized antibody and the parent antibody may possess similar immunogenicity properties in human subjects given the high sequence identity between human and rabbit antibodies versus other animals typically used for humanization such as rodents and guinea pigs.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “CDR” refers to the complement determining region within antibody variable sequences. There are three CDRs in each of the variable regions of the heavy chain and the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Leska, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs. As described below these CDRs contain discrete residues that are believed to be significant in antigen binding or recognition referred to as “selectivity determining residues”.

The expression “selectivity determining residues” in the present invention refers to specific amino acid residues contained in the rabbit variable heavy and light chain polypeptides that are believed to be significantly involved in antigen recognition and/or antigen binding. In the inventive humanization strategies these selectivity determining residues in the rabbit CDR regions are empirically identified by comparison of all of the rabbit CDR residues to the corresponding residue in a selected homologous human variable region and based on this comparison identifying putative “selectivity determining residues”. Essentially a particular CDR residue is viewed to be a selectivity determining residue if it differs substantially from the corresponding human CDR residue according to the Kabat numbering scheme.

“Substantially” herein refers to significant chemical or structural differences between the rabbit and human germline CDR amino acid residues, e.g., differences in charge, charged versus non-charged, presence or absence of bulk side chain and the like. For example if the rabbit CDR amino acid residue contains a bulky side chain and the corresponding human CDR amino acid residue does not then the rabbit CDR residue will be considered to be a selectivity determining residue and will be retained in the humanized variable region. In addition if the CDR amino acid residue in the rabbit variable region is a basic amino acid and the corresponding amino acid residue in the human CDR is an acidic amino acid residue than this residue in the rabbit CDR will be determined to be a selectivity determining residue and will be retained in the humanized variable region. By contrast, if the CDR residue in the rabbit CDR and the corresponding residue in the human CDR are both acidic or both contain analogous bulky side chains the residue will be determined not to be a selectivity determining residue and the human CDR residue will not be modified in the humanized variable region. This means of categorizing specific residues in the rabbit CDR regions as “selectivity determining” or “non-selectivity determining” used in the present humanization strategies in order to select specific rabbit CDR residues which should be retained in the humanized variable regions is analogous to the criteria used in protein mutagenesis for determining whether an amino acid substitution modification can be viewed to be conservative or non-conservative. It should be understood however that while the present invention typically retains all of such selectivity determining residues in the humanized variable region polypeptide based on the supposition that each of these residues is instrumental in antigen recognition and/or binding that in some instances it may be determined upon synthesis of different humanized variable chain variants that retention of a particular putative selectivity determining residue is non-essential with respect to the antigen binding of an antibody containing the humanized variable region. For example, if the parent rabbit antibody has very high antigen affinity for the target antigen the retention of all putative selectivity determining residues may not be essential to derive a humanized antibody possessing desirable antigen binding recognition and affinity. This may be determined empirically by synthesizing different humanized variable region polypeptides. In addition the identification of a particular CDR residue as selectivity determining or not may vary dependent upon the particular sequence or sequences of the selected homologous human variable regions.

The expression “variable region” or “VR” refers to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

The expression “framework region” or “FR” refers to one or more of the framework regions within the variable regions of the light and heavy chains of an antibody (See Kabat, E. A. et al., Sequences of Proteins of Immunological Interest, National Institutes of Health, Bethesda, Md., (1987)). Framework regions or FRs include the amino acid sequence regions which are interposed between the CDRs comprised within the variable regions of the light and heavy chains of an antibody.

As used herein, the expression “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the expression “derivative” in the context of proteinaceous agent (e.g., proteins, polypeptides, and peptides, such as antibodies) refers to a proteinaceous agent that comprises an amino acid sequence which has been altered by the introduction of amino acid residue substitutions, deletions, and/or additions. The expresssion “derivative” as used herein also refers to a proteinaceous agent which has been modified, i.e., by the covalent attachment of any type of molecule to the proteinaceous agent. For example, but not by way of limitation, an antibody may be modified, e.g., by aglycosylation, glycosylation, acetylation, pegylation, phosphorylation, amidation, derivatization by known protecting/blocking groups, proteolytic cleavage, linkage to a cellular ligand or other protein, etc. Preferably the antibody is aglycosylated. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the expression “disorder” or “disease” is used interchangeably for a condition in a subject.

As used herein, the expression “donor” or “donor antibody” refers to an antibody providing one or more CDRs. In a preferred embodiment, the donor antibody is an antibody from a species different from the antibody from which the framework regions are obtained or derived. In the context of a humanized antibody, the term “donor antibody” refers to a non-human (rabbit) antibody providing one or more CDRs.

As used herein, the expression “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

As used herein, the expression “epitope” refers to a fragment of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic. As mentioned the present invention preferably produces rabbit antibodies against a specific target antigen using a clonal B cell immunization approach which has been found to give rise to antibodies of high affinity to a range of different epitopes on the antigen target.

As used herein, the expression “fusion protein” refers to a polypeptide or protein (including, but not limited to an antibody) that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides or peptides with immunomodulatory activity may be fused together to form a fusion protein. In a preferred embodiment, fusion proteins retain or have improved activity relative to the activity of the original protein, polypeptide or peptide prior to being fused to a heterologous protein, polypeptide, or peptide.

As used herein, the expression “fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein. In a specific embodiment, a fragment of a protein or polypeptide retains at least one function of the protein or polypeptide.

As used herein, the expression “functional fragment” refers to a peptide or polypeptide (including, but not limited to an antibody) comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of second, different polypeptide or protein, wherein said polypeptide or protein retains at least one function of the second, different polypeptide or protein. In a specific embodiment, a fragment of a polypeptide or protein retains at least two, three, four, or five functions of the protein or polypeptide. Preferably, a fragment of an antibody that immunospecifically binds to a particular antigen retains the ability to immunospecifically bind to the antigen.

As used herein, the expression “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med. Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the expression “key” residues refer to certain residues within the variable region that have more impact on the binding specificity and/or affinity of an antibody, in particular a humanized antibody. This includes the afore-mentioned selectivity determining residues and further includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein the expression “Tumor Necrosis Factor-alpha” or (TNF-alpha) or TNF-alpha encompasses not only the following 233 amino acid sequence available as GenBank Protein Accession No. CAA26669 (homo sapien TNF-alpha):

MSTESMIRDVELAEEALPKKTGGPQGSRRCLFLSLFSFLIVAGATTLFCL LHFGVIGPQREEFPRDLSLISPLAQAVRSSSRTPSDKPVAHVVANPQAEGQLQ WLNRRANALLANGVELRDNQLVVPSEGLYLIYSQVLFKGQGCPSTHVLLTHT ISRIAVSYQTKVNLLSAIKSPCQRETPEGAEAKPWYEPIYLGGVFQLEKGDRLS AEINRPDYLDFAESGQVYFGIIAL (SEQ ID NO: 1), but also any pre-pro, pro-, mature, soluble, and/or membrane-bound forms of this TNF- [symbol] amino acid sequence, as well as mutants (mutiens), splice variants, orthologues, homologues and variants of this sequence.

The expression “Interleukin-6” or (IL-6) herein encompasses not only the following 212 amino acid sequence available as GenBank Protein Accession No. NP_{—}000591: MNSFSTSAFGPVAFSLGLLLVLPAAFPAPVPPGEDSKDVAAPHRQPLTSSERID KQIRYILDGISALRKETCNKSNMCESSKEALAENNLNLPKMAEKDGCFQSGF NEETCLVKIITGLLEFEVYLEYLQNRFES SEEQARAVQMSTKVLIQFLQKKAK NLDAITTPDPTTNASLLTKLQAQNQWLQDMTTHLILRSFKEFLQSSLRALRQ M (SEQ ID NO: 2), but also any pre-pro, pro- and mature forms of this IL-6 amino acid sequence, as well as mutants and variants including allelic variants of this sequence.

The expression “mating competent yeast species” herein is intended to broadly encompass any diploid yeast which can be stably maintained in culture. Such species of yeast exist in a haploid and a diploid form. The diploid cells may, under appropriate conditions, proliferate for indefinite number of generations in the diploid form. Diploid cells can also sporulate to form haploid cells. In addition, sequential mating can result in tetraploid strains through further mating of the auxotrophic diploids. In the present invention the diploid or polyploidal yeast cells are preferably produced by mating or spheroplast fusion.

In one embodiment of the invention, the mating competent yeast is a member of the Saccharomycetaceae family, which includes the genera Arxiozyma; Ascobotryozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; and Zygosaccharomyces. Other types of yeast potentially useful in the invention include Yarrowia, Rhodosporidium, Candida, Hansenula, Filobasium, Filobasidellla, Sporidiobolus, Bullera, Leucosporidium and Filobasidella.

In a preferred embodiment of the invention, the mating competent yeast is a member of the genus Pichia. In a further preferred embodiment of the invention, the mating competent yeast of the genus Pichia is one of the following species: Pichia pastoris, Pichia methanolica, and Hansenula polymorpha (Pichia angusta). In a particularly preferred embodiment of the invention, the mating competent yeast of the genus Pichia is the species Pichia pastoris.

The expression “haploid yeast cell” herein refers to a yeast cell having a single copy of each gene of its normal genomic (chromosomal) complement.

The expression “polyploid yeast cell” herein refers to a yeast cell having more than one copy of its normal genomic (chromosomal) complement.

The expression “diploid yeast cell” herein refers to a yeast cell having two copies (alleles) of every gene of its normal genomic complement, typically formed by the process of fusion (mating) of two haploid cells.

The expression “tetraploid yeast cell” herein refers to a cell having four copies (alleles) of every gene of its normal genomic complement, typically formed by the process of fusion (mating) of two haploid cells. Tetraploids may carry two, three, or four different cassettes. Such tetraploids might be obtained in S. cerevisiae by selective mating homozygotic heterothallic a/alpha and alpha/alpha or a/a diploids and in Pichia by sequential mating of haploids to obtain auxotrophic diploids. For example, a [met his] haploid can be mated with [ade his] haploid to obtain diploid [his]; and a [met arg] haploid can be mated with [ade arg] haploid to obtain diploid [arg]; then the diploid [his] x diploid [arg] to obtain a tetraploid prototroph. It will be understood by those of skill in the art that reference to the benefits and uses of diploid cells may also apply to tetraploid cells.

The expression “yeast mating” refers to the process by which two haploid yeast cells naturally fuse to form one diploid yeast cell.

The expression “meiosis” herein refers to the process by which a diploid yeast cell undergoes reductive division to form four haploid spore products. Each spore may then germinate and form a haploid vegetatively growing cell line.

The expression “selectable marker” herein refers to a selectable marker is a gene or gene fragment that confers a growth phenotype (physical growth characteristic) on a cell receiving that gene as, for example through a transformation event. The selectable marker allows that cell to survive and grow in a selective growth medium under conditions in which cells that do not receive that selectable marker gene cannot grow. Selectable marker genes generally fall into several types, including positive selectable marker genes such as a gene that confers on a cell resistance to an antibiotic or other drug, temperature when two ts mutants are crossed or a ts mutant is transformed; negative selectable marker genes such as a biosynthetic gene that confers on a cell the ability to grow in a medium without a specific nutrient needed by all cells that do not have that biosynthetic gene, or a mutagenized biosynthetic gene that confers on a cell inability to grow by cells that do not have the wild type gene; and the like. Suitable markers include but are not limited to: ZEO; G418; LYS3; MET1; MET3a; ADE1; ADE3; URA3; and the like.

The expression “expression vector” herein refers to DNA vectors containing elements that facilitate manipulation for the expression of a foreign protein within the target host cell. Conveniently, manipulation of sequences and production of DNA for transformation is first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media.

Expression vectors suitable for use in the methods of the invention further include yeast specific sequences, including a selectable auxotrophic or drug marker for identifying transformed yeast strains. A drug marker may further be used to amplify copy number of the vector in a yeast host cell.

The polypeptide coding sequence of interest is operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included, e.g. a signal sequence, and the like. A yeast origin of replication is optional, as expression vectors are often integrated into the yeast genome.

In one embodiment of the invention, the polypeptide of interest is operably linked, or fused, to sequences providing for optimized secretion of the polypeptide from yeast diploid cells.

The expression “operably linked” in connection with nucleic acid sequences means that these sequences are placed into a functional relationship with each another. For example, a DNA encoding a signal sequence may be operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (Gateway® Technology; Invitrogen, Carlsbad Calif.). If such sites do not exist, the synthetic oligonucleotide adapters or linkers are used in accordance with conventional practice.

The expression “promoter” refers to an untranslated sequence located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The yeast promoter fragment may also serve as the site for homologous recombination and integration of the expression vector into the same site in the yeast genome; alternatively a selectable marker is used as the site for homologous recombination. Pichia transformation is described in Cregg et al. (1985) Mol. Cell. Biol. 5:3376-3385.

Examples of suitable promoters from Pichia include the AOX1 and promoter (Cregg et al. (1989) Mol. Cell. Biol. 9:1316-1323); ICL1 promoter (Menendez et al. (2003) Yeast 20(13): 1097-108); glyceraldehyde-3-phosphate dehydrogenase promoter (GAP) (Waterham et al. (1997) Gene 186(1):37-44); and FLD1 promoter (Shen et al. (1998) Gene 216(1):93-102). The GAP promoter is a strong constitutive promoter and the AOX and FLD1 promoters are inducible.

Other yeast promoters include ADHI, alcohol dehydrogenase II, GAL4, PHO3, PHO5, Pyk, and chimeric promoters derived therefrom. Additionally, non-yeast promoters may be used in the invention such as mammalian, insect, plant, reptile, amphibian, viral, and avian promoters. Most typically the promoter will comprise a mammalian promoter (potentially endogenous to the expressed genes) or will comprise a yeast or viral promoter that provides for efficient transcription in yeast systems.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell. The S. cerevisiae alpha factor pre-pro signal has proven effective in the secretion of a variety of recombinant proteins from P. pastoris. Other yeast signal sequences include the mating factor alpha signal sequence, the invertase signal sequence, and signal sequences derived from other secreted yeast polypeptides. Additionally, these signal peptide sequences may be engineered to provide for enhanced secretion in diploid yeast expression systems. Other secretion signals of interest also include mammalian signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g. K28 preprotoxin sequence, PHA-E, FACE, human MCP-1, human serum albumin signal sequences, human Ig heavy chain, human Ig light chain, and the like. For example, see Hashimoto et. al. Protein Eng 11(2) 75 (1998); and Kobayashi et. al. Therapeutic Apheresis 2(4) 257 (1998).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion and/or sequenced.

As an alternative to restriction and ligation of fragments, recombination methods based on att sites and recombination enzymes may be used to insert DNA sequences into a vector. Such methods are described, for example, by Landy (1989) Ann. Rev. Biochem. 58:913-949; and are known to those of skill in the art. Such methods utilize intermolecular DNA recombination that is mediated by a mixture of lambda and E. coli-encoded recombination proteins. Recombination occurs between specific attachment (att) sites on the interacting DNA molecules. For a description of att sites see Weisberg and Landy (1983) Site-Specific Recombination in Phage Lambda, in Lambda II, Weisberg, ed. (Cold Spring Harbor, NY: Cold Spring Harbor Press), pp. 211-250. The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. While non-covalent interactions are important in determining structure, usually the proteins of interest will have intra- and/or intermolecular covalent disulfide bonds formed by two cysteine residues. For naturally occurring proteins and polypeptides or derivatives and variants thereof, the proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The expression host may be further modified by the introduction of sequences encoding one or more enzymes that enhance folding and disulfide bond formation, i.e. foldases, chaperoning, etc. Such sequences may be constitutively or inducibly expressed in the yeast host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic PDI is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of BIP (immunoglobulin heavy chain binding protein); cyclophilin; and the like. In one embodiment of the invention, each of the haploid parental strains expresses a distinct folding enzyme, e.g. one strain may express BIP, and the other strain may express PDI or combinations thereof.

The terms “desired protein” or “target protein” are used interchangeably and refer generally to a humanized antibody or a binding portion thereof described herein. In the present invention the source for producing antibodies useful as starting material according to the invention is rabbits. Numerous antibody coding sequences have been described; and others may be raised by methods well-known in the art. Examples thereof include chimeric antibodies, human antibodies and other non-human mammalian antibodies, humanized antibodies, single chain antibodies (scFvs), camelbodies, SIMPS, and antibody fragments such as Fabs, Fab′, F(ab′)₂ and the like.

For example, antibodies or antigen binding fragments may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions (V_{L} and V_{H}), obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG1, IgG2, IgG3, IgG4, IgG5, IgG6, IgG7, IgG8, IgG9, IgG10, IgG11, IgG12, IgG13, IgG14, IgG15, IgG16, IgG17, IgG18 or IgG19 constant regions.

The expression “polyploid yeast that stably expresses or expresses a desired secreted heterologous polypeptide for prolonged time” refers to a yeast culture that secretes said polypeptide for at least several days to a week, more preferably at least a month, still more preferably at least 1-6 months, and even more preferably for more than a year at threshold expression levels, typically at least 10-25 mg/liter and preferably substantially greater.

The expression “polyploidal yeast culture that secretes desired amounts of recombinant polypeptide” refers to cultures that stably or for prolonged periods secrete at least 10-25 mg/liter of heterologous polypeptide, more preferably at least 50-500 mg/liter, and most preferably 500-1000 mg/liter or more.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

The expression a “heterologous” region or “heterologous domain” of a DNA construct refers to an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The expression “coding sequence” refers to an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence.

The expression “vectors” herein refers to materials used to introduce a foreign substance, such as DNA, RNA or protein, into an organism or host cell. Typical vectors include recombinant viruses (for polynucleotides) and liposomes (for polypeptides). A “DNA vector” is a replicon, such as plasmid, phage or cosmid, to which another polynucleotide segment may be attached so as to bring about the replication of the attached segment. Herein an “expression vector” is a DNA vector which contains regulatory sequences which will direct polypeptide synthesis by an appropriate host cell. This usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypeptide encoded by said polynucleotide sequence.

The term “amplification” in the context of polynucleotide sequences is the in vitro production of multiple copies of a particular nucleic acid sequence. The amplified sequence is usually in the form of DNA. A variety of techniques for carrying out such amplification are described in a review article by Van Brunt (1990, Bio/Technol., 8(4):291-294). Polymerase chain reaction or PCR is a prototype of nucleic acid amplification, and use of PCR herein should be considered exemplary of other suitable amplification techniques.

### DETAILED DESCRIPTION OF THE INVENTION

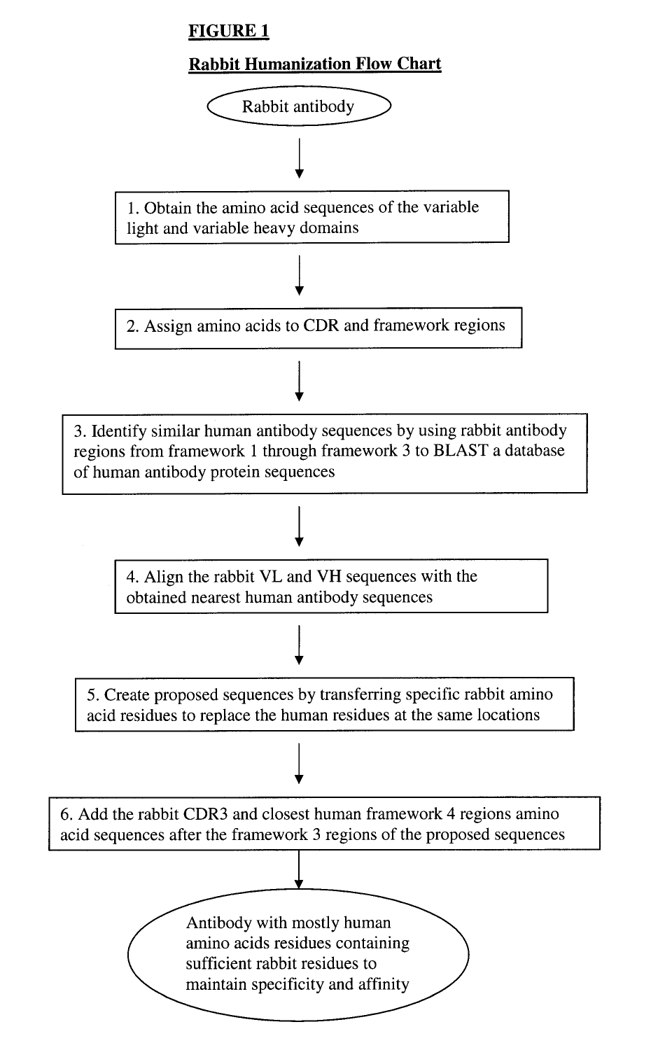

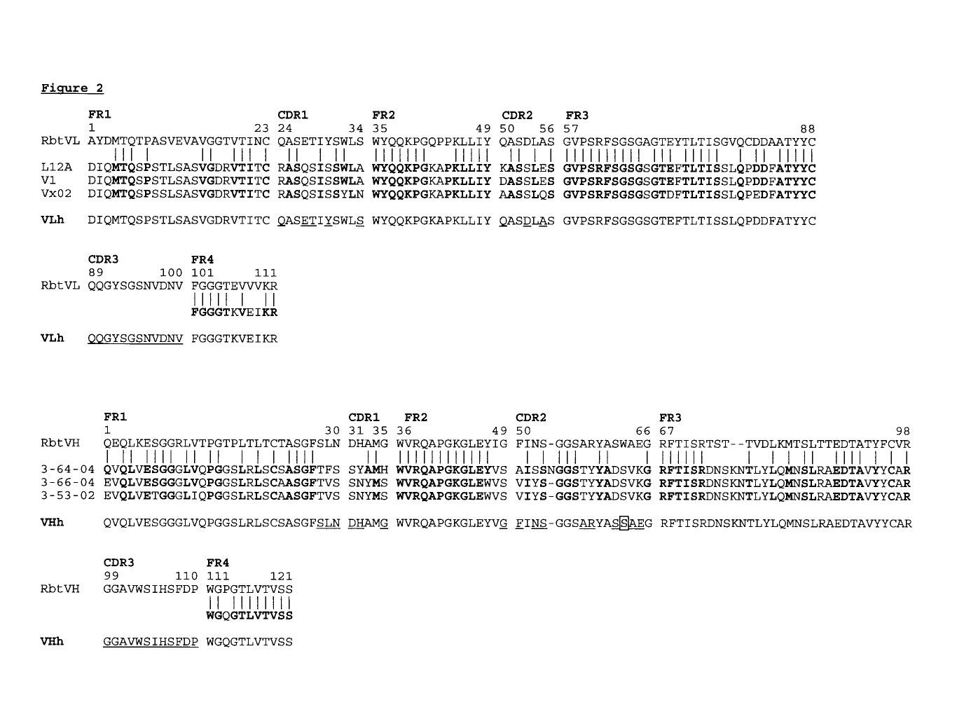

The subject humanization methods are generically applicable to humanizing any rabbit antibody or variable region thereof, i.e., these antibodies may specifically bind to different desired antigens. In addition the subject humanization approaches may be applicable for humanizing other species antibodies, e.g., antibodies from animals closely related to rabbits such as other mammals in the order Lagomorpha or family Leporidae which includes different rabbits and hares. These mammals since they are closely related to domesticated rabbits should possess variable sequences closely related to the domesticated rabbit species used herein as a source or rabbit antibodies for humanization. Accordingly, the description below corresponding to the synthesis of humanized anti-TNF-alpha or anti-IL-6 antibodies is exemplary. The inventive humanization protocol is depicted schematically in FIG. 1 and is described in detail infra. The description of the method disclosed infra provides both generally applicable rules as well as the application of those rules to a specific sequence shown in FIG. 2, as an example.

The invention contemplates the use of the subject humanization strategy to produce humanized heavy and light chains and antibodies and antibody fragments containing that are specific to any desired antigen. Examples of suitable antigens include human proteins such as growth factors, cytokines, enzymes, hormones, tumor specific antigens, oncogenes, et al., allergens, antigens from infectious agents such as bacteria, viruses, fungi, yeast, parasites, et al, toxins, etc. The examples infra exemplify methods useful to obtain humanized rabbit antibodies specific to TNF- [symbol] and IL-6 and illustrate the inventive methods and its intrinsic advantages.

The invention also contemplates antibody fragments which include one or more of the humanized heavy or light chains produced according to the invention. The invention specifically contemplates humanized antibody fragments having binding specificity to TNF- [symbol] or IL-6. Such antibody fragments may be present in one or more of the following non-limiting forms: Fab, Fab′, F(ab′)₂, Fv and single chain Fv antibody forms.

As mentioned previously, in a preferred and exemplified embodiment of the invention, the antibodies that are used for humanization originate or are selected from one or more clonal antigen specific rabbit B cell populations prior to initiation of the humanization process referenced herein.

As stated supra, antibodies and fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Regarding detectable moieties, further exemplary enzymes include, but are not limited to, horseradish peroxidase, acetylcholinesterase, alkaline phosphatase, beta-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²⁵I), Carbon 14 (¹⁴C), Sulfur 35 (³⁵S), Tritium (³H) and Phosphorus 32 (³²P)

Regarding functional moieties, exemplary cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, caminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine and bleomycin. Toxic enzymes from plants and bacteria such as ricin, diphtheria toxin and Pseudomonas toxin may be conjugated to the humanized antibodies, or binding fragments thereof, to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the antibody, or binding fragments thereof, with or without the use of a complex-forming agent. Such radionuclides include beta-emitters such as Phosphorus-32 (³²P), Scandium-47 (⁴⁷Sc), Copper-67 (⁶⁷Cu), Gallium-67 (⁶⁷Ga), Yttrium-88 (⁸⁸Y), Yttrium-90 (⁹⁰Y), Iodine-125 (¹²⁵I), Iodine-131 (¹³¹I), Samarium-153 (¹⁵³Sm), Lutetium-177 (¹⁷⁷Lu), Rhenium-186 (¹⁸⁶Re) or Rhenium-188 (¹⁸⁸Re), and alpha-emitters such as Astatine-211 (²¹¹At), Lead-212 (²²Pb), Bismuth-212 (²¹²Bi) or -213 (¹¹³Bi) or Actinium-225 (²²⁵Ac).

Methods are known in the art for conjugating an antibody or binding fragment thereof to a detectable moiety and the like, such as for example those methods described by Hunter et al, Nature 144:945 (1962); David et al, Biochemistry 13:1014 (1974); Pain et al, J. Immunol. Meth. 40:219 (1981); and Nygren, J., Histochem. and Cytochem. 30:407 (1982).

Embodiments described herein further include variants and equivalents that are substantially homologous to the antibodies, antibody fragments, polypeptides, variable regions and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

In another embodiment, the invention contemplates polypeptide sequences having at least 90% or greater sequence homology to any one or more of the humanized polypeptide sequences of antibody fragments, variable regions and CDRs set forth herein. More preferably, the invention contemplates humanized polypeptide sequences having at least 95% or greater sequence homology, even more preferably at least 98% or greater sequence homology, and still more preferably at least 99% or greater sequence homology to any one or more of the humanized antibody fragments produced according to the invention.

A significant advantage of the present humanization protocol is that the binding affinity of antibodies containing humanized variable sequences produced according to the invention relative to that of the parent rabbit antibody remains substantially intact (unchanged). Preferably, the humanized antibodies produced by the present invention such as humanized anti-IL-6 or TNF-alpha antibodies and fragments thereof will have binding specificity to IL-6, TNF-alpha, or another antigen useful in human therapy and will bind to their antigen with a dissociation constant (K_{D}) of less than or equal to 5×10⁻⁷ M⁻¹, 10⁻⁷ M⁻¹, 5×10⁻⁸ M⁻¹, 10⁻⁸ M⁻¹, 5×10⁻⁹ M⁻¹, 10⁻⁹ M⁻¹, 5×10⁻¹⁰ M⁻¹, 10⁻¹⁰ M⁻¹, 5×10⁻¹¹ M⁻¹, 10⁻¹¹ M⁻¹, 5×10⁻¹² M⁻¹, 10⁻¹² M⁻¹, 5×10⁻¹³ M⁻¹, 10⁻¹³ M⁻¹, 5×10⁻¹⁴ M⁻¹, 10⁻¹⁴ M⁻¹, 5×10⁻¹⁵ M⁻¹ or 10⁻¹⁵ M⁻¹. Preferably, the subject humanized antibodies will bind their antigen target such as IL-6 or TNF- [symbol] antibody with a dissociation constant of less than or equal to 5×10⁻¹⁰ M⁻¹.

In another embodiment of the invention, the humanized antibodies and fragments produced from rabbit antibodies will possess a binding specificity to an antigen such as IL-6 or TNF-alpha, with an off-rate of less than or equal to 10⁻⁴ S⁻¹ 10⁻⁵ S⁻, 5×10⁻⁶ S⁻¹, 10⁻⁶ S⁻¹, 5×10⁻⁷ S⁻¹, or 10⁻⁷ S⁻¹.

In a further embodiment of the invention, the activity of the subject humanized antibodies of the present invention, and fragments thereof will have binding specificity to an antigen such as TNF-alpha and exhibit activity that agonizes or antagonizes the functions of the particular antigen. Preferably the antigen will be a therapeutic target and the humanized antibody will ameliorate or reducing the symptoms of, or alternatively treating, diseases and disorders associated with the particular antigen such as IL-6 or TNF or another therapeutic target such as a human tumor polypeptide, autoantigen, allergen, or an antigen specific to an infectious agent

B-Cell Screening and Isolation

As noted, the invention provides general methods applicable for efficiently humanizing any rabbit antibody, i.e., specific to any desired antigen. These antibodies may be derived from hybridoma cells, sera, or from immune cells that secrete rabbit antibodies or antibodies of closely related species such as other Lagomorphs. If immune cells are used it is preferred that these cells constitute B cells secreting antibodies specific to a desired target antigen that are derived by the following B cell isolation protocol. It has been found that this protocol affords for a population of B cells that give rise on selection to antibodies with good binding affinities and moreover yields a full repertoire or diversity of antibodies, i.e. a population of antibodies that includes those that bind to a wide range of different epitopes. In this preferred embodiment, the present invention provides methods of isolating a clonal population of antigen-specific B cells obtained from an immune rabbit that may be used for isolating at least one antigen-specific cell. As described and exemplified infra, these methods contain a series of culture and selection steps that can be used separately, in combination, sequentially, repetitively, or periodically. Preferably, these methods are used for isolating at least one antigen-specific cell, which can be used to produce a monoclonal antibody, which is specific to a desired antigen, or a nucleic acid sequence corresponding to such an antibody.

Essentially, these methods comprise the steps of:

a. preparing a cell population comprising at least one antigen-specific B cell;

b. enriching the cell population, e.g., by chromatography, to form an enriched cell population comprising at least one antigen-specific B cell;

c. isolating a single B cell from the enriched B cell population; and

d. determining whether the single B cell produces an antibody specific to the antigen.

These methods provide an improvement to a method of isolating a single, antibody-producing B cell, the improvement comprising enriching a B cell population obtained from a host that has been immunized or naturally exposed to an antigen, wherein the enriching step precedes any selection steps, comprises at least one culturing step, and results in a clonal population of B cells that produces a single monoclonal antibody specific to said antigen.

With respect to such methods which are preferably used to derive rabbit B cells secreting antibodies which are employed in the inventive humanization approaches throughout this application, a “clonal population of B cells” refers to a population of B cells that only secrete a single antibody specific to a desired antigen. That is to say that these cells produce only one type of monoclonal antibody specific to the desired antigen.

In describing such methods the expression “enriching” a cell population cells means increasing the frequency of desired cells, typically antigen-specific cells, contained in a mixed cell population, e.g., a B cell-containing isolate derived from a host that is immunized against a desired antigen. Thus, an enriched cell population encompasses a cell population having a higher frequency of antigen-specific cells as a result of an enrichment step, but this population of cells may contain and produce different antibodies.

In further describing such methods the general expression “cell population” encompasses pre- and a post-enrichment cell populations, keeping in mind that when multiple enrichment steps are performed, a cell population can be both pre- and post-enrichment. More preferably these methods for deriving a clonal population of antigen specific B cells will comprise:

a. harvesting a cell population from an immunized host to obtain a harvested cell population;

b. creating at least one single cell suspension from the harvested cell population;

c. enriching at least one single cell suspension to form a first enriched cell population;

d. enriching the first enriched cell population to form a second enriched cell population;

e. enriching the second enriched cell population to form a third enriched cell population; and

f. selecting an antibody produced by an antigen-specific cell of the third enriched cell population.

Each cell population may be used directly in the next step, or it can be partially or wholly frozen for long- or short-term storage or for later steps. Also, cells from a cell population can be individually suspended to yield single cell suspensions. The single cell suspension can be enriched, such that a single cell suspension serves as the pre-enrichment cell population. Then, one or more antigen-specific single cell suspensions together form the enriched cell population; the antigen-specific single cell suspensions can be grouped together, e.g., re-plated for further analysis and/or antibody production.

Antigen-specificity can be measured with respect to any antigen. The antigen can be any substance to which an antibody can bind including, but not limited to, peptides, proteins or fragments thereof; carbohydrates; organic and inorganic molecules; receptors produced by animal cells, bacterial cells, and viruses; enzymes; agonists and antagonists of biological pathways; hormones; and cytokines. Exemplary antigens include, but are not limited to, IL-2, IL-4, IL-6, IL-10, IL-12, IL-13, IL-18, IFN-α, IFN-γ, BAFF, CXCL13, IP-10, VEGF, EPO, EGF, HRG, MIF, and colony stimulating factors, TPAs, interferons, tumor associated antigens, HIV antigens such as env and gag and pol, influenzae antigens, bird flu antigens, et al. Preferred antigens include IL-6, IL-13, TNF-α, VEGF-α, hepcidin and hepatocyte growth factor and tumor antigens specific to particular human cancers. In a method utilizing more than one enrichment step, the antigen used in each enrichment step can be the same as or different from one another. Multiple enrichment steps with the same antigen may yield a large and/or diverse population of antigen-specific cells; multiple enrichment steps with different antigens may yield an enriched cell population with cross-specificity to the different antigens.

Enriching a cell population can be performed by any cell-selection means known in the art for isolating antigen-specific cells. For example, a cell population can be enriched by chromatographic techniques, e.g., Miltenyi bead or magnetic bead technology. The beads can be directly or indirectly attached to the antigen of interest. In a preferred embodiment, the method of enriching a cell population includes at least one chromatographic enrichment step.

A cell population can also be enriched by performed by any antigen-specificity assay technique known in the art, e.g., an ELISA assay or a halo assay. ELISA assays include, but are not limited to, selective antigen immobilization (e.g., biotinylated antigen capture by streptavidin, avidin, or neutravidin coated plate), non-specific antigen plate coating, and through an antigen build-up strategy (e.g., selective antigen capture followed by binding partner addition to generate a heteromeric protein-antigen complex). The antigen can be directly or indirectly attached to a solid matrix or support, e.g., a column. A halo assay comprises contacting the cells with antigen-loaded beads and labeled anti-host antibody specific to the host used to harvest the B cells. The label can be, e.g., a fluorophore. In one embodiment, at least one assay enrichment step is performed on at least one single cell suspension. In another embodiment, the method of enriching a cell population includes at least one chromatographic enrichment step and at least one assay enrichment step.

Methods of “enriching” a cell population by size or density are known in the art. These steps can be used in the present method in addition to enriching the cell population by antigen-specificity.

The cell populations used in these methods will contain at least one cell capable of recognizing an antigen. Antigen-recognizing cells include, but are not limited to, B cells, plasma cells, and progeny thereof. Typically these methods will be effected under conditions giving rise to a clonal cell population containing a single type of antigen-specific B-cell, i.e., the cell population produces a single monoclonal antibody specific to a desired antigen. In the present invention these antigen-specific B cells will typically be rabbit or alternatively a B cell from a closely related mammalian species.

It is believed that a clonal antigen-specific population of B cells consisting predominantly of antigen-specific, antibody-secreting cells, is obtained by the novel culture and selection protocol provided herein.

In such methods the isolation of a single B cell can be effected by enriching a cell population obtained from a host before any selection steps, e.g., selecting a particular B cell from a cell population and/or selecting an antibody produced by a particular cell. The enrichment step can be performed as one, two, three, or more steps. In one embodiment, a single B cell is isolated from an enriched cell population before confirming whether the single B cell secretes an antibody with antigen-specificity and/or a desired property.

In a preferred embodiment of this invention an enriched cell population obtained from a rabbit immunized to a desired antigen is used in a method for antibody production and/or selection which are candidate starting materials for the subject humanization strategies. The method can include the steps of: preparing a cell population comprising at least one antigen-specific cell, enriching the cell population by isolating at least one antigen-specific cell to form an enriched cell population, and inducing antibody production from at least one antigen-specific cell. In a preferred embodiment, the enriched cell population contains more than one antigen-specific cell. In one embodiment, each antigen-specific cell of the enriched population is cultured under conditions that yield a clonal antigen-specific B cell population before isolating an antibody producing cell therefrom and/or producing an antibody using said B cell, or a nucleic acid sequence corresponding to such an antibody which is used in the present humanization strategies. In contrast to prior techniques where antibodies are produced from a cell population with a low frequency of antigen-specific cells, the present invention allows antibody selection from among a high frequency of antigen-specific cells. Because an enrichment step is used prior to antibody selection, the majority of the cells, preferably virtually all of the cells, used for antibody production are antigen-specific. By producing antibodies from a population of cells with an increased frequency of antigen specificity, the quantity and variety of antibodies are increased thus providing more starting materials for humanization.

When using these antibody selection methods are used to derive the rabbit antibodies for humanization, an antibody is preferably selected after an enrichment step and a culture step that results in a clonal population of antigen-specific B cells. The methods can further comprise a step of sequencing a selected antibody or portions thereof from one or more isolated, antigen-specific cells. Any method known in the art for sequencing can be employed and can include sequencing the heavy chain, light chain, variable region(s), and/or complementarity determining region(s) (CDR).

In addition to the enrichment step, the method for antibody selection can also include one or more steps of screening a cell population for antigen recognition and/or antibody functionality. For example, the desired antibodies may have specific structural features, such as binding to a particular epitope or mimicry of a particular structure; antagonist or agonist activity; or neutralizing activity, e.g., inhibiting binding between the antigen and a ligand. In one embodiment, the antibody functionality screen is ligand-dependent. Screening for antibody functionality includes, but is not limited to, an in vitro protein-protein interaction assay that recreates the natural interaction of the antigen ligand with recombinant receptor protein; and a cell-based response that is ligand dependent and easily monitored (e.g., proliferation response). In one embodiment, the method for antibody selection includes a step of screening the cell population for antibody functionality by measuring the inhibitory concentration (IC50). In one embodiment, at least one of the isolated, antigen-specific cells produces an antibody having an IC50 of less than about 100, 50, 30, 25, 10 μg/mL, or increments therein.

In addition to the enrichment step, the method for antibody selection can also include one or more steps of screening a cell population for antibody binding strength. Antibody binding strength can be measured by any method known in the art (e.g., Biacore). In one embodiment, at least one of the isolated, antigen-specific cells produces an antibody having a high antigen affinity, e.g., a dissociation constant (Kd) of less than about 5×10⁻¹⁰M⁻¹, preferably about 1×10⁻¹³ M⁻¹ to 5×10⁻¹⁰ M⁻¹, 1×10⁻¹² M⁻¹ to 7.5×10⁻¹¹ M⁻¹, 1×10⁻¹¹ M⁻¹ to 2×10⁻¹¹ M⁻¹ or about 1.5×10⁻¹¹ M⁻¹ or less, or increments therein. In this embodiment, the antibodies are said to be affinity mature.

In a preferred embodiment, the affinity of the antibodies used for humanization herein is comparable to or higher than the affinity of any one of Panorex® (edrecolomab), Rituxan® (rituximab), Herceptin® (traztuzumab), Mylotarg® (gentuzumab), Campath® (alemtuzumab), Zevalin™ (ibritumomab), Erbitux™ (cetuximab), Avastin™ (bevicizumab), Raptiva™ (efalizumab), Remicade® (infliximab), Humira™ (adalimumab), and Xolair™ (omalizumab). Preferably, the affinity of the antibodies is comparable to or higher than the affinity of Humira™. The affinity of an antibody can also be increased by known affinity maturation techniques. In one embodiment, at least one cell population is screened for at least one of, preferably both, antibody functionality and antibody binding strength.

In addition to the enrichment step, the method for antibody selection used to select candidates for humanization can also include one or more steps of screening a rabbit cell population for antibody sequence homology, especially human homology. In one embodiment, at least one of the isolated, antigen-specific cells produces an antibody that has a homology to a human antibody of about 50% to about 100%, or increments therein, or greater than about 60%, 70%, 80%, 85%, 90%, or 95% homologous.

In another preferred embodiment, the present invention also provides the rabbit derived humanized antibodies produced from antibodies according to any of the embodiments described above in terms of IC50, Kd, and/or homology.

The B cell selection protocol disclosed herein which is preferably used to identify B cells producing antibodies having an affinity and functional properties rendering them good candidates for humanization has a number of intrinsic advantages versus other methods for obtaining antibody-secreting B cells and monoclonal antibodies specific to desired target antigens. These advantages include, but are not restricted to, the following:

First, it has been found that when these selection procedures are utilized with a desired antigen such as IL-6 or TNF-α, the methods reproducibly result in antigen-specific B cells e.g., derived from rabbits capable of generating what appears to be a substantially comprehensive complement of antibodies, i.e., antibodies that bind to the various different epitopes of the antigen. Without being bound by theory, it is hypothesized that the comprehensive complement is attributable to the antigen enrichment step that is performed prior to initial B cell recovery. Moreover, this advantage allows for the isolation and selection of antibodies with different properties as these properties may vary depending on the epitopic specificity of the particular antibody. These antibodies are ideal starting materials for the inventive humanization strategies.

Second, it has been found that the inventive B cell selection protocol reproducibly yields a clonal B cell culture containing a single B cell, or its progeny, secreting a single monoclonal antibody that generally binds to the desired antigen with a relatively high binding affinity. By contrast, prior antibody selection methods tend to yield relatively few high affinity antibodies and therefore require extensive screening procedures to isolate an antibody with therapeutic potential. Without being bound by theory, it is hypothesized that the inventive protocol results in both in vivo B cell immunization of the host (primary immunization) followed by a second in vitro B cell stimulation (secondary antigen priming step) that may enhance the ability and propensity of the recovered clonal B cells to secrete a single high affinity monoclonal antibody specific to the antigen target.

Third, it has been observed that the inventive B cell selection protocol reproducibly yields enriched B cells producing IgG's that are, on average, highly selective (antigen specific) to the desired target. In part based thereon, antigen-enriched B cells recovered by the inventive methods are believed to contain B cells capable of yielding the desired full complement of epitopic specificities as discussed above.

Fourth, it has been observed that this B cell selection protocol, even when used with small antigens, i.e., peptides of 100 amino acids or less, e.g., 5-50 amino acids long, reproducibly give rise to a clonal B cell culture that secretes a single high affinity antibody to the small antigen, e.g., a peptide. This is highly surprising as it is generally quite difficult, labor intensive, and sometimes not even feasible to produce high affinity antibodies to small peptides. Accordingly, these methods can be used to produce ideal candidates for deriving humanized therapeutic antibodies to desired peptide targets, e.g., viral, bacterial or autoantigen peptides, thereby allowing for the production of monoclonal antibodies with very discrete binding properties or even the production of a cocktail of monoclonal antibodies to different peptide targets, e.g., different viral strains. This advantage may especially be useful in the context of the production of a therapeutic or prophylactic vaccine having a desired valency, such as an HPV vaccine that induces protective immunity to different HPV strains.

Fifth, this B cell selection protocol, particularly when used with B cells derived from rabbits, tends to reproducibly yield antigen-specific antibody sequences that are very similar to endogenous human immunoglobulins (around 90% similar at the amino acid level) and that contain CDRs that possess a length very analogous to human immunoglobulins and therefore require little or no sequence modification (typically as described previously at most only a few CDR residues need be modified in the parent antibody sequence and no framework exogenous residues introduced) in order to eliminate potential immunogenicity concerns. In particular, preferably the recombinant antibody will contain only the host (rabbit) CDR1 and CDR2 residues required for antigen recognition and the entire CDR3 as this seems to be important for antibody affinity maturation. Thereby, the high antigen binding affinity of the recovered antibody sequences produced according to the inventive B cell and antibody selection protocol remains intact or substantially intact even with humanization.

In sum, the inventive methods can be used to produce humanized antibodies exhibiting higher binding affinities to more distinct epitopes by the use of a more efficient protocol than was previously known.

In a specific embodiment, the present invention provides a method for identifying a single B cell that secretes an antibody specific to a desired antigen for humanization in the inventive protocols and which optionally possesses at least one desired functional property such as affinity, avidity, cytolytic activity, and the like by a process including the following steps:

a. immunizing a host against an antigen;

b. harvesting B cells from the host;

c. enriching the harvested B cells to increase the frequency of antigen-specific cells;

d. creating at least one single cell suspension;

e. culturing a sub-population from the single cell suspension under conditions that favor the survival of a single antigen-specific B cell per culture well;

f. isolating less than 10 to 12 B cells from the sub-population; and

g. determining whether the single B cell produces an antibody specific to the antigen.

The inventive methods will further comprise an additional step of isolating and sequencing, in whole or in part, the polypeptide and nucleic acid sequences encoding the desired antibody to identify the critical residues such as selectivity determining residues and in order to use this sequence as part of a BLAST search to identify candidate homologous human variable sequences to utilize for deriving an ideal humanized version thereof. These sequences or humanized versions or portions thereof can be expressed in desired host cells in order to produce recombinant antibodies to a desired antigen such as IL-6, TNF-α, hepatocyte growth factor, hepcidin et al.

As noted previously, it is believed that the clonal population of B cells predominantly comprises antibody-secreting B cells producing antibody against the desired antigen. It is also believed based on experimental results obtained with several antigens and with different B cell populations that the clonally produced B cells and the isolated antigen-specific B cells derived therefrom produced according to the invention secrete a monoclonal antibody that is typically of relatively high affinity and moreover is capable of efficiently and reproducibly producing a selection of monoclonal antibodies of greater epitopic variability as compared to other methods of deriving monoclonal antibodies from cultured antigen-specific B cells. In the subject invention the population of immune cells used in such B cell selection methods will be derived from a rabbit or an animal closely related thereto such as another Leporidae species. It is believed that the use of rabbits or closely related mammals as a source of B cells may enhance the diversity of monoclonal antibodies that may be used in the present invention to derive humanized versions. Also, the antibody sequences derived from rabbits according to the invention typically possess sequences having a high degree of sequence identity to human antibody sequences making them favored for use in humans since they should result in humanized variants that possess little antigenicity. In the course of humanization, the final humanized antibody contains a much lower foreign/host residue content, usually restricted to a subset of the host CDR residues that differ dramatically due to their nature versus the human target sequence used in the grafting. This enhances the probability of complete activity recovery in the humanized antibody protein produced using the inventive humanization strategy.

The methods of antibody selection using an enrichment step disclosed herein include a step of obtaining a immune cell-containing cell population from an immunized host. Methods of obtaining an immune cell-containing cell population from an immunized host are known in the art and generally include inducing an immune response in a host and harvesting cells from the host to obtain one or more cell populations. The response can be elicited by immunizing the host against a desired antigen. Alternatively, the host used as a source of such immune cells can be naturally exposed to the desired antigen such as an individual who has been infected with a particular pathogen such as a bacterium or virus or alternatively has mounted a specific antibody response to a cancer that the individual is afflicted with. In the present methods the hosts are rabbits.

As mentioned, the immune response can occur naturally, as a result of disease, or it can be induced by immunization with the antigen. Immunization can be performed by any method known in the art, such as, by one or more injections of the antigen with or without an agent to enhance immune response, such as complete or incomplete Freund's adjuvant. As an alternative to immunizing a host animal in vivo, the method can comprise immunizing a host cell culture in vitro.

After allowing time for the immune response (e.g., as measured by serum antibody detection), host animal cells are harvested to obtain one or more cell populations. In a preferred embodiment, a harvested cell population is screened for antibody binding strength and/or antibody functionality. A harvested cell population is preferably from at least one of the spleen, lymph nodes, bone marrow, and/or peripheral blood mononuclear cells (PBMCs). The cells can be harvested from more than one source and pooled. Certain sources may be preferred for certain antigens. For example, the spleen, lymph nodes, and PBMCs are preferred for IL-6; and the lymph nodes are preferred for TNF. The cell population is harvested about 20 to about 90 days or increments therein after immunization, preferably about 50 to about 60 days. A harvested cell population and/or a single cell suspension therefrom can be enriched, screened, and/or cultured for antibody selection. The frequency of antigen-specific cells within a harvested cell population is usually about 1% to about 5%, or increments therein.

In one embodiment, a single cell suspension from a harvested cell population is enriched, preferably by using Miltenyi beads. From the harvested cell population having a frequency of antigen-specific cells of about 1% to about 5%, an enriched cell population is thus derived having a frequency of antigen-specific cells approaching 100%.

The method of antibody selection using an enrichment step includes a step of producing antibodies from at least one antigen-specific cell from an enriched cell population. Methods of producing antibodies in vitro are well known in the art, and any suitable method can be employed. In one embodiment, an enriched cell population, such as an antigen-specific single cell suspension from a harvested cell population, is plated at various cell densities, such as 50, 100, 250, 500, or other increments between 1 and 1000 cells per well. Preferably, the sub-population comprises no more than about 10,000 antigen-specific, antibody-secreting cells, more preferably about 50-10,000, about 50-5,000, about 50-1,000, about 50-500, about 50-250 antigen-specific, antibody-secreting cells, or increments therein. Then, these sub-populations are cultured with suitable medium (e.g., an activated T cell conditioned medium, particularly 1-5% activated rabbit T cell conditioned medium) on a feeder layer, preferably under conditions that favor the survival of a single proliferating antibody-secreting cell per culture well. The feeder layer, generally comprised of irradiated cell matter, e.g., EL4B cells, does not constitute part of the cell population. The cells are cultured in a suitable media for a time sufficient for antibody production, for example about 1 day to about 2 weeks, about 1 day to about 10 days, at least about 3 days, about 3 to about 5 days, about 5 days to about 7 days, at least about 7 days, or other increments therein. In one embodiment, more than one sub-population is cultured simultaneously. Preferably, a single antibody-producing cell and progeny thereof survives in each well, thereby providing a clonal population of antigen-specific B cells in each well. At this stage, the immunoglobulin G (IgG) produced by the clonal population is highly correlative with antigen specificity. In a preferred embodiment, the IgGs exhibit a correlation with antigen specificity that is greater than about 50%, more preferably greater than 70%, 85%, 90%, 95%, 99%, or increments therein. The correlations have been demonstrated by setting up B cell cultures under limiting conditions to establish single antigen-specific antibody products per well. Antigen-specific versus general IgG synthesis was compared. Three populations were observed: IgG that recognized a single formate of antigen (biotinylated and direct coating), detectable IgG and antigen recognition irrespective of immobilization, and IgG production alone. IgG production was highly correlated with antigen-specificity.

A supernatant containing the antibodies is optionally collected, which can be can be enriched, screened, and/or cultured for antibody selection according to the steps described above. In one embodiment, the supernatant is enriched (preferably by an antigen-specificity assay, especially an ELISA assay) and/or screened for antibody functionality.

In another embodiment, the enriched, preferably clonal, antigen-specific B cell population from which a supernatant described above is optionally screened in order to detect the presence of the desired secreted monoclonal antibody is used for the isolation of a few B cells, preferably a single B cell, which is then tested in an appropriate assay in order to confirm the presence of a single antibody-producing B cell in the clonal B cell population. In one embodiment about 1 to about 20 cells are isolated from the clonal B cell population, preferably less than about 15, 12, 10, 5, or 3 cells, or increments therein, most preferably a single cell. The screen is preferably effected by an antigen-specificity assay, especially a halo assay. The halo assay can be performed with the full length protein, or a fragment thereof. The antibody-containing supernatant can also be screened for at least one of: antigen binding affinity; agonism or antagonism of antigen-ligand binding, induction or inhibition of the proliferation of a specific target cell type; induction or inhibition of lysis of a target cell, and induction or inhibition of a biological pathway involving the antigen.

The identified antigen-specific cell derived from a rabbit host can be used to derive the corresponding nucleic acid sequences encoding the desired monoclonal antibody which may used in the inventive humanization approaches. (An AluI digest can confirm that only a single monoclonal antibody type is produced per well.) As mentioned above, these sequences are then preferably mutated, by the inventive humanization protocols, in order to render them more suitable for use in human medicaments.

As mentioned, the enriched B cell population from rabbits used in the inventive process can also be further enriched, screened, and/or cultured for antibody selection according to the steps described above which can be repeated or performed in a different order. In a preferred embodiment, at least one cell of an enriched, preferably clonal, antigen-specific cell population is isolated, cultured, and used for antibody selection.

Thus, in another embodiment, the present invention provides a method of isolating antibody candidates for use in the subject humanization methods comprising:

a. harvesting a cell population from an immunized rabbit host to obtain a harvested cell population;

b. creating at least one single cell suspension from a harvested cell population;

c. enriching at least one single cell suspension, preferably by chromatography, to form a first enriched cell population;

d. enriching the first enriched cell population, preferably by ELISA assay, to form a second enriched cell population which preferably is clonal, i.e., it contains only a single type of antigen-specific B cell;

e. enriching the second enriched cell population, preferably by halo assay, to form a third enriched cell population containing a single or a few number of B cells that produce an antibody specific to a desired antigen; and

f. selecting an antibody produced by an antigen-specific cell isolated from the third enriched cell population.

The method can further include one or more steps of screening the harvested cell population for antibody binding strength (affinity, avidity) and/or antibody functionality. Suitable screening steps include, but are not limited to, assay methods that detect: whether the antibody produced by the identified antigen-specific B cell produces an antibody possessing a minimal antigen binding affinity, whether the antibody agonizes or antagonizes the binding of a desired antigen to a ligand; whether the antibody induces or inhibits the proliferation of a specific cell type; whether the antibody induces or elicits a cytolytic reaction against target cells; whether the antibody binds to a specific epitope; and whether the antibody modulates (inhibits or agonizes) a specific biological pathway or pathways involving the antigen.

Similarly, the method can include one or more steps of screening the second enriched cell population for antibody binding strength and/or antibody functionality.

The methods further include a step of sequencing the polypeptide sequence or the corresponding nucleic acid sequence of the selected antibody to identify critical residues and in order to conduct BLAST searches of appropriate homologous human germline antibody sequences for use in the subject humanization methods. The methods also include a step of producing a recombinant antibody using the sequence, a fragment thereof, or a genetically modified humanized version of the selected antibody. These humanization mutation methods can yield recombinant antibodies possessing desired effector function, immunogenicity, stability, removal or addition of glycosylation, and the like. The recombinant humanized antibody or humanized antibody fragments described herein can be produced by any suitable recombinant cell, including, but not limited to mammalian cells such as CHO, COS, BHK, HEK-293, bacterial cells, yeast cells, plant cells, insect cells, and amphibian cells. In a preferred embodiment, the parent rabbit antibody and humanized antibodies derived from these antibodies and homologous human variable sequences are expressed in polyploidal yeast cells, i.e., diploid yeast cells, particularly Pichia.

Essentially, the method may be effected as follows:

a. immunizing a rabbit host against an antigen to yield rabbit antibodies;

b. screening the obtained rabbit antibodies for antigen specificity and neutralization;

c. harvesting B cells from the rabbit;

d. enriching the harvested rabbit B cells to create an enriched cell population having an increased frequency of antigen-specific cells;

e. culturing one or more sub-populations from the enriched cell population under conditions that favor the survival of a single B cell to produce a clonal population in at least one culture well;

f. determining whether the clonal population produces a rabbit antibody specific to the antigen;

g. isolating a single rabbit B cell; and

h. sequencing the nucleic acid sequence of the rabbit antibody produced by the single B cell and

i. using this antibody sequence in order to derive humanized antibodies possessing the affinity and optionally other properties of the parent rabbit antibody using the inventive humanization strategies.

Methods of Humanizing Antibodies

As described, the present invention provides a novel and improved method for humanizing rabbit antibody heavy and light chains. The methods of the invention may be effected as follows for the humanization of the rabbit antibody heavy and light chains:

Humanization of Rabbit Antibody Light Chain

1. Identify the amino acid that is the first one following the signal peptide sequence. This is the start of Framework 1. The signal peptide starts at the first initiation methionine and is typically, but not necessarily 22 amino acids in length for rabbit light chain protein sequences. The start of the mature polypeptide can also be determined experimentally by N-terminal protein sequencing, or can be predicted using a prediction algorithm. This is also the start of Framework 1 as classically defined by those in the field.

Example: RbtVL Amino acid residue 1 in FIG. 2, starting ‘AYDM . . . ’

2. Identify the end of Framework 3. This is typically 86-90 amino acids following the start of Framework 1 and is typically a cysteine residue preceded by two tyrosine residues. This is the end of the Framework 3 as classically defined by those in the field.

Example: RbtVL amino acid residue 88 in FIG. 2, ending as ‘TYYC’

3. Use the rabbit light chain sequence of the polypeptide starting from the beginning of Framework 1 to the end of Framework 3 as defined above and perform a sequence homology search for the most similar human antibody protein sequences. This will typically be a search against human germline sequences prior to antibody maturation in order to reduce the possibility of immunogenicity, however any human sequences can be used. Typically a program like BLAST can be used to search a database of sequences for the most homologous. Databases of human antibody sequences can be found from various sources such as NCBI (National Center for Biotechnology Information).

Example: RbtVL amino acid sequence from residues numbered 1 through 88 in FIG. 2 is BLASTed against a human antibody germline database. The top three unique returned sequences are shown in FIG. 2 as L12A, V1 and Vx02.

4. Generally the most homologous human germline variable light chain sequence is then used as the basis for humanization. However those skilled in the art may decide to use another sequence that wasn't the highest homology as determined by the homology algorithm, based on other factors including sequence gaps and framework similarities.

Example: In FIG. 2, L12A was the most homologous human germline variable light chain sequence and is used as the basis for the humanization of RbtVL.

5. Determine the framework and CDR arrangement (FR1, FR2, FR3, CDR1 & CDR2) for the human homolog being used for the light chain humanization. This is using the traditional layout as described in the field. Align the rabbit variable light chain sequence with the human homolog, while maintaining the layout of the framework and CDR regions.

Example: In FIG. 2, the RbtVL sequence is aligned with the human homologous sequence L12A, and the framework and CDR domains are indicated.

6. Replace the human homologous light chain sequence CDR1 and CDR2 regions with the CDR1 and CDR2 sequences from the rabbit sequence. If there are differences in length between the rabbit and human CDR sequences then use the entire rabbit CDR sequences and their lengths. It is possible that the specificity, affinity and/or immunogenicity of the resulting humanized antibody may be unaltered if smaller or larger sequence exchanges are performed, or if specific residue(s) are altered, however the exchanges as described have been used successfully, but do not exclude the possibility that other changes may be permitted.

Example: In FIG. 2, the CDR1 and CDR2 amino acid residues of the human homologous variable light chain L12A are replaced with the CDR1 and CDR2 amino acid sequences from the RbtVL rabbit antibody light chain sequence. The human L12A frameworks 1, 2 and 3 are unaltered. The resulting humanized sequence is shown below as VLh from residues numbered 1 through 88. Note that the only residues that are different from the L12A human sequence are underlined, and are thus rabbit-derived amino acid residues. In this example only 8 of the 88 residues are different than the human sequence.

After framework 3 of the new hybrid sequence created in Step 6, attach the entire CDR3 of the rabbit light chain antibody sequence. The CDR3 sequence can be of various lengths, but is typically 9 to 15 amino acid residues in length. The CDR3 region and the beginning of the following framework 4 region are defined classically and identifiable by those skilled in the art. Typically the beginning of Framework 4, and thus after the end of CDR3 consists of the sequence ‘FGGG . . . ’, however some variation may exist in these residues.

Example: In FIG. 2, the CDR3 of RbtVL (amino acid residues numbered 89-100) is added after the end of framework 3 in the humanized sequence indicated as VLh.

8. The rabbit light chain framework 4, which is typically the final 11 amino acid residues of the variable light chain and begins as indicated in Step 7 above and typically ends with the amino acid sequence ‘ . . . VVKR’ is replaced with the nearest human light chain framework 4 homolog, usually from germline sequence. Frequently this human light chain framework 4 is of the sequence ‘FGGGTKVEIKR’. It is possible that other human light chain framework 4 sequences that are not the most homologous or otherwise different may be used without affecting the specificity, affinity and/or immunogenicity of the resulting humanized antibody. This human light chain framework 4 sequence is added to the end of the variable light chain humanized sequence immediately following the CDR3 sequence from Step 7 above. This is now the end of the variable light chain humanized amino acid sequence.

Example: In FIG. 2, Framework 4 (FR4) of the RbtVL rabbit light chain sequence is shown above a homologous human FR4 sequence. The human FR4 sequence is added to the humanized variable light chain sequence (VLh) right after the end of the CD3 region added in Step 7 above.

Humanization of Rabbit Antibody Heavy Chain

1. Identify the amino acid that is the first one following the signal peptide sequence. This is the start of Framework 1. The signal peptide starts at the first initiation methionine and is typically 19 amino acids in length for rabbit heavy chain protein sequences. Typically, but not necessarily always, the final 3 amino acid residues of a rabbit heavy chain signal peptide are ‘ . . . VQC’, followed by the start of Framework 1. The start of the mature polypeptide can also be determined experimentally by N-terminal protein sequencing, or can be predicted using a prediction algorithm. This is also the start of Framework 1 as classically defined by those in the field.

Example: RbtVH Amino acid residue 1 in FIG. 2, starting ‘QEQL . . . . ’

2. Identify the end of Framework 3. This is typically 95-100 amino acids following the start of Framework 1 and typically has the final sequence of ‘ . . . CAR’ (although the alanine can also be a valine). This is the end of the Framework 3 as classically defined by those in the field.

Example: RbtVH amino acid residue 98 in FIG. 2, ending as ‘ . . . FCVR’.

3. Use the rabbit heavy chain sequence of the polypeptide starting from the beginning of Framework 1 to the end of Framework 3 as defined above and perform a sequence homology search for the most similar human antibody protein sequences. This will typically be against a database of human germline sequences prior to antibody maturation in order to reduce the possibility of immunogenicity, however any human sequences can be used. Typically a program like BLAST can be used to search a database of sequences for the most homologous. Databases of human antibody sequences can be found from various sources such as NCBI (National Center for Biotechnology Information).

Example: RbtVH amino acid sequence from residues numbered 1 through 98 in FIG. 2 is BLASTed against a human antibody germline database. The top three unique returned sequences are shown in FIG. 2 as 3-64-04, 3-66-04, and 3-53-02.

4. Generally the most homologous human germline variable heavy chain sequence is then used as the basis for humanization. However those skilled in the art may decide to use another sequence that wasn't the most homologous as determined by the homology algorithm, based on other factors including sequence gaps and framework similarities.

Example: 3-64-04 in FIG. 2 was the most homologous human germline variable heavy chain sequence and is used as the basis for the humanization of RbtVH.

5. Determine the framework and CDR arrangement (FR1, FR2, FR3, CDR1 & CDR2) for the human homolog being used for the heavy chain humanization. This is using the traditional layout as described in the field. Align the rabbit variable heavy chain sequence with the human homolog, while maintaining the layout of the framework and CDR regions.

Example: In FIG. 2, the RbtVH sequence is aligned with the human homologous sequence 3-64-04, and the framework and CDR domains are indicated.

6. Replace the human homologous heavy chain sequence CDR1 and CDR2 regions with the CDR1 and CDR2 sequences from the rabbit sequence. If there are differences in length between the rabbit and human CDR sequences then use the entire rabbit CDR sequences and their lengths. In addition, it may be necessary to replace the final three amino acids of the human heavy chain Framework 1 region with the final three amino acids of the rabbit heavy chain Framework 1. Typically but not always, in rabbit heavy chain Framework 1 these three residues follow a Glycine residue preceded by a serine residue. In addition, it may be necessary replace the final amino acid of the human heavy chain Framework 2 region with the final amino acid of the rabbit heavy chain Framework 2. Typically, but not necessarily always, this is a Glycine residue preceded by an isoleucine residue in the rabbit heavy chain Framework 2. It is possible that the specificity, affinity and/or immunogenicity of the resulting humanized antibody may be unaltered if smaller or larger sequence exchanges are performed, or if specific residue(s) are altered, however the exchanges as described have been used successfully, but do not exclude the possibility that other changes may be permitted. For example, a tryptophan amino acid residue typically occurs four residues prior to the end of the rabbit heavy chain CDR2 region, whereas in human heavy chain CDR2 this residue is typically a serine residue. Changing this rabbit tryptophan residue to a the human serine residue at this position has been demonstrated to have minimal to no effect on the humanized antibody's specificity or affinity, and thus further minimizes the content of rabbit sequence-derived amino acid residues in the humanized sequence.

Example: In FIG. 2, The CDR1 and CDR2 amino acid residues of the human homologous variable heavy chain are replaced with the CDR1 and CDR2 amino acid sequences from the RbtVH rabbit antibody light chain sequence, except for the boxed residue, which is tryptophan in the rabbit sequence (position number 63) and serine at the same position in the human sequence, and is kept as the human serine residue. In addition to the CDR1 and CDR2 changes, the final three amino acids of Framework 1 (positions 28-30) as well as the final residue of Framework 2 (position 49) are retained as rabbit amino acid residues instead of human. The resulting humanized sequence is shown below as VHh from residues numbered 1 through 98. Note that the only residues that are different from the 3-64-04 human sequence are underlined, and are thus rabbit-derived amino acid residues. In this example only 15 of the 98 residues are different than the human sequence.

7. After framework 3 of the new hybrid sequence created in Step 6, attach the entire CDR3 of the rabbit heavy chain antibody sequence. The CDR3 sequence can be of various lengths, but is typically 5 to 19 amino acid residues in length. The CDR3 region and the beginning of the following framework 4 region are defined classically and are identifiable by those skilled in the art. Typically the beginning of framework 4, and thus after the end of CDR3 consists of the sequence WGXG . . . (where X is usually Q or P), however some variation may exist in these residues.

Example: The CDR3 of RbtVH (amino acid residues numbered 99-110) is added after the end of framework 3 in the humanized sequence indicated as VHh.

8. The rabbit heavy chain framework 4, which is typically the final 11 amino acid residues of the variable heavy chain and begins as indicated in Step 7 above and typically ends with the amino acid sequence ‘ . . . TVSS’ is replaced with the nearest human heavy chain framework 4 homolog, usually from germline sequence. Frequently this human heavy chain framework 4 is of the sequence ‘WGQGTLVTVSS’. It is possible that other human heavy chain framework 4 sequences that are not the most homologous or otherwise different may be used without affecting the specificity, affinity and/or immunogenicity of the resulting humanized antibody. This human heavy chain framework 4 sequence is added to the end of the variable heavy chain humanized sequence immediately following the CDR3 sequence from Step 7 above. This is now the end of the variable heavy chain humanized amino acid sequence.

Example: In FIG. 2, framework 4 (FR4) of the RbtVH rabbit heavy chain sequence is shown above a homologous human heavy FR4 sequence. The human FR4 sequence is added to the humanized variable heavy chain sequence (VHh) right after the end of the CD3 region added in Step 7 above.

The afore-described humanization methods afford significant benefits over prior humanization methods. For example the invention provides a method for humanizing antibody sequences from rabbit antibody sequences that replaces a very large percentage of the rabbit amino acid residues with human antibody residues from a selected homologous aligned human antibody sequences. Consequently they are less likely to be immunogenic in humans.

In addition the inventive method relies on a comparison of primary sequences only and does not rely on or need (i) an understanding of the three dimensional structure of the donor or acceptor antibody sequences; (ii) an understanding of the localization of residues with regards to surface versus buried residues; (iii) trying out different versions or variations of different framework residue alternatives at specific or random sites. Consequently the present invention is highly efficient relative to more complex humanization approaches without any compromise to the desired properties of the resultant humanized antibodies such as binding affinity and other functional properties.

Further, and related to the foregoing, the resulting humanized antibodies produced by the inventive methods possess identical or virtually identical binding specificity relative to the parent rabbit antibody.

Also, the inventive methods require no additional “affinity maturation” in order to optimize or enhance antigen affinity. By contrast, in most other humanization approaches it is necessary to significantly increase antigen affinity after humanization (to be therapeutically or diagnostically effective at feasible dosages) by effecting iterations of “affinity maturation” protocols that screen through a number of random or defined sequence variants in order to identify variants with increased binding affinity. Consequently, the present invention is simpler and more efficient than prior humanization approaches.

Still further the present humanization methods are advantageous since the resulting humanized variable light and heavy chain sequences can be used to produce full-length antibodies as well as humanized antibody fragments or fusion proteins containing. Therefore, these humanized antibodies, humanized antibody fragments and fusion proteins containing such as those attached to therapeutic or diagnostic agents are well suited for immunotherapy as well as in vivo immunodiagnosis and immunoprognosis such as for use in imaging of tumor tissue, metastases, atherosclerotic plaques, inflammatory sites and the like.

Preferred Methods of Producing the Inventive Humanized Antibodies and Fragments thereof Recombinantly

The invention is also directed to preferred methods for the production of the humanized rabbit antibodies described herein or fragments thereof. Recombinant polypeptides corresponding to the antibodies described herein or fragments thereof are preferably secreted from polyploidal, preferably diploid or tetraploid strains of mating competent yeast. The invention is directed to methods for producing these recombinant polypeptides in secreted form for prolonged periods using cultures comprising polyploid yeast, i.e., at least several days to a week, more preferably at least a month or several months, and even more preferably at least 6 months to a year or longer. These polyploid yeast cultures will express at least 10-25 mg/liter of the polypeptide, more preferably at least 50-250 mg/liter, still more preferably at least 500-1000 mg/liter, and most preferably a gram per liter or more of the recombinant polypeptide(s).

In one embodiment of the invention a pair of genetically marked yeast haploid cells are transformed with expression vectors comprising subunits of a desired heteromultimeric protein. One haploid cell comprises a first expression vector, and a second haploid cell comprises a second expression vector. In another embodiment diploid yeast cells will be transformed with one or more expression vectors that provide for the expression and secretion of one or more of the recombinant humanized polypeptides provided by the invention. In still another embodiment a single haploid cell may be transformed with one or more vectors and used to produce a polyploidal yeast by fusion or mating strategies. In yet another embodiment a diploid yeast culture may be transformed with one or more vectors providing for the expression and secretion of a desired humanized rabbit heavy or light chain or antibody polypeptide or polypeptides produced according to the invention. These vectors may comprise plasmids that are maintained extra-chromosomally or may comprise vectors e.g., linearized plasmids that integrate into the yeast cell's genome randomly or by homologous recombination. Optionally, additional expression vectors may be introduced into the haploid or diploid cells; or the first or second expression vectors may comprise additional coding sequences; for the synthesis of heterotrimers; heterotetramers; etc. The expression levels of the non-identical polypeptides may be individually calibrated, and adjusted through appropriate selection, vector copy number, promoter strength and/or induction and the like. The transformed haploid cells are genetically crossed or fused. The resulting diploid or tetraploid strains are utilized to produce and secrete fully assembled and biologically functional proteins, humanized antibodies described herein or fragments thereof.

The use of diploid or tetraploid cells for protein production provides for unexpected benefits. The cells can be grown for production purposes, i.e. scaled up, and for extended periods of time, in conditions that can be deleterious to the growth of haploid cells, which conditions may include high cell density; growth in minimal media; growth at low temperatures; stable growth in the absence of selective pressure; and which may provide for maintenance of heterologous gene sequence integrity and maintenance of high level expression over time. Without wishing to be bound thereby, the inventors theorize that these benefits may arise, at least in part, from the creation of diploid strains from two distinct parental haploid strains. Such haploid strains can comprise numerous minor autotrophic mutations, which mutations are complemented in the diploid or tetraploid, enabling growth under highly selective conditions.

Transformed mating competent haploid yeast cells provide a genetic method that enables subunit pairing of a desired humanized antibody protein. Haploid yeast strains are transformed with each of two expression vectors, a first vector to direct the synthesis of one polypeptide chain and a second vector to direct the synthesis of a second, non-identical polypeptide chain, i.e., humanized rabbit heavy and light chain polypeptides. The two haploid strains are mated to provide a diploid host where optimized target protein (humanized rabbit antibody or humanized rabbit antibody fragment) production can be obtained.

Optionally, additional non-identical coding sequence(s) are provided. Such sequences may be present on additional expression vectors or in the first or the second expression vectors. As is known in the art, multiple coding sequences may be independently expressed from individual promoters; or may be coordinately expressed through the inclusion of an “internal ribosome entry site” or “IRES”, which is an element that promotes direct internal ribosome entry to the initiation codon, such as ATG, of a cistron (a protein encoding region), thereby leading to the cap-independent translation of the gene. IRES elements functional in yeast are described by Thompson et al. (2001) P.N.A.S. 98:12866-12868.

In one embodiment of the invention, antibody sequences are produced in combination with a secretory J chain, which provides for enhanced stability of IgA (see U.S. Pat. Nos. 5,959,177; and 5,202,422).

In a preferred embodiment the two haploid yeast strains are each auxotrophic, and require supplementation of media for growth of the haploid cells. The pair of auxotrophs are complementary, such that the diploid product will grow in the absence of the supplements required for the haploid cells. Many such genetic markers are known in yeast, including requirements for amino acids (e.g. met, lys, his, arg, etc.), nucleosides (e.g. ura3, ade1, etc.); and the like. Amino acid markers may be preferred for the methods of the invention. Alternatively diploid cells which contain the desired vectors can be selected by other means, e.g., by use of other selectable markers, such as green fluorescent protein, various dominant selectable markers, and the like.

The two transformed haploid cells may be genetically crossed and diploid strains arising from this mating event selected by their hybrid nutritional requirements. Alternatively, populations of the two transformed haploid strains are spheroplasted and fused, and diploid progeny regenerated and selected. By either method, diploid strains can be identified and selectively grown because, unlike their haploid parents, they do not have the same nutritional requirements. For example, the diploid cells may be grown in minimal medium. The diploid synthesis strategy has certain advantages. Diploid strains have the potential to produce enhanced levels of heterologous protein through broader complementation to underlying mutations, which may impact the production and/or secretion of recombinant protein.

As noted above, in some embodiments a haploid yeast may be transformed with a single or multiple vectors and mated or fused with a non-transformed cell to produce a diploid cell containing the vector or vectors. In other embodiments, a diploid yeast cell may be transformed with one or more vectors that provide for the expression and secretion of a desired humanized rabbit antibody polypeptide or polypeptides by the diploid yeast cell.

In one embodiment of the invention, two haploid strains are transformed with a library of polypeptides, e.g. a library of humanized rabbit antibody heavy or light chains produced according to the invention. Transformed haploid cells that synthesize the polypeptides are mated with the complementary haploid cells. The resulting diploid cells are screened for functional protein. The diploid cells provide a means of rapidly, conveniently and inexpensively bringing together a large number of combinations of polypeptides for functional testing. This technology is especially applicable for the generation of heterodimeric protein products, where optimized subunit synthesis levels are critical for functional protein expression and secretion.

In another embodiment of the invention, the expression level ratio of the two subunits is regulated in order to maximize product generation. Heterodimer subunit protein levels have been shown previously to impact the final product generation (Simmons L C, J Immunol Methods. 2002 May 1; 263(1-2):133-47). Regulation can be achieved prior to the mating step by selection for a marker present on the expression vector. By stably increasing the copy number of the vector, the expression level can be increased. In some cases, it may be desirable to increase the level of one chain relative to the other, so as to reach a balanced proportion between the subunits of the polypeptide. Antibiotic resistance markers are useful for this purpose, e.g. Zeocin resistance marker, G418 resistance, etc. and provide a means of enrichment for strains that contain multiple integrated copies of an expression vector in a strain by selecting for transformants that are resistant to higher levels of Zeocin or G418. The proper ratio, e.g. 1:1; 1:2; etc. of the subunit genes may be important for efficient protein production. Even when the same promoter is used to transcribe both subunits, many other factors contribute to the final level of protein expressed and therefore, it can be useful to increase the number of copies of one encoded gene relative to the other. Alternatively, diploid strains that produce higher levels of a humanized antibody polypeptide, relative to single copy vector strains, are created by mating two haploid strains, both of which have multiple copies of the expression vectors.

Host cells are transformed with the above-described expression vectors, mated to form diploid strains, and cultured in conventional nutrient media modified as appropriate for inducing promoters, selecting transformants or amplifying the genes encoding the desired sequences. A number of minimal media suitable for the growth of yeast are known in the art. Any of these media may be supplemented as necessary with salts (such as sodium chloride, calcium, magnesium, and phosphate), buffers (such as HEPES, Potassium Phosphate, Sodium Phosphate), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Secreted proteins are recovered from the culture medium. A protease inhibitor, such as phenyl methyl sulfonyl fluoride (PMSF) may be useful to inhibit proteolytic degradation during purification, and antibiotics may be included to prevent the growth of adventitious contaminants. The composition may be concentrated, filtered, dialyzed, etc., using methods known in the art.

The diploid cells of the invention are grown for production purposes. Such production purposes desirably include growth in minimal media, which media lacks pre-formed amino acids and other complex biomolecules, e.g., media comprising ammonia as a nitrogen source, and glucose as an energy and carbon source, and salts as a source of phosphate, calcium and the like. Preferably such production media lacks selective agents such as antibiotics, amino acids, purines, pyrimidines, etc. The diploid cells can be grown to high cell density, for example at least about 50 g/L; more usually at least about 100 g/L; and may be at least about 300, about 400, about 500 g/L or more.

In one embodiment of the invention, the growth of the subject cells for production purposes is performed at low temperatures, which temperatures may be lowered during log phase, during stationary phase, or both. The term “low temperature” refers to temperatures of at least about 15° C., more usually at least about 17° C., and may be about 20° C., and is usually not more than about 25° C., more usually not more than about 22° C. Growth temperature can impact the production of full-length secreted proteins in production cultures, and decreasing the culture growth temperature can strongly enhance the intact product yield. The decreased temperature appears to assist intracellular trafficking through the folding and post-translational processing pathways used by the host to generate the target product, along with reduction of cellular protease degradation.

The methods of the invention provide for expression of secreted, active protein, preferably a mammalian protein. In one embodiment, secreted, “active antibodies”, as used herein, refers to a correctly folded multimer of at least two properly paired chains, which accurately binds to its cognate antigen. Expression levels of active protein are usually at least about 10-50 mg/liter culture, more usually at least about 100 mg/liter, preferably at least about 500 mg/liter, and may be 1000 mg/liter or more.

The methods of the invention can provide for increased stability of the host and heterologous coding sequences during production. The stability is evidenced, for example, by maintenance of high levels of expression of time, where the starting level of expression is decreased by not more than about 20%, usually not more than 10%, and may be decreased by not more than about 5% over about 20 doublings, 50 doublings, 100 doublings, or more.

The strain stability also provides for maintenance of heterologous gene sequence integrity over time, where the sequence of the active coding sequence and requisite transcriptional regulatory elements are maintained in at least about 99% of the diploid cells, usually in at least about 99.9% of the diploid cells, and preferably in at least about 99.99% of the diploid cells over about 20 doublings, 50 doublings, 100 doublings, or more. Preferably, substantially all of the diploid cells maintain the sequence of the active coding sequence and requisite transcriptional regulatory elements.

A second expression vector is produced using the same conventional means well known to those of ordinary skill in the art, said expression vector containing an operon and a DNA sequence encoding an antibody light chain in which the DNA sequence encoding the CDRs required for antibody specificity is derived from a rabbit B-cell source, while the DNA sequence encoding the remaining parts of the antibody chain is derived from a human cell source.

The expression vectors are transfected into a host cell by conventional techniques well known to those of ordinary skill in the art to produce a transfected host cell, said transfected host cell cultured by conventional techniques well known to those of ordinary skill in the art to produce said antibody polypeptides.

The host cell may be co-transfected with the two expression vectors described above, the first expression vector containing DNA encoding an operon and a humanized rabbit light chain-derived polypeptide and the second vector containing DNA encoding an operon and a humanized rabbit heavy chain-derived polypeptide. The two vectors contain different selectable markers, but preferably achieve substantially equal expression of the heavy and light chain polypeptides. Alternatively, a single vector may be used, the vector including DNA encoding both the humanized rabbit heavy and light chain polypeptides.

Two transformed haploid cells may be genetically crossed and diploid strains arising from this mating event selected by their hybrid nutritional requirements and/or antibiotic resistance spectra. Alternatively, populations of the two transformed haploid strains are spheroplasted and fused, and diploid progeny regenerated and selected. By either method, diploid strains can be identified and selectively grown based on their ability to grow in different media than their parents. For example, the diploid cells may be grown in minimal medium that may include antibiotics. The diploid synthesis strategy has certain advantages. Diploid strains have the potential to produce enhanced levels of heterologous protein through broader complementation to underlying mutations, which may impact the production and/or secretion of recombinant protein. Furthermore, once stable strains have been obtained, any antibiotics used to select those strains do not necessarily need to be continuously present in the growth media.

The host cells used to express the antibody polypeptides may be either a bacterial cell such as E. coli, or a eukaryotic cell. In a particularly preferred embodiment of the invention, a mammalian cell of a well-defined type for this purpose, such as a myeloma cell or a Chinese hamster ovary (CHO) cell line may be used.

The general methods by which the vectors may be constructed, transfection methods required to produce the host cell and culturing methods required to produce the antibody polypeptides from said host cells all include conventional techniques. Although preferably the cell line used to produce the antibody is a mammalian cell line, any other suitable cell line, such as a bacterial cell line such as an E. coli-derived bacterial strain, or a yeast cell line, may alternatively be used.

Similarly, once produced the humanized rabbit antibody polypeptides may be purified according to standard procedures in the art, such as for example cross-flow filtration, ammonium sulphate precipitation, affinity column chromatography and the like.

The humanized antibody polypeptides described herein may also be used for the design and synthesis of either peptide or non-peptide mimetics that would be useful for the same therapeutic applications as the antibody polypeptides of the invention. See, for example, Saragobi et al, Science, 253:792-795 (1991), the contents of which is herein incorporated by reference in its entirety.

Administration

Humanized rabbit antibodies and fragments and fusions containing produced according to the invention are preferably used for human therapy or for diagnostic methods such as in vivo imaging of tumor sites. In one embodiment of the invention, the humanized antibodies described herein, or humanized binding fragments thereof, as well as combinations of said antibody fragments, are administered to a subject at a concentration of between about 0.05 and 10.0 mg/kg of body weight of recipient subject. In a preferred embodiment of the invention, the humanized antibodies described herein, or humanized binding fragments thereof, as well as combinations of said antibody fragments, are administered to a subject at a concentration of about 0.1-1.0 mg/kg of body weight of recipient subject.

In another embodiment of the invention, the humanized rabbit antibodies described herein, or binding fragments thereof, as well as combinations of said antibody fragments, are administered to a subject in a pharmaceutical formulation.

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can occur by means of injection, powder, liquid, gel, drops, or other means of administration.

A “pharmaceutical excipient” or a “pharmaceutically acceptable excipient” is a carrier, usually a liquid, in which an active therapeutic agent is formulated. In one embodiment of the invention, the active therapeutic agent is a humanized antibody specific to IL-6 or TNF-□, or one or more fragments thereof. The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline polypeptide can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain teachings related to methods for obtaining a clonal population of antigen-specific B cells were disclosed in U.S. Provisional patent application No. 60/801,412, filed May 19, 2006, the disclosure of which is herein incorporated by reference in its entirety.

Certain teachings related to producing antibodies or fragments thereof using mating competent yeast and corresponding methods were disclosed in U.S. patent application Ser. No. 11/429,053, filed May 8, 2006, (U.S. Patent Application Publication No. US2006/0270045), the disclosure of which is herein incorporated by reference in its entirety.

The entire disclosure of each document cited (including patents, patent applications, journal articles, abstracts, manuals, books, or other disclosures) in the Background of the Invention, Detailed Description, and Examples is herein incorporated by reference in their entireties.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLES

### Example 1

### Production of Enriched Antigen-Specific B Cell Antibody Culture

Panels of antibodies are derived by immunizing traditional antibody host animals to exploit the native immune response to a target antigen of interest. Typically, the host used for immunization is a rabbit or other host that produces antibodies using a similar maturation process and provides for a population of antigen-specific B cells producing antibodies of comparable diversity, e.g., epitopic diversity. The initial antigen immunization can be conducted using complete Freund's adjuvant (CFA), and the subsequent boosts effected with incomplete adjuvant. At about 50-60 days after immunization, preferably at day 55, antibody titers are tested, and the Antibody Selection (ABS) process is initiated if appropriate titers are established. The two key criteria for ABS initiation are potent antigen recognition and function-modifying activity in the polyclonal sera.

At the time positive antibody titers are established, animals are sacrificed and B cell sources isolated. These sources include: the spleen, lymph nodes, bone marrow, and peripheral blood mononuclear cells (PBMCs). Single cell suspensions are generated, and the cell suspensions are washed to make them compatible for low temperature long term storage. The cells are then typically frozen.

To initiate the antibody identification process, a small fraction of the frozen cell suspensions are thawed, washed, and placed in tissue culture media. These suspensions are then mixed with a biotinylated form of the antigen that was used to generate the animal immune response, and antigen-specific cells are recovered using the Miltenyi magnetic bead cell selection methodology. Specific enrichment is conducted using streptavidin beads. The enriched population is recovered and progressed in the next phase of specific B cell isolation.

### Example 2

### Production of Clonal, Antigen-Specific B Cell-Containing Culture

Enriched B cells produced according to Example 1 are then plated at varying cell densities per well in a 96 well microtiter plate. Generally, this is at 50, 100, 250, or 500 cells per well with 10 plates per group. The media is supplemented with 4% activated rabbit T cell conditioned media along with 50K frozen irradiated EL4B feeder cells. These cultures are left undisturbed for 5-7 days at which time supernatant-containing secreted antibody is collected and evaluated for target properties in a separate assay setting. The remaining supernatant is left intact, and the plate is frozen at −70° C. Under these conditions, the culture process typically results in wells containing a mixed cell population that comprises a clonal population of antigen-specific B cells, i.e., a single well will only contain a single monoclonal antibody specific to the desired antigen.

### Example 3

### Screening of Antibody Supernatants for Monoclonal Antibody of Desired Specificity and/or Functional Properties

Antibody-containing supernatants derived from the well containing a clonal antigen-specific B cell population produced according to Example 2 are initially screened for antigen recognition using ELISA methods. This includes selective antigen immobilization (e.g., biotinylated antigen capture by streptavidin coated plate), non-specific antigen plate coating, or alternatively, through an antigen build-up strategy (e.g., selective antigen capture followed by binding partner addition to generate a heteromeric protein-antigen complex). Antigen-positive well supernatants are then optionally tested in a function-modifying assay that is strictly dependant on the ligand. One such example is an in vitro protein-protein interaction assay that recreates the natural interaction of the antigen ligand with recombinant receptor protein. Alternatively, a cell-based response that is ligand dependent and easily monitored (e.g., proliferation response) is utilized. Supernatant that displays significant antigen recognition and potency is deemed a positive well. Cells derived from the original positive well are then transitioned to the antibody recovery phase.

### Example 4

### Recovery of Single, Antibody-Producing B Cell of Desired Antigen Specificity

A few number of cells are isolated from a well that contains a clonal population of antigen-specific B cells (produced according to Example 2 or 3), which secrete a single antibody sequence. The isolated cells are then assayed to isolate a single, antibody-secreting cell. Dynal streptavidin beads are coated with biotinylated target antigen under buffered medium to prepare antigen-containing microbeads compatible with cell viability. Next antigen-loaded beads, antibody-producing cells from the positive well, and a fluorescein isothiocyanate (FITC)-labeled anti-host H&L IgG antibody (as noted, the host can be any mammalian host, e.g., rabbit, mouse, rat, etc.) are incubated together at 37° C. This mixture is then re-pipetted in aliquots onto a glass slide such that each aliquot has on average a single, antibody-producing B-cell. The antigen-specific, antibody-secreting cells are then detected through fluorescence microscopy. Secreted antibody is locally concentrated onto the adjacent beads due to the bound antigen and provides localization information based on the strong fluorescent signal. Antibody-secreting cells are identified via FITC detection of antibody-antigen complexes formed adjacent to the secreting cell. The single cell found in the center of this complex is then recovered using a micromanipulator. The cell is snap-frozen in an eppendorf PCR tube for storage at −80° C. until antibody sequence recovery is initiated.

### Example 5

### Isolation of Antibody Sequences From Antigen-Specific B Cell

Antibody sequences are recovered using a combined RT-PCR based method from a single isolated B-cell produced according to Example 4 or an antigenic specific B cell isolated from the clonal B cell population obtained according to Example 2. Primers are designed to anneal in conserved and constant regions of the target immunoglobulin genes (heavy and light), such as rabbit immunoglobulin sequences, and a two-step nested PCR recovery step is used to obtain the antibody sequence. Amplicons from each well are analyzed for recovery and size integrity. The resulting fragments are then digested with AluI to fingerprint the sequence clonality. Identical sequences display a common fragmentation pattern in their electrophoretic analysis. Significantly, this common fragmentation pattern which proves cell clonality is generally observed even in the wells originally plated up to 1000 cells/well. The original heavy and light chain amplicon fragments are then restriction enzyme digested with HindIII and XhoI or HindIII and BsiwI to prepare the respective pieces of DNA for cloning. The resulting digestions are then ligated into an expression vector and transformed into bacteria for plasmid propagation and production. Colonies are selected for sequence characterization.

### Example 6

### Recombinant Production of Monoclonal Antibody of Desired

Antigen Specificity and/or Functional Properties Correct full-length antibody sequences for each well containing a single monoclonal antibody is established and miniprep DNA is prepared using Qiagen solid-phase methodology. This DNA is then used to transfect mammalian cells to produce recombinant full-length antibody. Crude antibody product is tested for antigen recognition and functional properties to confirm the original characteristics are found in the recombinant antibody protein. Where appropriate, large-scale transient mammalian transfections are completed, and antibody is purified through Protein A affinity chromatography. Kd is assessed using standard methods (e.g., Biacore) as well as IC50 in a potency assay.

### Example 7

### Preparation of Antibodies that Bind a Desired Antigen Such as HuTNF-α or IL-6

By using the antibody selection protocol described herein, one can generate a collection of antibodies that exhibit potent functional antagonism of TNF-α or to IL-6 or another desired antigen. The antibodies elucidate a variety of epitopes and thus may provide useful alternatives to, or adjunctives with, antibodies that target previously identified epitopes for the particular antigen such as in the case of Hu-TNF-alpha, TNF-α epitopes, such as Remicade® (infliximab).

In the specific case of either IL-6 or TNF-α, a screening method can be employed to identify antibodies that bind alternative IL-6 or TNF-α epitopes, while retaining significant functional antagonism. For example, in the case of TNF-α after the primary antigen-recognition screen, positive BCC wells can be tested for functional antagonism towards TNF-α as well as for epitope competition, e.g., competition with infliximab. Unique epitope recognition can be established by ForteBio Octet antibody-TNF-α binding competition studies. BCC wells that display functional activity as well as lack of competition are pursued, and the coding sequences for the antibody present in these wells recovered. The majority of the recovered sequences will display the original target characteristics: potent antigen recognition, functional antagonism, and distinct epitope recognition. Thus, the resulting antibody collection establishes multiple novel epitope regions associated with potent functional antagonism. Similar results are demonstrated with IL-6 in the provisional application incorporated by reference herein.

Immunization Strategy:

Rabbits can be immunized with TNF-α (R&D # 210-TA) and to human IL-6 as described in the provisional patent applications U.S. Ser. No. 60/924,551 and 60/924,551 filed on May 21, 2007 and incorporated by reference in their entireties herein.

Antibody Selection Titer Assessment

Antigen recognition assay can be determined for TNF-α or to human IL-6 by the protocol described therein.

Functional Titer Assessment

The functional activities of the samples can be determined as described in the cited provisional applications. For example, in the case of TNF-α separately, in a round-bottom 96-well plate, serum samples were added at a 1:100 dilution (in the described media) followed by 1:10 dilution across the plate (columns 2-10, column 11 was media only for TNF-α control), 50 μl/well in replicates of 5 (rows B-F, row G was media only for background control). 50 μl/well of media containing TNF-α at a concentration 4 times the final EC50 (concentration was previously determined for each lot) and 1 μg/ml of Actinomycin D was added to all sample wells except row F. Plates were incubated for 1 h at 37° C.

At 1 h, 50 μl of the Serum/Ag complex and controls are transferred to the 96-well flat-bottom plates containing 50 μl/well of responder cells at a fixed density (final volume: 100 μl/well) and incubated for 24 h at 37° C. (Columns 1 and 12 and rows A and H are filled with 200 μl of media to prevent evaporation and cause edge effect.)

At 24 h, 201/well of CellTiter96 reagent (Promega) is added to all test wells per the manufacturer protocol, and plates were incubated for 2 h at 37° C. After 2 h, plates are gently shaken to allow homogeneity in the test wells. Plates were read at 490 nm wavelength. OD versus dilution were plotted using Graph Pad Prizm (non-linear sigmoid dose/response curve was used), and functional titer was determined.

Tissue Harvesting

Rabbit spleen, lymph nodes, and whole blood were harvested, processed, and frozen as described in the provisional applications cited above.

B Cell Culture (BCC)

B cell cultures are prepared as described in the incorporated by reference provisional patent applications.

Antigen Recognition Screening

Antigen recognition screening was performed as described above as single points.

Functional Activity Screening

Functional activity screening is performed as described in the cited provisional applications. For example, in the case of TNF-alpha it is determined by a WEHI cytotoxic assay. Supernatant from master plate(s) was tested in the TNF-α stimulated WEHI cytotoxic assay (as described above) as single points. Supernatants were tested as neat as described therein.

Secondary Functional Activity Assay for Recombinant Antibodies: Blocking of IL-6 Expression by HUVEC Cells Treated with huTNF-α

TNF-α or IL-6 specific assays can be effected as described in the same provisional patent applications incorporated by reference herein. For example in the case of TNF-α human umbilical vein endothelial cells (HUVECs) are routinely maintained in endothelial growth medium (EGM) medium and appropriate HUVEC supplements (Cambrex). On the day of the assay, HUVEC viability is determined by trypan blue. The cells are resuspended at 5E05/ml in the appropriate volume of medium necessary for the assay (100 μl/well). Cells were plated in middle wells of 96-well flat-bottom culture plates, and 200 μl medium was added to all outside wells to prevent evaporation. The plate was incubated for 24 h at 37° C.

At 24 h, the appropriate antibody dilutions are made in EGM at 4 times the desired final concentration. (Starting antibody concentration was 1 μg/ml; a 1:3 dilution was performed across the plate, except for last row.) The same volume of rhuTNF-α in EGM (4 times the desired final concentration) was added to the wells. The plate was incubated for 1 h at 37° C. to form the antibody/antigen complex. At 1 h, 50 μl of media from the HUVEC culture plate was removed and discarded. 50 μl Ab-Ag mixture was added, and the plate was incubated for 48 h at 37° C. Standard positive and negative controls were included:

At 48 h, conditioned medium IL-6 levels were assessed by ELISA. An Immulon plate was coated with 1 μg/ml goat anti-huIL-6 at 50 μl/well, overnight at 4° C., or room temperature for 1 hour. The plate was washed in PBS+0.5% Tween 20 in a plate washer (200 μl/well; 3 times). The plate was blocked with 200 μl/well FSG for 1 hour at room temperature. The blocking solution was aspirated, and the plate was blotted. The huIL-6 standard was set, starting at 1 μg/ml and diluted 1:3 across the plate (all dilutions made in FSG). Samples from HUVEC culture were added to the wells below standard curve and incubated for 1 hour at room temperature. Wash was repeated. 1 μg/ml of a humanized antibody (anti-huIL-6) was added at 50 μl/well to the plate and incubated for 1 hour at room temperature. Wash was repeated. Secondary anti-human IgG Fc HRP at 1:5000 dilution was added at 50 μl/well and incubated for 45 minutes at room temperature. Wash was repeated. Assay was developed with 50 μl/well 3,3′,5,5′ tetramethylbenzidine (TMB) for a minimum of 5 minutes. The reaction was stopped with 50 μl/well HCl, and the plate was read at 450 nm in a plate reader. Data was analyzed using Graph Pad Prizm.

B Cell Recovery

The foci protocol for huIL6 and for huTNF-α are performed as described in the above-cited provisional applications.

### Example 8

### Preparation of Exemplary Humanized Rabbit Antibody (Specific to IL-6) According to the Invention

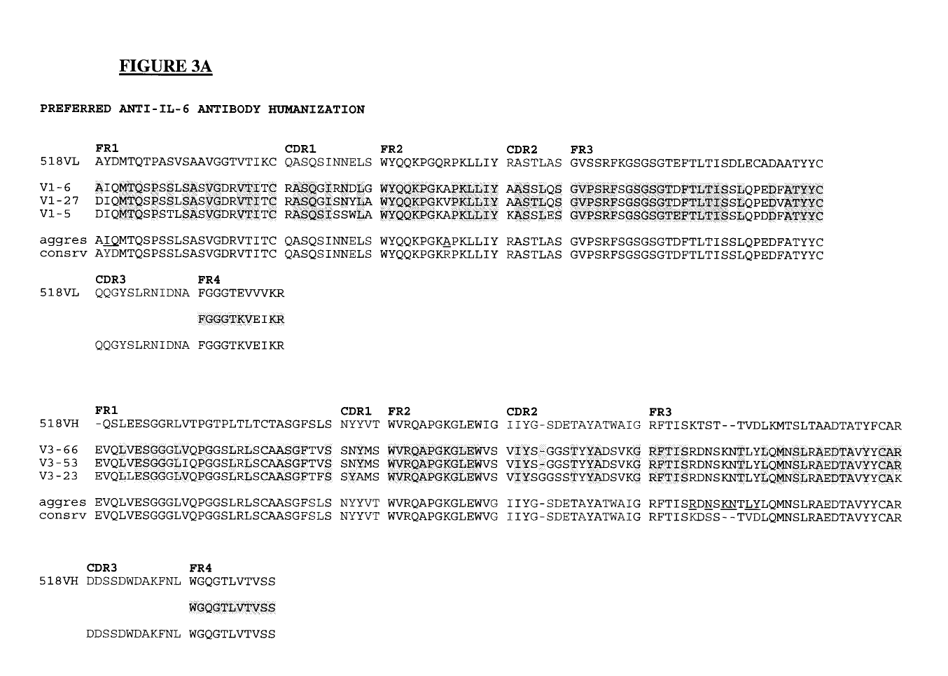

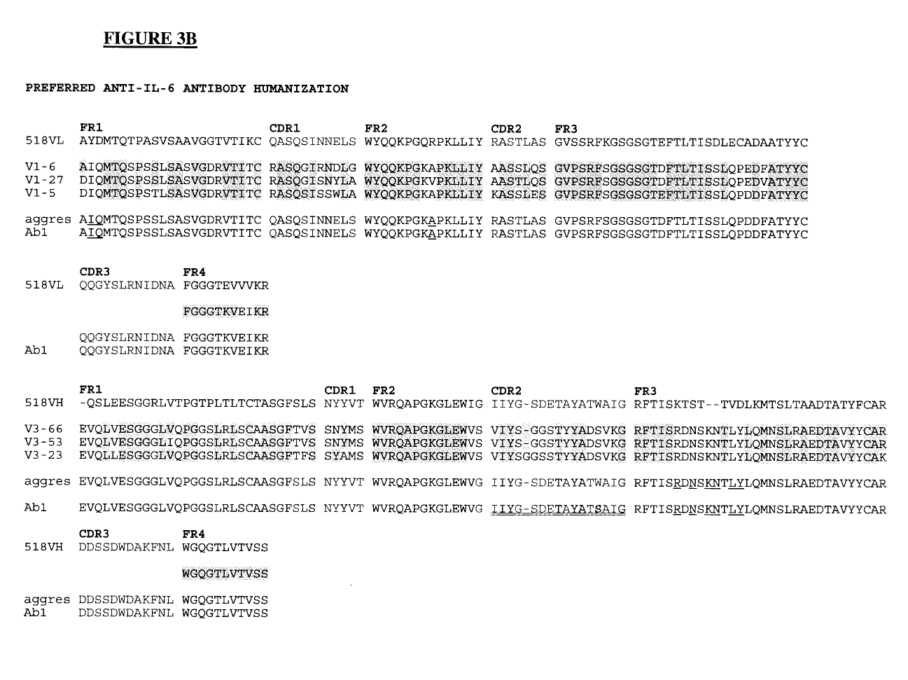

Heavy and light Chains derived from a rabbit anti-huIL-6 antibody produced as described in the incorporated by reference provisional patent applications and according to the foregoing examples were humanized using the humanization strategy described herein and depicted schematically in FIG. 1. The variable light chain region of an exemplary rabbit anti-IL-6 antibody (a region containing from FR1 through the terminus of FR3 of this IL-6 specific antibody) was screened against a library of human germline sequences using BLAST and identified three germline sequences having significant homology thereto, i.e., V1-6, V1-27 and V1-5 relative to the other human germline sequences in this library. The germline sequence V1-6 was found to exhibit the greatest sequence identity to the rabbit light chain variable sequence and therefore was selected as the starting material to produce two humanized light chain versions designated as “aggres” and “consrv” in FIG. 3A and an additional humanized light chain version in FIG. 3B These sequences were derived essentially by modifying the V1-6 human germline sequence with specific selectivity determining residues from the rabbit parent anti-IL-6 CDR1 and CDR2 regions as shown in the top half of FIG. 3A and by further incorporating few (consrv version) or no donor (rabbit) FR residues (aggres version). Particularly, as shown in FIG. 3A one humanized light chain was produced (referred to as “aggres” in the Figure) wherein no rabbit FR residues were incorporated and by fusion of the V1-6 sequence to the rabbit light chain CDR3 and a human FR4 sequence homologous to the rabbit light chain FR4 sequence. FR4. Another version referred to as “consrv” depicted in the same Figure was produced containing 2 FR residues from the rabbit light chain FR1. The light chain sequence shown in FIG. 3B was derived by the same procedure as that shown in FIG. 3A, except that a different human residue was chosen at one position in FR3, specifically at the eighth amino acid from the terminus of FR3, which is glutamic acid in the FIG. 3A sequences and aspartic acid in the FIG. 3B sequences. Note that in FIG. 3B, the light chain sequences shown with the labels “aggres” and “Ab1” are identical.

Using similar humanization methods and as depicted schematically in FIG. 1 the variable region of a preferred anti-IL-6 antibody containing CDR1 and CDR2 and associated FR regions was used to screen using BLAST methods against a library of human germline sequences in order to identify the human germline sequences most homologous thereto. As shown in the Figure this screening identified three homologous human germline sequences, V3-66, V3-53, and V3-23 containing the sequences in FIG. 2. The most homologous human germline sequence V3-23 was again used as a starting material to produce 2 humanized versions similarly modified by the incorporation of specific selectivity determining residues of the CDR1 and CDR2 regions of the heavy chain in favor of the corresponding human CDR1 and CDR2 residues, the further incorporation of a few discrete rabbit FR residues and fusion to the rabbit CDR3 region and a homologous human FR4 region. The resultant 2 humanized heavy chains again referred to as “aggres” and “consrv” are shown in the bottom of FIG. 3A. Based on the aligned sequences it can be seen that these humanized versions differ only in the presence of several rabbit FR3 residues in the “consrv” version which are not present in “aggres”. Both of these sequences vary at only a relatively few number of residues in comparison to human germline sequences and therefore should be substantially non-immunogenic in humans. FIG. 3B shows a heavy-chain sequence identical to that in FIG. 3A, together with a sequence labeled “Ab1” whose amino acid sequence differs from the “aggres” sequence by a single substitution within CDR2, specifically by replacement of the fourth amino acid from the terminus of the rabbit heavy chain CDR2 (tryptophan) with a corresponding human CDR2 residue (serine).

Humanized anti-IL-6 antibodies containing either the “aggres” humanized heavy and light chains and the “consrv” were found to possess IL-6 binding affinities very approximate to the parent rabbit antibody. This validates the efficacy of the inventive humanization strategies and further suggests that these methods may be used to produce humanized antibodies to different antigens having sequences which are very “human-like” which should be substantially non-immunogenic in human subjects.

### Example 9

### Retained Affinity Properties of Exemplary Humanized Rabbit Antibodies (Specific to hIL-6 and hTNF-alpha) Produced According to the Invention

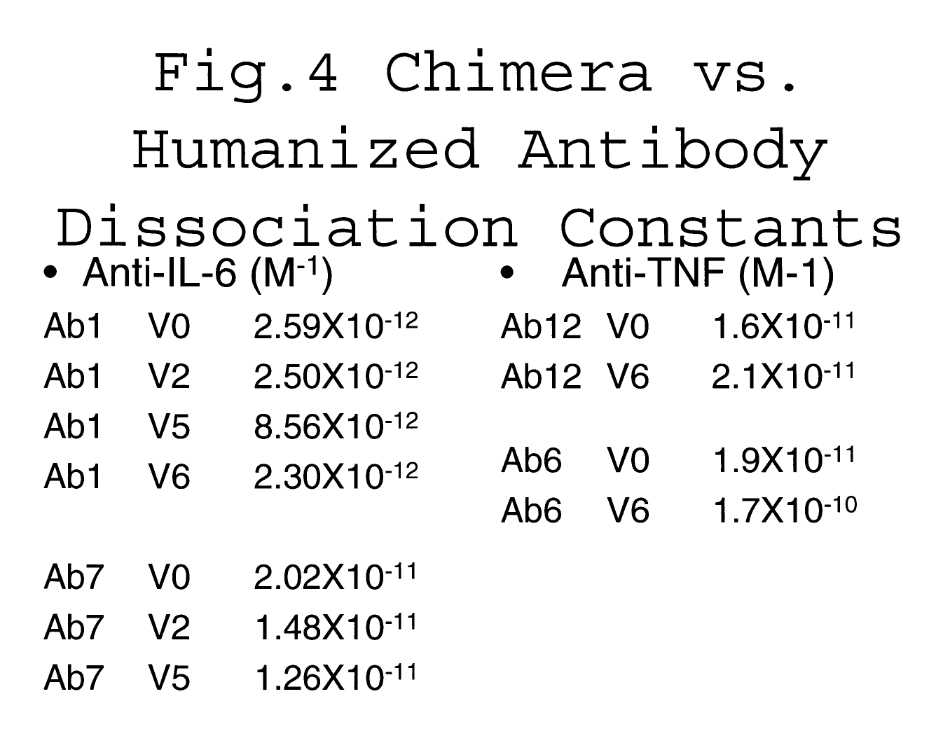

As discussed infra, a significant advantage of the present invention is that the subject humanization methods reproducibly gives rise to humanized antibodies possessing high affinities, i.e., the binding affinity is comparable to that of the parent rabbit or chimeric antibody derived therefrom. This is illustrated by the dissociation constants contained in FIG. 4. Therein, the dissociation constants of 2 different rabbit chimeric anti-hIL-6 antibodies are respectively compared to 3 and 2 different humanized antibodies derived therefrom which were all produced using the inventive methods. From the data contained in this Figure it can be seen that the dissociation constants are in most instances roughly unchanged from the chimeric to the humanized variant derived therefrom. In the worst instance the dissociation constant is reduced by roughly 3.5 fold. This is contrast to other humanization methods which typically result in substantial loss of antigen binding affinity, i.e., an order of magnitude or more from the parent relative to the humanized version.

In addition, the same FIG. 4 contains data comparing the dissociation constants of two different chimeric rabbit anti-hTNF-alpha antibodies to a humanized antibody derived therefrom using the inventive humanization methodologies. Similarly, the dissociation constants of the parent rabbit derived chimeric anti-hTNF-alpha antibody and the humanized antibodies are substantially the same. These results illustrate the reproducibility of the subject humanization methods, namely their broad applicability for humanizing different rabbit antibody sequences and for humanizing antibodies specific to different antigens.

### Example 10

### Retained Functional Properties of Exemplary Humanized Rabbit Antibodies (Specific to hIL-6 and hTNF-alpha) Produced According to the Invention

As shown in the prior example, the humanized antibodies produced according to the invention possess antigen binding constants comparable to the parent rabbit antibodies from which they are derived. Based thereon, it was predicted that the antagonistic properties of the parent chimeric antibody and the humanized variants derived therefrom would likewise be comparable. In fact these inventors' expectations have been confirmed.

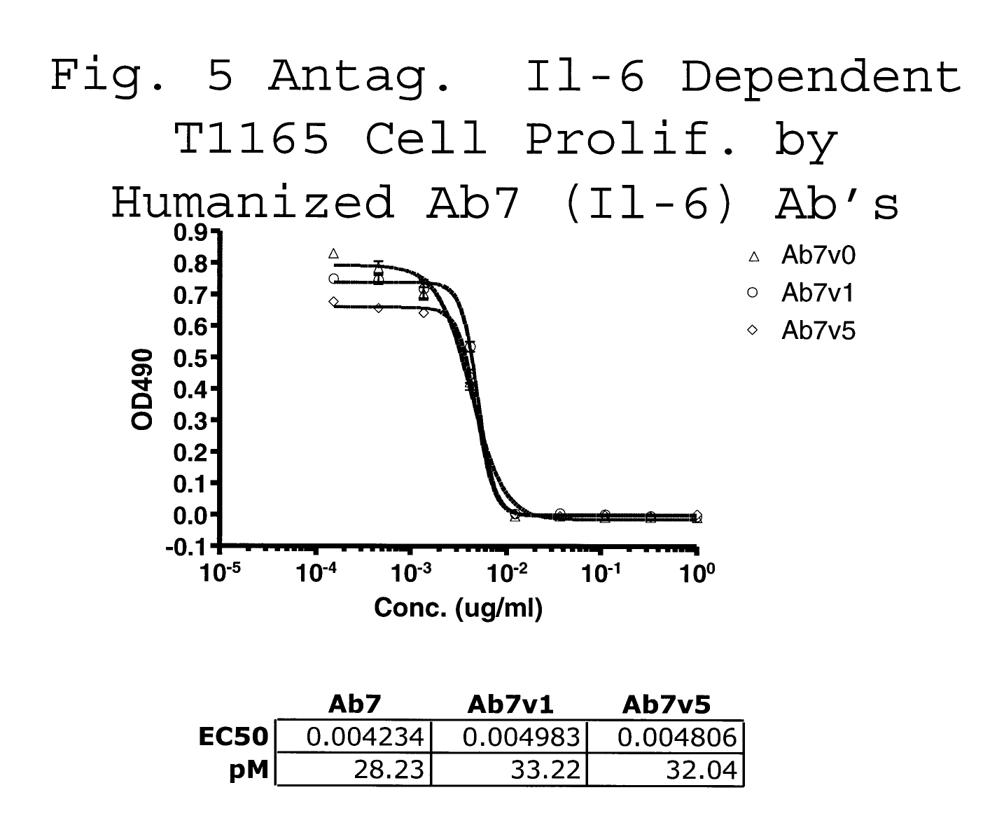

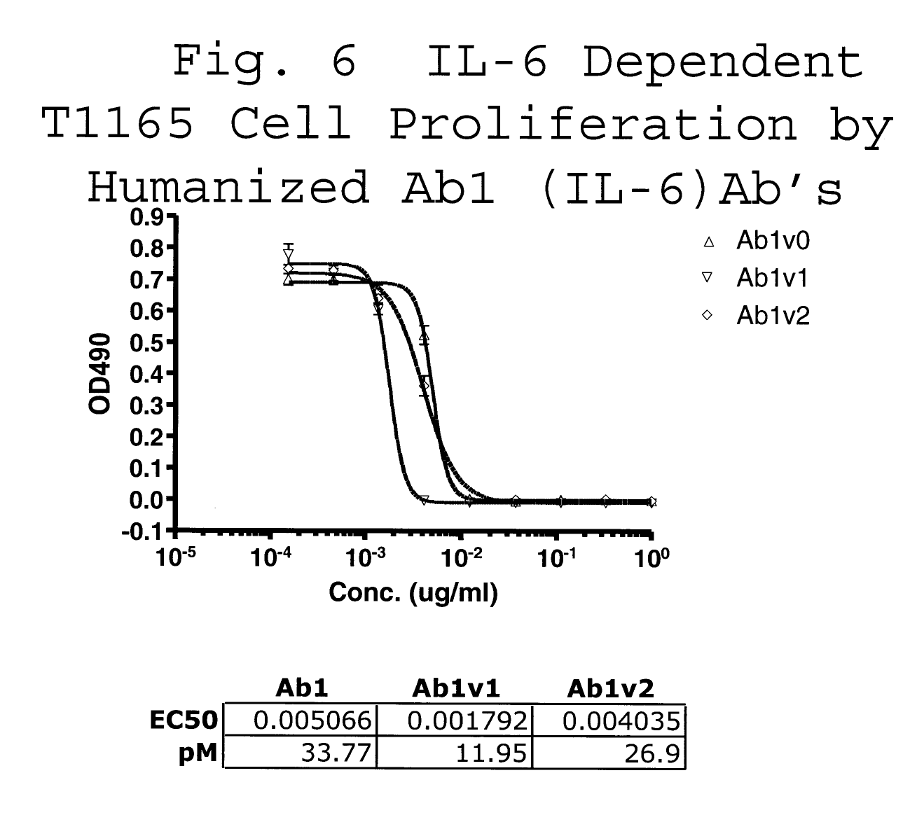

As shown in FIGS. 5 and 6 the inventors compared the antagonistic properties of two different chimeric antibodies derived from rabbit anti-IL-6 antibodies respectively to 2 different humanized antibodies derived from each. Antagonism was compared in an assay that detected the effect of these anti-hIL-6 antibodies on hIL-6 dependent cell proliferation, an accepted functional assay for detecting antagonistic activity. It can be seen from the data in FIGS. 5 and 6 that the inhibition of hIL-6 dependent cell proliferation for the chimeric and the humanized antibodies derived therefrom are substantially identical. (The cell proliferation data curves are substantially overlapping or very similar at different antibody concentrations.)

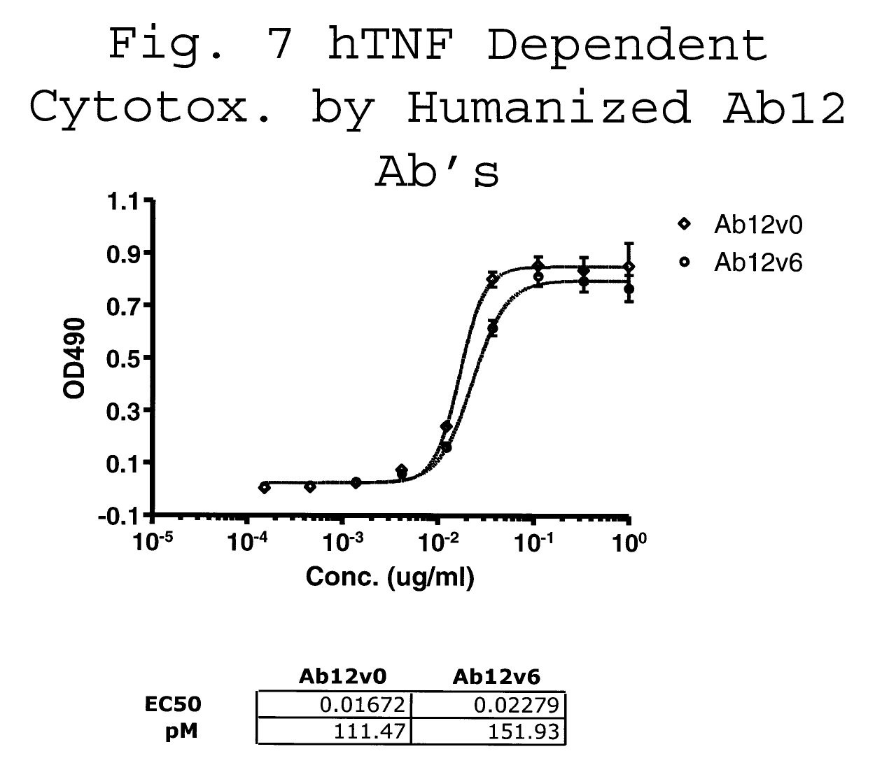

Moreover, as shown in FIG. 7 the inventors compared the antagonistic properties of a chimeric antibody specific to hTNF-alpha derived from a rabbit anti-hTNF-alpha antibody to a humanized antibody derived therefrom which was produced using the subject humanization methodologies. Antagonism was compared in an assay that detected the effect of these anti-hTNF-alpha antibodies on hTNF-alpha dependent cytotoxicity, an accepted functional assay for detecting anti-hTNF-alpha antibody antagonistic activity. It can be seen from the data in FIG. 7 that the inhibition of hTNF-alpha dependent cytotoxicity for the chimeric and the humanized anti-hTNF-alpha antibody derived therefrom are very similar. (The cytotoxicity data curves are substantially overlapping or very similar at different antibody concentrations.)

These examples are intended to be exemplary of the present invention and its intrinsic advantages. In fact the present humanization methods may be used to humanize any rabbit antibody (or that of a closely related species) having specificity to any desired antigen. Preferably these antibodies will be specific to a target antigen suitable for human therapy and possess high affinity to this target antigen. For example such antibodies may include in particular any of the rabbit antibody heavy and light chain sequences disclosed in U.S. Ser. No. 60/924,550 and 60/924,551 and the PCT applications filed on May 21, 2008 respectively having attorney docket number 67858.901902 and 67858.701802 entitled IL-6 Antibodies and Use Thereof and Anti-TNF Antibodies which provisional and PCT applications are incorporated by reference in their entirety herein including all the antibody sequences reported therein. In addition, in order to further describe and exemplify the claimed humanization methods and humanized antibody products obtainable thereby the Sequence Listings for both of these PCT applications precede the claims herein. These Sequence Listings contain rabbit antibody sequences and humanized versions which are specific to IL-6 and TNF-alpha produced according to the inventive methods.

Further, the inventive humanization protocols may be used to humanize any available rabbit heavy or light chain sequence, i.e. to any desired antigen such as peptides, proteins, glycoproteins, haptens, carbohydrates, et al. Preferrably the antigen is a human antigen or an antigen from an agent that infects or causes or correlates to a disease in humans. Preferably, the rabbit antibodies will be derived from rabbit B cells isolated by the afore-described ABS screening protocols.

1. A humanized antibody or antibody fragment containing at least one heavy and light chain polypeptide wherein the light chain polypeptide is a humanized light chain polypeptide which contains at least the following (i) the amino acid residues spanning the first residue of FR1 through the terminus of FR3 including the CDR1 and CDR2 regions of a human light chain germline sequence that is selected from a library of human germline sequences based on its greater homology (percent sequence identity) of the selected amino acid residues spanning FR1 through FR3 (relative to other human germline sequences in the library) to the corresponding amino acid residues of the light chain of a parent rabbit antibody having specificity to a desired antigen that is to be humanized and (ii) further wherein the CDR residues in CDR1 and CDR2 corresponding to “selectivity determining residues” in the light chain of the same parent rabbit antibody are replaced with the corresponding rabbit selectivity determining residues; (iii) the amino acid residues encompassing the entire CDR3 region of the same parent rabbit antibody; (iv) the amino acid residues encompassing the entire FR4 region of an antibody light chain derived from a library of human germline sequences based on its greater homology (sequence identity) to the corresponding FR4 region contained in the light chain of the same parent rabbit antibody; and (v) wherein few or none of the FR residues of the human FR1, FR2, FR3 and FR4 regions in the selected homologous human FR regions are substituted with the corresponding rabbit FR residues.

2. The humanized antibody claim 1 wherein the parent rabbit antibody is specific to a human, viral or bacterial antigen.

3. The humanized antibody of claim 2 wherein the human antigen is a cytokine, growth factor, hormone or cancer antigen.

4. The humanized antibody of claim 1 which is specific to IL-6, hepcidin, hepatocyte growth factor or a TNF polypeptide.

5. A nucleic acid sequence encoding the humanized antibody light chain contained in the humanized antibody recited in claim 1.

6. A vector containing a nucleic acid sequence according to claim 5.

7. A cell containing a vector according to claim 6.

8. The cell of claim 7 which is selected from yeast, bacteria and mammalian cells.

9. The cell of claim 8 which is a diploidal yeast cell.

10. The cell of claim 9 which is a Pichia or other methanol utilizing diploid yeast.

11. A humanized antibody or antibody fragment containing at least one heavy chain and light chain polypeptide wherein the heavy chain is a humanized heavy chain polypeptide which contains at least the following (i) the amino acid residues spanning the first residue of FR1 through the terminus of FR3 including the CDR1 and CDR2 regions encoded by a human germline sequence that is selected from a library of human germline sequences based on its greater homology (percent sequence identity) of the selected amino acid residues spanning FR1 through FR3 (relative to other human germline sequences in the library) to the corresponding amino acid residues of the heavy chain of a parent rabbit antibody having specificity to a desired antigen that is to be humanized and (ii) further wherein the CDR residues in the CDR1 and CDR2 regions of the human heavy chain corresponding to “selectivity determining residues” in the CDR1 and CDR2 regions of the heavy chain of the same parent rabbit antibody are replaced with the corresponding heavy chain selectivity determining residues contained in the CDR1 and CDR2 regions of the rabbit heavy chain; (iii) the amino acid residues encompassing the entire CDR3 region of the same parent rabbit antibody; (iv) the FR4 region derived from a library of human germline sequences based on its greater homology (sequence identity) to the corresponding FR4 region contained in the heavy chain of the same parent rabbit antibody; and (v) wherein the final 1-3 amino acids of the human heavy FR1 region are optionally replaced with the terminal 1-3 amino acids of the corresponding rabbit heavy chain FR1 residues; and/or the terminal amino acid of the human heavy chain framework 2 region is optionally replaced with the corresponding terminal amino acid residue of the rabbit heavy chain framework 2; and/or the fourth amino acid from the terminus of the rabbit heavy chain CDR2 (typically a tryptophan) is optionally replaced with the corresponding human CDR2 residue (typically a serine); and (vi) wherein few or none of the remaining FR residues of the selected homologous human FR regions are substituted with the corresponding rabbit FR residues.

12. The humanized antibody of claim 11 wherein the parent rabbit antibody is specific to a human, viral or bacterial antigen.

13. The humanized antibody of claim 12 wherein the human antigen is a cytokine, growth factor, hormone or cancer antigen.

14. The humanized antibody of claim 11 which is specific to IL-6, hepcidin, hepatocyte growth factor or a TNF polypeptide.

15. A nucleic acid sequence encoding the humanized antibody heavy chain contained in the humanized antibody claim 11.

16. A vector containing a nucleic acid sequence according to claim 15.

17. A cell containing a vector according to claim 16.

18. The cell of claim 17 which is selected from yeast, bacteria and mammalian cells.

19. The cell of claim 18 which is a diploidal yeast cell.

20. The cell of claim 19 which is a Pichia or other methanol utilizing diploid yeast.

21. The humanized antibody of claim 1 containing at least one humanized light chain polypeptide and further comprising at least one heavy chain polypeptide wherein the at least one heavy chain is a humanized heavy chain polypeptide which contains at least the following (i) the amino acid residues spanning the first residue of FR1 through the terminus of FR3 including the CDR1 and CDR2 regions encoded by a human germline sequence that is selected from a library of human germline sequences based on its greater homology (percent sequence identity) of the selected amino acid residues spanning FR1 through FR3 (relative to other human germline sequences in the library) to the corresponding amino acid residues of the heavy chain of a parent rabbit antibody having specificity to a desired antigen that is to be humanized and (ii) further wherein the CDR residues in the CDR1 and CDR2 regions of the human heavy chain corresponding to “selectivity determining residues” in the CDR1 and CDR2 regions of the heavy chain of the same parent rabbit antibody are replaced with the corresponding heavy chain selectivity determining residues contained in the CDR1 and CDR2 regions of the rabbit heavy chain; (iii) the amino acid residues encompassing the entire CDR3 region of the same parent rabbit antibody; (iv) the FR4 region derived from a library of human germline sequences based on its greater homology (sequence identity) to the corresponding FR4 region contained in the heavy chain of the same parent rabbit antibody; and (v) wherein the final 1-3 amino acids of the human heavy FR1 region are optionally replaced with the terminal 1-3 amino acids of the corresponding rabbit heavy chain FR1 residues; and/or the terminal amino acid of the human heavy chain framework 2 region is optionally replaced with the corresponding terminal amino acid residue of the rabbit heavy chain framework 2; and/or the fourth amino acid from the terminus of the rabbit heavy chain CDR2 (typically a tryptophan) is optionally replaced with the corresponding human CDR2 residue (typically a serine); and (vi) wherein few or none of the remaining FR residues of the selected homologous human FR regions are substituted with the corresponding rabbit FR residues.

22. The humanized antibody of claim 21 which is specific to IL-6, hepcidin, hepatocyte growth factor or a TNF polypeptide.

23. A humanization strategy for producing a humanized light chain antibody sequence comprising the following steps:
(i) obtaining a DNA encoding rabbit light chain antibody sequence from a rabbit antibody that specifically binds to a desired antigen and identifying the amino residues spanning the beginning of Framework 1 (FR1) to the end of Framework 3 (FR3) inclusive;
(ii) conducting a homology search using said rabbit light antibody amino acid sequence spanning the beginning of FR1 to the end of FR3 sequence against a library containing human light chain antibody sequences and identifying a human light chain antibody sequence that exhibits substantial sequence homology thereto relative to other human germline antibody light chain sequences;
(iii) identifying in both the rabbit and human light chain sequences the arrangement and the specific residues thereof that correspond to FR1. FR2, FR3, CDR1, CDR2 regions and aligning these discrete regions in the rabbit and selected human antibody light chain;
(iv) constructing a DNA or amino acid sequence wherein the CDR1 and CDR2 regions of the selected homologous human light chain sequence are substituted by the corresponding selectivity determining residues contained in the CDR1 and CDR2 regions of the rabbit light chain sequence;
(v) further attaching to the DNA or amino acid sequence obtained by step (iv) a DNA sequence encoding or polypeptide containing the corresponding amino acid residues of the rabbit CDR3 light chain antibody sequence;
(vi) further selecting a human light chain framework 4 region (FR4) that is homologous to the FR4 contained in the rabbit light chain and which preferably differs therefrom by at most 2-4 amino acid residues and attaching a DNA sequence encoding said human FR4 or the corresponding amino residues of said human FR4 onto the DNA or amino acid sequence obtained after step (v); and
(vii) synthesizing a DNA or amino acid sequence encoding or containing the humanized rabbit light chain sequence that results from steps (i) through (vi).

24. The humanization strategy of claim 23 wherein the amino acid initiating FR1 is the first amino acid after the rabbit light chain signal sequence.

25. The humanization strategy of claim 23 wherein the signal sequence comprises about 20-22 amino acid residues.

26. The humanization strategy of claim 23 wherein the human light chain sequence is identified from a library containing human germline variable light chain sequences.

27. The humanization strategy of claim 23 wherein the FR1, FR2, FR3 and CDR1 and CDR2 regions in the rabbit sequence are identified by aligning the rabbit FR1, FR2, FR3 and CDR1 and CDR2 regions with the corresponding human light chain FR1, FR2, FR3, CDR1 and CDR2 regions.

28. The humanization strategy of claim 23 wherein the rabbit CDR3 region comprises from 9 to 15 amino acid residues.

29. The humanization strategy of claim 23 wherein the rabbit light chain FR4 region comprises 11 amino acid residues.

30. The humanization strategy of claim 23 wherein FR3 ends with YYC.

31. The humanization strategy of claim 23 wherein the FR4 in the rabbit light chain starts with FGGGG.

32. The humanization strategy of claim 31 wherein said rabbit FR4 region starts with a VVKR amino acid sequence.

33. The humanization strategy of claim 23 wherein the selected human FR4 light chain sequence comprises FGGGTKVEIKR.

34. The humanization strategy of claim 23 wherein the resultant humanized rabbit light chain is used in the manufacture of a humanized antibody or humanized antibody fragment that binds a desired antigen.

35. A humanized rabbit light chain variable amino acid sequence or a DNA encoding produced according to claim 23.

36. The humanized rabbit light chain variable amino acid sequence or DNA sequence of claim 35 which is specific to an antigen selected from a microbial antigen, a human antigen, viral antigen, and an allergen.

37. The humanized rabbit light chain variable amino acid or DNA sequence of claim 36 wherein the human antigen is selected from a human autoantigen, cytokine, receptor protein, enzyme, hormone, receptor ligand, steroid, growth factor and an oncogene.

38. An antibody or antibody fragment containing a humanized rabbit light chain variable sequence produced according to claim 23.

39. The humanized rabbit light chain or antibody containing produced according to claim 23 which is attached to an effector moiety.

40. The humanized rabbit light chain polypeptide of claim 39 wherein the effector moiety is selected from a drug, a toxin, an enzyme, a radionuclide, a fluorophore, a cytokine, an affinity label, and a translocating polypeptide.

41. The humanized rabbit light chain polypeptide or antibody containing or a DNA encoding produced according to claim 23 which is derived from a rabbit antibody that specifically binds a cytokine, growth factor or a tumor specific polypeptide.

42. The humanized rabbit light chain polypeptide or antibody containing of claim 41 that is derived from a rabbit antibody that specifically binds IL-6, TNF, VEGF, IL-12, Hepcidin or Hepatocyte growth factor.

43. A humanization strategy for producing a humanized heavy chain antibody sequence from a rabbit heavy chain antibody sequence comprising the following steps:
(i) obtaining a rabbit heavy chain antibody sequence from an rabbit antibody that specifically binds to a desired antigen and identifying the amino residues spanning the beginning of Framework 1 (FR1) to the end of Framework 3 (FR3) inclusive;
(ii) conducting a homology search (e.g., by BLAST searching of human germline antibody sequence containing libraries) using said rabbit heavy antibody amino acid sequence spanning the beginning of FR1 to the end of FR3 sequence and identifying a human heavy chain antibody sequence that is homologous thereto, i.e. which preferably possesses at least 80%-90% identical thereto at the amino acid level;
(iii) identifying in both the rabbit and human heavy chain sequences the arrangement of and the specific residues thereof that correspond to FR1, FR2, FR3, CDR1, CDR2 regions and aligning these discrete regions of the rabbit against the corresponding regions of the selected homologous human antibody heavy chain;
(iv) constructing a DNA or amino acid sequence wherein the residues in the CDR1 and CDR2 regions of the selected homologous human heavy chain sequence are substituted by the selectivity determining residues contained in the corresponding CDR1 and CDR2 regions of the rabbit heavy chain sequence and optionally replacing the terminal 1-3 amino acids of the human heavy FR1 region with the corresponding terminal 1-3 amino acids of the rabbit heavy chain FR1; and/or optionally replacing the terminal amino acid of the human heavy chain framework 2 region with the corresponding terminal amino acid residue of the rabbit heavy chain framework 2 and/or optionally replacing the fourth amino acid from the terminus of the rabbit heavy chain CDR2 (typically a tryptophan) with the corresponding human CDR2 residue (typically a serine);
(v) further attaching to the DNA or amino acid sequence obtained by step (iv) a DNA sequence encoding or having the corresponding amino acid residues of the rabbit heavy chain CDR3 which is contained in the same rabbit heavy chain antibody sequence;
(vi) further selecting a human heavy chain framework 4 region (FR4) that is homologous thereto (preferably differs from the FR4 contained in the humanized rabbit antibody heavy chain sequence by at most 4 amino acid residues) and attaching a DNA sequence encoding said selected homologous human FR4 or the corresponding amino residues of said human FR4 onto the DNA or amino acid sequence obtained after step (v)); and
(vii) synthesizing a DNA or amino acid sequence encoding or containing the humanized rabbit heavy chain sequence that results from steps (i) through (vi).

44. The humanization strategy of claim 43 wherein the amino acid initiating FR1 is the first amino acid after the rabbit heavy chain signal sequence.

45. The humanization strategy of claim 43 wherein the end of FR3 is about 95-100 amino acid residues after the first residue of FR1.

46. The humanization strategy of claim 43 wherein the signal sequence comprises no more than 19 amino acid residues.

47. The humanization strategy of claim 43 wherein the homologous human heavy chain sequence is identified by a BLAST search of human germline sequences obtained prior to antibody maturation.

48. The humanization strategy of claim 43 wherein the selected homologous human heavy chain possesses at least 90-95% sequence identity to the corresponding region of the rabbit heavy chain.

49. The humanization strategy of claim 43 wherein the FR1, FR2, FR3 and CDR1 and CDR2 regions in the rabbit heavy chain sequence are identified by aligning the rabbit FR1, FR2, FR3 and CDR1 and CDR2 regions with the corresponding human heavy chain FR1, FR2, FR3, CDR1 and CDR2 regions.

50. The humanization strategy of claim 43 wherein the final 3 amino acid residues of the human FR1 are replaced with the corresponding 3 residues of the rabbit FR1.

51. The humanization strategy of claim 50 wherein the 3 residues in rabbit FR1 are preceded by ser-gly.

52. The humanization strategy of claim 43 which further comprises replacing the terminal amino acid residue of the human FR2 with the corresponding terminal amino acid residue of rabbit FR2.

53. The humanization strategy of claim 52 wherein the terminal rabbit FR2 residue comprises a glycine optionally preceded by a isoleucine residue.

54. The humanization strategy of claim 43 which further comprises changing the tryptophan residue that is located about 4 residues from the end of the rabbit CDR2 with a serine residue.

55. The method of claim 43 wherein the rabbit CDR3 comprises 5-19 amino acid residues.

56. The humanization strategy of claim 43 wherein the rabbit CDR3 is followed by the residues WG″X″G, where “X” is preferably Q or P.

57. The humanization strategy of claim 43 wherein the rabbit FR4 comprises 11 amino acid residues.

58. The humanization strategy of claim 57 wherein the rabbit FR4 comprises WGQGTLVTVSS.

59. The humanized rabbit heavy chain variable amino acid sequence or DNA sequence produced by claim 43 which is derived from a rabbit antibody specific to an antigen selected from a microbial antigen, a human antigen, viral antigen, and an allergen.

60. The humanized rabbit heavy chain variable amino acid sequence or DNA sequence of claim 59 which is specific to a human antigen.

61. The humanized rabbit heavy chain variable amino acid or DNA sequence of claim 59 wherein the human antigen is selected from a human autoantigen, cytokine, receptor protein, enzyme, hormone, receptor ligand, steroid, growth factor and an oncogene.

62. An antibody or antibody fragment containing a humanized rabbit heavy chain variable sequence produced according to claim 43.

63. The humanized rabbit heavy chain produced according to claim 43 which is attached to an effector moiety.

64. The humanized rabbit heavy chain polypeptide of claim 63 wherein the effector moiety is selected from a drug, a toxin, an enzyme, a radionuclide, a fluorophore, a cytokine, an affinity label, and a translocating polypeptide.

65. The humanized rabbit heavy chain polypeptide or a DNA encoding produced according to claim 43 which is derived from a rabbit antibody that specifically binds a cytokine, growth factor or a tumor specific polypeptide.

66. The humanized rabbit heavy chain polypeptide of claim 65 that is derived from a rabbit antibody that specifically binds IL-6, TNF-alpha, VEGF-alpha, IL-12, Hepcidin or Hepatocycte growth factor.

67. The humanized rabbit heavy chain polypeptide of claim 64 which is aglycosylated.

68. A humanized rabbit antibody comprising at least one humanized rabbit light chain produced according to claims 23 and at least one humanized rabbit heavy chain, wherein said humanized rabbit heavy chain is produced by a humanization strategy for producing a humanized heavy chain antibody sequence from a rabbit heavy chain antibody sequence comprising the following steps:
(i) obtaining a rabbit heavy chain antibody sequence from an rabbit antibody that specifically binds to a desired antigen and identifying the amino residues spanning the beginning of Framework 1 (FR1) to the end of Framework 3 (FR3) inclusive:
(ii) conducting a homology search (e.g., by BLAST searching of human germline antibody sequence containing libraries) using said rabbit heavy antibody amino acid sequence spanning the beginning of FR1 to the end of FR3 sequence and identifying a human heavy chain antibody sequence that is homologous thereto, i.e. which preferably possesses at least 80%-90% identical thereto at the amino acid level;
(iii) identifying in both the rabbit and human heavy chain sequences the arrangement of and the specific residues thereof that correspond to FR1, FR2, FR3, CDR1, CDR2 regions and aligning these discrete regions of the rabbit against the corresponding regions of the selected homologous human antibody heavy chain;
(iv) constructing a DNA or amino acid sequence wherein the residues in the CDR1 and CDR2 regions of the selected homologous human heavy chain sequence are substituted by the selectivity determining residues contained in the corresponding CDR1 and CDR2 regions of the rabbit heavy chain sequence and optionally replacing the terminal 1-3 amino acids of the human heavy FR1 region with the corresponding terminal 1-3 amino acids of the rabbit heavy chain FR11 and/or optionally replacing the terminal amino acid of the human heavy chain framework 2 region with the corresponding terminal amino acid residue of the rabbit heavy chain framework 2 and/or optionally replacing the fourth amino acid from the terminus of the rabbit heavy chain CDR2 (typically a tryptophan) with the corresponding human CDR2 residue (typically a serine);
(v) further attaching to the DNA or amino acid sequence obtained by step (iv) a DNA sequence encoding or having the corresponding amino acid residues of the rabbit heavy chain CDR3 which is contained in the same rabbit heavy chain antibody sequence:
(vi) further selecting a human heavy chain framework 4 region (FR4) that is homologous thereto (preferably differs from the FR4 contained in the humanized rabbit antibody heavy chain sequence by at most 4 amino acid residues) and attaching a DNA sequence encoding said selected homologous human FR4 or the corresponding amino residues of said human FR4 onto the DNA or amino acid sequence obtained after step (v)); and
(vii) synthesizing a DNA or amino acid sequence encoding or containing the humanized rabbit heavy chain sequence that results from steps (i) through (vi).

69. The humanized rabbit antibody of claim 68 which comprises human constant domains.

70. The humanized rabbit antibody of claim 69 which is selected from an IgG1, IgG2, IgG3 and IgG4.

71. The humanized rabbit antibody of claim 68 which binds an antigen selected from a human antigen, bacterial antigen, viral antigen, pathogen, parasite, yeast antigen and a fungal antigen.

72. A method of immunotherapy or immunodiagnosis which comprises the administration of a humanized antibody wherein the improvement comprises administering a humanized antibody or antibody fragment according to claim 1.

73. The method of claim 72 which comprises ameliorating or reducing symptoms of a disease or disorder associated with IL-6, or TNF.

74. The method of claim 73, wherein said disease or disorder associated with IL-6 or TNF-alpha is cancer or an inflammatory condition.

75. The method of claim 73 wherein the antibody is an anti-IL-6 antibody and is used to treat or diagnose the prognosis of IL-6 associated fatigue, cachexia or arthritis.

76. The method of claim 73, wherein said disease or disorder associated with IL-6 is selected from general fatigue, exercise-induced fatigue, cancer-related fatigue, inflammatory disease-related fatigue, chronic fatigue syndrome, cancer-related cachexia, cardiac-related cachexia, respiratory-related cachexia, renal-related cachexia, age-related cachexia, rheumatoid arthritis, systemic lupus erythematosis (SLE), systemic juvenile idiopathic arthritis, psoriasis, psoriatic arthropathy, ankylosing spondylitis, inflammatory bowel disease (IBD), polymyalgia rheumatica, giant cell arteritis, autoimmune vasculitis, graft versus host disease (GVHD), Sjogren's syndrome, adult onset Still's disease, rheumatoid arthritis, systemic juvenile idiopathic arthritis, osteoarthritis, osteoporosis, Paget's disease of bone, osteoarthritis, multiple myeloma, Hodgkin's lymphoma, non-Hodgkin's lymphoma, prostate cancer, leukemia, renal cell cancer, multicentric Castleman's disease, ovarian cancer, drug resistance in cancer chemotherapy, cancer chemotherapy toxicity, ischemic heart disease, atherosclerosis, obesity, diabetes, asthma, multiple sclerosis, Alzheimer's disease and cerebrovascular disease.

77. The method of claim 73, wherein said disease or disorder is associated with TNF and is selected from general fatigue, exercise-induced fatigue, cancer-related fatigue, inflammatory disease-related fatigue, chronic fatigue syndrome, cancer-related cachexia, cardiac-related cachexia, respiratory-related cachexia, renal-related cachexia, age-related cachexia, rheumatoid arthritis, systemic lupus erythematosis (SLE), systemic juvenile idiopathic arthritis, psoriasis, psoriatic arthropathy, ankylosing spondylitis, inflammatory bowel disease (IBD), polymyalgia rheumatica, giant cell arteritis, autoimmune vasculitis, graft versus host disease (GVHD), Sjogren's syndrome, adult onset Still's disease, rheumatoid arthritis, systemic juvenile idiopathic arthritis, osteoarthritis, osteoporosis, Paget's disease of bone, osteoarthritis, multiple myeloma, Hodgkin's lymphoma, non-Hodgkin's lymphoma, prostate cancer, leukemia, renal cell cancer, multicentric Castleman's disease, ovarian cancer, drug resistance in cancer chemotherapy, cancer chemotherapy toxicity, ischemic heart disease, atherosclerosis, obesity, diabetes, asthma, multiple sclerosis, Alzheimer's disease and cerebrovascular disease.

78. The method of claim 72 wherein the humanized antibody or antibody fragment is expressed in a polyploid yeast culture that stably expresses and secretes into the culture medium at least 10-25 mg/liter of said antibody, comprising:
(i) introducing at least one expression vector containing one or more heterologous polynucleotides encoding said humanized antibody or fragment operably linked to a promoter and a signal sequence into a haploid yeast cell;
(ii) producing by mating or spheroplast fusion a polyploidal yeast from said first and/or second haploid yeast cell;
(iii) selecting polyploidal yeast cells that stably express said humanized antibody or fragment; and
(iv) producing stable polyploidal yeast cultures from said polyploidal yeast cells that stably express at least 10-25 mg/liter of said humanized antibody or fragment into the culture medium.

79. The method of claim 78, wherein said yeast is selected from the following genera: Arxiozyma; Ascobotryozyma; Citeromyces; Debaryomyces; Dekkera; Eremothecium; Issatchenkia; Kazachstania; Kluyveromyces; Kodamaea; Lodderomyces; Pachysolen; Pichia; Saccharomyces; Saturnispora; Tetrapisispora; Torulaspora; Williopsis; and Zygosaccharomyces.

80. The method of claim 79, wherein said yeast genera is Pichia.

81. The method of claim 80, wherein the species of Pichia is selected from Pichia pastoris, Pichia methanolica and Hansenula polymorpha (Pichia angusta).

82. A humanized antibody or antibody fragment containing a humanized antibody polypeptide produced by the method of claim 68 wherein said humanized antibody or fragment binds to an antigen with a dissociation constant (K_{D}) of less than or equal to 5×10⁻⁷ M⁻¹, 10⁻⁷ M⁻¹, 5×10⁻⁸ M⁻¹, 10⁻⁸ M⁻¹, 5×10⁻⁹ M⁻¹, 10⁻⁹ M⁻¹, 5×10⁻¹⁰ M⁻¹, 10⁻¹⁰ M⁻¹, 5×10⁻¹¹ M⁻¹, 10⁻¹¹ M⁻¹, 5×10⁻¹² M⁻¹, 10⁻¹² M⁻¹, 5×10⁻¹³ M⁻¹, 10⁻¹³ M⁻¹, or 5×10⁻¹⁴ M⁻¹.

83. The humanized antibody of claim 82, wherein said antibody binds to an antigen with a dissociation constant (K_{D}) of less than or equal to 5×10⁻¹⁰ M⁻¹.

84. The humanized antibody of claim 82, wherein said antibody binds to an antigen with an off-rate (K_{off}) of less than or equal to 10⁻⁴ S⁻¹, 5×10⁻⁵ S⁻¹, 10⁻⁵ S⁻¹, 5×10⁻⁶ S⁻¹, 10⁻⁶ S⁻¹, 5×10⁻⁷S⁻¹, or 10⁻⁷ S⁻¹.

85. The humanized antibody of claim 82, wherein the parent rabbit antibody originated from one or more rabbit B cell populations.

86. The humanized antibody of claim 82 wherein said antibody inhibits the association of IL-6 with IL-6R or TNF and its receptor.

87. The humanized antibody of claim 86, wherein the IL-6R is soluble IL-6R (sIL-6R).

88. The humanized antibody of claim 86, wherein the TNF receptor (TNFR) is soluble.

89. A vector that expresses a humanized rabbit according to claim 1.

90. A host cell comprising the vector of claim 89.

91. The host cell of claim 90, wherein said host cell is a yeast cell belonging to the genus Pichia.


SEQUENCE LISTING (1067 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 125 aa, Oryctolagus cuniculus
  anti-TNF-alpha antibody Ab1 light chain variable domain
MDTRAPTQLLGLLLLWLPGARCADIVMTQTPASVEAAVGGTVTIKCQASQNIRSWLAWYQQKPGQPPKLLIYGASTLASGVPSRFQGSGSGTEYTLTIIDLDCADAATYYCQSNYGSNDNSYGNG

SEQ ID NO: 3 - protein, 121 aa, Oryctolagus cuniculus
  anti-TNF-alpha antibody Ab1 heavy chain variable domain
METGLRWLLLVAVLKGVQCQSVEESGGRLVTPGTPLTLTCTVSGFSLSTYNMGWVRQAPGKGLEYIGYVLGSGITYYASWAKGRFTISKTSTTVDLEITSPTTEDTATYFCARDAGGRASL

SEQ ID NO: 4 - protein, 11 aa, Oryctolagus cuniculus
  anti-TNF-alpha antibody Ab1 light chain CDR 1
QASQNIRSWLA

SEQ ID NO: 5 - protein, 7 aa, Oryctolagus cuniculus
  anti-TNF-alpha antibody Ab1 light chain CDR 2
GASTLAS

SEQ ID NO: 6 - protein, 14 aa, Oryctolagus cuniculus
  anti-TNF-alpha antibody Ab1 light chain CDR 3
QSNYGSNDNSYGNG

SEQ ID NO: 7 - protein, 5 aa, Oryctolagus cuniculus
  anti-TNF-alpha antibody Ab1 heavy chain CDR 1
TYNMG

SEQ ID NO: 8 - protein, 16 aa, Oryctolagus cuniculus
  anti-TNF-alpha antibody Ab1 heavy chain CDR 2
YVLGSGITYYASWAKG

SEQ ID NO: 9 - protein, 8 aa, Oryctolagus cuniculus
  anti-TNF-alpha antibody Ab1 heavy chain CDR 3
DAGGRASL

SEQ ID NO: 10 - DNA, 375 bp, Oryctolagus cuniculus
  anti-TNF-alpha antibody Ab1 light chain variable domain
atggacacgagggcccccactcagctgctggggctcctactgctctggctcccaggtgccagatgtgctgacattgtgatgacccagactccagcctccgtggaggcagctgtgggaggcacagtcaccatcaagtgccaggccagtcagaacattcgcagttggttagcctggtatcagcagaaaccagggcagcctcccaagctcctgatctatggtgcatccactctggcatctggggtcccatcgcgattccaaggcagtggatctgggacagagtacactctcaccatcatcgacctggactgtgccgatgctgccacttactactgtcaaagcaattatggtagtaatgataatagttatggtaatggt

SEQ ID NO: 11 - DNA, 363 bp, Oryctolagus cuniculus
  anti-TNF-alpha antibody Ab1 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctacaacatgggctgggtccgccaggctccagggaaggggctggaatacatcggatacgtgttgggaagtggtatcacatactacgcgagctgggcaaaaggccgattcaccatctccaaaacctcgaccacggtggatctggagatcactagtccgacaaccgaggacacggccacctatttctgtgccagagatgctggtggcagagcttccttg

SEQ ID NO: 12 - DNA, 33 bp, Oryctolagus cuniculus
  anti-TNF-alpha antibody Ab1 light chain CDR 1
caggccagtcagaacattcgcagttggttagcc

SEQ ID NO: 13 - DNA, 21 bp, Oryctolagus cuniculus
  anti-TNF-alpha antibody Ab1 light chain CDR 2
ggtgcatccactctggcatct

SEQ ID NO: 14 - DNA, 42 bp, Oryctolagus cuniculus
  anti-TNF-alpha antibody Ab1 light chain CDR 3
caaagcaattatggtagtaatgataatagttatggtaatggt

SEQ ID NO: 15 - DNA, 15 bp, Oryctolagus cuniculus
  anti-TNF-alpha antibody Ab1 heavy chain CDR 1
acctacaacatgggc

SEQ ID NO: 16 - DNA, 48 bp, Oryctolagus cuniculus
  anti-TNF-alpha antibody Ab1 heavy chain CDR 2
tacgtgttgggaagtggtatcacatactacgcgagctgggcaaaaggc

SEQ ID NO: 17 - DNA, 24 bp, Oryctolagus cuniculus
  anti-TNF-alpha antibody Ab1 heavy chain CDR 3
gatgctggtggcagagcttccttg

SEQ ID NO: 18 - protein, 125 aa, Oryctolagus cuniculus
  anti-TNF-alpha antibody Ab2 light chain variable domain
MDTRAPTQLLGLLLLWLPGARCADIVMTQTPSSVSEPVRGTVTIKCQASQNIYSYLSWYQQSPGQPPKLLIYKASTLASGVPSRFKGSGSGTDFTLTISDLECADAATYYCQSNYGSDSDSFGNA

SEQ ID NO: 19 - protein, 122 aa, Oryctolagus cuniculus
  anti-TNF-alpha antibody Ab2 heavy chain variable domain
METGLRWLLLVAVLKGVQCQSVEESGGRLVTPGTPLTLTCSVSGFSLNNYVMGWVRQAPGKGLEFIGYIAFGIGPYYASWAKGRFTISSTSSTTVDLKMTSLTPEDTATYFCARGDYSGNDI

SEQ ID NO: 20 - protein, 11 aa, Oryctolagus cuniculus
  anti-TNF-alpha antibody Ab2 light chain CDR 1
QASQNIYSYLS

SEQ ID NO: 21 - protein, 7 aa, Oryctolagus cuniculus
  anti-TNF-alpha antibody Ab2 light chain CDR 2
KASTLAS

SEQ ID NO: 22 - protein, 14 aa, Oryctolagus cuniculus
  anti-TNF-alpha antibody Ab2 light chain CDR 3
QSNYGSDSDSFGNA

SEQ ID NO: 23 - protein, 5 aa, Oryctolagus cuniculus
  anti-TNF-alpha antibody Ab2 heavy chain CDR 1
NYVMG

SEQ ID NO: 24 - protein, 16 aa, Oryctolagus cuniculus
  anti-TNF-alpha antibody Ab2 heavy chain CDR 2
YIAFGIGPYYASWAKG

SEQ ID NO: 25 - protein, 8 aa, Oryctolagus cuniculus
  anti-TNF-alpha antibody Ab2 heavy chain CDR 3
GDYSGNDI

SEQ ID NO: 26 - DNA, 375 bp, Oryctolagus cuniculus
  anti-TNF-alpha antibody Ab2 light chain variable domain
atggacacgagggcccccactcagctgctggggctcctgctgctctggctcccaggtgccagatgtgctgacattgtgatgacccagactccatcctccgtgtctgaacctgtgcgaggcacagtcaccatcaagtgccaggccagtcagaacatttacagctacttgtcctggtatcaacagagcccagggcagcctcccaagctcctgatctacaaggcatccactctggcatctggggtcccatcgcggttcaaaggcagtggatctgggacagatttcactctcaccatcagcgacctggagtgtgccgatgctgccacttactactgtcaatccaattatggtagtgatagtgatagttttgggaatgct

SEQ ID NO: 27 - DNA, 366 bp, Oryctolagus cuniculus
  anti-TNF-alpha antibody Ab2 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgctcagtctctggattctccctcaataattatgtaatgggctgggtccgccaggctccagggaaggggctggaattcatcggatacattgcttttggtattggcccatactacgcgagctgggcgaaaggccgattcaccatctccagcacctcgtcgaccacggtggatctgaaaatgaccagtctgacacccgaggacacggccacctatttctgtgccagaggtgattatagtggtaatgacatt

SEQ ID NO: 28 - DNA, 33 bp, Oryctolagus cuniculus
  anti-TNF-alpha antibody Ab2 light chain CDR 1
caggccagtcagaacatttacagctacttgtcc

SEQ ID NO: 29 - DNA, 21 bp, Oryctolagus cuniculus
  anti-TNF-alpha antibody Ab2 light chain CDR 2
aaggcatccactctggcatct

SEQ ID NO: 30 - DNA, 42 bp, Oryctolagus cuniculus
  anti-TNF-alpha antibody Ab2 light chain CDR 3
caatccaattatggtagtgatagtgatagttttgggaatgct

SEQ ID NO: 31 - DNA, 15 bp, Oryctolagus cuniculus
  anti-TNF-alpha antibody Ab2 heavy chain CDR 1
aattatgtaatgggc

SEQ ID NO: 32 - DNA, 48 bp, Oryctolagus cuniculus
  anti-TNF-alpha antibody Ab2 heavy chain CDR 2
tacattgcttttggtattggcccatactacgcgagctgggcgaaaggc

SEQ ID NO: 33 - DNA, 24 bp, Oryctolagus cuniculus
  anti-TNF-alpha antibody Ab2 heavy chain CDR 3
ggtgattatagtggtaatgacatt

SEQ ID NO: 34 - protein, 122 aa, Oryctolagus cuniculus
  anti-TNF-alpha antibody Ab3 light chain variable domain
MDTRAPTQLLGLLLLWLPGSTFAIKVTQTPASVSAAVGGTVSINCQASEDIESYLAWYQQKPGQPPKLLLYDASALASGVPSRFKGSGSGTEYTLTISGVECADAATYYCQQGYSYSNVDNS

SEQ ID NO: 35 - protein, 127 aa, Oryctolagus cuniculus
  anti-TNF-alpha antibody Ab3 heavy chain variable domain
METGLRWLLLVAVLKGVQCQSVEESGGRLVTPGTPLTLTCKVSGFSLSSYDMTWVRQAPGKGLEWIGYIWNDGSTAYASWATGRFTISKTSTTVDLKIASPTTEDTATYFCARGPVFATTLGYYFTI

SEQ ID NO: 36 - protein, 11 aa, Oryctolagus cuniculus
  anti-TNF-alpha antibody Ab3 light chain CDR 1
QASEDIESYLA

SEQ ID NO: 37 - protein, 7 aa, Oryctolagus cuniculus
  anti-TNF-alpha antibody Ab3 light chain CDR 2
DASALAS

SEQ ID NO: 38 - protein, 12 aa, Oryctolagus cuniculus
  anti-TNF-alpha antibody Ab3 light chain CDR 3
QQGYSYSNVDNS

SEQ ID NO: 39 - protein, 5 aa, Oryctolagus cuniculus
  anti-TNF-alpha antibody Ab3 heavy chain CDR 1
SYDMT

SEQ ID NO: 40 - protein, 16 aa, Oryctolagus cuniculus
  anti-TNF-alpha antibody Ab3 heavy chain CDR 2
YIWNDGSTAYASWATG

SEQ ID NO: 41 - protein, 14 aa, Oryctolagus cuniculus
  anti-TNF-alpha antibody Ab3 heavy chain CDR 3
GPVFATTLGYYFTI

SEQ ID NO: 42 - DNA, 366 bp, Oryctolagus cuniculus
  anti-TNF-alpha antibody Ab3 light chain variable domain
atggacacgagggcccccactcagctgctggggctcctgctgctctggctcccaggttccacatttgccatcaaagtgacccagacaccagcctccgtgtctgcagctgtgggaggcacagtcagcatcaattgccaggccagtgaggacattgaaagctatttggcctggtatcagcagaaaccagggcagcctcccaaactccttctctatgatgcatccgctctggcttctggggtcccatcgcggttcaaaggcagtggatctgggacagagtacactctcaccatcagcggcgtggagtgtgccgatgctgccacttactactgtcaacagggttatagttatagtaatgttgataattct

SEQ ID NO: 43 - DNA, 381 bp, Oryctolagus cuniculus
  anti-TNF-alpha antibody Ab3 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcaaagtctctggattctccctcagcagctacgacatgacctgggtccgccaggctccagggaaggggctggagtggatcggatacatttggaatgatggtagtacagcctacgcgagctgggcgacaggccgattcaccatctccaaaacctcgaccacggtggatctgaaaatcgccagtccgacaaccgaggacacggccacctatttctgtgccagaggtcctgtttttgcgactactcttgggtactactttaccatc

SEQ ID NO: 44 - DNA, 33 bp, Oryctolagus cuniculus
  anti-TNF-alpha antibody Ab3 light chain CDR 1
caggccagtgaggacattgaaagctatttggcc

SEQ ID NO: 45 - DNA, 21 bp, Oryctolagus cuniculus
  anti-TNF-alpha antibody Ab3 light chain CDR 2
gatgcatccgctctggcttct

SEQ ID NO: 46 - DNA, 36 bp, Oryctolagus cuniculus
  anti-TNF-alpha antibody Ab3 light chain CDR 3
caacagggttatagttatagtaatgttgataattct

SEQ ID NO: 47 - DNA, 15 bp, Oryctolagus cuniculus
  anti-TNF-alpha antibody Ab3 heavy chain CDR 1
agctacgacatgacc

SEQ ID NO: 48 - DNA, 48 bp, Oryctolagus cuniculus
  anti-TNF-alpha antibody Ab3 heavy chain CDR 2
tacatttggaatgatggtagtacagcctacgcgagctgggcgacaggc

SEQ ID NO: 49 - DNA, 42 bp, Oryctolagus cuniculus
  anti-TNF-alpha antibody Ab3 heavy chain CDR 3
ggtcctgtttttgcgactactcttgggtactactttaccatc

SEQ ID NO: 50 - protein, 123 aa, Oryctolagus cuniculus
  anti-TNF-alpha antibody Ab4 light chain variable domain
MDTRAPTQLLGLLLLWLTGATFAAVLTQTPSPVSAVVGGTVSISCQSSKRVVNSVALSWYQQKPGRSPKLLIYFASKLASGVPSRFKGSGSGTQFTLAISDVQCDDAATYYCAGHYTDSGDDA

SEQ ID NO: 51 - protein, 122 aa, Oryctolagus cuniculus
  anti-TNF-alpha antibody Ab4 heavy chain variable domain
METGLRWLLLVAVLKGVQCQSVEESGGRLVTPGTPLTLTCTVSGLSLSTETINWVRQAPGKGLEWIGYIDSSGGTGYANWARGRFTISKTSTTVDLKITSPTTGDTATYFCARGTITTGMNI

SEQ ID NO: 52 - protein, 13 aa, Oryctolagus cuniculus
  anti-TNF-alpha antibody Ab4 light chain CDR 1
QSSKRVVNSVALS

SEQ ID NO: 53 - protein, 7 aa, Oryctolagus cuniculus
  anti-TNF-alpha antibody Ab4 light chain CDR 2
FASKLAS

SEQ ID NO: 54 - protein, 11 aa, Oryctolagus cuniculus
  anti-TNF-alpha antibody Ab4 light chain CDR 3
AGHYTDSGDDA

SEQ ID NO: 55 - protein, 5 aa, Oryctolagus cuniculus
  anti-TNF-alpha antibody Ab4 heavy chain CDR 1
TETIN

SEQ ID NO: 56 - protein, 16 aa, Oryctolagus cuniculus
  anti-TNF-alpha antibody Ab4 heavy chain CDR 2
YIDSSGGTGYANWARG

SEQ ID NO: 57 - protein, 9 aa, Oryctolagus cuniculus
  anti-TNF-alpha antibody Ab4 heavy chain CDR 3
GTITTGMNI

SEQ ID NO: 58 - DNA, 369 bp, Oryctolagus cuniculus
  anti-TNF-alpha antibody Ab4 light chain variable domain
atggacacgagggcccccactcagctgctggggctcctgctgctctggctcacaggtgccacatttgccgccgtgctgacccagactccatctcccgtgtctgcagttgtgggaggcacagtcagcatcagttgccagtccagcaagagagttgttaatagcgttgccttatcctggtatcagcagaaaccagggcgctctcctaagctcctgatctattttgcatccaaactggcatctggggtcccatcgcggttcaaaggcagtggatctgggacacagttcactctcgccattagcgacgtgcagtgtgacgatgctgccacttactactgtgcaggccattatactgatagtggtgatgatgct

SEQ ID NO: 59 - DNA, 366 bp, Oryctolagus cuniculus
  anti-TNF-alpha antibody Ab4 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattatccctcagtaccgagacaattaactgggtccgccaggctccagggaagggactggagtggatcggatacattgatagttctggtggcacaggctacgcgaactgggcgagaggccgattcaccatctccaaaacctcgaccacggtggatttgaaaatcaccagtccgacaaccggggacacggccacctatttctgtgccagaggaactattactactggcatgaacatc

SEQ ID NO: 60 - DNA, 39 bp, Oryctolagus cuniculus
  anti-TNF-alpha antibody Ab4 light chain CDR 1
cagtccagcaagagagttgttaatagcgttgccttatcc

SEQ ID NO: 61 - DNA, 21 bp, Oryctolagus cuniculus
  anti-TNF-alpha antibody Ab4 light chain CDR 2
tttgcatccaaactggcatct

SEQ ID NO: 62 - DNA, 33 bp, Oryctolagus cuniculus
  anti-TNF-alpha antibody Ab4 light chain CDR 3
gcaggccattatactgatagtggtgatgatgct

SEQ ID NO: 63 - DNA, 15 bp, Oryctolagus cuniculus
  anti-TNF-alpha antibody Ab4 heavy chain CDR 1
accgagacaattaac

SEQ ID NO: 64 - DNA, 48 bp, Oryctolagus cuniculus
  anti-TNF-alpha antibody Ab4 heavy chain CDR 2
tacattgatagttctggtggcacaggctacgcgaactgggcgagaggc

SEQ ID NO: 65 - DNA, 27 bp, Oryctolagus cuniculus
  anti-TNF-alpha antibody Ab4 heavy chain CDR 3
ggaactattactactggcatgaacatc

SEQ ID NO: 66 - protein, 122 aa, Oryctolagus cuniculus
  anti-TNF-alpha antibody Ab5 light chain variable domain
MDTRAPTQLLGLLLLWLPGATLAQVVTQTPASVSAAVGGTVTISCQSSQNVYNNNDLVWFQQKPGQPPKRLVYWASTLASGVSSRFRGSGSGTQFILTISDLQCDDAATYYCAGAYDSEIRA

SEQ ID NO: 67 - protein, 120 aa, Oryctolagus cuniculus
  anti-TNF-alpha antibody Ab5 heavy chain variable domain
METGLRWLLLVAVLKGVQCQSVEESGGRLVTPGTPLTLTCAVSGFSLSVYWMTWVRQAPGKGLEWIGTISTDGITVYATWAKGRFTISKTSSTAVDLKLTSPTTEDTATYFCAGGGGMDP

SEQ ID NO: 68 - protein, 13 aa, Oryctolagus cuniculus
  anti-TNF-alpha antibody Ab5 light chain CDR 1
QSSQNVYNNNDLV

SEQ ID NO: 69 - protein, 7 aa, Oryctolagus cuniculus
  anti-TNF-alpha antibody Ab5 light chain CDR 2
WASTLAS

SEQ ID NO: 70 - protein, 10 aa, Oryctolagus cuniculus
  anti-TNF-alpha antibody Ab5 light chain CDR 3
AGAYDSEIRA

SEQ ID NO: 71 - protein, 5 aa, Oryctolagus cuniculus
  anti-TNF-alpha antibody Ab5 heavy chain CDR 1
VYWMT

SEQ ID NO: 72 - protein, 16 aa, Oryctolagus cuniculus
  anti-TNF-alpha antibody Ab5 heavy chain CDR 2
TISTDGITVYATWAKG

SEQ ID NO: 73 - protein, 6 aa, Oryctolagus cuniculus
  anti-TNF-alpha antibody Ab5 heavy chain CDR 3
GGGMDP

SEQ ID NO: 74 - DNA, 366 bp, Oryctolagus cuniculus
  anti-TNF-alpha antibody Ab5 light chain variable domain
atggacacgagggcccccactcagctgctggggctcctgctgctctggctcccaggtgccacacttgcgcaagtggtgacccagactccagcctccgtgtctgcagctgtgggaggcacagtcaccatcagttgccagtccagtcagaatgtttataataataatgacttagtctggtttcagcagaaaccaggtcagcctcccaagcgcctggtctactgggcatccactctggcatctggggtctcatcgcggttcagaggcagtggatctgggacacagttcattctcaccatcagcgacctgcagtgtgacgatgctgccacttactattgtgcaggcgcctatgatagtgaaattagggct

SEQ ID NO: 75 - DNA, 360 bp, Oryctolagus cuniculus
  anti-TNF-alpha antibody Ab5 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcgcagtctctggattctccctcagtgtttactggatgacctgggtccgccaggctccagggaaggggctggaatggatcggaaccattagtactgatggtatcactgtctacgcgacctgggcgaaaggccgattcaccatctccaaaacctcgtcgaccgcggtggatctgaaactcaccagtccgacaaccgaggacacggccacctatttctgtgccggagggggcggcatggacccc

SEQ ID NO: 76 - DNA, 39 bp, Oryctolagus cuniculus
  anti-TNF-alpha antibody Ab5 light chain CDR 1
cagtccagtcagaatgtttataataataatgacttagtc

SEQ ID NO: 77 - DNA, 21 bp, Oryctolagus cuniculus
  anti-TNF-alpha antibody Ab5 light chain CDR 2
tgggcatccactctggcatct

SEQ ID NO: 78 - DNA, 30 bp, Oryctolagus cuniculus
  anti-TNF-alpha antibody Ab5 light chain CDR 3
gcaggcgcctatgatagtgaaattagggct

SEQ ID NO: 79 - DNA, 15 bp, Oryctolagus cuniculus
  anti-TNF-alpha antibody Ab5 heavy chain CDR 1
gtttactggatgacc

SEQ ID NO: 80 - DNA, 48 bp, Oryctolagus cuniculus
  anti-TNF-alpha antibody Ab5 heavy chain CDR 2
accattagtactgatggtatcactgtctacgcgacctgggcgaaaggc

SEQ ID NO: 81 - DNA, 18 bp, Oryctolagus cuniculus
  anti-TNF-alpha antibody Ab5 heavy chain CDR 3
gggggcggcatggacccc

SEQ ID NO: 82 - protein, 122 aa, Oryctolagus cuniculus
  anti-TNF-alpha antibody Ab6 light chain variable domain
MDTRAPTQLLGLLLLWLPDARCAYDMTQTPASVEVAGGGTVTIKCQASQSIANRLAWYQQKPGQPPKLLIYYASTLASGVPSRFSGSGSGTEFTLTISGVQCDDAATYYCQQTYSDNNVDNA

SEQ ID NO: 83 - protein, 126 aa, Oryctolagus cuniculus
  anti-TNF-alpha antibody Ab6 heavy chain variable domain
METGLRWLLLVAVFKGVQCQSVEESGGRLVTPGTPLTLTCTVSGFSLSSNTISWVRQAPGKGLEWIGYIWRGVSTYYATWAKGRFTISKTSSTTVDLKITGPTTEDTATYFCARDAGDGGGYSLDL

SEQ ID NO: 84 - protein, 11 aa, Oryctolagus cuniculus
  anti-TNF-alpha antibody Ab6 light chain CDR 1
QASQSIANRLA

SEQ ID NO: 85 - protein, 7 aa, Oryctolagus cuniculus
  anti-TNF-alpha antibody Ab6 light chain CDR 2
YASTLAS

SEQ ID NO: 86 - protein, 12 aa, Oryctolagus cuniculus
  anti-TNF-alpha antibody Ab6 light chain CDR 3
QQTYSDNNVDNA

SEQ ID NO: 87 - protein, 5 aa, Oryctolagus cuniculus
  anti-TNF-alpha antibody Ab6 heavy chain CDR 1
SNTIS

SEQ ID NO: 88 - protein, 16 aa, Oryctolagus cuniculus
  anti-TNF-alpha antibody Ab6 heavy chain CDR 2
YIWRGVSTYYATWAKG

SEQ ID NO: 89 - protein, 12 aa, Oryctolagus cuniculus
  anti-TNF-alpha antibody Ab6 heavy chain CDR 3
DAGDGGGYSLDL

SEQ ID NO: 90 - DNA, 366 bp, Oryctolagus cuniculus
  anti-TNF-alpha antibody Ab6 light chain variable domain
atggacacgagggcccccactcagctgctggggctcctgctgctctggctcccagatgccagatgtgcctatgatatgacccagactccagcctctgtggaggtagctgggggaggcacagtcaccatcaagtgccaggccagtcagagcattgctaataggttagcctggtatcagcagaaaccagggcagcctcccaagctcctgatctattatgcatccacgctggcatctggggtcccatcgcggttcagcggcagtggatctgggacagagttcactctcaccatcagtggcgtgcagtgtgacgatgctgccacttactactgtcagcagacttatagtgataataatgtcgataatgct

SEQ ID NO: 91 - DNA, 378 bp, Oryctolagus cuniculus
  anti-TNF-alpha antibody Ab6 heavy chain variable domain
atggagactgggctgcgctggcttctcctggtcgctgtgttcaaaggtgtccagtgtcagtcggtggaggagtccgggggtcgcctggtcacgcctgggacacccctgacactcacctgcacagtctctggattctccctcagtagcaatacaataagctgggtccgccaggctccagggaaggggctggagtggatcggatacatttggcgtggtgttagcacatactacgcgacctgggcgaaaggccgattcaccatctccaaaacctcgtcgacgacggtggatctgaagatcaccggtccgacaaccgaggacacggccacctatttctgtgccagagatgctggtgatggtggtggatattccttggatctc

SEQ ID NO: 92 - DNA, 33 bp, Oryctolagus cuniculus
  anti-TNF-alpha antibody Ab6 light chain CDR 1
caggccagtcagagcattgctaataggttagcc

SEQ ID NO: 93 - DNA, 21 bp, Oryctolagus cuniculus
  anti-TNF-alpha antibody Ab6 light chain CDR 2
tatgcatccacgctggcatct

SEQ ID NO: 94 - DNA, 36 bp, Oryctolagus cuniculus
  anti-TNF-alpha antibody Ab6 light chain CDR 3
cagcagacttatagtgataataatgtcgataatgct

SEQ ID NO: 95 - DNA, 15 bp, Oryctolagus cuniculus
  anti-TNF-alpha antibody Ab6 heavy chain CDR 1
agcaatacaataagc

SEQ ID NO: 96 - DNA, 48 bp, Oryctolagus cuniculus
  anti-TNF-alpha antibody Ab6 heavy chain CDR 2
tacatttggcgtggtgttagcacatactacgcgacctgggcgaaaggc

SEQ ID NO: 97 - DNA, 36 bp, Oryctolagus cuniculus
  anti-TNF-alpha antibody Ab6 heavy chain CDR 3
gatgctggtgatggtggtggatattccttggatctc

SEQ ID NO: 98 - protein, 125 aa, Oryctolagus cuniculus
  anti-TNF-alpha antibody Ab7 light chain variable domain
MDTRAPTQLLGLLLLWLPGARCADIVMTQTPASVEAAVGGTVTINCQASQSIVSWLAWYQQKPGQPPKLLIYGASTLASGVPSRFKGSGSGTEYTLTISDLECADAATYYCQSNYGSNSHSFGNT

SEQ ID NO: 99 - protein, 121 aa, Oryctolagus cuniculus
  anti-TNF-alpha antibody Ab7 heavy chain variable domain
METGLRWLLLVAVLKGVQCQSVEESGGRLVTPGTPLTLTCTVSGFSLSSDNMGWVRQAPGKGLEYIGYITYGGFTYYATWAKGRFTISKTSTTVDLKMTSPTTEDTATYFCAREAGGRANV

SEQ ID NO: 100 - protein, 11 aa, Oryctolagus cuniculus
  anti-TNF-alpha antibody Ab7 light chain CDR 1
QASQSIVSWLA

SEQ ID NO: 101 - protein, 7 aa, Oryctolagus cuniculus
  anti-TNF-alpha antibody Ab7 light chain CDR 2
GASTLAS

SEQ ID NO: 102 - protein, 14 aa, Oryctolagus cuniculus
  anti-TNF-alpha antibody Ab7 light chain CDR 3
QSNYGSNSHSFGNT

SEQ ID NO: 103 - protein, 5 aa, Oryctolagus cuniculus
  anti-TNF-alpha antibody Ab7 heavy chain CDR 1
SDNMG

SEQ ID NO: 104 - protein, 16 aa, Oryctolagus cuniculus
  anti-TNF-alpha antibody Ab7 heavy chain CDR 2
YITYGGFTYYATWAKG

SEQ ID NO: 105 - protein, 8 aa, Oryctolagus cuniculus
  anti-TNF-alpha antibody Ab7 heavy chain CDR 3
EAGGRANV

SEQ ID NO: 106 - DNA, 375 bp, Oryctolagus cuniculus
  anti-TNF-alpha antibody Ab7 light chain variable domain
atggacacgagggcccccactcagctgctggggctcctactgctctggctcccaggtgccagatgtgctgacattgtgatgacccagactccagcctccgtggaggcagctgtgggaggcacagtcaccatcaattgccaggccagtcagagcattgtcagttggttagcctggtatcagcagaaaccagggcagcctcccaagctcctgatctatggtgcatccactctggcatctggggtcccatcgcggttcaaaggcagtggatctgggacagagtacactctcaccatcagcgacctggagtgtgccgatgctgccacttactactgtcaaagcaattatggtagtaatagtcatagttttgggaatact

SEQ ID NO: 107 - DNA, 363 bp, Oryctolagus cuniculus
  anti-TNF-alpha antibody Ab7 heavy chain variable domain
atggagactgggctgcgctggcttctcctggtcgctgtgctcaaaggtgtccagtgtcagtcagtggaggagtccgggggtcgcctggtcacgcctgggacacccctgacactcacctgcacagtctctggattctccctcagtagcgacaatatgggctgggtccgccaggctccagggaaggggctggaatacatcggatacattacttatggtggtttcacatactacgcgacctgggcgaaaggccgattcaccatctccaagacctcgaccacggtggatctgaaaatgaccagtccgacaaccgaggacacggccacctatttctgtgccagagaagctggtggtagggctaatgtc

SEQ ID NO: 108 - DNA, 33 bp, Oryctolagus cuniculus
  anti-TNF-alpha antibody Ab7 light chain CDR 1
caggccagtcagagcattgtcagttggttagcc

SEQ ID NO: 109 - DNA, 21 bp, Oryctolagus cuniculus
  anti-TNF-alpha antibody Ab7 light chain CDR 2
ggtgcatccactctggcatct

SEQ ID NO: 110 - DNA, 42 bp, Oryctolagus cuniculus
  anti-TNF-alpha antibody Ab7 light chain CDR 3
caaagcaattatggtagtaatagtcatagttttgggaatact

SEQ ID NO: 111 - DNA, 15 bp, Oryctolagus cuniculus
  anti-TNF-alpha antibody Ab7 heavy chain CDR 1
agcgacaatatgggc

SEQ ID NO: 112 - DNA, 48 bp, Oryctolagus cuniculus
  anti-TNF-alpha antibody Ab7 heavy chain CDR 2
tacattacttatggtggtttcacatactacgcgacctgggcgaaaggc

SEQ ID NO: 113 - DNA, 24 bp, Oryctolagus cuniculus
  anti-TNF-alpha antibody Ab7 heavy chain CDR 3
gaagctggtggtagggctaatgtc

SEQ ID NO: 114 - protein, 125 aa, Oryctolagus cuniculus
  anti-TNF-alpha antibody Ab8 light chain variable domain
MDTRAPTQLLGLLLLWLPGARCADIVMTQTPSSVSEPVGGTVTIMCQASQNIYSYLSWYQQKPGQPPKLLIYKASTLASGVPSRFAGSGSGTDFTLTISDLECADAATYYCQSNYGSNSDSFGNA

SEQ ID NO: 115 - protein, 122 aa, Oryctolagus cuniculus
  anti-TNF-alpha antibody Ab8 heavy chain variable domain
METGLRWLLLVAVLKGVQCQSVEESGGRLVTPGTPLTLTCTASGFSLSNYVMGWVRQAPGKGLEFIGYIAFGIGPYYATWAKGRFSISSTSSTTVDLTMTSLTPEDTATYFCARGDYSGNNI

SEQ ID NO: 116 - protein, 11 aa, Oryctolagus cuniculus
  anti-TNF-alpha antibody Ab8 light chain CDR 1
QASQNIYSYLS

SEQ ID NO: 117 - protein, 7 aa, Oryctolagus cuniculus
  anti-TNF-alpha antibody Ab8 light chain CDR 2
KASTLAS

SEQ ID NO: 118 - protein, 14 aa, Oryctolagus cuniculus
  anti-TNF-alpha antibody Ab8 light chain CDR 3
QSNYGSNSDSFGNA

SEQ ID NO: 119 - protein, 5 aa, Oryctolagus cuniculus
  anti-TNF-alpha antibody Ab8 heavy chain CDR 1
NYVMG

SEQ ID NO: 120 - protein, 16 aa, Oryctolagus cuniculus
  anti-TNF-alpha antibody Ab8 heavy chain CDR 2
YIAFGIGPYYATWAKG

SEQ ID NO: 121 - protein, 8 aa, Oryctolagus cuniculus
  anti-TNF-alpha antibody Ab8 heavy chain CDR 3
GDYSGNNI

SEQ ID NO: 122 - DNA, 375 bp, Oryctolagus cuniculus
  anti-TNF-alpha antibody Ab8 light chain variable domain
atggacacgagggcccccactcagctgctggggctcctgctgctctggctcccaggtgccagatgtgctgacattgtgatgacccagactccatcctccgtgtctgaacctgtgggaggcacagtcaccatcatgtgccaggccagtcagaacatttacagctacttatcctggtatcagcagaaaccagggcagcctcccaagctcctgatctacaaggcatccactctggcatctggggtcccatcgcggttcgcaggcagtggatctgggacagatttcactctcaccatcagcgacctggagtgtgccgatgctgccacttactactgtcaaagcaattatggtagtaatagtgatagttttgggaatgct

SEQ ID NO: 123 - DNA, 366 bp, Oryctolagus cuniculus
  anti-TNF-alpha antibody Ab8 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagcctctggattctccctcagtaattatgtaatgggctgggtccgccaggctccagggaaggggctggaattcatcggatacattgcttttggtattggcccatactacgcgacctgggcgaaaggccgattctccatctccagcacctcgtcgaccacggtggatctgacaatgaccagtctgacacccgaggacacggccacctatttctgtgccagaggtgattatagtggtaataacatt

SEQ ID NO: 124 - DNA, 33 bp, Oryctolagus cuniculus
  anti-TNF-alpha antibody Ab8 light chain CDR 1
caggccagtcagaacatttacagctacttatcc

SEQ ID NO: 125 - DNA, 21 bp, Oryctolagus cuniculus
  anti-TNF-alpha antibody Ab8 light chain CDR 2
aaggcatccactctggcatct

SEQ ID NO: 126 - DNA, 42 bp, Oryctolagus cuniculus
  anti-TNF-alpha antibody Ab8 light chain CDR 3
caaagcaattatggtagtaatagtgatagttttgggaatgct

SEQ ID NO: 127 - DNA, 15 bp, Oryctolagus cuniculus
  anti-TNF-alpha antibody Ab8 heavy chain CDR 1
aattatgtaatgggc

SEQ ID NO: 128 - DNA, 48 bp, Oryctolagus cuniculus
  anti-TNF-alpha antibody Ab8 heavy chain CDR 2
tacattgcttttggtattggcccatactacgcgacctgggcgaaaggc

SEQ ID NO: 129 - DNA, 24 bp, Oryctolagus cuniculus
  anti-TNF-alpha antibody Ab8 heavy chain CDR 3
ggtgattatagtggtaataacatt

SEQ ID NO: 130 - protein, 122 aa, Oryctolagus cuniculus
  anti-TNF-alpha antibody Ab9 light chain variable domain
MDTRAPTQLLGLLLLWLPGATFAQVLTQTPSSVSAAVGGTVTVSCQSSQNVYNNNDFVWFQQKPGQPPKRLIYWASTLASGVPSRFKGSGSGTQFTLTINDLECDDAATYYCAGAYITELRT

SEQ ID NO: 131 - protein, 120 aa, Oryctolagus cuniculus
  anti-TNF-alpha antibody Ab9 heavy chain variable domain
METGLRWLLLVAVLKGVQCQSVEESGGRLVTPGTPLTLTCTVSGFSLSIYWMTWVRQAPGKGLEWIGVISTDGSAYYATWAKGRFTISKTSSTTVDLRITSPTTEDTATYFCAGGGGMDP

SEQ ID NO: 132 - protein, 13 aa, Oryctolagus cuniculus
  anti-TNF-alpha antibody Ab9 light chain CDR 1
QSSQNVYNNNDFV

SEQ ID NO: 133 - protein, 7 aa, Oryctolagus cuniculus
  anti-TNF-alpha antibody Ab9 light chain CDR 2
WASTLAS

SEQ ID NO: 134 - protein, 10 aa, Oryctolagus cuniculus
  anti-TNF-alpha antibody Ab9 light chain CDR 3
AGAYITELRT

SEQ ID NO: 135 - protein, 5 aa, Oryctolagus cuniculus
  anti-TNF-alpha antibody Ab9 heavy chain CDR 1
IYWMT

SEQ ID NO: 136 - protein, 16 aa, Oryctolagus cuniculus
  anti-TNF-alpha antibody Ab9 heavy chain CDR 2
VISTDGSAYYATWAKG

SEQ ID NO: 137 - protein, 6 aa, Oryctolagus cuniculus
  anti-TNF-alpha antibody Ab9 heavy chain CDR 3
GGGMDP

SEQ ID NO: 138 - DNA, 366 bp, Oryctolagus cuniculus
  anti-TNF-alpha antibody Ab9 light chain variable domain
atggacacgagggcccccactcagctgctggggctcctgctgctctggctcccaggtgccacatttgcgcaagtgctgacccagactccatcctccgtgtctgcagctgtgggaggcacagtcaccgtcagttgccagtccagtcagaatgtttataataacaacgacttcgtctggtttcagcagaaaccagggcagcctcccaagcgcctaatctactgggcatccactctggcatctggggtcccatcgcggttcaaaggcagtggatctgggacacagttcactctcaccatcaacgacctggaatgtgacgatgctgccacttactactgtgcaggcgcttatattactgagcttaggact

SEQ ID NO: 139 - DNA, 360 bp, Oryctolagus cuniculus
  anti-TNF-alpha antibody Ab9 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtatctactggatgacctgggtccgccaggctccagggaaggggctggaatggatcggagtcattagtactgatggtagcgcatactacgcgacctgggcgaaaggccgattcaccatctccaaaacctcgtcgaccacggtggatctgaggatcaccagtccgacaaccgaggacacggccacctatttctgtgccggagggggcggcatggacccc

SEQ ID NO: 140 - DNA, 39 bp, Oryctolagus cuniculus
  anti-TNF-alpha antibody Ab9 light chain CDR 1
cagtccagtcagaatgtttataataacaacgacttcgtc

SEQ ID NO: 141 - DNA, 21 bp, Oryctolagus cuniculus
  anti-TNF-alpha antibody Ab9 light chain CDR 2
tgggcatccactctggcatct

SEQ ID NO: 142 - DNA, 30 bp, Oryctolagus cuniculus
  anti-TNF-alpha antibody Ab9 light chain CDR 3
gcaggcgcttatattactgagcttaggact

SEQ ID NO: 143 - DNA, 15 bp, Oryctolagus cuniculus
  anti-TNF-alpha antibody Ab9 heavy chain CDR 1
atctactggatgacc

SEQ ID NO: 144 - DNA, 48 bp, Oryctolagus cuniculus
  anti-TNF-alpha antibody Ab9 heavy chain CDR 2
gtcattagtactgatggtagcgcatactacgcgacctgggcgaaaggc

SEQ ID NO: 145 - DNA, 18 bp, Oryctolagus cuniculus
  anti-TNF-alpha antibody Ab9 heavy chain CDR 3
gggggcggcatggacccc

SEQ ID NO: 146 - protein, 122 aa, Oryctolagus cuniculus
  anti-TNF-alpha antibody Ab10 light chain variable domain
MDTRAPTQLLGLLLLWLPGATFAQVLTQTASSVSAAVGGTVTISCQSSQSVYNNNDFIWFQQKPGQPPKRLIYWASTLASGVSSRFKGSGSGTQFTLTINDLECDDAAVYYCAGAYDSEVRA

SEQ ID NO: 147 - protein, 120 aa, Oryctolagus cuniculus
  anti-TNF-alpha antibody Ab10 heavy chain variable domain
METGLRWLLLVAVLKGVQCQSVEESGGRLVTPGTPLTLTCTVSGFSLSIYWMTWVRQAPGRGLEWIGVISTDGTTYYANWAKGRFTISKASSTTVDLRITSPTTEDTATYFCAGGGGMDP

SEQ ID NO: 148 - protein, 13 aa, Oryctolagus cuniculus
  anti-TNF-alpha antibody Ab10 light chain CDR 1
QSSQSVYNNNDFI

SEQ ID NO: 149 - protein, 7 aa, Oryctolagus cuniculus
  anti-TNF-alpha antibody Ab10 light chain CDR 2
WASTLAS

SEQ ID NO: 150 - protein, 10 aa, Oryctolagus cuniculus
  anti-TNF-alpha antibody Ab10 light chain CDR 3
AGAYDSEVRA

SEQ ID NO: 151 - protein, 5 aa, Oryctolagus cuniculus
  anti-TNF-alpha antibody Ab10 heavy chain CDR 1
IYWMT

SEQ ID NO: 152 - protein, 16 aa, Oryctolagus cuniculus
  anti-TNF-alpha antibody Ab10 heavy chain CDR 2
VISTDGTTYYANWAKG

SEQ ID NO: 153 - protein, 6 aa, Oryctolagus cuniculus
  anti-TNF-alpha antibody Ab10 heavy chain CDR 3
GGGMDP

SEQ ID NO: 154 - DNA, 366 bp, Oryctolagus cuniculus
  anti-TNF-alpha antibody Ab10 light chain variable domain
atggacacgagggcccccactcagctgctggggctcctgctgctctggctcccaggtgccacatttgcgcaagtgctgacccagactgcatcgtccgtgtctgcagctgtgggaggcacagtcaccatcagttgccagtccagtcagagtgtttataataataacgacttcatctggtttcagcagaaaccagggcagcctcccaagcgcctcatctactgggcatccactctggcatctggggtctcatcgcggttcaaaggcagtggatctgggacacagttcactctcaccatcaacgacctggagtgtgacgatgctgccgtttactattgtgcaggcgcttatgatagtgaggttagggct

SEQ ID NO: 155 - DNA, 360 bp, Oryctolagus cuniculus
  anti-TNF-alpha antibody Ab10 heavy chain variable domain
atggagactgggctgcgctggcttctcctggtcgctgtgctcaaaggtgtccagtgtcagtcggtggaggagtctgggggtcgcctggtcacgcctgggacacccctgacactcacctgcacagtctctggattctccctcagtatctactggatgacctgggtccgccaggctccagggagggggctggaatggatcggggtcattagtactgatggtaccacatactacgcgaactgggcgaaaggccgattcaccatctccaaagcctcgtcgaccacggtggatctgagaatcaccagtccgacaaccgaggacacggccacctatttctgtgccggagggggcggcatggacccc

SEQ ID NO: 156 - DNA, 39 bp, Oryctolagus cuniculus
  anti-TNF-alpha antibody Ab10 light chain CDR 1
cagtccagtcagagtgtttataataataacgacttcatc

SEQ ID NO: 157 - DNA, 21 bp, Oryctolagus cuniculus
  anti-TNF-alpha antibody Ab10 light chain CDR 2
tgggcatccactctggcatct

SEQ ID NO: 158 - DNA, 30 bp, Oryctolagus cuniculus
  anti-TNF-alpha antibody Ab10 light chain CDR 3
gcaggcgcttatgatagtgaggttagggct

SEQ ID NO: 159 - DNA, 15 bp, Oryctolagus cuniculus
  anti-TNF-alpha antibody Ab10 heavy chain CDR 1
atctactggatgacc

SEQ ID NO: 160 - DNA, 48 bp, Oryctolagus cuniculus
  anti-TNF-alpha antibody Ab10 heavy chain CDR 2
gtcattagtactgatggtaccacatactacgcgaactgggcgaaaggc

SEQ ID NO: 161 - DNA, 18 bp, Oryctolagus cuniculus
  anti-TNF-alpha antibody Ab10 heavy chain CDR 3
gggggcggcatggacccc

SEQ ID NO: 162 - protein, 122 aa, Oryctolagus cuniculus
  anti-TNF-alpha antibody Ab11 light chain variable domain
MDTRAPTQLLGLLLLWLPGATFAQVMTQTPASVSAAVGGTVTISCQSSESVYNNNDLIWFRQKPGQPPKRLIYWASQLASGVSSRFKGSGSGTQFTLTINDLECDDAATYYCAGAYDSEIRA

SEQ ID NO: 163 - protein, 120 aa, Oryctolagus cuniculus
  anti-TNF-alpha antibody Ab11 heavy chain variable domain
METGLRWLLLVAVLKGVQCQSVEESGGRLVTPGTPLTLTCTVSGFSLSIYWMTWVRQAPGKGLEWIGVIASDGSTYYASWAKGRFTISKASSTTVDLKIASPTIEDTATYFCAGGGGMDP

SEQ ID NO: 164 - protein, 13 aa, Oryctolagus cuniculus
  anti-TNF-alpha antibody Ab11 light chain CDR 1
QSSESVYNNNDLI

SEQ ID NO: 165 - protein, 7 aa, Oryctolagus cuniculus
  anti-TNF-alpha antibody Ab11 light chain CDR 2
WASQLAS

SEQ ID NO: 166 - protein, 10 aa, Oryctolagus cuniculus
  anti-TNF-alpha antibody Ab11 light chain CDR 3
AGAYDSEIRA

SEQ ID NO: 167 - protein, 5 aa, Oryctolagus cuniculus
  anti-TNF-alpha antibody Ab11 heavy chain CDR 1
IYWMT

SEQ ID NO: 168 - protein, 16 aa, Oryctolagus cuniculus
  anti-TNF-alpha antibody Ab11 heavy chain CDR 2
VIASDGSTYYASWAKG

SEQ ID NO: 169 - protein, 6 aa, Oryctolagus cuniculus
  anti-TNF-alpha antibody Ab11 heavy chain CDR 3
GGGMDP

SEQ ID NO: 170 - DNA, 366 bp, Oryctolagus cuniculus
  anti-TNF-alpha antibody Ab11 light chain variable domain
atggacacgagggcccccactcagctgctggggctcctgctgctctggctcccaggtgccacatttgcgcaagtgatgacccagactccagcctccgtgtctgcagctgtgggaggcacagtcaccatcagttgccagtccagtgagagtgtttataataataatgacttaatctggttccggcagaaaccagggcagcctcccaagcgcctaatttactgggcatcccaactggcatctggggtctcatcgcggttcaaaggcagtggatctgggacacagttcactctcaccatcaacgacctggagtgtgacgatgctgccacttactactgtgcaggcgcttatgatagtgagattagggct

SEQ ID NO: 171 - DNA, 360 bp, Oryctolagus cuniculus
  anti-TNF-alpha antibody Ab11 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtatctactggatgacctgggtccgccaggctccagggaaggggctggaatggatcggagtcattgcttctgatggtagcacatactacgcgagctgggcgaaaggccgattcaccatctccaaagcctcgtcgaccacggtggatctgaagattgccagcccgacaattgaggacacggccacctatttctgtgccggagggggcggcatggacccc

SEQ ID NO: 172 - DNA, 39 bp, Oryctolagus cuniculus
  anti-TNF-alpha antibody Ab11 light chain CDR 1
cagtccagtgagagtgtttataataataatgacttaatc

SEQ ID NO: 173 - DNA, 21 bp, Oryctolagus cuniculus
  anti-TNF-alpha antibody Ab11 light chain CDR 2
tgggcatcccaactggcatct

SEQ ID NO: 174 - DNA, 30 bp, Oryctolagus cuniculus
  anti-TNF-alpha antibody Ab11 light chain CDR 3
gcaggcgcttatgatagtgagattagggct

SEQ ID NO: 175 - DNA, 15 bp, Oryctolagus cuniculus
  anti-TNF-alpha antibody Ab11 heavy chain CDR 1
atctactggatgacc

SEQ ID NO: 176 - DNA, 48 bp, Oryctolagus cuniculus
  anti-TNF-alpha antibody Ab11 heavy chain CDR 2
gtcattgcttctgatggtagcacatactacgcgagctgggcgaaaggc

SEQ ID NO: 177 - DNA, 18 bp, Oryctolagus cuniculus
  anti-TNF-alpha antibody Ab11 heavy chain CDR 3
gggggcggcatggacccc

SEQ ID NO: 178 - protein, 124 aa, Oryctolagus cuniculus
  anti-TNF-alpha antibody Ab12 light chain variable domain
MDTRAPTQLLGLLLLWLPGARCALVMTQTPSPVSAAVGGTVTISCQSSESVVFNNRLSWYQQKPGQPPKLLIYWASTLASGVPSRFKGSGSGTQFTLTISGVECDDAATYYCAGYKSYSNDDFA

SEQ ID NO: 179 - protein, 128 aa, Oryctolagus cuniculus
  anti-TNF-alpha antibody Ab12 heavy chain variable domain
METGLRWLLLVAVLKGVQCQSVEESGGRLVTPGTPLTLTCTVSGFSLSHYAMGWVRQAPGKGLEWIGIISSNGVTYYATWASGRFTISKTSTTVDLKITSPTTEDTATYFCARGDDTSIIYYIYAFDL

SEQ ID NO: 180 - protein, 13 aa, Oryctolagus cuniculus
  anti-TNF-alpha antibody Ab12 light chain CDR 1
QSSESVVFNNRLS

SEQ ID NO: 181 - protein, 7 aa, Oryctolagus cuniculus
  anti-TNF-alpha antibody Ab12 light chain CDR 2
WASTLAS

SEQ ID NO: 182 - protein, 12 aa, Oryctolagus cuniculus
  anti-TNF-alpha antibody Ab12 light chain CDR 3
AGYKSYSNDDFA

SEQ ID NO: 183 - protein, 5 aa, Oryctolagus cuniculus
  anti-TNF-alpha antibody Ab12 heavy chain CDR 1
HYAMG

SEQ ID NO: 184 - protein, 16 aa, Oryctolagus cuniculus
  anti-TNF-alpha antibody Ab12 heavy chain CDR 2
IISSNGVTYYATWASG

SEQ ID NO: 185 - protein, 15 aa, Oryctolagus cuniculus
  anti-TNF-alpha antibody Ab12 heavy chain CDR 3
GDDTSIIYYIYAFDL

SEQ ID NO: 186 - DNA, 372 bp, Oryctolagus cuniculus
  anti-TNF-alpha antibody Ab12 light chain variable domain
atggacacgagggcccccactcagctgctggggctcctgctgctctggctcccaggtgccagatgtgcgcttgtgatgacccagactccatcccctgtgtctgcagctgtgggaggcacagtcaccatcagttgccagtctagtgagagcgttgtttttaacaaccgcttatcctggtatcagcagaaaccagggcagcctcccaagctcctgatctactgggcatccactctggcatctggggtcccatcgcggttcaaaggcagtggatctgggacacagttcactctcaccatcagtggcgtggagtgtgacgatgctgccacttactactgtgcaggatataaaagttatagtaatgatgattttgct

SEQ ID NO: 187 - DNA, 384 bp, Oryctolagus cuniculus
  anti-TNF-alpha antibody Ab12 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtcactatgcaatgggctgggtccgccaggctccagggaaggggctggaatggatcggaatcattagtagtaatggtgtcacatactacgcgacctgggcgagcggccgattcaccatctccaaaacctcgaccacggtggatctgaaaatcaccagtccgacaaccgaggacacggccacctatttctgtgccagaggagatgatactagtattatttattacatttacgcctttgatctc

SEQ ID NO: 188 - DNA, 39 bp, Oryctolagus cuniculus
  anti-TNF-alpha antibody Ab12 light chain CDR 1
cagtctagtgagagcgttgtttttaacaaccgcttatcc

SEQ ID NO: 189 - DNA, 21 bp, Oryctolagus cuniculus
  anti-TNF-alpha antibody Ab12 light chain CDR 2
tgggcatccactctggcatct

SEQ ID NO: 190 - DNA, 36 bp, Oryctolagus cuniculus
  anti-TNF-alpha antibody Ab12 light chain CDR 3
gcaggatataaaagttatagtaatgatgattttgct

SEQ ID NO: 191 - DNA, 15 bp, Oryctolagus cuniculus
  anti-TNF-alpha antibody Ab12 heavy chain CDR 1
cactatgcaatgggc

SEQ ID NO: 192 - DNA, 48 bp, Oryctolagus cuniculus
  anti-TNF-alpha antibody Ab12 heavy chain CDR 2
atcattagtagtaatggtgtcacatactacgcgacctgggcgagcggc

SEQ ID NO: 193 - DNA, 45 bp, Oryctolagus cuniculus
  anti-TNF-alpha antibody Ab12 heavy chain CDR 3
ggagatgatactagtattatttattacatttacgcctttgatctc

SEQ ID NO: 194 - protein, 125 aa, Oryctolagus cuniculus
  anti-TNF-alpha antibody Ab13 light chain variable domain
MDTRAPTQLLGLLLLWLPGARCADIVMTQTPASVSEPVGGTVTIKCQASQNIYSTLAWYQQKPGQPPKLLIYLASTLASGVPSRFKGSGSGTQFTLTISDLECADAATYYCQTSHGSNSDSFGYA

SEQ ID NO: 195 - protein, 124 aa, Oryctolagus cuniculus
  anti-TNF-alpha antibody Ab13 heavy chain variable domain
METGLRWLLLVAVLKGVQCQSVEESGGRLVTPGTPLTLTCTVSGIDLSSYAMGWVRQAPGKGLEYIGYILSSGITYYASWARGRFTISKTSSTTVDLKMTSLTTEDTATYFCARNGNYNSGTDI

SEQ ID NO: 196 - protein, 11 aa, Oryctolagus cuniculus
  anti-TNF-alpha antibody Ab13 light chain CDR 1
QASQNIYSTLA

SEQ ID NO: 197 - protein, 7 aa, Oryctolagus cuniculus
  anti-TNF-alpha antibody Ab13 light chain CDR 2
LASTLAS

SEQ ID NO: 198 - protein, 14 aa, Oryctolagus cuniculus
  anti-TNF-alpha antibody Ab13 light chain CDR 3
QTSHGSNSDSFGYA

SEQ ID NO: 199 - protein, 5 aa, Oryctolagus cuniculus
  anti-TNF-alpha antibody Ab13 heavy chain CDR 1
SYAMG

SEQ ID NO: 200 - protein, 16 aa, Oryctolagus cuniculus
  anti-TNF-alpha antibody Ab13 heavy chain CDR 2
YILSSGITYYASWARG

SEQ ID NO: 201 - protein, 10 aa, Oryctolagus cuniculus
  anti-TNF-alpha antibody Ab13 heavy chain CDR 3
NGNYNSGTDI

SEQ ID NO: 202 - DNA, 375 bp, Oryctolagus cuniculus
  anti-TNF-alpha antibody Ab13 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagaacatttacagcaccttagcctggtatcagcagaaaccagggcagcctcccaagctcctgatctatctggcatccactctggcatctggggtcccatcgcggttcaaaggcagtggatctgggacacagttcactctcaccatcagcgacctggagtgtgccgatgctgccacttattactgtcaaaccagtcatggtagtaatagtgatagttttggttatgct

SEQ ID NO: 203 - DNA, 372 bp, Oryctolagus cuniculus
  anti-TNF-alpha antibody Ab13 heavy chain variable domain
atggagactgggctgcgctggcttctcctggtcgctgtgctcaaaggtgtccagtgtcagtcggtggaggagtccgggggtcgcctggtcacgcctgggacacccctgacactcacttgcacagtctctggaatcgacctcagtagctatgcaatgggctgggtccgccaggctccagggaaggggctggaatacatcggatacattcttagtagtggtatcacatactacgcgagttgggcgagaggccgattcaccatctccaaaacctcgtcgaccacggtggatctgaaaatgaccagtctgacaaccgaggacacggccacctatttctgtgccagaaatggtaattataatagtggtacggacatc

SEQ ID NO: 204 - DNA, 33 bp, Oryctolagus cuniculus
  anti-TNF-alpha antibody Ab13 light chain CDR 1
caggccagtcagaacatttacagcaccttagcc

SEQ ID NO: 205 - DNA, 21 bp, Oryctolagus cuniculus
  anti-TNF-alpha antibody Ab13 light chain CDR 2
ctggcatccactctggcatct

SEQ ID NO: 206 - DNA, 42 bp, Oryctolagus cuniculus
  anti-TNF-alpha antibody Ab13 light chain CDR 3
caaaccagtcatggtagtaatagtgatagttttggttatgct

SEQ ID NO: 207 - DNA, 15 bp, Oryctolagus cuniculus
  anti-TNF-alpha antibody Ab13 heavy chain CDR 1
agctatgcaatgggc

SEQ ID NO: 208 - DNA, 48 bp, Oryctolagus cuniculus
  anti-TNF-alpha antibody Ab13 heavy chain CDR 2
tacattcttagtagtggtatcacatactacgcgagttgggcgagaggc

SEQ ID NO: 209 - DNA, 30 bp, Oryctolagus cuniculus
  anti-TNF-alpha antibody Ab13 heavy chain CDR 3
aatggtaattataatagtggtacggacatc

SEQ ID NO: 210 - protein, 125 aa, Oryctolagus cuniculus
  anti-TNF-alpha antibody Ab14 light chain variable domain
MDTRAPTQLLGLLLLWLPGARCADIVMTQTPASVSEPVGGTVTIKCQASQNIYSTLAWYQQKPGQPPKLLIYLASTLASGVPSRFKGSGSGTQFTLTISDLECADAATYYCQTNHGSNSDSFGYA

SEQ ID NO: 211 - protein, 124 aa, Oryctolagus cuniculus
  anti-TNF-alpha antibody Ab14 heavy chain variable domain
METGLRWLLLVAVLKGVQCQSVEESGGRLVTPGTPLTLTCTVSGFSLSSYAMGWVRQAPGKGLEYIGYIGSSGITYYTSWARGRFTISKPSSTTVDLKMTSLTTEDTATYFCARNGNYNSGTDI

SEQ ID NO: 212 - protein, 11 aa, Oryctolagus cuniculus
  anti-TNF-alpha antibody Ab14 light chain CDR 1
QASQNIYSTLA

SEQ ID NO: 213 - protein, 7 aa, Oryctolagus cuniculus
  anti-TNF-alpha antibody Ab14 light chain CDR 2
LASTLAS

SEQ ID NO: 214 - protein, 14 aa, Oryctolagus cuniculus
  anti-TNF-alpha antibody Ab14 light chain CDR 3
QTNHGSNSDSFGYA

SEQ ID NO: 215 - protein, 5 aa, Oryctolagus cuniculus
  anti-TNF-alpha antibody Ab14 heavy chain CDR 1
SYAMG

SEQ ID NO: 216 - protein, 16 aa, Oryctolagus cuniculus
  anti-TNF-alpha antibody Ab14 heavy chain CDR 2
YIGSSGITYYTSWARG

SEQ ID NO: 217 - protein, 10 aa, Oryctolagus cuniculus
  anti-TNF-alpha antibody Ab14 heavy chain CDR 3
NGNYNSGTDI

SEQ ID NO: 218 - DNA, 375 bp, Oryctolagus cuniculus
  anti-TNF-alpha antibody Ab14 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagaacatttacagcaccttagcctggtatcagcagaaaccagggcagcctcccaagctcctgatctatctggcatccactctggcatctggggtcccatcgcggttcaaaggcagtggatctgggacacagttcactctcaccatcagcgacctggagtgtgccgatgctgccacctattactgtcaaaccaatcatggtagtaatagtgatagttttggttatgct

SEQ ID NO: 219 - DNA, 372 bp, Oryctolagus cuniculus
  anti-TNF-alpha antibody Ab14 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtagctatgcaatgggctgggtccgccaggctccagggaaggggctggaatacatcggatacattggtagtagtggtatcacatactacacgagttgggcgagaggccgtttcaccatctccaaaccctcgtcgaccacggtggatctgaaaatgaccagtctgacaaccgaggacacggccacctatttctgtgccagaaatggtaattataatagtggtacggacatc

SEQ ID NO: 220 - DNA, 33 bp, Oryctolagus cuniculus; identical to SEQ ID NO: 204
  anti-TNF-alpha antibody Ab14 light chain CDR 1

SEQ ID NO: 221 - DNA, 21 bp, Oryctolagus cuniculus
  anti-TNF-alpha antibody Ab14 light chain CDR 2
ctggcatccactctggcatct

SEQ ID NO: 222 - DNA, 42 bp, Oryctolagus cuniculus
  anti-TNF-alpha antibody Ab14 light chain CDR 3
caaaccaatcatggtagtaatagtgatagttttggttatgct

SEQ ID NO: 223 - DNA, 15 bp, Oryctolagus cuniculus
  anti-TNF-alpha antibody Ab14 heavy chain CDR 1
agctatgcaatgggc

SEQ ID NO: 224 - DNA, 48 bp, Oryctolagus cuniculus
  anti-TNF-alpha antibody Ab14 heavy chain CDR 2
tacattggtagtagtggtatcacatactacacgagttgggcgagaggc

SEQ ID NO: 225 - DNA, 30 bp, Oryctolagus cuniculus; identical to SEQ ID NO: 209
  anti-TNF-alpha antibody Ab14 heavy chain CDR 3

SEQ ID NO: 226 - protein, 125 aa, Oryctolagus cuniculus
  anti-TNF-alpha antibody Ab15 light chain variable domain
MDTRAPTQLLGLLLLWLPGARCADIVMTQTPASVSEPVGGTVTIKCQASQSIYSSFSWYQQIPGQRPKLLIYYASTLASGVPSRFSGSGSGTDFTLTISDLECADAATYYCQSNHGSNGDSFGNA

SEQ ID NO: 227 - protein, 121 aa, Oryctolagus cuniculus
  anti-TNF-alpha antibody Ab15 heavy chain variable domain
METGLRWLLLVAVLKGVQCQSVEESGGRLVSPGTPLTLTCTVSGIDLSSYGMGWVRQAPGKGLEYIGYMIASGITYYAAWAKGRFTISKTSSTTVDLKITSPTTEDTATYFCARNYYGMDP

SEQ ID NO: 228 - protein, 11 aa, Oryctolagus cuniculus
  anti-TNF-alpha antibody Ab15 light chain CDR 1
QASQSIYSSFS

SEQ ID NO: 229 - protein, 7 aa, Oryctolagus cuniculus
  anti-TNF-alpha antibody Ab15 light chain CDR 2
YASTLAS

SEQ ID NO: 230 - protein, 14 aa, Oryctolagus cuniculus
  anti-TNF-alpha antibody Ab15 light chain CDR 3
QSNHGSNGDSFGNA

SEQ ID NO: 231 - protein, 5 aa, Oryctolagus cuniculus
  anti-TNF-alpha antibody Ab15 heavy chain CDR 1
SYGMG

SEQ ID NO: 232 - protein, 16 aa, Oryctolagus cuniculus
  anti-TNF-alpha antibody Ab15 heavy chain CDR 2
YMIASGITYYAAWAKG

SEQ ID NO: 233 - protein, 7 aa, Oryctolagus cuniculus
  anti-TNF-alpha antibody Ab15 heavy chain CDR 3
NYYGMDP

SEQ ID NO: 234 - DNA, 375 bp, Oryctolagus cuniculus
  anti-TNF-alpha antibody Ab15 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagagcatttacagctccttttcctggtatcaacagataccaggccagcgtcccaagctcctgatctattatgcatccactctggcctctggggtcccatcgcgattcagcggcagtggatctgggacagatttcactctcaccatcagcgacctggagtgtgccgatgctgccacttactactgtcaaagcaatcatggtagtaatggtgatagttttggtaatgct

SEQ ID NO: 235 - DNA, 363 bp, Oryctolagus cuniculus
  anti-TNF-alpha antibody Ab15 heavy chain variable domain
atggagactgggctgcgctggcttctcctggtcgctgtgctcaaaggtgtccagtgtcagtcggtggaggagtccgggggtcgcctggtgtcgcctgggacacccctgacactcacctgcacagtctctggaatcgacctcagtagttatggaatgggctgggtccgccaggctccagggaaggggctggaatacatcggatacatgattgctagtggtatcacatattacgcggcctgggcgaaaggccgattcaccatctccaaaacctcgtcgaccacggtggatctgaaaatcaccagtccgacaaccgaggacacggccacctatttctgtgccagaaattactacggcatggacccc

SEQ ID NO: 236 - DNA, 33 bp, Oryctolagus cuniculus
  anti-TNF-alpha antibody Ab15 light chain CDR 1
caggccagtcagagcatttacagctccttttcc

SEQ ID NO: 237 - DNA, 21 bp, Oryctolagus cuniculus
  anti-TNF-alpha antibody Ab15 light chain CDR 2
tatgcatccactctggcctct

SEQ ID NO: 238 - DNA, 42 bp, Oryctolagus cuniculus
  anti-TNF-alpha antibody Ab15 light chain CDR 3
caaagcaatcatggtagtaatggtgatagttttggtaatgct

SEQ ID NO: 239 - DNA, 15 bp, Oryctolagus cuniculus
  anti-TNF-alpha antibody Ab15 heavy chain CDR 1
agttatggaatgggc

SEQ ID NO: 240 - DNA, 48 bp, Oryctolagus cuniculus
  anti-TNF-alpha antibody Ab15 heavy chain CDR 2
tacatgattgctagtggtatcacatattacgcggcctgggcgaaaggc

SEQ ID NO: 241 - DNA, 21 bp, Oryctolagus cuniculus
  anti-TNF-alpha antibody Ab15 heavy chain CDR 3
aattactacggcatggacccc

SEQ ID NO: 242 - protein, 125 aa, Oryctolagus cuniculus
  anti-TNF-alpha antibody Ab16 light chain variable domain
MDTRAPTQLLGLLLLWLPGARCADIVMTQTPASVSEPVGGTVTIKCQASQTIYSSLSWYQQKPGQRPKLLIYAASTLASGVPSRFKGSGSGTDFTLTISDLECADAATYYCQSNHGSNSDSYGNA

SEQ ID NO: 243 - protein, 125 aa, Oryctolagus cuniculus
  anti-TNF-alpha antibody Ab16 heavy chain variable domain
METGLRWLLLVAVLKGVQCQSLEESGGRLVKPDETLTITCTVSGIDLNNYNMGWVRQAPGKGLEYIGYILGSGITYYATWAKGRFTISKTSSTTVDLKMTSLTTEDTATYFCAGSIYYRGYGMDP

SEQ ID NO: 244 - protein, 11 aa, Oryctolagus cuniculus
  anti-TNF-alpha antibody Ab16 light chain CDR 1
QASQTIYSSLS

SEQ ID NO: 245 - protein, 7 aa, Oryctolagus cuniculus
  anti-TNF-alpha antibody Ab16 light chain CDR 2
AASTLAS

SEQ ID NO: 246 - protein, 14 aa, Oryctolagus cuniculus
  anti-TNF-alpha antibody Ab16 light chain CDR 3
QSNHGSNSDSYGNA

SEQ ID NO: 247 - protein, 5 aa, Oryctolagus cuniculus
  anti-TNF-alpha antibody Ab16 heavy chain CDR 1
NYNMG

SEQ ID NO: 248 - protein, 16 aa, Oryctolagus cuniculus
  anti-TNF-alpha antibody Ab16 heavy chain CDR 2
YILGSGITYYATWAKG

SEQ ID NO: 249 - protein, 11 aa, Oryctolagus cuniculus
  anti-TNF-alpha antibody Ab16 heavy chain CDR 3
SIYYRGYGMDP

SEQ ID NO: 250 - DNA, 375 bp, Oryctolagus cuniculus
  anti-TNF-alpha antibody Ab16 light chain variable domain
atggacacgagggcccccactcagctgctggggctcctgctgctctggctcccaggtgccagatgtgctgacattgtgatgacgcagactccagcctccgtgtccgaacctgtgggaggcacagtcaccatcaagtgtcaggccagtcagaccatttacagtagcttatcctggtatcagcagaaaccagggcagcgtcccaagctcctgatctatgctgcatccactctggcatctggggtcccatcgcggttcaaaggcagtggatctgggacagatttcactctcaccataagcgacctggagtgtgccgatgctgccacttactactgtcaaagtaatcatggtagtaatagtgatagttatggcaatgct

SEQ ID NO: 251 - DNA, 375 bp, Oryctolagus cuniculus
  anti-TNF-alpha antibody Ab16 heavy chain variable domain
atggagactgggctgcgctggcttctcctggtcgctgtgctcaaaggtgtccagtgtcagtcgctggaggagtccgggggtcgcctggtcaagcctgacgaaaccctgacaatcacctgcacagtctctggaatcgacctcaataactacaacatgggctgggtccgccaggctccagggaaggggctggaatacatcggatacattcttggtagtggtatcacatactacgcgacctgggcgaaaggccgattcaccatctcgaaaacctcgtcgaccacggtggatctgaaaatgaccagtctgacaaccgaggacacggccacgtatttctgtgctggtagtatttattatagggggtacggcatggacccc

SEQ ID NO: 252 - DNA, 33 bp, Oryctolagus cuniculus
  anti-TNF-alpha antibody Ab16 light chain CDR 1
caggccagtcagaccatttacagtagcttatcc

SEQ ID NO: 253 - DNA, 21 bp, Oryctolagus cuniculus
  anti-TNF-alpha antibody Ab16 light chain CDR 2
gctgcatccactctggcatct

SEQ ID NO: 254 - DNA, 42 bp, Oryctolagus cuniculus
  anti-TNF-alpha antibody Ab16 light chain CDR 3
caaagtaatcatggtagtaatagtgatagttatggcaatgct

SEQ ID NO: 255 - DNA, 15 bp, Oryctolagus cuniculus
  anti-TNF-alpha antibody Ab16 heavy chain CDR 1
aactacaacatgggc

SEQ ID NO: 256 - DNA, 48 bp, Oryctolagus cuniculus
  anti-TNF-alpha antibody Ab16 heavy chain CDR 2
tacattcttggtagtggtatcacatactacgcgacctgggcgaaaggc

SEQ ID NO: 257 - DNA, 33 bp, Oryctolagus cuniculus
  anti-TNF-alpha antibody Ab16 heavy chain CDR 3
agtatttattatagggggtacggcatggacccc

SEQ ID NO: 258 - protein, 125 aa, Oryctolagus cuniculus
  anti-TNF-alpha antibody Ab17 light chain variable domain
MDTRAPTQLLGLLLLWLPGARCADIVMTQTPASVSEPVGGTVTIKCQASQSIYSTLAWYQQKPGQPPKLLISLASTLASGVPSRFKGSGSGTQFTLTISDLECADAATYYCQTNHGSNSDSFGYA

SEQ ID NO: 259 - protein, 124 aa, Oryctolagus cuniculus
  anti-TNF-alpha antibody Ab17 heavy chain variable domain
METGLRWLLLVAVLKGVQCQSLEESGGRLVTPGGSLTLTCTVSGIDLSSYAMGWVRQAPGKGLEYIGYVLGSGITYYASWARGRFTISKTSSTTVDLKMTSLTTEDTATYFCVRNDNYNSGTDI

SEQ ID NO: 260 - protein, 11 aa, Oryctolagus cuniculus
  anti-TNF-alpha antibody Ab17 light chain CDR 1
QASQSIYSTLA

SEQ ID NO: 261 - protein, 7 aa, Oryctolagus cuniculus
  anti-TNF-alpha antibody Ab17 light chain CDR 2
LASTLAS

SEQ ID NO: 262 - protein, 14 aa, Oryctolagus cuniculus
  anti-TNF-alpha antibody Ab17 light chain CDR 3
QTNHGSNSDSFGYA

SEQ ID NO: 263 - protein, 5 aa, Oryctolagus cuniculus
  anti-TNF-alpha antibody Ab17 heavy chain CDR 1
SYAMG

SEQ ID NO: 264 - protein, 16 aa, Oryctolagus cuniculus
  anti-TNF-alpha antibody Ab17 heavy chain CDR 2
YVLGSGITYYASWARG

SEQ ID NO: 265 - protein, 10 aa, Oryctolagus cuniculus
  anti-TNF-alpha antibody Ab17 heavy chain CDR 3
NDNYNSGTDI

SEQ ID NO: 266 - DNA, 375 bp, Oryctolagus cuniculus
  anti-TNF-alpha antibody Ab17 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagagcatttacagcaccttagcctggtatcagcagaaaccagggcagcctcccaaactcctgatctcgctggcatccactctggcatctggggtcccatcgcggttcaaaggcagtggatctgggacacagttcactctcaccatcagcgacctggagtgtgccgatgctgccacttattactgtcaaaccaatcatggtagtaatagtgatagttttggttatgct

SEQ ID NO: 267 - DNA, 372 bp, Oryctolagus cuniculus
  anti-TNF-alpha antibody Ab17 heavy chain variable domain
atggagactgggctgcgctggcttctcctggtcgctgtgctcaaaggtgtccagtgtcagtcgctggaggagtccgggggtcgcctggtaacgcctggaggatccctgacactcacctgcacagtctctggaatcgacctcagtagctatgcaatgggctgggtccgccaggctccagggaaggggctggaatacatcggatacgttcttggtagtggtatcacatactacgcgagttgggcgagaggccgattcaccatctccaaaacctcgtcgaccacggtggatctgaagatgaccagtctgacaaccgaggacacggccacctatttctgtgtcagaaatgataattataatagtggcacggacatc

SEQ ID NO: 268 - DNA, 33 bp, Oryctolagus cuniculus
  anti-TNF-alpha antibody Ab17 light chain CDR 1
caggccagtcagagcatttacagcaccttagcc

SEQ ID NO: 269 - DNA, 21 bp, Oryctolagus cuniculus
  anti-TNF-alpha antibody Ab17 light chain CDR 2
ctggcatccactctggcatct

SEQ ID NO: 270 - DNA, 42 bp, Oryctolagus cuniculus; identical to SEQ ID NO: 222
  anti-TNF-alpha antibody Ab17 light chain CDR 3

SEQ ID NO: 271 - DNA, 15 bp, Oryctolagus cuniculus
  anti-TNF-alpha antibody Ab17 heavy chain CDR 1
agctatgcaatgggc

SEQ ID NO: 272 - DNA, 48 bp, Oryctolagus cuniculus
  anti-TNF-alpha antibody Ab17 heavy chain CDR 2
tacgttcttggtagtggtatcacatactacgcgagttgggcgagaggc

SEQ ID NO: 273 - DNA, 30 bp, Oryctolagus cuniculus
  anti-TNF-alpha antibody Ab17 heavy chain CDR 3
aatgataattataatagtggcacggacatc

SEQ ID NO: 274 - protein, 125 aa, Oryctolagus cuniculus
  anti-TNF-alpha antibody Ab18 light chain variable domain
MDTRAPTQLLGLLLLWLPGARCADIVMTQTPASVSEPVGGTVTIKCQASQNIYSTLAWYQQKPGQPPKLLIYLASTLESGVPSRFKGSGSGTEFTLTISDLECADAATYYCQTSHGSNSESFGYA

SEQ ID NO: 275 - protein, 124 aa, Oryctolagus cuniculus
  anti-TNF-alpha antibody Ab18 heavy chain variable domain
METGLRWLLLVAVLKGVQCQSVEESGGRLVTPGTPLTLTCTVSGIDLSSYAMGWVRQAPGKGLEYIGYILSSGITYYASWARGRFTISKTSSTTVDLKMTSLTTEDTATYFCVRNGNYNVGTDI

SEQ ID NO: 276 - protein, 11 aa, Oryctolagus cuniculus
  anti-TNF-alpha antibody Ab18 light chain CDR 1
QASQNIYSTLA

SEQ ID NO: 277 - protein, 7 aa, Oryctolagus cuniculus
  anti-TNF-alpha antibody Ab18 light chain CDR 2
LASTLES

SEQ ID NO: 278 - protein, 14 aa, Oryctolagus cuniculus
  anti-TNF-alpha antibody Ab18 light chain CDR 3
QTSHGSNSESFGYA

SEQ ID NO: 279 - protein, 5 aa, Oryctolagus cuniculus
  anti-TNF-alpha antibody Ab18 heavy chain CDR 1
SYAMG

SEQ ID NO: 280 - protein, 16 aa, Oryctolagus cuniculus
  anti-TNF-alpha antibody Ab18 heavy chain CDR 2
YILSSGITYYASWARG

SEQ ID NO: 281 - protein, 10 aa, Oryctolagus cuniculus
  anti-TNF-alpha antibody Ab18 heavy chain CDR 3
NGNYNVGTDI

SEQ ID NO: 282 - DNA, 375 bp, Oryctolagus cuniculus
  anti-TNF-alpha antibody Ab18 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagaacatttacagcaccttagcctggtatcagcagaaaccagggcagcctcccaagctcctgatctatctggcatccactctggaatctggggtcccatcgcggttcaaaggcagtggatctgggacagagttcactctcaccatcagcgacctggagtgtgccgatgctgccacttattactgtcaaaccagtcatggtagtaatagtgaaagttttggttatgct

SEQ ID NO: 283 - DNA, 372 bp, Oryctolagus cuniculus
  anti-TNF-alpha antibody Ab18 heavy chain variable domain
atggagactgggctgcgctggcttctcctggtcgctgtgctcaaaggtgtccagtgtcagtcggtggaggagtccgggggtcgcctggtcacgcctgggacacccctgacactcacttgcacggtctctggaatcgacctcagtagctatgcaatgggctgggtccgccaggctccagggaaggggctggaatacatcggatacattcttagtagtggtatcacatactacgcgagttgggcgagaggccgattcaccatctccaaaacctcgtcgaccacggtggatctgaaaatgaccagtctgacaaccgaggacacggccacctatttctgtgtcagaaatggtaattataatgttggtacggacatc

SEQ ID NO: 284 - DNA, 33 bp, Oryctolagus cuniculus; identical to SEQ ID NO: 204
  anti-TNF-alpha antibody Ab18 light chain CDR 1

SEQ ID NO: 285 - DNA, 21 bp, Oryctolagus cuniculus
  anti-TNF-alpha antibody Ab18 light chain CDR 2
ctggcatccactctggaatct

SEQ ID NO: 286 - DNA, 42 bp, Oryctolagus cuniculus
  anti-TNF-alpha antibody Ab18 light chain CDR 3
caaaccagtcatggtagtaatagtgaaagttttggttatgct

SEQ ID NO: 287 - DNA, 15 bp, Oryctolagus cuniculus
  anti-TNF-alpha antibody Ab18 heavy chain CDR 1
agctatgcaatgggc

SEQ ID NO: 288 - DNA, 48 bp, Oryctolagus cuniculus; identical to SEQ ID NO: 208
  anti-TNF-alpha antibody Ab18 heavy chain CDR 2

SEQ ID NO: 289 - DNA, 30 bp, Oryctolagus cuniculus
  anti-TNF-alpha antibody Ab18 heavy chain CDR 3
aatggtaattataatgttggtacggacatc

SEQ ID NO: 290 - protein, 125 aa, Oryctolagus cuniculus
  anti-TNF-alpha antibody Ab19 light chain variable domain
MDTRAPTQLLGLLLLWLPGARCADIVMTQTPSSVSEPVRGTVTIKCQASQNIYSYLSWYRQSPGQPPNLLIYKASTLASGVPSRFKGSGSGTDFTLTISDLECADAATYYCQSNYGSNSDSFGNA

SEQ ID NO: 291 - protein, 122 aa, Oryctolagus cuniculus
  anti-TNF-alpha antibody Ab19 heavy chain variable domain
METGLRWLLLVAVLKGVQCQSVEESGGRLVTPGTPLTLTCSVSGFSLNNYIMGWVRQAPGKGLEFIGYIAFGIGPYYASWAKGRFTSSSTSSTTVDLKMTSLTPEDTATYFCARGDVSGNDI

SEQ ID NO: 292 - protein, 11 aa, Oryctolagus cuniculus
  anti-TNF-alpha antibody Ab19 light chain CDR 1
QASQNIYSYLS

SEQ ID NO: 293 - protein, 7 aa, Oryctolagus cuniculus
  anti-TNF-alpha antibody Ab19 light chain CDR 2
KASTLAS

SEQ ID NO: 294 - protein, 14 aa, Oryctolagus cuniculus
  anti-TNF-alpha antibody Ab19 light chain CDR 3
QSNYGSNSDSFGNA

SEQ ID NO: 295 - protein, 5 aa, Oryctolagus cuniculus
  anti-TNF-alpha antibody Ab19 heavy chain CDR 1
NYIMG

SEQ ID NO: 296 - protein, 16 aa, Oryctolagus cuniculus
  anti-TNF-alpha antibody Ab19 heavy chain CDR 2
YIAFGIGPYYASWAKG

SEQ ID NO: 297 - protein, 8 aa, Oryctolagus cuniculus
  anti-TNF-alpha antibody Ab19 heavy chain CDR 3
GDVSGNDI

SEQ ID NO: 298 - DNA, 375 bp, Oryctolagus cuniculus
  anti-TNF-alpha antibody Ab19 light chain variable domain
atggacacgagggcccccactcagctgctggggctcctgctgctctggctcccaggtgccagatgtgctgacattgtgatgacccagactccatcctccgtgtctgaacctgtgcgaggcacagtcaccatcaagtgccaggccagtcagaacatttacagctacttgtcctggtatcgacagagcccagggcagcctcccaacctcctgatctacaaggcatccactctggcatctggggtcccatcgcggttcaaaggcagtggatctgggacagatttcactctcaccatcagcgacctggagtgtgccgatgctgccacttactactgtcaaagcaattatggtagtaatagtgatagttttgggaatgct

SEQ ID NO: 299 - DNA, 366 bp, Oryctolagus cuniculus
  anti-TNF-alpha antibody Ab19 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgctcagtctctggattctccctcaataactatataatgggctgggtccgccaggctccagggaaggggctggaattcatcggatacattgcttttggtattggcccatactacgcgagctgggcgaaaggccgattcaccagctccagcacctcgtcgaccacggtggatctgaaaatgaccagtctgacacccgaggacacggccacctatttctgtgccagaggtgatgttagtggtaatgacatt

SEQ ID NO: 300 - DNA, 33 bp, Oryctolagus cuniculus; identical to SEQ ID NO: 28
  anti-TNF-alpha antibody Ab19 light chain CDR 1

SEQ ID NO: 301 - DNA, 21 bp, Oryctolagus cuniculus
  anti-TNF-alpha antibody Ab19 light chain CDR 2
aaggcatccactctggcatct

SEQ ID NO: 302 - DNA, 42 bp, Oryctolagus cuniculus; identical to SEQ ID NO: 126
  anti-TNF-alpha antibody Ab19 light chain CDR 3

SEQ ID NO: 303 - DNA, 15 bp, Oryctolagus cuniculus
  anti-TNF-alpha antibody Ab19 heavy chain CDR 1
aactatataatgggc

SEQ ID NO: 304 - DNA, 48 bp, Oryctolagus cuniculus; identical to SEQ ID NO: 32
  anti-TNF-alpha antibody Ab19 heavy chain CDR 2

SEQ ID NO: 305 - DNA, 24 bp, Oryctolagus cuniculus
  anti-TNF-alpha antibody Ab19 heavy chain CDR 3
ggtgatgttagtggtaatgacatt

SEQ ID NO: 306 - protein, 125 aa, Oryctolagus cuniculus
  anti-TNF-alpha antibody Ab20 light chain variable domain
MDTRAPTQLLGLLLLWLPGARCADIVMTQTPASVSEPVGGTVTIKCQASQNIYTTLAWYQQKPGQPPKLLIYLASTLASGVPSRFKGSGSETQFTLTISDLECADAATYYCQTSHGSNSDSFGYV

SEQ ID NO: 307 - protein, 124 aa, Oryctolagus cuniculus
  anti-TNF-alpha antibody Ab20 heavy chain variable domain
METGLRWLLLVAVLKGVQCQSVEESGGRLVTPGTPLTLTCTVSGIDLNSYAMGWVRQAPGKGLEYIGYILSSGITYYATWAKGRFTISKTSSTTVDLKMTSLTTEDTATYFCVRNGNYNSGTDI

SEQ ID NO: 308 - protein, 11 aa, Oryctolagus cuniculus
  anti-TNF-alpha antibody Ab20 light chain CDR 1
QASQNIYTTLA

SEQ ID NO: 309 - protein, 7 aa, Oryctolagus cuniculus
  anti-TNF-alpha antibody Ab20 light chain CDR 2
LASTLAS

SEQ ID NO: 310 - protein, 14 aa, Oryctolagus cuniculus
  anti-TNF-alpha antibody Ab20 light chain CDR 3
QTSHGSNSDSFGYV

SEQ ID NO: 311 - protein, 5 aa, Oryctolagus cuniculus
  anti-TNF-alpha antibody Ab20 heavy chain CDR 1
SYAMG

SEQ ID NO: 312 - protein, 16 aa, Oryctolagus cuniculus
  anti-TNF-alpha antibody Ab20 heavy chain CDR 2
YILSSGITYYATWAKG

SEQ ID NO: 313 - protein, 10 aa, Oryctolagus cuniculus
  anti-TNF-alpha antibody Ab20 heavy chain CDR 3
NGNYNSGTDI

SEQ ID NO: 314 - DNA, 375 bp, Oryctolagus cuniculus
  anti-TNF-alpha antibody Ab20 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaaatgccaggccagtcagaacatttacaccaccttagcctggtatcagcagaaaccagggcagcctcccaagctcctgatctatctggcatccactctggcatctggggtcccatcgcggttcaaaggcagtggatctgagacacagttcactctcaccatcagcgacctggagtgtgccgatgctgccacttattactgtcaaaccagtcatggtagtaatagtgatagttttggttatgtt

SEQ ID NO: 315 - DNA, 372 bp, Oryctolagus cuniculus
  anti-TNF-alpha antibody Ab20 heavy chain variable domain
atggagactgggctgcgctggcttctcctggtcgctgtgctcaaaggtgtccagtgtcagtcagtggaggagtccgggggtcgcctggtcacgcctgggacacccctgacactcacttgcacagtctctggaatcgacctcaatagctatgcaatgggctgggtccgccaggctccagggaaggggctggaatacatcggatacattcttagtagtggtatcacatactacgcgacctgggcgaaaggccgattcaccatctccaaaacctcgtcgaccacggtggatctgaaaatgaccagtctgacaaccgaggacacggccacctatttctgtgtcaggaatggtaattataatagtggtacggacatc

SEQ ID NO: 316 - DNA, 33 bp, Oryctolagus cuniculus
  anti-TNF-alpha antibody Ab20 light chain CDR 1
caggccagtcagaacatttacaccaccttagcc

SEQ ID NO: 317 - DNA, 21 bp, Oryctolagus cuniculus
  anti-TNF-alpha antibody Ab20 light chain CDR 2
ctggcatccactctggcatct

SEQ ID NO: 318 - DNA, 42 bp, Oryctolagus cuniculus
  anti-TNF-alpha antibody Ab20 light chain CDR 3
caaaccagtcatggtagtaatagtgatagttttggttatgtt

SEQ ID NO: 319 - DNA, 15 bp, Oryctolagus cuniculus
  anti-TNF-alpha antibody Ab20 heavy chain CDR 1
agctatgcaatgggc

SEQ ID NO: 320 - DNA, 48 bp, Oryctolagus cuniculus
  anti-TNF-alpha antibody Ab20 heavy chain CDR 2
tacattcttagtagtggtatcacatactacgcgacctgggcgaaaggc

SEQ ID NO: 321 - DNA, 30 bp, Oryctolagus cuniculus; identical to SEQ ID NO: 209
  anti-TNF-alpha antibody Ab20 heavy chain CDR 3

SEQ ID NO: 322 - protein, 125 aa, Oryctolagus cuniculus
  anti-TNF-alpha antibody Ab21 light chain variable domain
MDTRAPTQLLGLLLLWLPGARCADIVMTQTPSSVSAAVGGTVTIKCQASQSIDTYLAWYQQKPGQRPKLLIYGASNLASGVSSRFKGSGSGTEFALTISDLECADAATYYCQSNYGSNSDSFGNG

SEQ ID NO: 323 - protein, 127 aa, Oryctolagus cuniculus
  anti-TNF-alpha antibody Ab21 heavy chain variable domain
METGLRWLLLVAVFKGVQCQSVEESGGRLVTPGTPLTLTCTVSGFSLSTYTMGWVRQAPGKGLEYIGYISYGGLAYYATWVNGRFTISKTSTTVDLKMTSLTASDTATYFCARAASGAWGHAYGLDL

SEQ ID NO: 324 - protein, 11 aa, Oryctolagus cuniculus
  anti-TNF-alpha antibody Ab21 light chain CDR 1
QASQSIDTYLA

SEQ ID NO: 325 - protein, 7 aa, Oryctolagus cuniculus
  anti-TNF-alpha antibody Ab21 light chain CDR 2
GASNLAS

SEQ ID NO: 326 - protein, 14 aa, Oryctolagus cuniculus
  anti-TNF-alpha antibody Ab21 light chain CDR 3
QSNYGSNSDSFGNG

SEQ ID NO: 327 - protein, 5 aa, Oryctolagus cuniculus
  anti-TNF-alpha antibody Ab21 heavy chain CDR 1
TYTMG

SEQ ID NO: 328 - protein, 16 aa, Oryctolagus cuniculus
  anti-TNF-alpha antibody Ab21 heavy chain CDR 2
YISYGGLAYYATWVNG

SEQ ID NO: 329 - protein, 14 aa, Oryctolagus cuniculus
  anti-TNF-alpha antibody Ab21 heavy chain CDR 3
AASGAWGHAYGLDL

SEQ ID NO: 330 - DNA, 375 bp, Oryctolagus cuniculus
  anti-TNF-alpha antibody Ab21 light chain variable domain
atggacacgagggcccccactcagctgctggggctcctgctgctctggctcccaggtgccagatgtgctgacattgtgatgacccagactccatcctccgtgtctgcagctgtgggaggcacagtcaccatcaagtgccaggccagtcagagcattgatacctacttagcctggtatcagcagaaaccagggcagcgtcccaagctcctgatctatggtgcatccaatctggcatctggggtctcatcgcggttcaaaggcagtggatctgggacagaattcgctctcaccatcagcgacctggagtgtgccgatgctgccacttactactgtcaaagcaattatggtagtaatagtgatagttttggtaatggt

SEQ ID NO: 331 - DNA, 381 bp, Oryctolagus cuniculus
  anti-TNF-alpha antibody Ab21 heavy chain variable domain
atggagactgggctgcgctggcttctcctggtcgctgtgttcaaaggtgtccagtgtcagtcggtggaggagtccgggggtcgcctggtcacgcctgggacacccctgacactcacctgcacagtctctggattctccctcagtacctatacaatgggctgggtccgccaggctccagggaaggggctggaatacatcgggtacattagttatggtggtctcgcatactacgcgacctgggtgaatggccgattcaccatctccaaaacctcgaccacggtggatctgaaaatgaccagtctgacagcttcagacacggccacctatttctgtgccagagcggctagtggtgcctggggtcatgcctacggcttggacctc

SEQ ID NO: 332 - DNA, 33 bp, Oryctolagus cuniculus
  anti-TNF-alpha antibody Ab21 light chain CDR 1
caggccagtcagagcattgatacctacttagcc

SEQ ID NO: 333 - DNA, 21 bp, Oryctolagus cuniculus
  anti-TNF-alpha antibody Ab21 light chain CDR 2
ggtgcatccaatctggcatct

SEQ ID NO: 334 - DNA, 42 bp, Oryctolagus cuniculus
  anti-TNF-alpha antibody Ab21 light chain CDR 3
caaagcaattatggtagtaatagtgatagttttggtaatggt

SEQ ID NO: 335 - DNA, 15 bp, Oryctolagus cuniculus
  anti-TNF-alpha antibody Ab21 heavy chain CDR 1
acctatacaatgggc

SEQ ID NO: 336 - DNA, 48 bp, Oryctolagus cuniculus
  anti-TNF-alpha antibody Ab21 heavy chain CDR 2
tacattagttatggtggtctcgcatactacgcgacctgggtgaatggc

SEQ ID NO: 337 - DNA, 42 bp, Oryctolagus cuniculus
  anti-TNF-alpha antibody Ab21 heavy chain CDR 3
gcggctagtggtgcctggggtcatgcctacggcttggacctc

SEQ ID NO: 338 - protein, 125 aa, Oryctolagus cuniculus
  anti-TNF-alpha antibody Ab22 light chain variable domain
MDTRAPTQLLGLLLLWLPGARCADIVMTQTPASVSGPVGGTVTIKCQASQNIYSSFSWYQQIPGQRPKLLIYYASTLASGVPSRFSGSGSGTDFTLTISDLECADAATYYCQSNHGSNGDSFGNA

SEQ ID NO: 339 - protein, 121 aa, Oryctolagus cuniculus
  anti-TNF-alpha antibody Ab22 heavy chain variable domain
METGLRWLLLVAVLKGVQCQSVEESGGRLVSPGTPLTLTCTVSGIDLSSYGMGWVRQAPGKGLDYIGYMLPSGITYYAAWAKGRFTISKTSSTTVDLKITSPTTEDTATYFCARNYYGMDP

SEQ ID NO: 340 - protein, 11 aa, Oryctolagus cuniculus
  anti-TNF-alpha antibody Ab22 light chain CDR 1
QASQNIYSSFS

SEQ ID NO: 341 - protein, 7 aa, Oryctolagus cuniculus
  anti-TNF-alpha antibody Ab22 light chain CDR 2
YASTLAS

SEQ ID NO: 342 - protein, 14 aa, Oryctolagus cuniculus
  anti-TNF-alpha antibody Ab22 light chain CDR 3
QSNHGSNGDSFGNA

SEQ ID NO: 343 - protein, 5 aa, Oryctolagus cuniculus
  anti-TNF-alpha antibody Ab22 heavy chain CDR 1
SYGMG

SEQ ID NO: 344 - protein, 16 aa, Oryctolagus cuniculus
  anti-TNF-alpha antibody Ab22 heavy chain CDR 2
YMLPSGITYYAAWAKG

SEQ ID NO: 345 - protein, 7 aa, Oryctolagus cuniculus
  anti-TNF-alpha antibody Ab22 heavy chain CDR 3
NYYGMDP

SEQ ID NO: 346 - DNA, 375 bp, Oryctolagus cuniculus
  anti-TNF-alpha antibody Ab22 light chain variable domain
atggacacgagggcccccactcagctgctggggctcctgctgctctggctcccaggtgccagatgtgctgacattgtgatgacccagactccagcctccgtgtctggacctgtgggaggcacagtcaccatcaagtgccaggccagtcagaacatttacagctccttttcctggtatcaacaaataccaggccagcgtcccaagctcctgatctattatgcatccactctggcctctggggtcccatcgcggttcagcggcagtggatctgggacagatttcactctcaccatcagcgacctggagtgtgccgatgctgccacttactactgtcaaagcaatcatggtagtaatggtgatagttttggtaatgct

SEQ ID NO: 347 - DNA, 363 bp, Oryctolagus cuniculus
  anti-TNF-alpha antibody Ab22 heavy chain variable domain
atggagactgggctgcgctggcttctcctggtcgctgtgctcaaaggtgtccagtgtcagtcggtggaggagtccgggggtcgcctggtgtcgcctgggacacccctgacactcacctgcacagtctctggaatcgacctcagtagctatggaatgggctgggtccgccaggctccagggaaggggctggattacatcggatacatgcttcctagtggtatcacatattacgcggcctgggcgaaaggccgattcaccatctccaaaacctcgtcgaccacggtggatctgaaaatcaccagtccgacaaccgaggacacggccacctatttctgtgccagaaattactacggcatggacccc

SEQ ID NO: 348 - DNA, 33 bp, Oryctolagus cuniculus
  anti-TNF-alpha antibody Ab22 light chain CDR 1
caggccagtcagaacatttacagctccttttcc

SEQ ID NO: 349 - DNA, 21 bp, Oryctolagus cuniculus
  anti-TNF-alpha antibody Ab22 light chain CDR 2
tatgcatccactctggcctct

SEQ ID NO: 350 - DNA, 42 bp, Oryctolagus cuniculus; identical to SEQ ID NO: 238
  anti-TNF-alpha antibody Ab22 light chain CDR 3

SEQ ID NO: 351 - DNA, 15 bp, Oryctolagus cuniculus
  anti-TNF-alpha antibody Ab22 heavy chain CDR 1
agctatggaatgggc

SEQ ID NO: 352 - DNA, 48 bp, Oryctolagus cuniculus
  anti-TNF-alpha antibody Ab22 heavy chain CDR 2
tacatgcttcctagtggtatcacatattacgcggcctgggcgaaaggc

SEQ ID NO: 353 - DNA, 21 bp, Oryctolagus cuniculus
  anti-TNF-alpha antibody Ab22 heavy chain CDR 3
aattactacggcatggacccc

SEQ ID NO: 354 - protein, 125 aa, Oryctolagus cuniculus
  anti-TNF-alpha antibody Ab23 light chain variable domain
MDTRAPTQLLGLLLLWLPGARCADIVMTQTPASVSEPVGGTVTIKCQASQSIYRYLSWYHHKPGQPPKLLIYGASNLESGVPSRFKGSGSGTEYTLTISDLECDDAATYYCQSNYGANSDSYGDA

SEQ ID NO: 355 - protein, 129 aa, Oryctolagus cuniculus
  anti-TNF-alpha antibody Ab23 heavy chain variable domain
METGLRWLLLVAVLKGVQCQEQLEESGGDLVKPGASLTLTCKASGFSFSSGYYMGWVRQAPGKGLQYIGYIDYGGSAYYASWAKGRFTISKTSSTTVTLQMTSLTAADTATFFCTRRDYTGGVVRGLDL

SEQ ID NO: 356 - protein, 11 aa, Oryctolagus cuniculus
  anti-TNF-alpha antibody Ab23 light chain CDR 1
QASQSIYRYLS

SEQ ID NO: 357 - protein, 7 aa, Oryctolagus cuniculus
  anti-TNF-alpha antibody Ab23 light chain CDR 2
GASNLES

SEQ ID NO: 358 - protein, 14 aa, Oryctolagus cuniculus
  anti-TNF-alpha antibody Ab23 light chain CDR 3
QSNYGANSDSYGDA

SEQ ID NO: 359 - protein, 6 aa, Oryctolagus cuniculus
  anti-TNF-alpha antibody Ab23 heavy chain CDR 1
SGYYMG

SEQ ID NO: 360 - protein, 16 aa, Oryctolagus cuniculus
  anti-TNF-alpha antibody Ab23 heavy chain CDR 2
YIDYGGSAYYASWAKG

SEQ ID NO: 361 - protein, 13 aa, Oryctolagus cuniculus
  anti-TNF-alpha antibody Ab23 heavy chain CDR 3
RDYTGGVVRGLDL

SEQ ID NO: 362 - DNA, 375 bp, Oryctolagus cuniculus
  anti-TNF-alpha antibody Ab23 light chain variable domain
atggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagagcatttacaggtacttatcctggtatcaccacaaaccagggcagcctcccaagctcctgatctatggtgcatccaatctggaatctggggtcccatcgcggttcaaaggcagtggatctgggacagagtacactctcaccatcagcgacctggagtgtgacgatgctgccacttattactgtcagagcaattatggtgctaatagtgatagttatggggatgct

SEQ ID NO: 363 - DNA, 387 bp, Oryctolagus cuniculus
  anti-TNF-alpha antibody Ab23 heavy chain variable domain
atggagactgggctgcgctggcttctcctggtcgctgtgctcaaaggtgtccagtgtcaggagcagctggaggagtccgggggagacctggtcaagcctggggcatccctgacactcacctgcaaagcctctggattctccttcagtagcggctactacatgggctgggtccgccaggctccagggaaagggctgcaatacatcggttacattgattatggtggtagcgcatactacgcgagctgggcgaaaggccgattcaccatctccaaaacctcgtcgaccacggtgactctgcaaatgaccagtctgacagccgcggacacggccacctttttctgtacgagacgtgactatactggtggtgttgtcagagggctggatctc

SEQ ID NO: 364 - DNA, 33 bp, Oryctolagus cuniculus
  anti-TNF-alpha antibody Ab23 light chain CDR 1
caggccagtcagagcatttacaggtacttatcc

SEQ ID NO: 365 - DNA, 21 bp, Oryctolagus cuniculus
  anti-TNF-alpha antibody Ab23 light chain CDR 2
ggtgcatccaatctggaatct

SEQ ID NO: 366 - DNA, 42 bp, Oryctolagus cuniculus
  anti-TNF-alpha antibody Ab23 light chain CDR 3
cagagcaattatggtgctaatagtgatagttatggggatgct

SEQ ID NO: 367 - DNA, 18 bp, Oryctolagus cuniculus
  anti-TNF-alpha antibody Ab23 heavy chain CDR 1
agcggctactacatgggc

SEQ ID NO: 368 - DNA, 48 bp, Oryctolagus cuniculus
  anti-TNF-alpha antibody Ab23 heavy chain CDR 2
tacattgattatggtggtagcgcatactacgcgagctgggcgaaaggc

SEQ ID NO: 369 - DNA, 39 bp, Oryctolagus cuniculus
  anti-TNF-alpha antibody Ab23 heavy chain CDR 3
cgtgactatactggtggtgttgtcagagggctggatctc

SEQ ID NO: 370 - protein, 125 aa, Oryctolagus cuniculus
  anti-TNF-alpha antibody Ab24 light chain variable domain
MDTRAPTQLLGLLLLWLPGARCADIVMTQTPSSVSAAVGGTVTINCQASQNIYSSLAWYQQKPGQPPKLLIFGASNLESGVPSRFKGSGSGTEFTLTISDLECADAAAYYCQSHHGSNSDSYGNA

SEQ ID NO: 371 - protein, 123 aa, Oryctolagus cuniculus
  anti-TNF-alpha antibody Ab24 heavy chain variable domain
METGLRWLLLVAVLKGVQCQSVEESGGRLVTPGTPLTLTCTASGFSLNNYYMTWVRQAPGKGLESIGYFASGGGTYYANWAKGRFTISKTSTTVDLKITSPTTDDTATYFCARGGAYLGTGSL

SEQ ID NO: 372 - protein, 11 aa, Oryctolagus cuniculus
  anti-TNF-alpha antibody Ab24 light chain CDR 1
QASQNIYSSLA

SEQ ID NO: 373 - protein, 7 aa, Oryctolagus cuniculus
  anti-TNF-alpha antibody Ab24 light chain CDR 2
GASNLES

SEQ ID NO: 374 - protein, 14 aa, Oryctolagus cuniculus
  anti-TNF-alpha antibody Ab24 light chain CDR 3
QSHHGSNSDSYGNA

SEQ ID NO: 375 - protein, 5 aa, Oryctolagus cuniculus
  anti-TNF-alpha antibody Ab24 heavy chain CDR 1
NYYMT

SEQ ID NO: 376 - protein, 16 aa, Oryctolagus cuniculus
  anti-TNF-alpha antibody Ab24 heavy chain CDR 2
YFASGGGTYYANWAKG

SEQ ID NO: 377 - protein, 10 aa, Oryctolagus cuniculus
  anti-TNF-alpha antibody Ab24 heavy chain CDR 3
GGAYLGTGSL

SEQ ID NO: 378 - DNA, 375 bp, Oryctolagus cuniculus
  anti-TNF-alpha antibody Ab24 light chain variable domain
atggacacgagggcccccactcagctgctggggctcctgctgctctggctcccaggtgccagatgtgccgacattgtgatgacccagactccatcctccgtgtctgcagctgtgggaggcacagtcaccatcaattgccaggccagtcagaacatttacagctctttagcctggtatcagcagaaaccagggcagcctcccaagctcctgatctttggtgcatccaatctggaatctggggtcccatcgcggttcaaaggcagtggatctgggacagagttcactctcaccatcagcgacctggagtgtgccgatgctgccgcttactactgtcagagccatcatggtagtaatagtgatagttatggtaatgct

SEQ ID NO: 379 - DNA, 369 bp, Oryctolagus cuniculus
  anti-TNF-alpha antibody Ab24 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagcctctggattctcccttaataactactacatgacctgggtccgccaggctccagggaaggggctggaatccatcggatattttgcttctggtggtggcacatactacgcgaactgggcgaaaggccgattcaccatctccaaaacctcgaccacggtggatctgaagatcaccagtccgacaaccgacgatacggccacctatttctgtgccaggggtggtgcttatttgggtactgggagtttg

SEQ ID NO: 380 - DNA, 33 bp, Oryctolagus cuniculus
  anti-TNF-alpha antibody Ab24 light chain CDR 1
caggccagtcagaacatttacagctctttagcc

SEQ ID NO: 381 - DNA, 21 bp, Oryctolagus cuniculus
  anti-TNF-alpha antibody Ab24 light chain CDR 2
ggtgcatccaatctggaatct

SEQ ID NO: 382 - DNA, 42 bp, Oryctolagus cuniculus
  anti-TNF-alpha antibody Ab24 light chain CDR 3
cagagccatcatggtagtaatagtgatagttatggtaatgct

SEQ ID NO: 383 - DNA, 15 bp, Oryctolagus cuniculus
  anti-TNF-alpha antibody Ab24 heavy chain CDR 1
aactactacatgacc

SEQ ID NO: 384 - DNA, 48 bp, Oryctolagus cuniculus
  anti-TNF-alpha antibody Ab24 heavy chain CDR 2
tattttgcttctggtggtggcacatactacgcgaactgggcgaaaggc

SEQ ID NO: 385 - DNA, 30 bp, Oryctolagus cuniculus
  anti-TNF-alpha antibody Ab24 heavy chain CDR 3
ggtggtgcttatttgggtactgggagtttg

SEQ ID NO: 386 - protein, 125 aa, Oryctolagus cuniculus
  anti-TNF-alpha antibody Ab25 light chain variable domain
MDTRAPTQLLGLLLLWLPGARCADIVMTQTPSSVSVPVGGTVTIKCQASQNIYSSLAWYQQKPGQPPKRLIYYAATLASGVPSRFKGSGSGTDFTLTISDLECADAATYYCQSNHGSNSDSYGNP

SEQ ID NO: 387 - protein, 123 aa, Oryctolagus cuniculus
  anti-TNF-alpha antibody Ab25 heavy chain variable domain
METGLRWLLLVAVLKGVQCQSVEESGGRLVTPGTPLTLTCTVAGFSLSTYGVTWVRQAPGKGLESIGYITYGNIKYYATWAKGRFTISKTSTTVDLKMTSPTTEDTATYFCTRYGGSGIGEDL

SEQ ID NO: 388 - protein, 11 aa, Oryctolagus cuniculus
  anti-TNF-alpha antibody Ab25 light chain CDR 1
QASQNIYSSLA

SEQ ID NO: 389 - protein, 7 aa, Oryctolagus cuniculus
  anti-TNF-alpha antibody Ab25 light chain CDR 2
YAATLAS

SEQ ID NO: 390 - protein, 14 aa, Oryctolagus cuniculus
  anti-TNF-alpha antibody Ab25 light chain CDR 3
QSNHGSNSDSYGNP

SEQ ID NO: 391 - protein, 5 aa, Oryctolagus cuniculus
  anti-TNF-alpha antibody Ab25 heavy chain CDR 1
TYGVT

SEQ ID NO: 392 - protein, 16 aa, Oryctolagus cuniculus
  anti-TNF-alpha antibody Ab25 heavy chain CDR 2
YITYGNIKYYATWAKG

SEQ ID NO: 393 - protein, 10 aa, Oryctolagus cuniculus
  anti-TNF-alpha antibody Ab25 heavy chain CDR 3
YGGSGIGEDL

SEQ ID NO: 394 - DNA, 375 bp, Oryctolagus cuniculus
  anti-TNF-alpha antibody Ab25 light chain variable domain
atggacacgagggcccccactcagctgctggggctcctgctgctctggctcccaggtgccagatgtgctgacattgtgatgacccagactccatcctccgtgtctgtacctgtgggaggcacagtcaccatcaagtgccaggccagtcagaacatttacagctctttagcctggtatcagcagaaaccaggacagcctcccaagcgcctgatctattatgccgccactctggcatctggggtcccatcgcggttcaaaggcagtggatctgggacagatttcactctcaccatcagcgacctggagtgtgccgatgctgccacttactattgtcaaagcaatcatggtagtaatagtgatagttatggtaatcct

SEQ ID NO: 395 - DNA, 369 bp, Oryctolagus cuniculus
  anti-TNF-alpha antibody Ab25 heavy chain variable domain
atggagactgggctgcgctggcttctcctggtcgctgtgctcaagggtgtccagtgtcagtcggtggaggagtccgggggtcgcctggtcacgcctgggacacccctgacactcacctgcacagtcgctggattctccctcagtacctatggagtgacctgggtccgccaggctccagggaaggggctggaatccatcggatacattacttatggtaatattaaatactacgcgacctgggcgaaaggccgattcaccatctccaaaacctcgaccacggtggatctgaaaatgaccagtccgacaaccgaggacacggccacctatttctgtaccagatatggtggtagtgggattggtgaggacttg

SEQ ID NO: 396 - DNA, 33 bp, Oryctolagus cuniculus; identical to SEQ ID NO: 380
  anti-TNF-alpha antibody Ab25 light chain CDR 1

SEQ ID NO: 397 - DNA, 21 bp, Oryctolagus cuniculus
  anti-TNF-alpha antibody Ab25 light chain CDR 2
tatgccgccactctggcatct

SEQ ID NO: 398 - DNA, 42 bp, Oryctolagus cuniculus
  anti-TNF-alpha antibody Ab25 light chain CDR 3
caaagcaatcatggtagtaatagtgatagttatggtaatcct

SEQ ID NO: 399 - DNA, 15 bp, Oryctolagus cuniculus
  anti-TNF-alpha antibody Ab25 heavy chain CDR 1
acctatggagtgacc

SEQ ID NO: 400 - DNA, 48 bp, Oryctolagus cuniculus
  anti-TNF-alpha antibody Ab25 heavy chain CDR 2
tacattacttatggtaatattaaatactacgcgacctgggcgaaaggc

SEQ ID NO: 401 - DNA, 30 bp, Oryctolagus cuniculus
  anti-TNF-alpha antibody Ab25 heavy chain CDR 3
tatggtggtagtgggattggtgaggacttg

SEQ ID NO: 402 - protein, 124 aa, Oryctolagus cuniculus
  anti-TNF-alpha antibody Ab26 light chain variable domain
MDTRAPTQLLGLLLLWLPGARCADVVMTQTPSSVSEPVGGTVTIKCQASETIGNYLSWYQQKPGQPPKRLIYYASTLSSGVPSRFKGSGSGTDFTLTISDLECADAATYYCQKNYGSGASSLGA

SEQ ID NO: 403 - protein, 125 aa, Oryctolagus cuniculus
  anti-TNF-alpha antibody Ab26 heavy chain variable domain
METGLRWLLLVAVLKGVQCQSVEESGGRLVTPGTPLTLTCTVSGFSLSSYYMAWVRQAPGKGLEWIGYIGFGGSTYYATWAKGRVTISRTSTTVDLQITSPTTEDTATYFCARGVYGDFRTGADL

SEQ ID NO: 404 - protein, 11 aa, Oryctolagus cuniculus
  anti-TNF-alpha antibody Ab26 light chain CDR 1
QASETIGNYLS

SEQ ID NO: 405 - protein, 7 aa, Oryctolagus cuniculus
  anti-TNF-alpha antibody Ab26 light chain CDR 2
YASTLSS

SEQ ID NO: 406 - protein, 13 aa, Oryctolagus cuniculus
  anti-TNF-alpha antibody Ab26 light chain CDR 3
QKNYGSGASSLGA

SEQ ID NO: 407 - protein, 5 aa, Oryctolagus cuniculus
  anti-TNF-alpha antibody Ab26 heavy chain CDR 1
SYYMA

SEQ ID NO: 408 - protein, 16 aa, Oryctolagus cuniculus
  anti-TNF-alpha antibody Ab26 heavy chain CDR 2
YIGFGGSTYYATWAKG

SEQ ID NO: 409 - protein, 12 aa, Oryctolagus cuniculus
  anti-TNF-alpha antibody Ab26 heavy chain CDR 3
GVYGDFRTGADL

SEQ ID NO: 410 - DNA, 372 bp, Oryctolagus cuniculus
  anti-TNF-alpha antibody Ab26 light chain variable domain
atggacacgagggcccccactcagctgctggggctcctgctgctctggctcccaggtgccagatgtgccgacgtcgtgatgacccagactccatcctccgtgtctgaacctgtgggaggcacagtcaccatcaagtgccaggccagtgaaaccattggtaactacttatcctggtatcagcagaaaccagggcagcctcccaagcgcctgatctattatgcatccactctgtcatctggggtcccatcgcggttcaaaggcagtggatctgggacagatttcactctcaccatcagcgacctggagtgtgccgatgctgccacttactactgccaaaagaattatggtagtggtgctagtagtttgggtgct

SEQ ID NO: 411 - DNA, 375 bp, Oryctolagus cuniculus
  anti-TNF-alpha antibody Ab26 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtagctactacatggcctgggtccgccaggctccagggaaggggctggagtggatcggatatattggttttggtggtagcacatactacgcgacctgggcgaaaggccgggtcaccatctccaggacctcgaccacggtggatctgcaaatcaccagtccgacaaccgaggacacggccacctatttctgtgccagaggagtttatggtgattttcgtactggtgccgacttg

SEQ ID NO: 412 - DNA, 33 bp, Oryctolagus cuniculus
  anti-TNF-alpha antibody Ab26 light chain CDR 1
caggccagtgaaaccattggtaactacttatcc

SEQ ID NO: 413 - DNA, 21 bp, Oryctolagus cuniculus
  anti-TNF-alpha antibody Ab26 light chain CDR 2
tatgcatccactctgtcatct

SEQ ID NO: 414 - DNA, 39 bp, Oryctolagus cuniculus
  anti-TNF-alpha antibody Ab26 light chain CDR 3
caaaagaattatggtagtggtgctagtagtttgggtgct

SEQ ID NO: 415 - DNA, 15 bp, Oryctolagus cuniculus
  anti-TNF-alpha antibody Ab26 heavy chain CDR 1
agctactacatggcc

SEQ ID NO: 416 - DNA, 48 bp, Oryctolagus cuniculus
  anti-TNF-alpha antibody Ab26 heavy chain CDR 2
tatattggttttggtggtagcacatactacgcgacctgggcgaaaggc

SEQ ID NO: 417 - DNA, 36 bp, Oryctolagus cuniculus
  anti-TNF-alpha antibody Ab26 heavy chain CDR 3
ggagtttatggtgattttcgtactggtgccgacttg

SEQ ID NO: 418 - protein, 105 aa, Artificial Sequence [sequence omitted]

SEQ ID NO: 419 - DNA, 315 bp, Artificial sequence
  Kappa constant domain
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 420 - protein, 330 aa, Artificial sequence
  Gamma-1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 421 - DNA, 990 bp, Artificial sequence
  Gamma-1 constant domain
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 422 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 423 - protein, 122 aa, Oryctolagus cuniculus
  anti-IL-6 antibody Ab1 light chain variable domain
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNA

SEQ ID NO: 424 - protein, 125 aa, Oryctolagus cuniculus
  anti-IL-6 antibody Ab1 heavy chain variable domain
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNL

SEQ ID NO: 425 - protein, 11 aa, Oryctolagus cuniculus
  anti-IL-6 antibody Ab1 light chain CDR 1
QASQSINNELS

SEQ ID NO: 426 - protein, 7 aa, Oryctolagus cuniculus
  anti-IL-6 antibody Ab1 light chain CDR 2
RASTLAS

SEQ ID NO: 427 - protein, 12 aa, Oryctolagus cuniculus
  anti-IL-6 antibody Ab1 light chain CDR 3
QQGYSLRNIDNA

SEQ ID NO: 428 - protein, 5 aa, Oryctolagus cuniculus
  anti-IL-6 antibody Ab1 heavy chain CDR 1
NYYVT

SEQ ID NO: 429 - protein, 16 aa, Oryctolagus cuniculus
  anti-IL-6 antibody Ab1 heavy chain CDR 2
IIYGSDETAYATWAIG

SEQ ID NO: 430 - protein, 12 aa, Oryctolagus cuniculus
  anti-IL-6 antibody Ab1 heavy chain CDR 3
DDSSDWDAKFNL

SEQ ID NO: 431 - DNA, 366 bp, Oryctolagus cuniculus
  anti-IL-6 antibody Ab1 light chain variable domain
atggacacgagggcccccactcagctgctggggctcctgctgctctggctcccaggtgccagatgtgcctatgatatgacccagactccagcctcggtgtctgcagctgtgggaggcacagtcaccatcaagtgccaggccagtcagagcattaacaatgaattatcctggtatcagcagaaaccagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 432 - DNA, 375 bp, Oryctolagus cuniculus
  anti-IL-6 antibody Ab1 heavy chain variable domain
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtaactactacgtgacctgggtccgccaggctccagggaaggggctggaatggatcggaatcatttatggtagtgatgaaacggcctacgcgacctgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 433 - DNA, 33 bp, Oryctolagus cuniculus
  anti-IL-6 antibody Ab1 light chain CDR 1
caggccagtcagagcattaacaatgaattatcc

SEQ ID NO: 434 - DNA, 21 bp, Oryctolagus cuniculus
  anti-IL-6 antibody Ab1 light chain CDR 2
agggcatccactctggcatct

SEQ ID NO: 435 - DNA, 36 bp, Oryctolagus cuniculus
  anti-IL-6 antibody Ab1 light chain CDR 3
caacagggttatagtctgaggaatattgataatgct

SEQ ID NO: 436 - DNA, 15 bp, Oryctolagus cuniculus
  anti-IL-6 antibody Ab1 heavy chain CDR 1
aactactacgtgacc

SEQ ID NO: 437 - DNA, 48 bp, Oryctolagus cuniculus
  anti-IL-6 antibody Ab1 heavy chain CDR 2
atcatttatggtagtgatgaaacggcctacgcgacctgggcgataggc

SEQ ID NO: 438 - DNA, 36 bp, Oryctolagus cuniculus
  anti-IL-6 antibody Ab1 heavy chain CDR 3
gatgatagtagtgactgggatgcaaaatttaacttg

SEQ ID NO: 439 - protein, 109 aa, Oryctolagus cuniculus
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 440 - protein, 109 aa, Oryctolagus cuniculus
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 441 - protein, 99 aa, Oryctolagus cuniculus
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA

SEQ ID NO: 442 - protein, 122 aa, Oryctolagus cuniculus
  anti-IL-6 antibody Ab2 light chain variable domain
MDTRAPTQLLGLLLLWLPGARCAYDMTQTPASVEVAVGGTVTINCQASETIYSWLSWYQQKPGQPPKLLIYQASDLASGVPSRFSGSGAGTEYTLTISGVQCDDAATYYCQQGYSGSNVDNV

SEQ ID NO: 443 - protein, 126 aa, Oryctolagus cuniculus
  anti-IL-6 antibody Ab2 heavy chain variable domain
METGLRWLLLVAVLKGVQCQEQLKESGGRLVTPGTPLTLTCTASGFSLNDHAMGWVRQAPGKGLEYIGFINSGGSARYASWAEGRFTISRTSTTVDLKMTSLTTEDTATYFCVRGGAVWSIHSFDP

SEQ ID NO: 444 - protein, 11 aa, Oryctolagus cuniculus
  anti-IL-6 antibody Ab2 light chain CDR 1
QASETIYSWLS

SEQ ID NO: 445 - protein, 7 aa, Oryctolagus cuniculus
  anti-IL-6 antibody Ab2 light chain CDR 2
QASDLAS

SEQ ID NO: 446 - protein, 12 aa, Oryctolagus cuniculus
  anti-IL-6 antibody Ab2 light chain CDR 3
QQGYSGSNVDNV

SEQ ID NO: 447 - protein, 5 aa, Oryctolagus cuniculus
  anti-IL-6 antibody Ab2 heavy chain CDR 1
DHAMG

SEQ ID NO: 448 - protein, 16 aa, Oryctolagus cuniculus
  anti-IL-6 antibody Ab2 heavy chain CDR 2
FINSGGSARYASWAEG

SEQ ID NO: 449 - protein, 12 aa, Oryctolagus cuniculus
  anti-IL-6 antibody Ab2 heavy chain CDR 3
GGAVWSIHSFDP

SEQ ID NO: 450 - DNA, 366 bp, Oryctolagus cuniculus
  anti-IL-6 antibody Ab2 light chain variable domain
atggacacgagggcccccactcagctgctggggctcctgctgctctggctcccaggtgccagatgtgcctatgatatgacccagactccagcctctgtggaggtagctgtgggaggcacagtcaccatcaattgccaggccagtgagaccatttacagttggttatcctggtatcagcagaagccagggcagcctcccaagctcctgatctaccaggcatccgatctggcatctggggtcccatcgcgattcagcggcagtggggctgggacagagtacactctcaccatcagcggcgtgcagtgtgacgatgctgccacttactactgtcaacagggttatagtggtagtaatgttgataatgtt

SEQ ID NO: 451 - DNA, 378 bp, Oryctolagus cuniculus
  anti-IL-6 antibody Ab2 heavy chain variable domain
atggagactgggctgcgctggcttctcctggtcgctgtgctcaaaggtgtccagtgtcaggagcagctgaaggagtccgggggtcgcctggtcacgcctgggacacccctgacacttacctgcacagcctctggattctccctcaatgaccatgcaatgggctgggtccgccaggctccagggaaggggctggaatacatcggattcattaatagtggtggtagcgcacgctacgcgagctgggcagaaggccgattcaccatctccagaacctcgaccacggtggatctgaaaatgaccagtctgacaaccgaggacacggccacctatttctgtgtcagagggggtgctgtttggagtattcatagttttgatccc

SEQ ID NO: 452 - DNA, 33 bp, Oryctolagus cuniculus
  anti-IL-6 antibody Ab2 light chain CDR 1
caggccagtgagaccatttacagttggttatcc

SEQ ID NO: 453 - DNA, 21 bp, Oryctolagus cuniculus
  anti-IL-6 antibody Ab2 light chain CDR 2
caggcatccgatctggcatct

SEQ ID NO: 454 - DNA, 36 bp, Oryctolagus cuniculus
  anti-IL-6 antibody Ab2 light chain CDR 3
caacagggttatagtggtagtaatgttgataatgtt

SEQ ID NO: 455 - DNA, 15 bp, Oryctolagus cuniculus
  anti-IL-6 antibody Ab2 heavy chain CDR 1
gaccatgcaatgggc

SEQ ID NO: 456 - DNA, 48 bp, Oryctolagus cuniculus
  anti-IL-6 antibody Ab2 heavy chain CDR 2
ttcattaatagtggtggtagcgcacgctacgcgagctgggcagaaggc

SEQ ID NO: 457 - DNA, 36 bp, Oryctolagus cuniculus
  anti-IL-6 antibody Ab2 heavy chain CDR 3
gggggtgctgtttggagtattcatagttttgatccc

SEQ ID NO: 458 - protein, 123 aa, Oryctolagus cuniculus
  anti-IL-6 antibody Ab3 light chain variable domain
MDTRAPTQLLGLLLLWLPGATFAAVLTQTPSPVSAAVGGTVSISCQASQSVYDNNYLSWFQQKPGQPPKLLIYGASTLASGVPSRFVGSGSGTQFTLTITDVQCDDAATYYCAGVYDDDSDNA

SEQ ID NO: 459 - protein, 125 aa, Oryctolagus cuniculus
  anti-IL-6 antibody Ab3 heavy chain variable domain
METGLRWLLLVAVLKGVQCQSLEESGGRLVTPGTPLTLTCTASGFSLSVYYMNWVRQAPGKGLEWIGFITMSDNINYASWAKGRFTISKTSTTVDLKMTSPTTEDTATYFCARSRGWGTMGRLDL

SEQ ID NO: 460 - protein, 13 aa, Oryctolagus cuniculus
  anti-IL-6 antibody Ab3 light chain CDR 1
QASQSVYDNNYLS

SEQ ID NO: 461 - protein, 7 aa, Oryctolagus cuniculus
  anti-IL-6 antibody Ab3 light chain CDR 2
GASTLAS

SEQ ID NO: 462 - protein, 11 aa, Oryctolagus cuniculus
  anti-IL-6 antibody Ab3 light chain CDR 3
AGVYDDDSDNA

SEQ ID NO: 463 - protein, 5 aa, Oryctolagus cuniculus
  anti-IL-6 antibody Ab3 heavy chain CDR 1
VYYMN

SEQ ID NO: 464 - protein, 16 aa, Oryctolagus cuniculus
  anti-IL-6 antibody Ab3 heavy chain CDR 2
FITMSDNINYASWAKG

SEQ ID NO: 465 - protein, 12 aa, Oryctolagus cuniculus
  anti-IL-6 antibody Ab3 heavy chain CDR 3
SRGWGTMGRLDL

SEQ ID NO: 466 - DNA, 369 bp, Oryctolagus cuniculus
  anti-IL-6 antibody Ab3 light chain variable domain
atggacacgagggcccccactcagctgctggggctcctgctgctctggctcccaggtgccacatttgccgccgtgctgacccagactccatctcccgtgtctgcagctgtgggaggcacagtcagcatcagttgccaggccagtcagagtgtttatgacaacaactacttatcctggtttcagcagaaaccagggcagcctcccaagctcctgatctatggtgcatccactctggcatctggggtcccatcgcggttcgtgggcagtggatctgggacacagttcactctcaccatcacagacgtgcagtgtgacgatgctgccacttactattgtgcaggcgtttatgatgatgatagtgataatgcc

SEQ ID NO: 467 - DNA, 375 bp, Oryctolagus cuniculus
  anti-IL-6 antibody Ab3 heavy chain variable domain
atggagactgggctgcgctggcttctcctggtggctgtgctcaaaggtgtccagtgtcagtcgctggaggagtccgggggtcgcctggtcacccctgggacacccctgacactcacctgcacagcctctggattctccctcagtgtctactacatgaactgggtccgccaggctccagggaaggggctggaatggatcggattcattacaatgagtgataatataaattacgcgagctgggcgaaaggccgattcaccatctccaaaacctcgaccacggtggatctgaaaatgaccagtccgacaaccgaggacacggccacctatttctgtgccaggagtcgtggctggggtacaatgggtcggttggatctc

SEQ ID NO: 468 - DNA, 39 bp, Oryctolagus cuniculus
  anti-IL-6 antibody Ab3 light chain CDR 1
caggccagtcagagtgtttatgacaacaactacttatcc

SEQ ID NO: 469 - DNA, 21 bp, Oryctolagus cuniculus
  anti-IL-6 antibody Ab3 light chain CDR 2
ggtgcatccactctggcatct

SEQ ID NO: 470 - DNA, 33 bp, Oryctolagus cuniculus
  anti-IL-6 antibody Ab3 light chain CDR 3
gcaggcgtttatgatgatgatagtgataatgcc

SEQ ID NO: 471 - DNA, 15 bp, Oryctolagus cuniculus
  anti-IL-6 antibody Ab3 heavy chain CDR 1
gtctactacatgaac

SEQ ID NO: 472 - DNA, 48 bp, Oryctolagus cuniculus
  anti-IL-6 antibody Ab3 heavy chain CDR 2
ttcattacaatgagtgataatataaattacgcgagctgggcgaaaggc

SEQ ID NO: 473 - DNA, 36 bp, Oryctolagus cuniculus
  anti-IL-6 antibody Ab3 heavy chain CDR 3
agtcgtggctggggtacaatgggtcggttggatctc

SEQ ID NO: 474 - protein, 123 aa, Oryctolagus cuniculus
  anti-IL-6 antibody Ab4 light chain variable domain
MDTRAPTQLLGLLLLWLPGAICDPVLTQTPSPVSAPVGGTVSISCQASQSVYENNYLSWFQQKPGQPPKLLIYGASTLDSGVPSRFKGSGSGTQFTLTITDVQCDDAATYYCAGVYDDDSDDA

SEQ ID NO: 475 - protein, 126 aa, Oryctolagus cuniculus
  anti-IL-6 antibody Ab4 heavy chain variable domain
METGLRWLLLVAVLKGVQCQEQLKESGGGLVTPGGTLTLTCTASGFSLNAYYMNWVRQAPGKGLEWIGFITLNNNVAYANWAKGRFTFSKTSTTVDLKMTSPTPEDTATYFCARSRGWGAMGRLDL

SEQ ID NO: 476 - protein, 13 aa, Oryctolagus cuniculus
  anti-IL-6 antibody Ab4 light chain CDR 1
QASQSVYENNYLS

SEQ ID NO: 477 - protein, 7 aa, Oryctolagus cuniculus
  anti-IL-6 antibody Ab4 light chain CDR 2
GASTLDS

SEQ ID NO: 478 - protein, 11 aa, Oryctolagus cuniculus
  anti-IL-6 antibody Ab4 light chain CDR 3
AGVYDDDSDDA

SEQ ID NO: 479 - protein, 5 aa, Oryctolagus cuniculus
  anti-IL-6 antibody Ab4 heavy chain CDR 1
AYYMN

SEQ ID NO: 480 - protein, 16 aa, Oryctolagus cuniculus
  anti-IL-6 antibody Ab4 heavy chain CDR 2
FITLNNNVAYANWAKG

SEQ ID NO: 481 - protein, 12 aa, Oryctolagus cuniculus
  anti-IL-6 antibody Ab4 heavy chain CDR 3
SRGWGAMGRLDL

SEQ ID NO: 482 - DNA, 369 bp, Oryctolagus cuniculus
  anti-IL-6 antibody Ab4 light chain variable domain
atggacacgagggcccccactcagctgctggggctcctgctgctctggctcccaggtgccatatgtgaccctgtgctgacccagactccatctcccgtatctgcacctgtgggaggcacagtcagcatcagttgccaggccagtcagagtgtttatgagaacaactatttatcctggtttcagcagaaaccagggcagcctcccaagctcctgatctatggtgcatccactctggattctggggtcccatcgcggttcaaaggcagtggatctgggacacagttcactctcaccattacagacgtgcagtgtgacgatgctgccacttactattgtgcaggcgtttatgatgatgatagtgatgatgcc

SEQ ID NO: 483 - DNA, 378 bp, Oryctolagus cuniculus
  anti-IL-6 antibody Ab4 heavy chain variable domain
atggagactgggctgcgctggcttctcctggtggctgtgctcaaaggtgtccagtgtcaggagcagctgaaggagtccggaggaggcctggtaacgcctggaggaaccctgacactcacctgcacagcctctggattctccctcaatgcctactacatgaactgggtccgccaggctccagggaaggggctggaatggatcggattcattactctgaataataatgtagcttacgcgaactgggcgaaaggccgattcaccttctccaaaacctcgaccacggtggatctgaaaatgaccagtccgacacccgaggacacggccacctatttctgtgccaggagtcgtggctggggtgcaatgggtcggttggatctc

SEQ ID NO: 484 - DNA, 39 bp, Oryctolagus cuniculus
  anti-IL-6 antibody Ab4 light chain CDR 1
caggccagtcagagtgtttatgagaacaactatttatcc

SEQ ID NO: 485 - DNA, 21 bp, Oryctolagus cuniculus
  anti-IL-6 antibody Ab4 light chain CDR 2
ggtgcatccactctggattct

SEQ ID NO: 486 - DNA, 33 bp, Oryctolagus cuniculus
  anti-IL-6 antibody Ab4 light chain CDR 3
gcaggcgtttatgatgatgatagtgatgatgcc

SEQ ID NO: 487 - DNA, 15 bp, Oryctolagus cuniculus
  anti-IL-6 antibody Ab4 heavy chain CDR 1
gcctactacatgaac

SEQ ID NO: 488 - DNA, 48 bp, Oryctolagus cuniculus
  anti-IL-6 antibody Ab4 heavy chain CDR 2
ttcattactctgaataataatgtagcttacgcgaactgggcgaaaggc

SEQ ID NO: 489 - DNA, 36 bp, Oryctolagus cuniculus
  anti-IL-6 antibody Ab4 heavy chain CDR 3
agtcgtggctggggtgcaatgggtcggttggatctc

SEQ ID NO: 490 - protein, 122 aa, Oryctolagus cuniculus
  anti-IL-6 antibody Ab5 light chain variable domain
MDTRAPTQLLGLLLLWLPGATFAQVLTQTPSPVSAAVGGTVTINCQASQSVDDNNWLGWYQQKRGQPPKYLIYSASTLASGVPSRFKGSGSGTQFTLTISDLECDDAATYYCAGGFSGNIFA

SEQ ID NO: 491 - protein, 122 aa, Oryctolagus cuniculus
  anti-IL-6 antibody Ab5 heavy chain variable domain
METGLRWLLLVAVLKGVQCQSVEESGGRLVTPGTPLTLTCTVSGFSLSSYAMSWVRQAPGKGLEWIGIIGGFGTTYYATWAKGRFTISKTSTTVDLRITSPTTEDTATYFCARGGPGNGGDI

SEQ ID NO: 492 - protein, 13 aa, Oryctolagus cuniculus
  anti-IL-6 antibody Ab5 light chain CDR 1
QASQSVDDNNWLG

SEQ ID NO: 493 - protein, 7 aa, Oryctolagus cuniculus
  anti-IL-6 antibody Ab5 light chain CDR 2
SASTLAS

SEQ ID NO: 494 - protein, 10 aa, Oryctolagus cuniculus
  anti-IL-6 antibody Ab5 light chain CDR 3
AGGFSGNIFA

SEQ ID NO: 495 - protein, 5 aa, Oryctolagus cuniculus
  anti-IL-6 antibody Ab5 heavy chain CDR 1
SYAMS

SEQ ID NO: 496 - protein, 16 aa, Oryctolagus cuniculus
  anti-IL-6 antibody Ab5 heavy chain CDR 2
IIGGFGTTYYATWAKG

SEQ ID NO: 497 - protein, 9 aa, Oryctolagus cuniculus
  anti-IL-6 antibody Ab5 heavy chain CDR 3
GGPGNGGDI

SEQ ID NO: 498 - DNA, 366 bp, Oryctolagus cuniculus
  anti-IL-6 antibody Ab5 light chain variable domain
atggacacgagggcccccactcagctgctggggctcctgctgctctggctcccaggtgccacatttgcccaagtgctgacccagactccatcgcctgtgtctgcagctgtgggaggcacagtcaccatcaactgccaggccagtcagagtgttgatgataacaactggttaggctggtatcagcagaaacgagggcagcctcccaagtacctgatctattctgcatccactctggcatctggggtcccatcgcggttcaaaggcagtggatctgggacacagttcactctcaccatcagcgacctggagtgtgacgatgctgccacttactactgtgcaggcggttttagtggtaatatctttgct

SEQ ID NO: 499 - DNA, 366 bp, Oryctolagus cuniculus
  anti-IL-6 antibody Ab5 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggcttctccctcagtagctatgcaatgagctgggtccgccaggctccaggaaaggggctggagtggatcggaatcattggtggttttggtaccacatactacgcgacctgggcgaaaggccgattcaccatctccaaaacctcgaccacggtggatctgagaatcaccagtccgacaaccgaggacacggccacctatttctgtgccagaggtggtcctggtaatggtggtgacatc

SEQ ID NO: 500 - DNA, 39 bp, Oryctolagus cuniculus
  anti-IL-6 antibody Ab5 light chain CDR 1
caggccagtcagagtgttgatgataacaactggttaggc

SEQ ID NO: 501 - DNA, 21 bp, Oryctolagus cuniculus
  anti-IL-6 antibody Ab5 light chain CDR 2
tctgcatccactctggcatct

SEQ ID NO: 502 - DNA, 30 bp, Oryctolagus cuniculus
  anti-IL-6 antibody Ab5 light chain CDR 3
gcaggcggttttagtggtaatatctttgct

SEQ ID NO: 503 - DNA, 15 bp, Oryctolagus cuniculus
  anti-IL-6 antibody Ab5 heavy chain CDR 1
agctatgcaatgagc

SEQ ID NO: 504 - DNA, 48 bp, Oryctolagus cuniculus
  anti-IL-6 antibody Ab5 heavy chain CDR 2
atcattggtggttttggtaccacatactacgcgacctgggcgaaaggc

SEQ ID NO: 505 - DNA, 27 bp, Oryctolagus cuniculus
  anti-IL-6 antibody Ab5 heavy chain CDR 3
ggtggtcctggtaatggtggtgacatc

SEQ ID NO: 506 - protein, 122 aa, Oryctolagus cuniculus
  anti-IL-6 antibody Ab6 light chain variable domain
MDTRAPTQLLGLLLLWLPGATFAAVLTQTPSPVSVPVGGTVTIKCQSSQSVYNNFLSWYQQKPGQPPKLLIYQASKLASGVPDRFSGSGSGTQFTLTISGVQCDDAATYYCLGGYDDDADNA

SEQ ID NO: 507 - protein, 128 aa, Oryctolagus cuniculus
  anti-IL-6 antibody Ab6 heavy chain variable domain
METGLRWLLLVAVLKGVQCQSVEESGGRLVTPGTPLTLTCTVSGIDLSDYAMSWVRQAPGKGLEWIGIIYAGSGSTWYASWAKGRFTISKTSTTVDLKITSPTTEDTATYFCARDGYDDYGDFDRLDL

SEQ ID NO: 508 - protein, 12 aa, Oryctolagus cuniculus
  anti-IL-6 antibody Ab6 light chain CDR 1
QSSQSVYNNFLS

SEQ ID NO: 509 - protein, 7 aa, Oryctolagus cuniculus
  anti-IL-6 antibody Ab6 light chain CDR 2
QASKLAS

SEQ ID NO: 510 - protein, 11 aa, Oryctolagus cuniculus
  anti-IL-6 antibody Ab6 light chain CDR 3
LGGYDDDADNA

SEQ ID NO: 511 - protein, 5 aa, Oryctolagus cuniculus
  anti-IL-6 antibody Ab6 heavy chain CDR 1
DYAMS

SEQ ID NO: 512 - protein, 17 aa, Oryctolagus cuniculus
  anti-IL-6 antibody Ab6 heavy chain CDR 2
IIYAGSGSTWYASWAKG

SEQ ID NO: 513 - protein, 14 aa, Oryctolagus cuniculus
  anti-IL-6 antibody Ab6 heavy chain CDR 3
DGYDDYGDFDRLDL

SEQ ID NO: 514 - DNA, 366 bp, Oryctolagus cuniculus
  anti-IL-6 antibody Ab6 light chain variable domain
atggacacgagggcccccactcagctgctggggctcctgctgctctggctcccaggtgccacatttgcagccgtgctgacccagacaccatcgcccgtgtctgtacctgtgggaggcacagtcaccatcaagtgccagtccagtcagagtgtttataataatttcttatcgtggtatcagcagaaaccagggcagcctcccaagctcctgatctaccaggcatccaaactggcatctggggtcccagataggttcagcggcagtggatctgggacacagttcactctcaccatcagcggcgtgcagtgtgacgatgctgccacttactactgtctaggcggttatgatgatgatgctgataatgct

SEQ ID NO: 515 - DNA, 384 bp, Oryctolagus cuniculus
  anti-IL-6 antibody Ab6 heavy chain variable domain
atggagactgggctgcgctggcttctcctggtcgctgtgctcaaaggtgtccagtgtcagtcggtggaggagtccgggggtcgcctggtcacgcctgggacacccctgacgctcacctgcacagtctctggaatcgacctcagtgactatgcaatgagctgggtccgccaggctccagggaaggggctggaatggatcggaatcatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatggatacgatgactatggtgatttcgatcgattggatctc

SEQ ID NO: 516 - DNA, 36 bp, Oryctolagus cuniculus
  anti-IL-6 antibody Ab6 light chain CDR 1
cagtccagtcagagtgtttataataatttcttatcg

SEQ ID NO: 517 - DNA, 21 bp, Oryctolagus cuniculus
  anti-IL-6 antibody Ab6 light chain CDR 2
caggcatccaaactggcatct

SEQ ID NO: 518 - DNA, 33 bp, Oryctolagus cuniculus
  anti-IL-6 antibody Ab6 light chain CDR 3
ctaggcggttatgatgatgatgctgataatgct

SEQ ID NO: 519 - DNA, 15 bp, Oryctolagus cuniculus
  anti-IL-6 antibody Ab6 heavy chain CDR 1
gactatgcaatgagc

SEQ ID NO: 520 - DNA, 51 bp, Oryctolagus cuniculus
  anti-IL-6 antibody Ab6 heavy chain CDR 2
atcatttatgctggtagtggtagcacatggtacgcgagctgggcgaaaggc

SEQ ID NO: 521 - DNA, 42 bp, Oryctolagus cuniculus
  anti-IL-6 antibody Ab6 heavy chain CDR 3
gatggatacgatgactatggtgatttcgatcgattggatctc

SEQ ID NO: 522 - protein, 122 aa, Oryctolagus cuniculus
  anti-IL-6 antibody Ab7 light chain variable domain
MDTRAPTQLLGLLLLWLPGARCAYDMTQTPASVSAAVGGTVTIKCQASQSINNELSWYQQKSGQRPKLLIYRASTLASGVSSRFKGSGSGTEFTLTISDLECADAATYYCQQGYSLRNIDNA

SEQ ID NO: 523 - protein, 125 aa, Oryctolagus cuniculus
  anti-IL-6 antibody Ab7 heavy chain variable domain
METGLRWLLLVAVLSGVQCQSLEESGGRLVTPGTPLTLTCTASGFSLSNYYMTWVRQAPGKGLEWIGMIYGSDETAYANWAIGRFTISKTSTTVDLKMTSLTAADTATYFCARDDSSDWDAKFNL

SEQ ID NO: 524 - protein, 11 aa, Oryctolagus cuniculus
  anti-IL-6 antibody Ab7 light chain CDR 1
QASQSINNELS

SEQ ID NO: 525 - protein, 7 aa, Oryctolagus cuniculus
  anti-IL-6 antibody Ab7 light chain CDR 2
RASTLAS

SEQ ID NO: 526 - protein, 12 aa, Oryctolagus cuniculus
  anti-IL-6 antibody Ab7 light chain CDR 3
QQGYSLRNIDNA

SEQ ID NO: 527 - protein, 5 aa, Oryctolagus cuniculus
  anti-IL-6 antibody Ab7 heavy chain CDR 1
NYYMT

SEQ ID NO: 528 - protein, 16 aa, Oryctolagus cuniculus
  anti-IL-6 antibody Ab7 heavy chain CDR 2
MIYGSDETAYANWAIG

SEQ ID NO: 529 - protein, 12 aa, Oryctolagus cuniculus
  anti-IL-6 antibody Ab7 heavy chain CDR 3
DDSSDWDAKFNL

SEQ ID NO: 530 - DNA, 366 bp, Oryctolagus cuniculus
  anti-IL-6 antibody Ab7 light chain variable domain
atggacacgagggcccccactcagctgctggggctcctgctgctctggctcccaggtgccagatgtgcctatgatatgacccagactccagcctcggtgtctgcagctgtgggaggcacagtcaccatcaaatgccaggccagtcagagcattaacaatgaattatcctggtatcagcagaaatcagggcagcgtcccaagctcctgatctatagggcatccactctggcatctggggtctcatcgcggttcaaaggcagtggatctgggacagagttcactctcaccatcagcgacctggagtgtgccgatgctgccacttactactgtcaacagggttatagtctgaggaatattgataatgct

SEQ ID NO: 531 - DNA, 375 bp, Oryctolagus cuniculus
  anti-IL-6 antibody Ab7 heavy chain variable domain
atggagactgggctgcgctggcttctcctggtcgctgtgctctcaggtgtccagtgtcagtcgctggaggagtccgggggtcgcctggtcacgcctgggacacccctgacactcacctgcacagcctctggattctccctcagtaactactacatgacctgggtccgccaggctccagggaaggggctggaatggatcggaatgatttatggtagtgatgaaacagcctacgcgaactgggcgataggccgattcaccatctccaaaacctcgaccacggtggatctgaaaatgaccagtctgacagccgcggacacggccacctatttctgtgccagagatgatagtagtgactgggatgcaaaatttaacttg

SEQ ID NO: 532 - DNA, 33 bp, Oryctolagus cuniculus; identical to SEQ ID NO: 433
  anti-IL-6 antibody Ab7 light chain CDR 1

SEQ ID NO: 533 - DNA, 21 bp, Oryctolagus cuniculus
  anti-IL-6 antibody Ab7 light chain CDR 2
agggcatccactctggcatct

SEQ ID NO: 534 - DNA, 36 bp, Oryctolagus cuniculus; identical to SEQ ID NO: 435
  anti-IL-6 antibody Ab7 light chain CDR 3

SEQ ID NO: 535 - DNA, 15 bp, Oryctolagus cuniculus
  anti-IL-6 antibody Ab7 heavy chain CDR 1
aactactacatgacc

SEQ ID NO: 536 - DNA, 48 bp, Oryctolagus cuniculus
  anti-IL-6 antibody Ab7 heavy chain CDR 2
atgatttatggtagtgatgaaacagcctacgcgaactgggcgataggc

SEQ ID NO: 537 - DNA, 36 bp, Oryctolagus cuniculus; identical to SEQ ID NO: 438
  anti-IL-6 antibody Ab7 heavy chain CDR 3

SEQ ID NO: 538 - protein, 109 aa, Oryctolagus cuniculus
EVQLVESGGGLVQPGGSLRLSCAASGFSLSNYYMTWVRQAPGKGLEWVGMIYGSDETAYANWAIGRFTISRDNSKNTLYLQMNSLRAEDTAVYYCARDDSSDWDAKFNL

SEQ ID NO: 539 - protein, 109 aa, Oryctolagus cuniculus
EVQLVESGGGLVQPGGSLRLSCAASGFSLSNYYMTWVRQAPGKGLEWVGMIYGSDETAYANSAIGRFTISRDNSKNTLYLQMNSLRAEDTAVYYCARDDSSDWDAKFNL

SEQ ID NO: 540 - protein, 100 aa, Oryctolagus cuniculus
DIQMTQSPSTLSASVGDRVTITCQASQSINNELSWYQQKPGKAPKLLIYRASTLASGVPSRFSGSGSGTEFTLTISSLQPDDFATYYCQQGYSLRNIDNA

SEQ ID NO: 541 - protein, 16 aa, Oryctolagus cuniculus
IIYGSDETAYATSAIG

SEQ ID NO: 542 - protein, 16 aa, Oryctolagus cuniculus
MIYGSDETAYANSAIG

SEQ ID NO: 543 - protein, 123 aa, Oryctolagus cuniculus
  anti-IL-6 antibody Ab8 light chain variable domain
MDTRAPTQLLGLLLLWLPGATFAAVLTQTPSPVSAAVGGTVTISCQSSQSVGNNQDLSWFQQRPGQPPKLLIYEISKLESGVPSRFSGSGSGTHFTLTISGVQCDDAATYYCLGGYDDDADNA

SEQ ID NO: 544 - protein, 128 aa, Oryctolagus cuniculus
  anti-IL-6 antibody Ab8 heavy chain variable domain
METGLRWLLLVAVLKGVQCHSVEESGGRLVTPGTPLTLTCTVSGFSLSSRTMSWVRQAPGKGLEWIGYIWSGGSTYYATWAKGRFTISKTSTTVDLKITSPTTEDTATYFCARLGDTGGHAYATRLNL

SEQ ID NO: 545 - protein, 13 aa, Oryctolagus cuniculus
  anti-IL-6 antibody Ab8 light chain CDR 1
QSSQSVGNNQDLS

SEQ ID NO: 546 - protein, 7 aa, Oryctolagus cuniculus
  anti-IL-6 antibody Ab8 light chain CDR 2
EISKLES

SEQ ID NO: 547 - protein, 11 aa, Oryctolagus cuniculus
  anti-IL-6 antibody Ab8 light chain CDR 3
LGGYDDDADNA

SEQ ID NO: 548 - protein, 5 aa, Oryctolagus cuniculus
  anti-IL-6 antibody Ab8 heavy chain CDR 1
SRTMS

SEQ ID NO: 549 - protein, 16 aa, Oryctolagus cuniculus
  anti-IL-6 antibody Ab8 heavy chain CDR 2
YIWSGGSTYYATWAKG

SEQ ID NO: 550 - protein, 15 aa, Oryctolagus cuniculus
  anti-IL-6 antibody Ab8 heavy chain CDR 3
LGDTGGHAYATRLNL

SEQ ID NO: 551 - DNA, 369 bp, Oryctolagus cuniculus
  anti-IL-6 antibody Ab8 light chain variable domain
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgttggtaataaccaggacttatcctggtttcagcagagaccagggcagcctcccaagctcctgatctacgaaatatccaaactggaatctggggtcccatcgcggttcagcggcagtggatctgggacacacttcactctcaccatcagcggcgtacagtgtgacgatgctgccacttactactgtctaggcggttatgatgatgatgctgataatgct

SEQ ID NO: 552 - DNA, 384 bp, Oryctolagus cuniculus
  anti-IL-6 antibody Ab8 heavy chain variable domain
atggagactgggctgcgctggcttctcctggtcgctgtgctcaaaggtgtccagtgtcactcggtggaggagtccgggggtcgcctggtcacgcctgggacacccctgacactcacctgcacagtctctggattctccctcagtagtcgtacaatgtcctgggtccgccaggctccagggaaggggctggagtggatcggatacatttggagtggtggtagcacatactacgcgacctgggcgaaaggccgattcaccatctccaaaacctcgaccacggtggatctgaaaatcaccagtccgacaaccgaggacacggccacctatttctgtgccagattgggcgatactggtggtcacgcttatgctactcgcttaaatctc

SEQ ID NO: 553 - DNA, 39 bp, Oryctolagus cuniculus
  anti-IL-6 antibody Ab8 light chain CDR 1
cagtccagtcagagtgttggtaataaccaggacttatcc

SEQ ID NO: 554 - DNA, 21 bp, Oryctolagus cuniculus
  anti-IL-6 antibody Ab8 light chain CDR 2
gaaatatccaaactggaatct

SEQ ID NO: 555 - DNA, 33 bp, Oryctolagus cuniculus; identical to SEQ ID NO: 518
  anti-IL-6 antibody Ab8 light chain CDR 3

SEQ ID NO: 556 - DNA, 15 bp, Oryctolagus cuniculus
  anti-IL-6 antibody Ab8 heavy chain CDR 1
agtcgtacaatgtcc

SEQ ID NO: 557 - DNA, 48 bp, Oryctolagus cuniculus
  anti-IL-6 antibody Ab8 heavy chain CDR 2
tacatttggagtggtggtagcacatactacgcgacctgggcgaaaggc

SEQ ID NO: 558 - DNA, 45 bp, Oryctolagus cuniculus
  anti-IL-6 antibody Ab8 heavy chain CDR 3
ttgggcgatactggtggtcacgcttatgctactcgcttaaatctc

SEQ ID NO: 559 - protein, 123 aa, Oryctolagus cuniculus
  anti-IL-6 antibody Ab9 light chain variable domain
MDTRAPTQLLGLLLLWLPGATFAAVLTQTPSSVSAAVGGTVSISCQSSQSVYSNKYLAWYQQKPGQPPKLLIYWTSKLASGAPSRFSGSGSGTQFTLTISGVQCDDAATYYCLGAYDDDADNA

SEQ ID NO: 560 - protein, 126 aa, Oryctolagus cuniculus
  anti-IL-6 antibody Ab9 heavy chain variable domain
METGLRWLLLVAVLKGVQCQSVEESGGRLVKPDETLTLTCTASGFSLEGGYMTWVRQAPGKGLEWIGISYDSGSTYYASWAKGRFTISKTSSTTVDLKMTSLTTEDTATYFCVRSLKYPTVTSDDL

SEQ ID NO: 561 - protein, 13 aa, Oryctolagus cuniculus
  anti-IL-6 antibody Ab9 light chain CDR 1
QSSQSVYSNKYLA

SEQ ID NO: 562 - protein, 7 aa, Oryctolagus cuniculus
  anti-IL-6 antibody Ab9 light chain CDR 2
WTSKLAS

SEQ ID NO: 563 - protein, 11 aa, Oryctolagus cuniculus
  anti-IL-6 antibody Ab9 light chain CDR 3
LGAYDDDADNA

SEQ ID NO: 564 - protein, 5 aa, Oryctolagus cuniculus
  anti-IL-6 antibody Ab9 heavy chain CDR 1
GGYMT

SEQ ID NO: 565 - protein, 16 aa, Oryctolagus cuniculus
  anti-IL-6 antibody Ab9 heavy chain CDR 2
ISYDSGSTYYASWAKG

SEQ ID NO: 566 - protein, 12 aa, Oryctolagus cuniculus
  anti-IL-6 antibody Ab9 heavy chain CDR 3
SLKYPTVTSDDL

SEQ ID NO: 567 - DNA, 369 bp, Oryctolagus cuniculus
  anti-IL-6 antibody Ab9 light chain variable domain
atggacacgagggcccccactcagctgctggggctcctgctgctctggctcccaggtgccacatttgcagccgtgctgacccagacaccatcgtccgtgtctgcagctgtgggaggcacagtcagcatcagttgccagtccagtcagagtgtttatagtaataagtacctagcctggtatcagcagaaaccagggcagcctcccaagctcctgatctactggacatccaaactggcatctggggccccatcacggttcagcggcagtggatctgggacacaattcactctcaccatcagcggcgtgcagtgtgacgatgctgccacttactactgtctaggcgcttatgatgatgatgctgataatgct

SEQ ID NO: 568 - DNA, 378 bp, Oryctolagus cuniculus
  anti-IL-6 antibody Ab9 heavy chain variable domain
atggagactgggctgcgctggcttctcctggtcgctgtgctcaaaggtgtccagtgtcagtcggtggaagagtccgggggtcgcctggtcaagcctgacgaaaccctgacactcacctgcacagcctctggattctccctggagggcggctacatgacctgggtccgccaggctccagggaaggggctggaatggatcggaatcagttatgatagtggtagcacatactacgcgagctgggcgaaaggccgattcaccatctccaagacctcgtcgaccacggtggatctgaaaatgaccagtctgacaaccgaggacacggccacctatttctgcgtcagatcactaaaatatcctactgttacttctgatgacttg

SEQ ID NO: 569 - DNA, 39 bp, Oryctolagus cuniculus
  anti-IL-6 antibody Ab9 light chain CDR 1
cagtccagtcagagtgtttatagtaataagtacctagcc

SEQ ID NO: 570 - DNA, 21 bp, Oryctolagus cuniculus
  anti-IL-6 antibody Ab9 light chain CDR 2
tggacatccaaactggcatct

SEQ ID NO: 571 - DNA, 33 bp, Oryctolagus cuniculus
  anti-IL-6 antibody Ab9 light chain CDR 3
ctaggcgcttatgatgatgatgctgataatgct

SEQ ID NO: 572 - DNA, 15 bp, Oryctolagus cuniculus
  anti-IL-6 antibody Ab9 heavy chain CDR 1
ggcggctacatgacc

SEQ ID NO: 573 - DNA, 48 bp, Oryctolagus cuniculus
  anti-IL-6 antibody Ab9 heavy chain CDR 2
atcagttatgatagtggtagcacatactacgcgagctgggcgaaaggc

SEQ ID NO: 574 - DNA, 36 bp, Oryctolagus cuniculus
  anti-IL-6 antibody Ab9 heavy chain CDR 3
tcactaaaatatcctactgttacttctgatgacttg

SEQ ID NO: 575 - protein, 123 aa, Oryctolagus cuniculus
  anti-IL-6 antibody Ab10 light chain variable domain
MDTRAPTQLLGLLLLWLPGATFAAVLTQTPSPVSAAVGGTVTISCQSSQSVYNNNDLAWYQQKPGQPPKLLIYYASTLASGVPSRFKGSGSGTQFTLTISGVQCDDAAAYYCLGGYDDDADNA

SEQ ID NO: 576 - protein, 129 aa, Oryctolagus cuniculus
  anti-IL-6 antibody Ab10 heavy chain variable domain
METGLRWLLLVAVLKGVQCQSVEESGGRLVTPGTPLTLTCTVSGLSLSSNTINWVRQAPGKGLEWIGYIWSGGSTYYASWVNGRFTISKTSTTVDLKITSPTTEDTATYFCARGGYASGGYPYATRLDL

SEQ ID NO: 577 - protein, 13 aa, Oryctolagus cuniculus
  anti-IL-6 antibody Ab10 light chain CDR 1
QSSQSVYNNNDLA

SEQ ID NO: 578 - protein, 7 aa, Oryctolagus cuniculus
  anti-IL-6 antibody Ab10 light chain CDR 2
YASTLAS

SEQ ID NO: 579 - protein, 11 aa, Oryctolagus cuniculus
  anti-IL-6 antibody Ab10 light chain CDR 3
LGGYDDDADNA

SEQ ID NO: 580 - protein, 5 aa, Oryctolagus cuniculus
  anti-IL-6 antibody Ab10 heavy chain CDR 1
SNTIN

SEQ ID NO: 581 - protein, 16 aa, Oryctolagus cuniculus
  anti-IL-6 antibody Ab10 heavy chain CDR 2
YIWSGGSTYYASWVNG

SEQ ID NO: 582 - protein, 16 aa, Oryctolagus cuniculus
  anti-IL-6 antibody Ab10 heavy chain CDR 3
GGYASGGYPYATRLDL

SEQ ID NO: 583 - DNA, 369 bp, Oryctolagus cuniculus
  anti-IL-6 antibody Ab10 light chain variable domain
atggacacgagggcccccactcagctgctggggctcctgctgctctggctcccaggtgccacatttgcagccgtgctgacccagacaccatcacccgtgtctgcagctgtgggaggcacagtcaccatcagttgccagtccagtcagagtgtttataataataacgacttagcctggtatcagcagaaaccagggcagcctcctaaactcctgatctattatgcatccactctggcatctggggtcccatcgcggttcaaaggcagtggatctgggacacagttcactctcaccatcagcggcgtgcagtgtgacgatgctgccgcttactactgtctaggcggttatgatgatgatgctgataatgct

SEQ ID NO: 584 - DNA, 387 bp, Oryctolagus cuniculus
  anti-IL-6 antibody Ab10 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtatctggattatccctcagtagcaatacaataaactgggtccgccaggctccagggaaggggctggagtggatcggatacatttggagtggtggtagtacatactacgcgagctgggtgaatggtcgattcaccatctccaaaacctcgaccacggtggatctgaaaatcaccagtccgacaaccgaggacacggccacctatttctgtgccagagggggttacgctagtggtggttatccttatgccactcggttggatctc

SEQ ID NO: 585 - DNA, 39 bp, Oryctolagus cuniculus
  anti-IL-6 antibody Ab10 light chain CDR 1
cagtccagtcagagtgtttataataataacgacttagcc

SEQ ID NO: 586 - DNA, 21 bp, Oryctolagus cuniculus
  anti-IL-6 antibody Ab10 light chain CDR 2
tatgcatccactctggcatct

SEQ ID NO: 587 - DNA, 33 bp, Oryctolagus cuniculus; identical to SEQ ID NO: 518
  anti-IL-6 antibody Ab10 light chain CDR 3

SEQ ID NO: 588 - DNA, 15 bp, Oryctolagus cuniculus
  anti-IL-6 antibody Ab10 heavy chain CDR 1
agcaatacaataaac

SEQ ID NO: 589 - DNA, 48 bp, Oryctolagus cuniculus
  anti-IL-6 antibody Ab10 heavy chain CDR 2
tacatttggagtggtggtagtacatactacgcgagctgggtgaatggt

SEQ ID NO: 590 - DNA, 48 bp, Oryctolagus cuniculus
  anti-IL-6 antibody Ab10 heavy chain CDR 3
gggggttacgctagtggtggttatccttatgccactcggttggatctc

SEQ ID NO: 591 - protein, 123 aa, Oryctolagus cuniculus
  anti-IL-6 antibody Ab11 light chain variable domain
MDTRAPTQLLGLLLLWLPGATFAAVLTQTPSSVSAAVGGTVTINCQSSQSVYNNDYLSWYQQRPGQRPKLLIYGASKLASGVPSRFKGSGSGKQFTLTISGVQCDDAATYYCLGDYDDDADNT

SEQ ID NO: 592 - protein, 123 aa, Oryctolagus cuniculus
  anti-IL-6 antibody Ab11 heavy chain variable domain
METGLRWLLLVAVLKGVQCQSLEESGGRLVTPGTPLTLTCTVSGFTLSTNYYLSWVRQAPGKGLEWIGIIYPSGNTYCAKWAKGRFTISKTSSTTVDLKMTSPTTEDTATYFCARNYGGDESL

SEQ ID NO: 593 - protein, 13 aa, Oryctolagus cuniculus
  anti-IL-6 antibody Ab11 light chain CDR 1
QSSQSVYNNDYLS

SEQ ID NO: 594 - protein, 7 aa, Oryctolagus cuniculus
  anti-IL-6 antibody Ab11 light chain CDR 2
GASKLAS

SEQ ID NO: 595 - protein, 11 aa, Oryctolagus cuniculus
  anti-IL-6 antibody Ab11 light chain CDR 3
LGDYDDDADNT

SEQ ID NO: 596 - protein, 6 aa, Oryctolagus cuniculus
  anti-IL-6 antibody Ab11 heavy chain CDR 1
TNYYLS

SEQ ID NO: 597 - protein, 16 aa, Oryctolagus cuniculus
  anti-IL-6 antibody Ab11 heavy chain CDR 2
IIYPSGNTYCAKWAKG

SEQ ID NO: 598 - protein, 8 aa, Oryctolagus cuniculus
  anti-IL-6 antibody Ab11 heavy chain CDR 3
NYGGDESL

SEQ ID NO: 599 - DNA, 369 bp, Oryctolagus cuniculus
  anti-IL-6 antibody Ab11 light chain variable domain
atggacacgagggcccccactcagctgctggggctcctgctgctctggctcccaggtgccacatttgcagccgtgctgacccagacaccatcctccgtgtctgcagctgtgggaggcacagtcaccatcaattgccagtccagtcagagtgtttataataacgactacttatcctggtatcaacagaggccagggcaacgtcccaagctcctaatctatggtgcttccaaactggcatctggggtcccgtcacggttcaaaggcagtggatctgggaaacagtttactctcaccatcagcggcgtgcagtgtgacgatgctgccacttactactgtctgggcgattatgatgatgatgctgataatact

SEQ ID NO: 600 - DNA, 369 bp, Oryctolagus cuniculus
  anti-IL-6 antibody Ab11 heavy chain variable domain
atggagactgggctgcgctggcttctcctggtcgctgtgctcaaaggtgtccagtgtcagtcgctggaggagtccgggggtcgcctggtcacgcctgggacacccctgacactcacttgcacagtctctggattcaccctcagtaccaactactacctgagctgggtccgccaggctccagggaaggggctagaatggatcggaatcatttatcctagtggtaacacatattgcgcgaagtgggcgaaaggccgattcaccatctccaaaacctcgtcgaccacggtggatctgaaaatgaccagtccgacaaccgaggacacagccacgtatttctgtgccagaaattatggtggtgatgaaagtttg

SEQ ID NO: 601 - DNA, 39 bp, Oryctolagus cuniculus
  anti-IL-6 antibody Ab11 light chain CDR 1
cagtccagtcagagtgtttataataacgactacttatcc

SEQ ID NO: 602 - DNA, 21 bp, Oryctolagus cuniculus
  anti-IL-6 antibody Ab11 light chain CDR 2
ggtgcttccaaactggcatct

SEQ ID NO: 603 - DNA, 33 bp, Oryctolagus cuniculus
  anti-IL-6 antibody Ab11 light chain CDR 3
ctgggcgattatgatgatgatgctgataatact

SEQ ID NO: 604 - DNA, 18 bp, Oryctolagus cuniculus
  anti-IL-6 antibody Ab11 heavy chain CDR 1
accaactactacctgagc

SEQ ID NO: 605 - DNA, 48 bp, Oryctolagus cuniculus
  anti-IL-6 antibody Ab11 heavy chain CDR 2
atcatttatcctagtggtaacacatattgcgcgaagtgggcgaaaggc

SEQ ID NO: 606 - DNA, 24 bp, Oryctolagus cuniculus
  anti-IL-6 antibody Ab11 heavy chain CDR 3
aattatggtggtgatgaaagtttg

SEQ ID NO: 607 - protein, 119 aa, Oryctolagus cuniculus
  anti-IL-6 antibody Ab12 light chain variable domain
MDTRAPTQLLGLLLLWLPGARCDVVMTQTPASVEAAVGGTVTIKCQASETIGNALAWYQQKSGQPPKLLIYKASKLASGVPSRFKGSGSGTEYTLTISDLECADAATYYCQWCYFGDSV

SEQ ID NO: 608 - protein, 128 aa, Oryctolagus cuniculus
  anti-IL-6 antibody Ab12 heavy chain variable domain
METGLRWLLLVTVLKGVQCQEQLVESGGGLVQPEGSLTLTCTASGFDFSSGYYMCWVRQAPGKGLEWIACIFTITTNTYYASWAKGRFTISKTSSTTVTLQMTSLTAADTATYLCARGIYSDNNYYAL

SEQ ID NO: 609 - protein, 11 aa, Oryctolagus cuniculus
  anti-IL-6 antibody Ab12 light chain CDR 1
QASETIGNALA

SEQ ID NO: 610 - protein, 7 aa, Oryctolagus cuniculus
  anti-IL-6 antibody Ab12 light chain CDR 2
KASKLAS

SEQ ID NO: 611 - protein, 9 aa, Oryctolagus cuniculus
  anti-IL-6 antibody Ab12 light chain CDR 3
QWCYFGDSV

SEQ ID NO: 612 - protein, 6 aa, Oryctolagus cuniculus
  anti-IL-6 antibody Ab12 heavy chain CDR 1
SGYYMC

SEQ ID NO: 613 - protein, 17 aa, Oryctolagus cuniculus
  anti-IL-6 antibody Ab12 heavy chain CDR 2
CIFTITTNTYYASWAKG

SEQ ID NO: 614 - protein, 11 aa, Oryctolagus cuniculus
  anti-IL-6 antibody Ab12 heavy chain CDR 3
GIYSDNNYYAL

SEQ ID NO: 615 - DNA, 357 bp, Oryctolagus cuniculus
  anti-IL-6 antibody Ab12 light chain variable domain
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagaccattggcaatgcattagcctggtatcagcagaaatcagggcagcctcccaagctcctgatctacaaggcatccaaactggcatctggggtcccatcgcggttcaaaggcagtggatctgggacagagtacactctcaccatcagcgacctggagtgtgccgatgctgccacttactactgtcaatggtgttattttggtgatagtgtt

SEQ ID NO: 616 - DNA, 384 bp, Oryctolagus cuniculus
  anti-IL-6 antibody Ab12 heavy chain variable domain
atggagactgggctgcgctggcttctcctggtcactgtgctcaaaggtgtccagtgtcaggagcagctggtggagtccgggggaggcctggtccagcctgagggatccctgacactcacctgcacagcctctggattcgacttcagtagcggctactacatgtgctgggtccgccaggctccagggaaggggctggagtggatcgcgtgtattttcactattactactaacacttactacgcgagctgggcgaaaggccgattcaccatctccaagacctcgtcgaccacggtgactctgcaaatgaccagtctgacagccgcggacacggccacctatctctgtgcgagagggatttattctgataataattattatgccttg

SEQ ID NO: 617 - DNA, 33 bp, Oryctolagus cuniculus
  anti-IL-6 antibody Ab12 light chain CDR 1
caggccagtgagaccattggcaatgcattagcc

SEQ ID NO: 618 - DNA, 21 bp, Oryctolagus cuniculus
  anti-IL-6 antibody Ab12 light chain CDR 2
aaggcatccaaactggcatct

SEQ ID NO: 619 - DNA, 27 bp, Oryctolagus cuniculus
  anti-IL-6 antibody Ab12 light chain CDR 3
caatggtgttattttggtgatagtgtt

SEQ ID NO: 620 - DNA, 18 bp, Oryctolagus cuniculus
  anti-IL-6 antibody Ab12 heavy chain CDR 1
agcggctactacatgtgc

SEQ ID NO: 621 - DNA, 51 bp, Oryctolagus cuniculus
  anti-IL-6 antibody Ab12 heavy chain CDR 2
tgtattttcactattactactaacacttactacgcgagctgggcgaaaggc

SEQ ID NO: 622 - DNA, 33 bp, Oryctolagus cuniculus
  anti-IL-6 antibody Ab12 heavy chain CDR 3
gggatttattctgataataattattatgccttg

SEQ ID NO: 623 - protein, 119 aa, Oryctolagus cuniculus
  anti-IL-6 antibody Ab13 light chain variable domain
MDTRAPTQLLGLLLLWLPGARCDVVMTQTPASVEAAVGGTVTIKCQASESIGNALAWYQQKPGQPPKLLIYKASTLASGVPSRFSGSGSGTEFTLTISGVQCADAAAYYCQWCYFGDSV

SEQ ID NO: 624 - protein, 128 aa, Oryctolagus cuniculus
  anti-IL-6 antibody Ab13 heavy chain variable domain
METGLRWLLLVAVLKGVQCQQQLVESGGGLVKPGASLTLTCKASGFSFSSGYYMCWVRQAPGKGLESIACIFTITDNTYYANWAKGRFTISKPSSPTVTLQMTSLTAADTATYFCARGIYSTDNYYAL

SEQ ID NO: 625 - protein, 11 aa, Oryctolagus cuniculus
  anti-IL-6 antibody Ab13 light chain CDR 1
QASESIGNALA

SEQ ID NO: 626 - protein, 7 aa, Oryctolagus cuniculus
  anti-IL-6 antibody Ab13 light chain CDR 2
KASTLAS

SEQ ID NO: 627 - protein, 9 aa, Oryctolagus cuniculus
  anti-IL-6 antibody Ab13 light chain CDR 3
QWCYFGDSV

SEQ ID NO: 628 - protein, 6 aa, Oryctolagus cuniculus
  anti-IL-6 antibody Ab13 heavy chain CDR 1
SGYYMC

SEQ ID NO: 629 - protein, 17 aa, Oryctolagus cuniculus
  anti-IL-6 antibody Ab13 heavy chain CDR 2
CIFTITDNTYYANWAKG

SEQ ID NO: 630 - protein, 11 aa, Oryctolagus cuniculus
  anti-IL-6 antibody Ab13 heavy chain CDR 3
GIYSTDNYYAL

SEQ ID NO: 631 - DNA, 357 bp, Oryctolagus cuniculus
  anti-IL-6 antibody Ab13 light chain variable domain
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcattggcaatgcattagcctggtatcagcagaaaccagggcagcctcccaagctcctgatctacaaggcatccactctggcatctggggtcccatcgcggttcagcggcagtggatctgggacagagttcactctcaccatcagcggcgtgcagtgtgccgatgctgccgcttactactgtcaatggtgttattttggtgatagtgtt

SEQ ID NO: 632 - DNA, 384 bp, Oryctolagus cuniculus
  anti-IL-6 antibody Ab13 heavy chain variable domain
atggagactgggctgcgctggcttctcctggtcgctgtgctcaaaggtgtccagtgtcagcagcagctggtggagtccgggggaggcctggtcaagccgggggcatccctgacactcacctgcaaagcctctggattctccttcagtagcggctactacatgtgctgggtccgccaggctccagggaaggggctggagtcgatcgcatgcatttttactattactgataacacttactacgcgaactgggcgaaaggccgattcaccatctccaagccctcgtcgcccacggtgactctgcaaatgaccagtctgacagccgcggacacggccacctatttctgtgcgagggggatttattctactgataattattatgccttg

SEQ ID NO: 633 - DNA, 33 bp, Oryctolagus cuniculus
  anti-IL-6 antibody Ab13 light chain CDR 1
caggccagtgagagcattggcaatgcattagcc

SEQ ID NO: 634 - DNA, 21 bp, Oryctolagus cuniculus
  anti-IL-6 antibody Ab13 light chain CDR 2
aaggcatccactctggcatct

SEQ ID NO: 635 - DNA, 27 bp, Oryctolagus cuniculus
  anti-IL-6 antibody Ab13 light chain CDR 3
caatggtgttattttggtgatagtgtt

SEQ ID NO: 636 - DNA, 18 bp, Oryctolagus cuniculus
  anti-IL-6 antibody Ab13 heavy chain CDR 1
agcggctactacatgtgc

SEQ ID NO: 637 - DNA, 51 bp, Oryctolagus cuniculus
  anti-IL-6 antibody Ab13 heavy chain CDR 2
tgcatttttactattactgataacacttactacgcgaactgggcgaaaggc

SEQ ID NO: 638 - DNA, 33 bp, Oryctolagus cuniculus
  anti-IL-6 antibody Ab13 heavy chain CDR 3
gggatttattctactgataattattatgccttg

SEQ ID NO: 639 - protein, 123 aa, Oryctolagus cuniculus
  anti-IL-6 antibody Ab14 light chain variable domain
MDTRAPTQLLGLLLLWLPGARCDVVMTQTPASVEAAVGGTVTIKCQASQSVSSYLNWYQQKPGQPPKLLIYRASTLESGVPSRFKGSGSGTEFTLTISDLECADAATYYCQCTYGTSSSYGAA

SEQ ID NO: 640 - protein, 133 aa, Oryctolagus cuniculus
  anti-IL-6 antibody Ab14 heavy chain variable domain
METGLRWLLLVAVLKGVQCQSVEESGGRLVTPGTPLTLTCTVSGISLSSNAISWVRQAPGKGLEWIGIISYSGTTYYASWAKGRFTISKTSSTTVDLKITSPTTEDTATYFCARDDPTTVMVMLIPFGAGMDL

SEQ ID NO: 641 - protein, 11 aa, Oryctolagus cuniculus
  anti-IL-6 antibody Ab14 light chain CDR 1
QASQSVSSYLN

SEQ ID NO: 642 - protein, 7 aa, Oryctolagus cuniculus
  anti-IL-6 antibody Ab14 light chain CDR 2
RASTLES

SEQ ID NO: 643 - protein, 13 aa, Oryctolagus cuniculus
  anti-IL-6 antibody Ab14 light chain CDR 3
QCTYGTSSSYGAA

SEQ ID NO: 644 - protein, 5 aa, Oryctolagus cuniculus
  anti-IL-6 antibody Ab14 heavy chain CDR 1
SNAIS

SEQ ID NO: 645 - protein, 16 aa, Oryctolagus cuniculus
  anti-IL-6 antibody Ab14 heavy chain CDR 2
IISYSGTTYYASWAKG

SEQ ID NO: 646 - protein, 19 aa, Oryctolagus cuniculus
  anti-IL-6 antibody Ab14 heavy chain CDR 3
DDPTTVMVMLIPFGAGMDL

SEQ ID NO: 647 - DNA, 369 bp, Oryctolagus cuniculus
  anti-IL-6 antibody Ab14 light chain variable domain
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtcagagcgttagtagctacttaaactggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaatgtacttatggtactagtagtagttatggtgctgct

SEQ ID NO: 648 - DNA, 399 bp, Oryctolagus cuniculus
  anti-IL-6 antibody Ab14 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcaccgtctctggtatctccctcagtagcaatgcaataagctgggtccgccaggctccagggaaggggctggaatggatcggaatcattagttatagtggtaccacatactacgcgagctgggcgaaaggccgattcaccatctccaaaacctcgtcgaccacggtggatctgaaaatcactagtccgacaaccgaggacacggccacctacttctgtgccagagatgaccctacgacagttatggttatgttgataccttttggagccggcatggacctc

SEQ ID NO: 649 - DNA, 33 bp, Oryctolagus cuniculus
  anti-IL-6 antibody Ab14 light chain CDR 1
caggccagtcagagcgttagtagctacttaaac

SEQ ID NO: 650 - DNA, 21 bp, Oryctolagus cuniculus
  anti-IL-6 antibody Ab14 light chain CDR 2
agggcatccactctggaatct

SEQ ID NO: 651 - DNA, 39 bp, Oryctolagus cuniculus
  anti-IL-6 antibody Ab14 light chain CDR 3
caatgtacttatggtactagtagtagttatggtgctgct

SEQ ID NO: 652 - DNA, 15 bp, Oryctolagus cuniculus
  anti-IL-6 antibody Ab14 heavy chain CDR 1
agcaatgcaataagc

SEQ ID NO: 653 - DNA, 48 bp, Oryctolagus cuniculus
  anti-IL-6 antibody Ab14 heavy chain CDR 2
atcattagttatagtggtaccacatactacgcgagctgggcgaaaggc

SEQ ID NO: 654 - DNA, 57 bp, Oryctolagus cuniculus
  anti-IL-6 antibody Ab14 heavy chain CDR 3
gatgaccctacgacagttatggttatgttgataccttttggagccggcatggacctc

SEQ ID NO: 655 - protein, 125 aa, Oryctolagus cuniculus
  anti-IL-6 antibody Ab15 light chain variable domain
MDTRAPTQLLGLLLLWLPGATFAQVLTQTASPVSAAVGGTVTINCQASQSVYKNNYLSWYQQKPGQPPKGLIYSASTLDSGVPLRFSGSGSGTQFTLTISDVQCDDAATYYCLGSYDCSSGDCYA

SEQ ID NO: 656 - protein, 119 aa, Oryctolagus cuniculus
  anti-IL-6 antibody Ab15 heavy chain variable domain
METGLRWLLLVAVLKGVQCQSLEESGGDLVKPEGSLTLTCTASGFSFSSYWMCWVRQAPGKGLEWIACIVTGNGNTYYANWAKGRFTISKTSSTTVTLQMTSLTAADTATYFCAKAYDL

SEQ ID NO: 657 - protein, 13 aa, Oryctolagus cuniculus
  anti-IL-6 antibody Ab15 light chain CDR 1
QASQSVYKNNYLS

SEQ ID NO: 658 - protein, 7 aa, Oryctolagus cuniculus
  anti-IL-6 antibody Ab15 light chain CDR 2
SASTLDS

SEQ ID NO: 659 - protein, 13 aa, Oryctolagus cuniculus
  anti-IL-6 antibody Ab15 light chain CDR 3
LGSYDCSSGDCYA

SEQ ID NO: 660 - protein, 5 aa, Oryctolagus cuniculus
  anti-IL-6 antibody Ab15 heavy chain CDR 1
SYWMC

SEQ ID NO: 661 - protein, 17 aa, Oryctolagus cuniculus
  anti-IL-6 antibody Ab15 heavy chain CDR 2
CIVTGNGNTYYANWAKG

SEQ ID NO: 662 - protein, 4 aa, Oryctolagus cuniculus
  anti-IL-6 antibody Ab15 heavy chain CDR 3
AYDL

SEQ ID NO: 663 - DNA, 375 bp, Oryctolagus cuniculus
  anti-IL-6 antibody Ab15 light chain variable domain
atggacacgagggcccccactcagctgctggggctcctgctgctctggctcccaggtgccacatttgcccaagtgctgacccagactgcatcgcccgtgtctgcagctgtgggaggcacagtcaccatcaactgccaggccagtcagagtgtttataagaacaactacttatcctggtatcagcagaaaccagggcagcctcccaaaggcctgatctattctgcatcgactctagattctggggtcccattgcggttcagcggcagtggatctgggacacagttcactctcaccatcagcgacgtgcagtgtgacgatgctgccacttactactgtctaggcagttatgattgtagtagtggtgattgttatgct

SEQ ID NO: 664 - DNA, 357 bp, Oryctolagus cuniculus
  anti-IL-6 antibody Ab15 heavy chain variable domain
atggagactgggctgcgctggcttctcctggtcgctgtgctcaaaggtgtccagtgtcagtcgttggaggagtccgggggagacctggtcaagcctgagggatccctgacactcacctgcacagcctctggattctccttcagtagctactggatgtgctgggtccgccaggctccagggaaggggctggagtggatcgcatgcattgttactggtaatggtaacacttactacgcgaactgggcgaaaggccgattcaccatctccaaaacctcgtcgaccacggtgactctgcaaatgaccagtctgacagccgcggacacggccacctatttttgtgcgaaagcctatgacttg

SEQ ID NO: 665 - DNA, 39 bp, Oryctolagus cuniculus
  anti-IL-6 antibody Ab15 light chain CDR 1
caggccagtcagagtgtttataagaacaactacttatcc

SEQ ID NO: 666 - DNA, 21 bp, Oryctolagus cuniculus
  anti-IL-6 antibody Ab15 light chain CDR 2
tctgcatcgactctagattct

SEQ ID NO: 667 - DNA, 39 bp, Oryctolagus cuniculus
  anti-IL-6 antibody Ab15 light chain CDR 3
ctaggcagttatgattgtagtagtggtgattgttatgct

SEQ ID NO: 668 - DNA, 15 bp, Oryctolagus cuniculus
  anti-IL-6 antibody Ab15 heavy chain CDR 1
agctactggatgtgc

SEQ ID NO: 669 - DNA, 51 bp, Oryctolagus cuniculus
  anti-IL-6 antibody Ab15 heavy chain CDR 2
tgcattgttactggtaatggtaacacttactacgcgaactgggcgaaaggc

SEQ ID NO: 670 - DNA, 12 bp, Oryctolagus cuniculus
  anti-IL-6 antibody Ab15 heavy chain CDR 3
gcctatgacttg

SEQ ID NO: 671 - protein, 123 aa, Oryctolagus cuniculus
  anti-IL-6 antibody Ab16 light chain variable domain
MDTRAPTQLLGLLLLWLPGSTFAAVLTQTPSPVSAAVGGTVSISCQASQSVYDNNYLSWYQQKPGQPPKLLIYGASTLASGVPSRFKGTGSGTQFTLTITDVQCDDAATYYCAGVFNDDSDDA

SEQ ID NO: 672 - protein, 125 aa, Oryctolagus cuniculus
  anti-IL-6 antibody Ab16 heavy chain variable domain
METGLRWLLLVAVPKGVQCQSLEESGGRLVTPGTPLTLTCTLSGFSLSAYYMSWVRQAPGKGLEWIGFITLSDHISYARWAKGRFTISKTSTTVDLKMTSPTTEDTATYFCARSRGWGAMGRLDL

SEQ ID NO: 673 - protein, 13 aa, Oryctolagus cuniculus
  anti-IL-6 antibody Ab16 light chain CDR 1
QASQSVYDNNYLS

SEQ ID NO: 674 - protein, 7 aa, Oryctolagus cuniculus
  anti-IL-6 antibody Ab16 light chain CDR 2
GASTLAS

SEQ ID NO: 675 - protein, 11 aa, Oryctolagus cuniculus
  anti-IL-6 antibody Ab16 light chain CDR 3
AGVFNDDSDDA

SEQ ID NO: 676 - protein, 5 aa, Oryctolagus cuniculus
  anti-IL-6 antibody Ab16 heavy chain CDR 1
AYYMS

SEQ ID NO: 677 - protein, 16 aa, Oryctolagus cuniculus
  anti-IL-6 antibody Ab16 heavy chain CDR 2
FITLSDHISYARWAKG

SEQ ID NO: 678 - protein, 12 aa, Oryctolagus cuniculus
  anti-IL-6 antibody Ab16 heavy chain CDR 3
SRGWGAMGRLDL

SEQ ID NO: 679 - DNA, 369 bp, Oryctolagus cuniculus
  anti-IL-6 antibody Ab16 light chain variable domain
atggacacgagggcccccactcagctgctggggctcctgctgctctggctcccaggttccacatttgccgccgtgctgacccagactccatctcccgtgtctgcagctgtgggaggcacagtcagcatcagttgccaggccagtcagagtgtttatgacaacaactatttatcctggtatcagcagaaaccaggacagcctcccaagctcctgatctatggtgcatccactctggcatctggggtcccatcgcggttcaaaggcacgggatctgggacacagttcactctcaccatcacagacgtgcagtgtgacgatgctgccacttactattgtgcaggcgtttttaatgatgatagtgatgatgcc

SEQ ID NO: 680 - DNA, 375 bp, Oryctolagus cuniculus
  anti-IL-6 antibody Ab16 heavy chain variable domain
atggagactgggctgcgctggcttctcctggtcgctgtgcccaaaggtgtccagtgtcagtcgctggaggagtccgggggtcgcctggtcacgcctgggacacccctgacactcacctgcacactctctggattctccctcagtgcatactatatgagctgggtccgccaggctccagggaaggggctggaatggatcggattcattactctgagtgatcatatatcttacgcgaggtgggcgaaaggccgattcaccatctccaaaacctcgaccacggtggatctgaaaatgaccagtccgacaaccgaggacacggccacctatttctgtgccaggagtcgtggctggggtgcaatgggtcggttggatctc

SEQ ID NO: 681 - DNA, 39 bp, Oryctolagus cuniculus
  anti-IL-6 antibody Ab16 light chain CDR 1
caggccagtcagagtgtttatgacaacaactatttatcc

SEQ ID NO: 682 - DNA, 21 bp, Oryctolagus cuniculus
  anti-IL-6 antibody Ab16 light chain CDR 2
ggtgcatccactctggcatct

SEQ ID NO: 683 - DNA, 33 bp, Oryctolagus cuniculus
  anti-IL-6 antibody Ab16 light chain CDR 3
gcaggcgtttttaatgatgatagtgatgatgcc

SEQ ID NO: 684 - DNA, 15 bp, Oryctolagus cuniculus
  anti-IL-6 antibody Ab16 heavy chain CDR 1
gcatactatatgagc

SEQ ID NO: 685 - DNA, 48 bp, Oryctolagus cuniculus
  anti-IL-6 antibody Ab16 heavy chain CDR 2
ttcattactctgagtgatcatatatcttacgcgaggtgggcgaaaggc

SEQ ID NO: 686 - DNA, 36 bp, Oryctolagus cuniculus; identical to SEQ ID NO: 489
  anti-IL-6 antibody Ab16 heavy chain CDR 3

SEQ ID NO: 687 - protein, 123 aa, Oryctolagus cuniculus
  anti-IL-6 antibody Ab17 light chain variable domain
MDTRAPTQLLGLLLLWLPGATFAAVLTQTPSPVSAAVGGTVTISCQASQSVYNNKNLAWYQQKSGQPPKLLIYWASTLASGVSSRFSGSGSGTQFTLTVSGVQCDDAATYYCLGVFDDDADNA

SEQ ID NO: 688 - protein, 121 aa, Oryctolagus cuniculus
  anti-IL-6 antibody Ab17 heavy chain variable domain
METGLRWLLLVAVLKGVQCQSVEESGGRLVTPGTPLTLTCTASGFSLSSYSMTWVRQAPGKGLEYIGVIGTSGSTYYATWAKGRFTISRTSTTVALKITSPTTEDTATYFCVRSLSSITFL

SEQ ID NO: 689 - protein, 13 aa, Oryctolagus cuniculus
  anti-IL-6 antibody Ab17 light chain CDR 1
QASQSVYNNKNLA

SEQ ID NO: 690 - protein, 7 aa, Oryctolagus cuniculus
  anti-IL-6 antibody Ab17 light chain CDR 2
WASTLAS

SEQ ID NO: 691 - protein, 11 aa, Oryctolagus cuniculus
  anti-IL-6 antibody Ab17 light chain CDR 3
LGVFDDDADNA

SEQ ID NO: 692 - protein, 5 aa, Oryctolagus cuniculus
  anti-IL-6 antibody Ab17 heavy chain CDR 1
SYSMT

SEQ ID NO: 693 - protein, 16 aa, Oryctolagus cuniculus
  anti-IL-6 antibody Ab17 heavy chain CDR 2
VIGTSGSTYYATWAKG

SEQ ID NO: 694 - protein, 8 aa, Oryctolagus cuniculus
  anti-IL-6 antibody Ab17 heavy chain CDR 3
SLSSITFL

SEQ ID NO: 695 - DNA, 369 bp, Oryctolagus cuniculus
  anti-IL-6 antibody Ab17 light chain variable domain
atggacacgagggcccccactcagctgctggggctcctgctgctctggctcccaggtgccacattcgcagccgtgctgacccagacaccatcgcccgtgtctgcggctgtgggaggcacagtcaccatcagttgccaggccagtcagagtgtttataacaacaaaaatttagcctggtatcagcagaaatcagggcagcctcccaagctcctgatctactgggcatccactctggcatctggggtctcatcgcggttcagcggcagtggatctgggacacagttcactctcaccgtcagcggcgtgcagtgtgacgatgctgccacttactactgtctaggcgtttttgatgatgatgctgataatgct

SEQ ID NO: 696 - DNA, 363 bp, Oryctolagus cuniculus
  anti-IL-6 antibody Ab17 heavy chain variable domain
atggagactgggctgcgctggcttctcctggtcgctgtgctcaaaggtgtccaatgtcagtcggtggaggagtccgggggtcgcctggtcacgcctgggacacccctgacactcacctgcacagcctctggattctccctcagtagctactccatgacctgggtccgccaggctccagggaaggggctggaatatatcggagtcattggtactagtggtagcacatactacgcgacctgggcgaaaggccgattcaccatctccagaacctcgaccacggtggctctgaaaatcaccagtccgacaaccgaggacacggccacctatttctgtgtcaggagtctttcttctattactttcttg

SEQ ID NO: 697 - DNA, 39 bp, Oryctolagus cuniculus
  anti-IL-6 antibody Ab17 light chain CDR 1
caggccagtcagagtgtttataacaacaaaaatttagcc

SEQ ID NO: 698 - DNA, 21 bp, Oryctolagus cuniculus
  anti-IL-6 antibody Ab17 light chain CDR 2
tgggcatccactctggcatct

SEQ ID NO: 699 - DNA, 33 bp, Oryctolagus cuniculus
  anti-IL-6 antibody Ab17 light chain CDR 3
ctaggcgtttttgatgatgatgctgataatgct

SEQ ID NO: 700 - DNA, 15 bp, Oryctolagus cuniculus
  anti-IL-6 antibody Ab17 heavy chain CDR 1
agctactccatgacc

SEQ ID NO: 701 - DNA, 48 bp, Oryctolagus cuniculus
  anti-IL-6 antibody Ab17 heavy chain CDR 2
gtcattggtactagtggtagcacatactacgcgacctgggcgaaaggc

SEQ ID NO: 702 - DNA, 24 bp, Oryctolagus cuniculus
  anti-IL-6 antibody Ab17 heavy chain CDR 3
agtctttcttctattactttcttg

SEQ ID NO: 703 - protein, 120 aa, Oryctolagus cuniculus
  anti-IL-6 antibody Ab18 light chain variable domain
MDTRAPTQLLGLLLLWLPGARCAFELTQTPASVEAAVGGTVTINCQASQNIYRYLAWYQQKPGQPPKFLIYLASTLASGVPSRFKGSGSGTEFTLTISDLECADAATYYCQSYYSSNSVA

SEQ ID NO: 704 - protein, 128 aa, Oryctolagus cuniculus
  anti-IL-6 antibody Ab18 heavy chain variable domain
METGLRWLLLVAVLKGVQCQEQLVESGGDLVQPEGSLTLTCTASELDFSSGYWICWVRQVPGKGLEWIGCIYTGSSGSTFYASWAKGRFTISKTSSTTVTLQMTSLTAADTATYFCARGYSGFGYFKL

SEQ ID NO: 705 - protein, 11 aa, Oryctolagus cuniculus
  anti-IL-6 antibody Ab18 light chain CDR 1
QASQNIYRYLA

SEQ ID NO: 706 - protein, 7 aa, Oryctolagus cuniculus
  anti-IL-6 antibody Ab18 light chain CDR 2
LASTLAS

SEQ ID NO: 707 - protein, 10 aa, Oryctolagus cuniculus
  anti-IL-6 antibody Ab18 light chain CDR 3
QSYYSSNSVA

SEQ ID NO: 708 - protein, 6 aa, Oryctolagus cuniculus
  anti-IL-6 antibody Ab18 heavy chain CDR 1
SGYWIC

SEQ ID NO: 709 - protein, 18 aa, Oryctolagus cuniculus
  anti-IL-6 antibody Ab18 heavy chain CDR 2
CIYTGSSGSTFYASWAKG

SEQ ID NO: 710 - protein, 10 aa, Oryctolagus cuniculus
  anti-IL-6 antibody Ab18 heavy chain CDR 3
GYSGFGYFKL

SEQ ID NO: 711 - DNA, 360 bp, Oryctolagus cuniculus
  anti-IL-6 antibody Ab18 light chain variable domain
atggacacgagggcccccactcagctgctggggctcctgctgctctggctcccaggtgccagatgtgcattcgaattgacccagactccagcctccgtggaggcagctgtgggaggcacagtcaccatcaattgccaggccagtcagaacatttatagatacttagcctggtatcagcagaaaccagggcagcctcccaagttcctgatctatctggcatctactctggcatctggggtcccatcgcggtttaaaggcagtggatctgggacagagttcactctcaccatcagcgacctggagtgtgccgatgctgccacttactactgtcaaagttattatagtagtaatagtgtcgct

SEQ ID NO: 712 - DNA, 384 bp, Oryctolagus cuniculus
  anti-IL-6 antibody Ab18 heavy chain variable domain
atggagactgggctgcgctggcttctcctggtcgctgtgctcaaaggtgtccagtgtcaggagcagctggtggagtccgggggagacctggtccagcctgagggatccctgacactcacctgcacagcttctgagttagacttcagtagcggctactggatatgctgggtccgccaggttccagggaaggggctggagtggatcggatgcatttatactggtagtagtggtagcactttttacgcgagttgggcgaaaggccgattcaccatctccaaaacctcgtcgaccacggtgactctgcaaatgaccagtctgacagccgcggacacggccacctatttctgtgcgagaggttatagtggctttggttactttaagttg

SEQ ID NO: 713 - DNA, 33 bp, Oryctolagus cuniculus
  anti-IL-6 antibody Ab18 light chain CDR 1
caggccagtcagaacatttatagatacttagcc

SEQ ID NO: 714 - DNA, 21 bp, Oryctolagus cuniculus
  anti-IL-6 antibody Ab18 light chain CDR 2
ctggcatctactctggcatct

SEQ ID NO: 715 - DNA, 30 bp, Oryctolagus cuniculus
  anti-IL-6 antibody Ab18 light chain CDR 3
caaagttattatagtagtaatagtgtcgct

SEQ ID NO: 716 - DNA, 18 bp, Oryctolagus cuniculus
  anti-IL-6 antibody Ab18 heavy chain CDR 1
agcggctactggatatgc

SEQ ID NO: 717 - DNA, 54 bp, Oryctolagus cuniculus
  anti-IL-6 antibody Ab18 heavy chain CDR 2
tgcatttatactggtagtagtggtagcactttttacgcgagttgggcgaaaggc

SEQ ID NO: 718 - DNA, 30 bp, Oryctolagus cuniculus
  anti-IL-6 antibody Ab18 heavy chain CDR 3
ggttatagtggctttggttactttaagttg

SEQ ID NO: 719 - protein, 122 aa, Oryctolagus cuniculus
  anti-IL-6 antibody Ab19 light chain variable domain
MDTRAPTQLLGLLLLWLPGARCAYDMTQTPASVEVAVGGTVTIKCQASEDIYRLLAWYQQKPGQPPKLLIYDSSDLASGVPSRFKGSGSGTEFTLAISGVQCDDAATYYCQQAWSYSDIDNA

SEQ ID NO: 720 - protein, 123 aa, Oryctolagus cuniculus
  anti-IL-6 antibody Ab19 heavy chain variable domain
METGLRWLLLVAVLKGVQCQSVEESGGRLVTPGTPLTLTCTASGFSLSSYYMSWVRQAPGKGLEWIGIITTSGNTFYASWAKGRLTISRTSTTVDLKITSPTTEDTATYFCARTSDIFYYRNL

SEQ ID NO: 721 - protein, 11 aa, Oryctolagus cuniculus
  anti-IL-6 antibody Ab19 light chain CDR 1
QASEDIYRLLA

SEQ ID NO: 722 - protein, 7 aa, Oryctolagus cuniculus
  anti-IL-6 antibody Ab19 light chain CDR 2
DSSDLAS

SEQ ID NO: 723 - protein, 12 aa, Oryctolagus cuniculus
  anti-IL-6 antibody Ab19 light chain CDR 3
QQAWSYSDIDNA

SEQ ID NO: 724 - protein, 5 aa, Oryctolagus cuniculus
  anti-IL-6 antibody Ab19 heavy chain CDR 1
SYYMS

SEQ ID NO: 725 - protein, 16 aa, Oryctolagus cuniculus
  anti-IL-6 antibody Ab19 heavy chain CDR 2
IITTSGNTFYASWAKG

SEQ ID NO: 726 - protein, 10 aa, Oryctolagus cuniculus
  anti-IL-6 antibody Ab19 heavy chain CDR 3
TSDIFYYRNL

SEQ ID NO: 727 - DNA, 366 bp, Oryctolagus cuniculus
  anti-IL-6 antibody Ab19 light chain variable domain
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtgaggacatttataggttattggcctggtatcaacagaaaccagggcagcctcccaagctcctgatctatgattcatccgatctggcatctggggtcccatcgcggttcaaaggcagtggatctgggacagagttcactctcgccatcagcggtgtgcagtgtgacgatgctgccacttactactgtcaacaggcttggagttatagtgatattgataatgct

SEQ ID NO: 728 - DNA, 369 bp, Oryctolagus cuniculus
  anti-IL-6 antibody Ab19 heavy chain variable domain
atggagactgggctgcgctggcttctcctggtcgctgtgctcaaaggtgtccagtgtcagtcggtggaggagtccgggggtcgcctggtcacgccggggacacccctgacactcacctgcacagcctctggattctccctcagtagctactacatgagctgggtccgccaggctccagggaaggggctggaatggatcggaatcattactactagtggtaatacattttacgcgagctgggcgaaaggccggctcaccatctccagaacctcgaccacggtggatctgaaaatcaccagtccgacaaccgaggacacggccacctatttctgtgccagaacttctgatattttttattatcgtaacttg

SEQ ID NO: 729 - DNA, 33 bp, Oryctolagus cuniculus
  anti-IL-6 antibody Ab19 light chain CDR 1
caggccagtgaggacatttataggttattggcc

SEQ ID NO: 730 - DNA, 21 bp, Oryctolagus cuniculus
  anti-IL-6 antibody Ab19 light chain CDR 2
gattcatccgatctggcatct

SEQ ID NO: 731 - DNA, 36 bp, Oryctolagus cuniculus
  anti-IL-6 antibody Ab19 light chain CDR 3
caacaggcttggagttatagtgatattgataatgct

SEQ ID NO: 732 - DNA, 15 bp, Oryctolagus cuniculus
  anti-IL-6 antibody Ab19 heavy chain CDR 1
agctactacatgagc

SEQ ID NO: 733 - DNA, 48 bp, Oryctolagus cuniculus
  anti-IL-6 antibody Ab19 heavy chain CDR 2
atcattactactagtggtaatacattttacgcgagctgggcgaaaggc

SEQ ID NO: 734 - DNA, 30 bp, Oryctolagus cuniculus
  anti-IL-6 antibody Ab19 heavy chain CDR 3
acttctgatattttttattatcgtaacttg

SEQ ID NO: 735 - protein, 123 aa, Oryctolagus cuniculus
  anti-IL-6 antibody Ab20 light chain variable domain
MDTRAPTQLLGLLLLWLPGATFAAVLTQTASPVSAAVGATVTINCQSSQSVYNDMDLAWFQQKPGQPPKLLIYSASTLASGVPSRFSGSGSGTEFTLTISGVQCDDAATYYCLGAFDDDADNT

SEQ ID NO: 736 - protein, 129 aa, Oryctolagus cuniculus
  anti-IL-6 antibody Ab20 heavy chain variable domain
METGLRWLLLVAVLKGVQCQSVEESGGRLVTPGTPLTLTCTVSGFSLTRHAITWVRQAPGKGLEWIGCIWSGGSTYYATWAKGRFTISKTSTTVDLRITSPTTEDTATYFCARVIGDTAGYAYFTGLDL

SEQ ID NO: 737 - protein, 13 aa, Oryctolagus cuniculus
  anti-IL-6 antibody Ab20 light chain CDR 1
QSSQSVYNDMDLA

SEQ ID NO: 738 - protein, 7 aa, Oryctolagus cuniculus
  anti-IL-6 antibody Ab20 light chain CDR 2
SASTLAS

SEQ ID NO: 739 - protein, 11 aa, Oryctolagus cuniculus
  anti-IL-6 antibody Ab20 light chain CDR 3
LGAFDDDADNT

SEQ ID NO: 740 - protein, 5 aa, Oryctolagus cuniculus
  anti-IL-6 antibody Ab20 heavy chain CDR 1
RHAIT

SEQ ID NO: 741 - protein, 16 aa, Oryctolagus cuniculus
  anti-IL-6 antibody Ab20 heavy chain CDR 2
CIWSGGSTYYATWAKG

SEQ ID NO: 742 - protein, 16 aa, Oryctolagus cuniculus
  anti-IL-6 antibody Ab20 heavy chain CDR 3
VIGDTAGYAYFTGLDL

SEQ ID NO: 743 - DNA, 369 bp, Oryctolagus cuniculus
  anti-IL-6 antibody Ab20 light chain variable domain
atggacacgagggcccccactcagctgctggggctcctgctgctctggctcccaggtgccacgtttgcagccgtgctgacccagactgcatcacccgtgtctgccgctgtgggagccacagtcaccatcaactgccagtccagtcagagtgtttataatgacatggacttagcctggtttcagcagaaaccagggcagcctcccaagctcctgatctattctgcatccactctggcatctggggtcccatcgcggttcagcggcagtggatctgggacagagttcactctcaccatcagcggcgtgcagtgtgacgatgctgccacttactactgtctaggcgcttttgatgatgatgctgataatact

SEQ ID NO: 744 - DNA, 387 bp, Oryctolagus cuniculus
  anti-IL-6 antibody Ab20 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcactaggcatgcaataacctgggtccgccaggctccagggaaggggctggaatggatcggatgcatttggagtggtggtagcacatactacgcgacctgggcgaaaggccgattcaccatctccaaaacctcgaccacggtggatctcagaatcaccagtccgacaaccgaggacacggccacctacttctgtgccagagtcattggcgatactgctggttatgcttattttacggggcttgacttg

SEQ ID NO: 745 - DNA, 39 bp, Oryctolagus cuniculus
  anti-IL-6 antibody Ab20 light chain CDR 1
cagtccagtcagagtgtttataatgacatggacttagcc

SEQ ID NO: 746 - DNA, 21 bp, Oryctolagus cuniculus
  anti-IL-6 antibody Ab20 light chain CDR 2
tctgcatccactctggcatct

SEQ ID NO: 747 - DNA, 33 bp, Oryctolagus cuniculus
  anti-IL-6 antibody Ab20 light chain CDR 3
ctaggcgcttttgatgatgatgctgataatact

SEQ ID NO: 748 - DNA, 15 bp, Oryctolagus cuniculus
  anti-IL-6 antibody Ab20 heavy chain CDR 1
aggcatgcaataacc

SEQ ID NO: 749 - DNA, 48 bp, Oryctolagus cuniculus
  anti-IL-6 antibody Ab20 heavy chain CDR 2
tgcatttggagtggtggtagcacatactacgcgacctgggcgaaaggc

SEQ ID NO: 750 - DNA, 48 bp, Oryctolagus cuniculus
  anti-IL-6 antibody Ab20 heavy chain CDR 3
gtcattggcgatactgctggttatgcttattttacggggcttgacttg

SEQ ID NO: 751 - protein, 121 aa, Oryctolagus cuniculus
  anti-IL-6 antibody Ab21 light chain variable domain
MDTRAPTQLLGLLLLWLPGARCAYDMTQTPASVEVAVGGTVTIKCQASQSVYNWLSWYQQKPGQPPKLLIYTASSLASGVPSRFSGSGSGTEFTLTISGVECADAATYYCQQGYTSDVDNV

SEQ ID NO: 752 - protein, 130 aa, Oryctolagus cuniculus
  anti-IL-6 antibody Ab21 heavy chain variable domain
METGLRWLLLVAVLKGVQCQSLEEAGGRLVTPGTPLTLTCTVSGIDLSSYAMGWVRQAPGKGLEYIGIISSSGSTYYATWAKGRFTISQASSTTVDLKITSPTTEDSATYFCARGGAGSGGVWLLDGFDP

SEQ ID NO: 753 - protein, 11 aa, Oryctolagus cuniculus
  anti-IL-6 antibody Ab21 light chain CDR 1
QASQSVYNWLS

SEQ ID NO: 754 - protein, 7 aa, Oryctolagus cuniculus
  anti-IL-6 antibody Ab21 light chain CDR 2
TASSLAS

SEQ ID NO: 755 - protein, 11 aa, Oryctolagus cuniculus
  anti-IL-6 antibody Ab21 light chain CDR 3
QQGYTSDVDNV

SEQ ID NO: 756 - protein, 5 aa, Oryctolagus cuniculus
  anti-IL-6 antibody Ab21 heavy chain CDR 1
SYAMG

SEQ ID NO: 757 - protein, 16 aa, Oryctolagus cuniculus
  anti-IL-6 antibody Ab21 heavy chain CDR 2
IISSSGSTYYATWAKG

SEQ ID NO: 758 - protein, 16 aa, Oryctolagus cuniculus
  anti-IL-6 antibody Ab21 heavy chain CDR 3
GGAGSGGVWLLDGFDP

SEQ ID NO: 759 - DNA, 363 bp, Oryctolagus cuniculus
  anti-IL-6 antibody Ab21 light chain variable domain
atggacacgagggcccccactcagctgctggggctcctgctgctctggctcccaggtgccagatgtgcctatgatatgacccagactccagcctctgtggaggtagctgtgggaggcacagtcaccatcaagtgccaggccagtcagagtgtttataattggttatcctggtatcagcagaaaccagggcagcctcccaagctcctgatctatactgcatccagtctggcatctggggtcccatcgcggttcagtggcagtggatctgggacagagttcactctcaccatcagcggcgtggagtgtgccgatgctgccacttactactgtcaacagggttatactagtgatgttgataatgtt

SEQ ID NO: 760 - DNA, 390 bp, Oryctolagus cuniculus
  anti-IL-6 antibody Ab21 heavy chain variable domain
atggagactgggctgcgctggcttctcctggtcgctgtgctcaaaggtgtccagtgtcagtcgctggaggaggccgggggtcgcctggtcacgcctgggacacccctgacactcacctgcacagtctctggaatcgacctcagtagctatgcaatgggctgggtccgccaggctccagggaaggggctggaatacatcggaatcattagtagtagtggtagcacatactacgcgacctgggcgaaaggccgattcaccatctcacaagcctcgtcgaccacggtggatctgaaaattaccagtccgacaaccgaggactcggccacatatttctgtgccagagggggtgctggtagtggtggtgtttggctgcttgatggttttgatccc

SEQ ID NO: 761 - DNA, 33 bp, Oryctolagus cuniculus
  anti-IL-6 antibody Ab21 light chain CDR 1
caggccagtcagagtgtttataattggttatcc

SEQ ID NO: 762 - DNA, 21 bp, Oryctolagus cuniculus
  anti-IL-6 antibody Ab21 light chain CDR 2
actgcatccagtctggcatct

SEQ ID NO: 763 - DNA, 33 bp, Oryctolagus cuniculus
  anti-IL-6 antibody Ab21 light chain CDR 3
caacagggttatactagtgatgttgataatgtt

SEQ ID NO: 764 - DNA, 15 bp, Oryctolagus cuniculus
  anti-IL-6 antibody Ab21 heavy chain CDR 1
agctatgcaatgggc

SEQ ID NO: 765 - DNA, 48 bp, Oryctolagus cuniculus
  anti-IL-6 antibody Ab21 heavy chain CDR 2
atcattagtagtagtggtagcacatactacgcgacctgggcgaaaggc

SEQ ID NO: 766 - DNA, 48 bp, Oryctolagus cuniculus
  anti-IL-6 antibody Ab21 heavy chain CDR 3
gggggtgctggtagtggtggtgtttggctgcttgatggttttgatccc

SEQ ID NO: 767 - protein, 123 aa, Oryctolagus cuniculus
  anti-IL-6 antibody Ab22 light chain variable domain
MDTRAPTQLLGLLLLWLPGAKCADVVMTQTPASVSAAVGGTVTINCQASENIYNWLAWYQQKPGQPPKLLIYTVGDLASGVSSRFKGSGSGTEFTLTISDLECADAATYYCQQGYSSSYVDNV

SEQ ID NO: 768 - protein, 130 aa, Oryctolagus cuniculus
  anti-IL-6 antibody Ab22 heavy chain variable domain
METGLRWLLLVAVLKGVQCQEQLKESGGRLVTPGTPLTLTCTVSGFSLNDYAVGWFRQAPGKGLEWIGYIRSSGTTAYATWAKGRFTISATSTTVDLKITSPTTEDTATYFCARGGAGSSGVWILDGFAP

SEQ ID NO: 769 - protein, 11 aa, Oryctolagus cuniculus
  anti-IL-6 antibody Ab22 light chain CDR 1
QASENIYNWLA

SEQ ID NO: 770 - protein, 7 aa, Oryctolagus cuniculus
  anti-IL-6 antibody Ab22 light chain CDR 2
TVGDLAS

SEQ ID NO: 771 - protein, 12 aa, Oryctolagus cuniculus
  anti-IL-6 antibody Ab22 light chain CDR 3
QQGYSSSYVDNV

SEQ ID NO: 772 - protein, 5 aa, Oryctolagus cuniculus
  anti-IL-6 antibody Ab22 heavy chain CDR 1
DYAVG

SEQ ID NO: 773 - protein, 16 aa, Oryctolagus cuniculus
  anti-IL-6 antibody Ab22 heavy chain CDR 2
YIRSSGTTAYATWAKG

SEQ ID NO: 774 - protein, 16 aa, Oryctolagus cuniculus
  anti-IL-6 antibody Ab22 heavy chain CDR 3
GGAGSSGVWILDGFAP

SEQ ID NO: 775 - DNA, 369 bp, Oryctolagus cuniculus
  anti-IL-6 antibody Ab22 light chain variable domain
atggacacgagggcccccactcagctgctggggctcctgctgctctggctcccaggtgccaaatgtgccgatgttgtgatgacccagactccagcctccgtgtctgcagctgtgggaggcacagtcaccatcaattgccaggccagtgagaacatttataattggttagcctggtatcagcagaaaccagggcagcctcccaagctcctgatctatactgtaggcgatctggcatctggggtctcatcgcggttcaaaggcagtggatctgggacagagttcactctcaccatcagcgacctggagtgtgccgatgctgccacttactattgtcaacagggttatagtagtagttatgttgataatgtt

SEQ ID NO: 776 - DNA, 390 bp, Oryctolagus cuniculus
  anti-IL-6 antibody Ab22 heavy chain variable domain
atggagactgggctgcgctggcttctcctggtcgctgtgctcaaaggtgtccagtgtcaggagcagctgaaggagtccgggggtcgcctggtcacgcctgggacacccctgacactcacctgcacagtctctggattctccctcaatgactatgcagtgggctggttccgccaggctccagggaaggggctggaatggatcggatacattcgtagtagtggtaccacagcctacgcgacctgggcgaaaggccgattcaccatctccgctacctcgaccacggtggatctgaaaatcaccagtccgacaaccgaggacacggccacctatttctgtgccagagggggtgctggtagtagtggtgtgtggatccttgatggttttgctccc

SEQ ID NO: 777 - DNA, 33 bp, Oryctolagus cuniculus
  anti-IL-6 antibody Ab22 light chain CDR 1
caggccagtgagaacatttataattggttagcc

SEQ ID NO: 778 - DNA, 21 bp, Oryctolagus cuniculus
  anti-IL-6 antibody Ab22 light chain CDR 2
actgtaggcgatctggcatct

SEQ ID NO: 779 - DNA, 36 bp, Oryctolagus cuniculus
  anti-IL-6 antibody Ab22 light chain CDR 3
caacagggttatagtagtagttatgttgataatgtt

SEQ ID NO: 780 - DNA, 15 bp, Oryctolagus cuniculus
  anti-IL-6 antibody Ab22 heavy chain CDR 1
gactatgcagtgggc

SEQ ID NO: 781 - DNA, 48 bp, Oryctolagus cuniculus
  anti-IL-6 antibody Ab22 heavy chain CDR 2
tacattcgtagtagtggtaccacagcctacgcgacctgggcgaaaggc

SEQ ID NO: 782 - DNA, 48 bp, Oryctolagus cuniculus
  anti-IL-6 antibody Ab22 heavy chain CDR 3
gggggtgctggtagtagtggtgtgtggatccttgatggttttgctccc

SEQ ID NO: 783 - protein, 121 aa, Oryctolagus cuniculus
  anti-IL-6 antibody Ab23 light chain variable domain
MDTRAPTQLLGLLLLWLPGATFAQVLTQTPSSVSAAVGGTVTINCQASQSVYQNNYLSWFQQKPGQPPKLLIYGAATLASGVPSRFKGSGSGTQFTLTISDLECDDAATYYCAGAYRDVDS

SEQ ID NO: 784 - protein, 130 aa, Oryctolagus cuniculus
  anti-IL-6 antibody Ab23 heavy chain variable domain
METGLRWLLLVAVLKGVQCQSLEESGGDLVKPGASLTLTCTASGFSFTSTYYIYWVRQAPGKGLEWIACIDAGSSGSTYYATWVNGRFTISKTSSTTVTLQMTSLTAADTATYFCAKWDYGGNVGWGYDL

SEQ ID NO: 785 - protein, 13 aa, Oryctolagus cuniculus
  anti-IL-6 antibody Ab23 light chain CDR 1
QASQSVYQNNYLS

SEQ ID NO: 786 - protein, 7 aa, Oryctolagus cuniculus
  anti-IL-6 antibody Ab23 light chain CDR 2
GAATLAS

SEQ ID NO: 787 - protein, 9 aa, Oryctolagus cuniculus
  anti-IL-6 antibody Ab23 light chain CDR 3
AGAYRDVDS

SEQ ID NO: 788 - protein, 6 aa, Oryctolagus cuniculus
  anti-IL-6 antibody Ab23 heavy chain CDR 1
STYYIY

SEQ ID NO: 789 - protein, 18 aa, Oryctolagus cuniculus
  anti-IL-6 antibody Ab23 heavy chain CDR 2
CIDAGSSGSTYYATWVNG

SEQ ID NO: 790 - protein, 13 aa, Oryctolagus cuniculus
  anti-IL-6 antibody Ab23 heavy chain CDR 3
WDYGGNVGWGYDL

SEQ ID NO: 791 - DNA, 363 bp, Oryctolagus cuniculus
  anti-IL-6 antibody Ab23 light chain variable domain
atggacacgagggcccccactcagctgctggggctcctgctgctctggctcccaggtgccacatttgctcaagtgctgacccagactccatcctccgtgtctgcagctgtgggaggcacagtcaccatcaattgccaggccagtcagagtgtttatcagaacaactacttatcctggtttcagcagaaaccagggcagcctcccaagctcctgatctatggtgcggccactctggcatctggggtcccatcgcggttcaaaggcagtggatctgggacacagttcactctcaccatcagcgacctggagtgtgacgatgctgccacttactactgtgcaggcgcttatagggatgtggattct

SEQ ID NO: 792 - DNA, 390 bp, Oryctolagus cuniculus
  anti-IL-6 antibody Ab23 heavy chain variable domain
atggagactgggctgcgctggcttctcctggtcgctgtgctcaaaggtgtccagtgtcagtcgttggaggagtccgggggagacctggtcaagcctggggcatccctgacactcacctgcacagcctctggattctcctttactagtacctactacatctactgggtccgccaggctccagggaaggggctggagtggatcgcatgtattgatgctggtagtagtggtagcacttactacgcgacctgggtgaatggccgattcaccatctccaaaacctcgtcgaccacggtgactctgcaaatgaccagtctgacagccgcggacacggccacctatttctgtgcgaaatgggattatggtggtaatgttggttggggttatgacttg

SEQ ID NO: 793 - DNA, 39 bp, Oryctolagus cuniculus
  anti-IL-6 antibody Ab23 light chain CDR 1
caggccagtcagagtgtttatcagaacaactacttatcc

SEQ ID NO: 794 - DNA, 21 bp, Oryctolagus cuniculus
  anti-IL-6 antibody Ab23 light chain CDR 2
ggtgcggccactctggcatct

SEQ ID NO: 795 - DNA, 27 bp, Oryctolagus cuniculus
  anti-IL-6 antibody Ab23 light chain CDR 3
gcaggcgcttatagggatgtggattct

SEQ ID NO: 796 - DNA, 18 bp, Oryctolagus cuniculus
  anti-IL-6 antibody Ab23 heavy chain CDR 1
agtacctactacatctac

SEQ ID NO: 797 - DNA, 54 bp, Oryctolagus cuniculus
  anti-IL-6 antibody Ab23 heavy chain CDR 2
tgtattgatgctggtagtagtggtagcacttactacgcgacctgggtgaatggc

SEQ ID NO: 798 - DNA, 39 bp, Oryctolagus cuniculus
  anti-IL-6 antibody Ab23 heavy chain CDR 3
tgggattatggtggtaatgttggttggggttatgacttg

SEQ ID NO: 799 - protein, 120 aa, Oryctolagus cuniculus
  anti-IL-6 antibody Ab24 light chain variable domain
MDTRAPTQLLGLLLLWLPGARCAFELTQTPSSVEAAVGGTVTIKCQASQSISSYLAWYQQKPGQPPKFLIYRASTLASGVPSRFKGSGSGTEFTLTISDLECADAATYYCQSYYDSVSNP

SEQ ID NO: 800 - protein, 127 aa, Oryctolagus cuniculus
  anti-IL-6 antibody Ab24 heavy chain variable domain
METGLRWLLLVAVLKGVQCQSLEESGGDLVKPEGSLTLTCKASGLDLGTYWFMCWVRQAPGKGLEWIACIYTGSSGSTFYASWVNGRFTISKTSSTTVTLQMTSLTAADTATYFCARGYSGYGYFKL

SEQ ID NO: 801 - protein, 11 aa, Oryctolagus cuniculus
  anti-IL-6 antibody Ab24 light chain CDR 1
QASQSISSYLA

SEQ ID NO: 802 - protein, 7 aa, Oryctolagus cuniculus
  anti-IL-6 antibody Ab24 light chain CDR 2
RASTLAS

SEQ ID NO: 803 - protein, 10 aa, Oryctolagus cuniculus
  anti-IL-6 antibody Ab24 light chain CDR 3
QSYYDSVSNP

SEQ ID NO: 804 - protein, 6 aa, Oryctolagus cuniculus
  anti-IL-6 antibody Ab24 heavy chain CDR 1
TYWFMC

SEQ ID NO: 805 - protein, 18 aa, Oryctolagus cuniculus
  anti-IL-6 antibody Ab24 heavy chain CDR 2
CIYTGSSGSTFYASWVNG

SEQ ID NO: 806 - protein, 10 aa, Oryctolagus cuniculus
  anti-IL-6 antibody Ab24 heavy chain CDR 3
GYSGYGYFKL

SEQ ID NO: 807 - DNA, 360 bp, Oryctolagus cuniculus
  anti-IL-6 antibody Ab24 light chain variable domain
atggacacgagggcccccactcagctgctggggctcctgctgctctggctcccaggtgccagatgtgcattcgaattgacccagactccatcctccgtggaggcagctgtgggaggcacagtcaccatcaagtgccaggccagtcagagcattagtagttacttagcctggtatcagcagaaaccagggcagcctcccaagttcctgatctacagggcgtccactctggcatctggggtcccatcgcgattcaaaggcagtggatctgggacagagttcactctcaccatcagcgacctggagtgtgccgatgctgccacttactactgtcaaagctattatgatagtgtttcaaatcct

SEQ ID NO: 808 - DNA, 381 bp, Oryctolagus cuniculus
  anti-IL-6 antibody Ab24 heavy chain variable domain
atggagactgggctgcgctggcttctcctggtcgctgtgctcaaaggtgtccagtgtcagtcgttggaggagtccgggggagacctggtcaagcctgagggatccctgacactcacctgcaaagcctctggactcgacctcggtacctactggttcatgtgctgggtccgccaggctccagggaaggggctggagtggatcgcttgtatttatactggtagtagtggttccactttctacgcgagctgggtgaatggccgattcaccatctccaaaacctcgtcgaccacggtgactctgcaaatgaccagtctgacagccgcggacacggccacttatttttgtgcgagaggttatagtggttatggttattttaagttg

SEQ ID NO: 809 - DNA, 33 bp, Oryctolagus cuniculus
  anti-IL-6 antibody Ab24 light chain CDR 1
caggccagtcagagcattagtagttacttagcc

SEQ ID NO: 810 - DNA, 21 bp, Oryctolagus cuniculus
  anti-IL-6 antibody Ab24 light chain CDR 2
agggcgtccactctggcatct

SEQ ID NO: 811 - DNA, 30 bp, Oryctolagus cuniculus
  anti-IL-6 antibody Ab24 light chain CDR 3
caaagctattatgatagtgtttcaaatcct

SEQ ID NO: 812 - DNA, 18 bp, Oryctolagus cuniculus
  anti-IL-6 antibody Ab24 heavy chain CDR 1
acctactggttcatgtgc

SEQ ID NO: 813 - DNA, 54 bp, Oryctolagus cuniculus
  anti-IL-6 antibody Ab24 heavy chain CDR 2
tgtatttatactggtagtagtggttccactttctacgcgagctgggtgaatggc

SEQ ID NO: 814 - DNA, 30 bp, Oryctolagus cuniculus
  anti-IL-6 antibody Ab24 heavy chain CDR 3
ggttatagtggttatggttattttaagttg

SEQ ID NO: 815 - protein, 124 aa, Oryctolagus cuniculus
  anti-IL-6 antibody Ab25 light chain variable domain
MDTRAPTQLLGLLLLWLPGVTFAIEMTQSPFSVSAAVGGTVSISCQASQSVYKNNQLSWYQQKSGQPPKLLIYGASALASGVPSRFKGSGSGTEFTLTISDVQCDDAATYYCAGAITGSIDTDG

SEQ ID NO: 816 - protein, 130 aa, Oryctolagus cuniculus
  anti-IL-6 antibody Ab25 heavy chain variable domain
METGLRWLLLVAVLKGVQCQSLEESGGDLVKPGASLTLTCTTSGFSFSSSYFICWVRQAPGKGLEWIACIYGGDGSTYYASWAKGRFTISKTSSTTVTLQMTSLTAADTATYFCAREWAYSQGYFGAFDL

SEQ ID NO: 817 - protein, 13 aa, Oryctolagus cuniculus
  anti-IL-6 antibody Ab25 light chain CDR 1
QASQSVYKNNQLS

SEQ ID NO: 818 - protein, 7 aa, Oryctolagus cuniculus
  anti-IL-6 antibody Ab25 light chain CDR 2
GASALAS

SEQ ID NO: 819 - protein, 12 aa, Oryctolagus cuniculus
  anti-IL-6 antibody Ab25 light chain CDR 3
AGAITGSIDTDG

SEQ ID NO: 820 - protein, 6 aa, Oryctolagus cuniculus
  anti-IL-6 antibody Ab25 heavy chain CDR 1
SSYFIC

SEQ ID NO: 821 - protein, 17 aa, Oryctolagus cuniculus
  anti-IL-6 antibody Ab25 heavy chain CDR 2
CIYGGDGSTYYASWAKG

SEQ ID NO: 822 - protein, 14 aa, Oryctolagus cuniculus
  anti-IL-6 antibody Ab25 heavy chain CDR 3
EWAYSQGYFGAFDL

SEQ ID NO: 823 - DNA, 372 bp, Oryctolagus cuniculus
  anti-IL-6 antibody Ab25 light chain variable domain
atggacacgagggcccccactcagctgctggggctcctgctgctctggctcccaggtgtcacatttgccatcgaaatgacccagagtccattctccgtgtctgcagctgtgggaggcacagtcagcatcagttgccaggccagtcagagtgtttataagaacaaccaattatcctggtatcagcagaaatcagggcagcctcccaagctcctgatctatggtgcatcggctctggcatctggggtcccatcgcggttcaaaggcagtggatctgggacagagttcactctcaccatcagcgacgtgcagtgtgacgatgctgccacttactactgtgcaggcgctattactggtagtattgatacggatggt

SEQ ID NO: 824 - DNA, 390 bp, Oryctolagus cuniculus
  anti-IL-6 antibody Ab25 heavy chain variable domain
atggagactgggctgcgctggcttctcctggtcgctgtgctcaaaggtgtccagtgtcagtcgttggaggagtccgggggagacctggtcaagcctggggcatccctgacactcacctgcacaacttctggattctccttcagtagcagctacttcatttgctgggtccgccaggctccagggaaggggctggagtggatcgcatgcatttatggtggtgatggcagcacatactacgcgagctgggcgaaaggccgattcaccatctccaaaacctcgtcgaccacggtgacgctgcaaatgaccagtctgacagccgcggacacggccacctatttctgtgcgagagaatgggcatatagtcaaggttattttggtgcttttgatctc

SEQ ID NO: 825 - DNA, 39 bp, Oryctolagus cuniculus
  anti-IL-6 antibody Ab25 light chain CDR 1
caggccagtcagagtgtttataagaacaaccaattatcc

SEQ ID NO: 826 - DNA, 21 bp, Oryctolagus cuniculus
  anti-IL-6 antibody Ab25 light chain CDR 2
ggtgcatcggctctggcatct

SEQ ID NO: 827 - DNA, 36 bp, Oryctolagus cuniculus
  anti-IL-6 antibody Ab25 light chain CDR 3
gcaggcgctattactggtagtattgatacggatggt

SEQ ID NO: 828 - DNA, 18 bp, Oryctolagus cuniculus
  anti-IL-6 antibody Ab25 heavy chain CDR 1
agcagctacttcatttgc

SEQ ID NO: 829 - DNA, 51 bp, Oryctolagus cuniculus
  anti-IL-6 antibody Ab25 heavy chain CDR 2
tgcatttatggtggtgatggcagcacatactacgcgagctgggcgaaaggc

SEQ ID NO: 830 - DNA, 42 bp, Oryctolagus cuniculus
  anti-IL-6 antibody Ab25 heavy chain CDR 3
gaatgggcatatagtcaaggttattttggtgcttttgatctc

SEQ ID NO: 831 - protein, 124 aa, Oryctolagus cuniculus
  anti-IL-6 antibody Ab26 light chain variable domain
MDTRAPTQLLGLLLLWLPGARCDVVMTQTPASVEAAVGGTVTIKCQASEDISSYLAWYQQKPGQPPKLLIYAASNLESGVSSRFKGSGSGTEYTLTISDLECADAATYYCQCTYGTISISDGNA

SEQ ID NO: 832 - protein, 124 aa, Oryctolagus cuniculus
  anti-IL-6 antibody Ab26 heavy chain variable domain
METGLRWLLLVAVLKGVQCQSVEESGGRLVTPGTPLTLTCTVSGFSLSSYFMTWVRQAPGEGLEYIGFINPGGSAYYASWVKGRFTISKSSTTVDLKITSPTTEDTATYFCARVLIVSYGAFTI

SEQ ID NO: 833 - protein, 11 aa, Oryctolagus cuniculus
  anti-IL-6 antibody Ab26 light chain CDR 1
QASEDISSYLA

SEQ ID NO: 834 - protein, 7 aa, Oryctolagus cuniculus
  anti-IL-6 antibody Ab26 light chain CDR 2
AASNLES

SEQ ID NO: 835 - protein, 14 aa, Oryctolagus cuniculus
  anti-IL-6 antibody Ab26 light chain CDR 3
QCTYGTISISDGNA

SEQ ID NO: 836 - protein, 5 aa, Oryctolagus cuniculus
  anti-IL-6 antibody Ab26 heavy chain CDR 1
SYFMT

SEQ ID NO: 837 - protein, 16 aa, Oryctolagus cuniculus
  anti-IL-6 antibody Ab26 heavy chain CDR 2
FINPGGSAYYASWVKG

SEQ ID NO: 838 - protein, 11 aa, Oryctolagus cuniculus
  anti-IL-6 antibody Ab26 heavy chain CDR 3
VLIVSYGAFTI

SEQ ID NO: 839 - DNA, 372 bp, Oryctolagus cuniculus
  anti-IL-6 antibody Ab26 light chain variable domain
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgaggatattagtagctacttagcctggtatcagcagaaaccagggcagcctcccaagctcctgatctatgctgcatccaatctggaatctggggtctcatcgcgattcaaaggcagtggatctgggacagagtacactctcaccatcagcgacctggagtgtgccgatgctgccacctattactgtcaatgtacttatggtactatttctattagtgatggtaatgct

SEQ ID NO: 840 - DNA, 372 bp, Oryctolagus cuniculus
  anti-IL-6 antibody Ab26 heavy chain variable domain
atggagactgggctgcgctggcttctcctggtcgctgtgctcaaaggtgtccaatgtcagtcggtggaggagtccgggggtcgcctggtcacgcctgggacacccctgacactcacctgcacagtctctggattctccctcagtagctacttcatgacctgggtccgccaggctccaggggaggggctggaatacatcggattcattaatcctggtggtagcgcttactacgcgagctgggtgaaaggccgattcaccatctccaagtcctcgaccacggtagatctgaaaatcaccagtccgacaaccgaggacacggccacctatttctgtgccagggttctgattgtttcttatggagcctttaccatc

SEQ ID NO: 841 - DNA, 33 bp, Oryctolagus cuniculus
  anti-IL-6 antibody Ab26 light chain CDR 1
caggccagtgaggatattagtagctacttagcc

SEQ ID NO: 842 - DNA, 21 bp, Oryctolagus cuniculus
  anti-IL-6 antibody Ab26 light chain CDR 2
gctgcatccaatctggaatct

SEQ ID NO: 843 - DNA, 42 bp, Oryctolagus cuniculus
  anti-IL-6 antibody Ab26 light chain CDR 3
caatgtacttatggtactatttctattagtgatggtaatgct

SEQ ID NO: 844 - DNA, 15 bp, Oryctolagus cuniculus
  anti-IL-6 antibody Ab26 heavy chain CDR 1
agctacttcatgacc

SEQ ID NO: 845 - DNA, 48 bp, Oryctolagus cuniculus
  anti-IL-6 antibody Ab26 heavy chain CDR 2
ttcattaatcctggtggtagcgcttactacgcgagctgggtgaaaggc

SEQ ID NO: 846 - DNA, 33 bp, Oryctolagus cuniculus
  anti-IL-6 antibody Ab26 heavy chain CDR 3
gttctgattgtttcttatggagcctttaccatc

SEQ ID NO: 847 - protein, 124 aa, Oryctolagus cuniculus
  anti-IL-6 antibody Ab27 light chain variable domain
MDTRAPTQLLGLLLLWLPGARCDVVMTQTPASVSAAVGGTVTIKCQASEDIESYLAWYQQKPGQPPKLLIYGASNLESGVSSRFKGSGSGTEFTLTISDLECADAATYYCQCTYGIISISDGNA

SEQ ID NO: 848 - protein, 124 aa, Oryctolagus cuniculus
  anti-IL-6 antibody Ab27 heavy chain variable domain
METGLRWLLLVAVLKGVQCQSVEESGGRLVTPGTPLTLTCTVSGFSLSSYFMTWVRQAPGEGLEYIGFMNTGDNAYYASWAKGRFTISKTSTTVDLKITSPTTEDTATYFCARVLVVAYGAFNI

SEQ ID NO: 849 - protein, 11 aa, Oryctolagus cuniculus
  anti-IL-6 antibody Ab27 light chain CDR 1
QASEDIESYLA

SEQ ID NO: 850 - protein, 7 aa, Oryctolagus cuniculus
  anti-IL-6 antibody Ab27 light chain CDR 2
GASNLES

SEQ ID NO: 851 - protein, 14 aa, Oryctolagus cuniculus
  anti-IL-6 antibody Ab27 light chain CDR 3
QCTYGIISISDGNA

SEQ ID NO: 852 - protein, 5 aa, Oryctolagus cuniculus
  anti-IL-6 antibody Ab27 heavy chain CDR 1
SYFMT

SEQ ID NO: 853 - protein, 16 aa, Oryctolagus cuniculus
  anti-IL-6 antibody Ab27 heavy chain CDR 2
FMNTGDNAYYASWAKG

SEQ ID NO: 854 - protein, 11 aa, Oryctolagus cuniculus
  anti-IL-6 antibody Ab27 heavy chain CDR 3
VLVVAYGAFNI

SEQ ID NO: 855 - DNA, 372 bp, Oryctolagus cuniculus
  anti-IL-6 antibody Ab27 light chain variable domain
atggacacgagggcccccactcagctgctggggctcctgctgctctggctcccaggtgccagatgtgatgttgtgatgacccagactccagcctccgtgtctgcagctgtgggaggcacagtcaccatcaagtgccaggccagtgaggacattgaaagctatctagcctggtatcagcagaaaccagggcagcctcccaagctcctgatctatggtgcatccaatctggaatctggggtctcatcgcggttcaaaggcagtggatctgggacagagttcactctcaccatcagcgacctggagtgtgccgatgctgccacttactattgtcaatgcacttatggtattattagtattagtgatggtaatgct

SEQ ID NO: 856 - DNA, 372 bp, Oryctolagus cuniculus
  anti-IL-6 antibody Ab27 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgacctgggtccgccaggctccaggggaggggctggaatacatcggattcatgaatactggtgataacgcatactacgcgagctgggcgaaaggccgattcaccatctccaaaacctcgaccacggtggatctgaaaatcaccagtccgacaaccgaggacacggccacctatttctgtgccagggttcttgttgttgcttatggagcctttaacatc

SEQ ID NO: 857 - DNA, 33 bp, Oryctolagus cuniculus
  anti-IL-6 antibody Ab27 light chain CDR 1
caggccagtgaggacattgaaagctatctagcc

SEQ ID NO: 858 - DNA, 21 bp, Oryctolagus cuniculus
  anti-IL-6 antibody Ab27 light chain CDR 2
ggtgcatccaatctggaatct

SEQ ID NO: 859 - DNA, 42 bp, Oryctolagus cuniculus
  anti-IL-6 antibody Ab27 light chain CDR 3
caatgcacttatggtattattagtattagtgatggtaatgct

SEQ ID NO: 860 - DNA, 15 bp, Oryctolagus cuniculus
  anti-IL-6 antibody Ab27 heavy chain CDR 1
agctacttcatgacc

SEQ ID NO: 861 - DNA, 48 bp, Oryctolagus cuniculus
  anti-IL-6 antibody Ab27 heavy chain CDR 2
ttcatgaatactggtgataacgcatactacgcgagctgggcgaaaggc

SEQ ID NO: 862 - DNA, 33 bp, Oryctolagus cuniculus
  anti-IL-6 antibody Ab27 heavy chain CDR 3
gttcttgttgttgcttatggagcctttaacatc

SEQ ID NO: 863 - protein, 124 aa, Oryctolagus cuniculus
  anti-IL-6 antibody Ab28 light chain variable domain
MDTRAPTQLLGLLLLWLPGATFAAVLTQTPSPVSEPVGGTVSISCQSSKSVMNNNYLAWYQQKPGQPPKLLIYGASNLASGVPSRFSGSGSGTQFTLTISDVQCDDAATYYCQGGYTGYSDHGT

SEQ ID NO: 864 - protein, 127 aa, Oryctolagus cuniculus
  anti-IL-6 antibody Ab28 heavy chain variable domain
METGLRWLLLVAVLKGVQCQSVEESGGRLVKPDETLTLTCTVSGIDLSSYPMNWVRQAPGKGLEWIGFINTGGTIVYASWAKGRFTISKTSTTVDLKMTSPTTEDTATYFCARGSYVSSGYAYYFNV

SEQ ID NO: 865 - protein, 13 aa, Oryctolagus cuniculus
  anti-IL-6 antibody Ab28 light chain CDR 1
QSSKSVMNNNYLA

SEQ ID NO: 866 - protein, 7 aa, Oryctolagus cuniculus
  anti-IL-6 antibody Ab28 light chain CDR 2
GASNLAS

SEQ ID NO: 867 - protein, 12 aa, Oryctolagus cuniculus
  anti-IL-6 antibody Ab28 light chain CDR 3
QGGYTGYSDHGT

SEQ ID NO: 868 - protein, 5 aa, Oryctolagus cuniculus
  anti-IL-6 antibody Ab28 heavy chain CDR 1
SYPMN

SEQ ID NO: 869 - protein, 16 aa, Oryctolagus cuniculus
  anti-IL-6 antibody Ab28 heavy chain CDR 2
FINTGGTIVYASWAKG

SEQ ID NO: 870 - protein, 14 aa, Oryctolagus cuniculus
  anti-IL-6 antibody Ab28 heavy chain CDR 3
GSYVSSGYAYYFNV

SEQ ID NO: 871 - DNA, 372 bp, Oryctolagus cuniculus
  anti-IL-6 antibody Ab28 light chain variable domain
atggacacgagggcccccactcagctgctggggctcctgctgctctggctcccaggtgccacatttgccgccgtgctgacccagactccatctcccgtgtctgaacctgtgggaggcacagtcagcatcagttgccagtccagtaagagtgttatgaataacaactacttagcctggtatcagcagaaaccagggcagcctcccaagctcctgatctatggtgcatccaatctggcatctggggtcccatcacggttcagcggcagtggatctgggacacagttcactctcaccatcagcgacgtgcagtgtgacgatgctgccacttactactgtcaaggcggttatactggttatagtgatcatgggact

SEQ ID NO: 872 - DNA, 381 bp, Oryctolagus cuniculus
  anti-IL-6 antibody Ab28 heavy chain variable domain
atggagactgggctgcgctggcttctcctggtcgctgtgctcaaaggtgtccagtgtcagtcggtggaggagtccgggggtcgcctggtcaagcctgacgaaaccctgacactcacctgcacagtctctggaatcgacctcagtagctatccaatgaactgggtccgccaggctccagggaaggggctggaatggatcggattcattaatactggtggtaccatagtctacgcgagctgggcaaaaggccgattcaccatctccaaaacctcgaccacggtggatctgaaaatgaccagtccgacaaccgaggacacggccacctatttctgtgccagaggcagttatgtttcatctggttatgcctactattttaatgtc

SEQ ID NO: 873 - DNA, 39 bp, Oryctolagus cuniculus
  anti-IL-6 antibody Ab28 light chain CDR 1
cagtccagtaagagtgttatgaataacaactacttagcc

SEQ ID NO: 874 - DNA, 21 bp, Oryctolagus cuniculus
  anti-IL-6 antibody Ab28 light chain CDR 2
ggtgcatccaatctggcatct

SEQ ID NO: 875 - DNA, 36 bp, Oryctolagus cuniculus
  anti-IL-6 antibody Ab28 light chain CDR 3
caaggcggttatactggttatagtgatcatgggact

SEQ ID NO: 876 - DNA, 15 bp, Oryctolagus cuniculus
  anti-IL-6 antibody Ab28 heavy chain CDR 1
agctatccaatgaac

SEQ ID NO: 877 - DNA, 48 bp, Oryctolagus cuniculus
  anti-IL-6 antibody Ab28 heavy chain CDR 2
ttcattaatactggtggtaccatagtctacgcgagctgggcaaaaggc

SEQ ID NO: 878 - DNA, 42 bp, Oryctolagus cuniculus
  anti-IL-6 antibody Ab28 heavy chain CDR 3
ggcagttatgtttcatctggttatgcctactattttaatgtc

SEQ ID NO: 879 - protein, 121 aa, Oryctolagus cuniculus
  anti-IL-6 antibody Ab29 light chain variable domain
MDTRAPTQLLGLLLLWLPGATFAAVLTQTPSPVSAAVGGTVSISCQSSQSVYNNNWLSWFQQKPGQPPKLLIYKASTLASGVPSRFKGSGSGTQFTLTISDVQCDDVATYYCAGGYLDSVI

SEQ ID NO: 880 - protein, 126 aa, Oryctolagus cuniculus
  anti-IL-6 antibody Ab29 heavy chain variable domain
METGLRWLLLVAVLKGVQCQSVEESGGRLVTPGTPLTLTCTVSGFSLSTYSINWVRQAPGKGLEWIGIIANSGTTFYANWAKGRFTVSKTSTTVDLKITSPTTEDTATYFCARESGMYNEYGKFNI

SEQ ID NO: 881 - protein, 13 aa, Oryctolagus cuniculus
  anti-IL-6 antibody Ab29 light chain CDR 1
QSSQSVYNNNWLS

SEQ ID NO: 882 - protein, 7 aa, Oryctolagus cuniculus
  anti-IL-6 antibody Ab29 light chain CDR 2
KASTLAS

SEQ ID NO: 883 - protein, 9 aa, Oryctolagus cuniculus
  anti-IL-6 antibody Ab29 light chain CDR 3
AGGYLDSVI

SEQ ID NO: 884 - protein, 5 aa, Oryctolagus cuniculus
  anti-IL-6 antibody Ab29 heavy chain CDR 1
TYSIN

SEQ ID NO: 885 - protein, 16 aa, Oryctolagus cuniculus
  anti-IL-6 antibody Ab29 heavy chain CDR 2
IIANSGTTFYANWAKG

SEQ ID NO: 886 - protein, 13 aa, Oryctolagus cuniculus
  anti-IL-6 antibody Ab29 heavy chain CDR 3
ESGMYNEYGKFNI

SEQ ID NO: 887 - DNA, 363 bp, Oryctolagus cuniculus
  anti-IL-6 antibody Ab29 light chain variable domain
atggacacgagggcccccactcagctgctggggctcctgctgctctggctcccaggtgccacatttgccgccgtgctgacccagactccatctcccgtgtctgcagctgtgggaggcacagtcagcatcagttgccagtccagtcagagtgtttataataacaactggttatcctggtttcagcagaaaccagggcagcctcccaagctcctgatctacaaggcatccactctggcatctggggtcccatcgcggttcaaaggcagtggatctgggacacagttcactctcaccatcagcgacgtgcagtgtgacgatgttgccacttactactgtgcgggcggttatcttgatagtgttatt

SEQ ID NO: 888 - DNA, 378 bp, Oryctolagus cuniculus
  anti-IL-6 antibody Ab29 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctattcaataaactgggtccgccaggctccagggaagggcctggaatggatcggaatcattgctaatagtggtaccacattctacgcgaactgggcgaaaggccgattcaccgtctccaaaacctcgaccacggtggatctgaaaatcaccagtccgacaaccgaggacacggccacctatttctgtgccagagagagtggaatgtacaatgaatatggtaaatttaacatc

SEQ ID NO: 889 - DNA, 39 bp, Oryctolagus cuniculus
  anti-IL-6 antibody Ab29 light chain CDR 1
cagtccagtcagagtgtttataataacaactggttatcc

SEQ ID NO: 890 - DNA, 21 bp, Oryctolagus cuniculus
  anti-IL-6 antibody Ab29 light chain CDR 2
aaggcatccactctggcatct

SEQ ID NO: 891 - DNA, 27 bp, Oryctolagus cuniculus
  anti-IL-6 antibody Ab29 light chain CDR 3
gcgggcggttatcttgatagtgttatt

SEQ ID NO: 892 - DNA, 15 bp, Oryctolagus cuniculus
  anti-IL-6 antibody Ab29 heavy chain CDR 1
acctattcaataaac

SEQ ID NO: 893 - DNA, 48 bp, Oryctolagus cuniculus
  anti-IL-6 antibody Ab29 heavy chain CDR 2
atcattgctaatagtggtaccacattctacgcgaactgggcgaaaggc

SEQ ID NO: 894 - DNA, 39 bp, Oryctolagus cuniculus
  anti-IL-6 antibody Ab29 heavy chain CDR 3
gagagtggaatgtacaatgaatatggtaaatttaacatc

SEQ ID NO: 895 - protein, 122 aa, Oryctolagus cuniculus
  anti-IL-6 antibody Ab30 light chain variable domain
MDTRAPTQLLGLLLLWLPGARCASDMTQTPSSVSAAVGGTVTINCQASENIYSFLAWYQQKPGQPPKLLIFKASTLASGVSSRFKGSGSGTQFTLTISDLECDDAATYYCQQGATVYDIDNN

SEQ ID NO: 896 - protein, 128 aa, Oryctolagus cuniculus
  anti-IL-6 antibody Ab30 heavy chain variable domain
METGLRWLLLVAVLKGVQCQSLEESGGRLVTPGTPLTLTCTVSGIDLSAYAMIWVRQAPGEGLEWITIIYPNGITYYANWAKGRFTVSKTSTAMDLKITSPTTEDTATYFCARDAESSKNAYWGYFNV

SEQ ID NO: 897 - protein, 11 aa, Oryctolagus cuniculus
  anti-IL-6 antibody Ab30 light chain CDR 1
QASENIYSFLA

SEQ ID NO: 898 - protein, 7 aa, Oryctolagus cuniculus
  anti-IL-6 antibody Ab30 light chain CDR 2
KASTLAS

SEQ ID NO: 899 - protein, 12 aa, Oryctolagus cuniculus
  anti-IL-6 antibody Ab30 light chain CDR 3
QQGATVYDIDNN

SEQ ID NO: 900 - protein, 5 aa, Oryctolagus cuniculus
  anti-IL-6 antibody Ab30 heavy chain CDR 1
AYAMI

SEQ ID NO: 901 - protein, 16 aa, Oryctolagus cuniculus
  anti-IL-6 antibody Ab30 heavy chain CDR 2
IIYPNGITYYANWAKG

SEQ ID NO: 902 - protein, 15 aa, Oryctolagus cuniculus
  anti-IL-6 antibody Ab30 heavy chain CDR 3
DAESSKNAYWGYFNV

SEQ ID NO: 903 - DNA, 366 bp, Oryctolagus cuniculus
  anti-IL-6 antibody Ab30 light chain variable domain
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcaaggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 904 - DNA, 384 bp, Oryctolagus cuniculus
  anti-IL-6 antibody Ab30 heavy chain variable domain
atggagactgggctgcgctggcttctcctggtcgctgtgctcaaaggtgtccagtgtcagtcgctggaggagtccgggggtcgcctggtcacgcctgggacacccctgacactcacctgcacagtttctggaatcgacctcagtgcctatgcaatgatctgggtccgccaggctccaggggaggggctggaatggatcacaatcatttatcctaatggtatcacatactacgcgaactgggcgaaaggccgattcaccgtctccaaaacctcgaccgcgatggatctgaaaatcaccagtccgacaaccgaggacacggccacctatttctgtgccagagatgcagaaagtagtaagaatgcttattggggctactttaacgtc

SEQ ID NO: 905 - DNA, 33 bp, Oryctolagus cuniculus
  anti-IL-6 antibody Ab30 light chain CDR 1
caggccagtgagaacatttatagctttttggcc

SEQ ID NO: 906 - DNA, 21 bp, Oryctolagus cuniculus
  anti-IL-6 antibody Ab30 light chain CDR 2
aaggcttccactctggcatct

SEQ ID NO: 907 - DNA, 36 bp, Oryctolagus cuniculus
  anti-IL-6 antibody Ab30 light chain CDR 3
caacagggtgctactgtgtatgatattgataataat

SEQ ID NO: 908 - DNA, 15 bp, Oryctolagus cuniculus
  anti-IL-6 antibody Ab30 heavy chain CDR 1
gcctatgcaatgatc

SEQ ID NO: 909 - DNA, 48 bp, Oryctolagus cuniculus
  anti-IL-6 antibody Ab30 heavy chain CDR 2
atcatttatcctaatggtatcacatactacgcgaactgggcgaaaggc

SEQ ID NO: 910 - DNA, 45 bp, Oryctolagus cuniculus
  anti-IL-6 antibody Ab30 heavy chain CDR 3
gatgcagaaagtagtaagaatgcttattggggctactttaacgtc

SEQ ID NO: 911 - protein, 122 aa, Oryctolagus cuniculus
  anti-IL-6 antibody Ab31 light chain variable domain
MDTRAPTQLLGLLLLWLPGARCASDMTQTPSSVSAAVGGTVTINCQASENIYSFLAWYQQKPGQPPKLLIFRASTLASGVSSRFKGSGSGTQFTLTISDLECDDAATYYCQQGATVYDIDNN

SEQ ID NO: 912 - protein, 128 aa, Oryctolagus cuniculus; identical to SEQ ID NO: 896
  anti-IL-6 antibody Ab31 heavy chain variable domain

SEQ ID NO: 913 - protein, 11 aa, Oryctolagus cuniculus
  anti-IL-6 antibody Ab31 light chain CDR 1
QASENIYSFLA

SEQ ID NO: 914 - protein, 7 aa, Oryctolagus cuniculus
  anti-IL-6 antibody Ab31 light chain CDR 2
RASTLAS

SEQ ID NO: 915 - protein, 12 aa, Oryctolagus cuniculus
  anti-IL-6 antibody Ab31 light chain CDR 3
QQGATVYDIDNN

SEQ ID NO: 916 - protein, 5 aa, Oryctolagus cuniculus
  anti-IL-6 antibody Ab31 heavy chain CDR 1
AYAMI

SEQ ID NO: 917 - protein, 16 aa, Oryctolagus cuniculus
  anti-IL-6 antibody Ab31 heavy chain CDR 2
IIYPNGITYYANWAKG

SEQ ID NO: 918 - protein, 15 aa, Oryctolagus cuniculus
  anti-IL-6 antibody Ab31 heavy chain CDR 3
DAESSKNAYWGYFNV

SEQ ID NO: 919 - DNA, 366 bp, Oryctolagus cuniculus
  anti-IL-6 antibody Ab31 light chain variable domain
atggacacgagggcccccactcagctgctggggctcctgctgctctggctcccaggtgccagatgtgcctctgatatgacccagactccatcctccgtgtctgcagctgtgggaggcacagtcaccatcaattgccaggccagtgagaacatttatagctttttggcctggtatcagcagaaaccagggcagcctcccaagctcctgatcttcagggcttccactctggcatctggggtctcatcgcggttcaaaggcagtggatctgggacacagttcactctcaccatcagcgacctggagtgtgacgatgctgccacttactactgtcaacagggtgctactgtgtatgatattgataataat

SEQ ID NO: 920 - DNA, 384 bp, Oryctolagus cuniculus; identical to SEQ ID NO: 904
  anti-IL-6 antibody Ab31 heavy chain variable domain

SEQ ID NO: 921 - DNA, 33 bp, Oryctolagus cuniculus; identical to SEQ ID NO: 905
  anti-IL-6 antibody Ab31 light chain CDR 1

SEQ ID NO: 922 - DNA, 21 bp, Oryctolagus cuniculus
  anti-IL-6 antibody Ab31 light chain CDR 2
agggcttccactctggcatct

SEQ ID NO: 923 - DNA, 36 bp, Oryctolagus cuniculus; identical to SEQ ID NO: 907
  anti-IL-6 antibody Ab31 light chain CDR 3

SEQ ID NO: 924 - DNA, 15 bp, Oryctolagus cuniculus
  anti-IL-6 antibody Ab31 heavy chain CDR 1
gcctatgcaatgatc

SEQ ID NO: 925 - DNA, 48 bp, Oryctolagus cuniculus; identical to SEQ ID NO: 909
  anti-IL-6 antibody Ab31 heavy chain CDR 2

SEQ ID NO: 926 - DNA, 45 bp, Oryctolagus cuniculus; identical to SEQ ID NO: 910
  anti-IL-6 antibody Ab31 heavy chain CDR 3

SEQ ID NO: 927 - protein, 124 aa, Oryctolagus cuniculus
  anti-IL-6 antibody Ab32 light chain variable domain
MDTRAPTQLLGLLLLWLPGATFAIEMTQTPSPVSAAVGGTVTINCQASESVFNNMLSWYQQKPGHSPKLLIYDASDLASGVPSRFKGSGSGTQFTLTISGVECDDAATYYCAGYKSDSNDGDNV

SEQ ID NO: 928 - protein, 123 aa, Oryctolagus cuniculus
  anti-IL-6 antibody Ab32 heavy chain variable domain
METGLRWLLLVAVLKGVQCQSLEESGGRLVTPGTPLTLTCTVSGFSLNRNSITWVRQAPGEGLEWIGIITGSGRTYYANWAKGRFTISKTSTTVDLKMTSPTTEDTATYFCARGHPGLGSGNI

SEQ ID NO: 929 - protein, 12 aa, Oryctolagus cuniculus
  anti-IL-6 antibody Ab32 light chain CDR 1
QASESVFNNMLS

SEQ ID NO: 930 - protein, 7 aa, Oryctolagus cuniculus
  anti-IL-6 antibody Ab32 light chain CDR 2
DASDLAS

SEQ ID NO: 931 - protein, 13 aa, Oryctolagus cuniculus
  anti-IL-6 antibody Ab32 light chain CDR 3
AGYKSDSNDGDNV

SEQ ID NO: 932 - protein, 5 aa, Oryctolagus cuniculus
  anti-IL-6 antibody Ab32 heavy chain CDR 1
RNSIT

SEQ ID NO: 933 - protein, 16 aa, Oryctolagus cuniculus
  anti-IL-6 antibody Ab32 heavy chain CDR 2
IITGSGRTYYANWAKG

SEQ ID NO: 934 - protein, 10 aa, Oryctolagus cuniculus
  anti-IL-6 antibody Ab32 heavy chain CDR 3
GHPGLGSGNI

SEQ ID NO: 935 - DNA, 372 bp, Oryctolagus cuniculus
  anti-IL-6 antibody Ab32 light chain variable domain
atggacacgagggcccccactcagctgctggggctcctgctgctctggctcccaggtgccacatttgccattgaaatgacccagactccatcccccgtgtctgccgctgtgggaggcacagtcaccatcaattgccaggccagtgagagtgtttttaataatatgttatcctggtatcagcagaaaccagggcactctcctaagctcctgatctatgatgcatccgatctggcatctggggtcccatcgcggttcaaaggcagtggatctgggacacagttcactctcaccatcagtggcgtggagtgtgacgatgctgccacttactattgtgcagggtataaaagtgatagtaatgatggcgataatgtt

SEQ ID NO: 936 - DNA, 369 bp, Oryctolagus cuniculus
  anti-IL-6 antibody Ab32 heavy chain variable domain
atggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcaacaggaattcaataacctgggtccgccaggctccaggggaggggctggaatggatcggaatcattactggtagtggtagaacgtactacgcgaactgggcaaaaggccgattcaccatctccaaaacctcgaccacggtggatctgaaaatgaccagtccgacaaccgaggacacggccacctatttctgtgccagaggccatcctggtcttggtagtggtaacatc

SEQ ID NO: 937 - DNA, 36 bp, Oryctolagus cuniculus
  anti-IL-6 antibody Ab32 light chain CDR 1
caggccagtgagagtgtttttaataatatgttatcc

SEQ ID NO: 938 - DNA, 21 bp, Oryctolagus cuniculus
  anti-IL-6 antibody Ab32 light chain CDR 2
gatgcatccgatctggcatct

SEQ ID NO: 939 - DNA, 39 bp, Oryctolagus cuniculus
  anti-IL-6 antibody Ab32 light chain CDR 3
gcagggtataaaagtgatagtaatgatggcgataatgtt

SEQ ID NO: 940 - DNA, 15 bp, Oryctolagus cuniculus
  anti-IL-6 antibody Ab32 heavy chain CDR 1
aggaattcaataacc

SEQ ID NO: 941 - DNA, 48 bp, Oryctolagus cuniculus
  anti-IL-6 antibody Ab32 heavy chain CDR 2
atcattactggtagtggtagaacgtactacgcgaactgggcaaaaggc

SEQ ID NO: 942 - DNA, 30 bp, Oryctolagus cuniculus
  anti-IL-6 antibody Ab32 heavy chain CDR 3
ggccatcctggtcttggtagtggtaacatc

SEQ ID NO: 943 - protein, 121 aa, Oryctolagus cuniculus
  anti-IL-6 antibody Ab33 light chain variable domain
MDTRAPTQLLGLLLLWLPGATFAQVLTQTASSVSAAVGGTVTINCQSSQSVYNNYLSWYQQKPGQPPKLLIYTASSLASGVPSRFKGSGSGTQFTLTISEVQCDDAATYYCQGYYSGPIIT

SEQ ID NO: 944 - protein, 122 aa, Oryctolagus cuniculus
  anti-IL-6 antibody Ab33 heavy chain variable domain
METGLRWLLLVAVLKGVQCQSLEESGGRLVTPGTPLTLTCTASGFSLNNYYIQWVRQAPGEGLEWIGIIYAGGSAYYATWANGRFTIAKTSSTTVDLKMTSLTTEDTATYFCARGTFDGYEL

SEQ ID NO: 945 - protein, 12 aa, Oryctolagus cuniculus
  anti-IL-6 antibody Ab33 light chain CDR 1
QSSQSVYNNYLS

SEQ ID NO: 946 - protein, 7 aa, Oryctolagus cuniculus
  anti-IL-6 antibody Ab33 light chain CDR 2
TASSLAS

SEQ ID NO: 947 - protein, 10 aa, Oryctolagus cuniculus
  anti-IL-6 antibody Ab33 light chain CDR 3
QGYYSGPIIT

SEQ ID NO: 948 - protein, 5 aa, Oryctolagus cuniculus
  anti-IL-6 antibody Ab33 heavy chain CDR 1
NYYIQ

SEQ ID NO: 949 - protein, 16 aa, Oryctolagus cuniculus
  anti-IL-6 antibody Ab33 heavy chain CDR 2
IIYAGGSAYYATWANG

SEQ ID NO: 950 - protein, 8 aa, Oryctolagus cuniculus
  anti-IL-6 antibody Ab33 heavy chain CDR 3
GTFDGYEL

SEQ ID NO: 951 - DNA, 363 bp, Oryctolagus cuniculus
  anti-IL-6 antibody Ab33 light chain variable domain
atggacacgagggcccccactcagctgctggggctcctgctgctctggctcccaggtgccacatttgcgcaagtgctgacccagactgcatcgtccgtgtctgcagctgtgggaggcacagtcaccatcaattgccagtccagtcagagtgtttataataactacttatcctggtatcagcagaaaccagggcagcctcccaagctcctgatctatactgcatccagcctggcatctggggtcccatcgcggttcaaaggcagtggatctgggacacagttcactctcaccatcagcgaagtgcagtgtgacgatgctgccacttactactgtcaaggctattatagtggtcctataattact

SEQ ID NO: 952 - DNA, 366 bp, Oryctolagus cuniculus
  anti-IL-6 antibody Ab33 heavy chain variable domain
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcaataactactacatacaatgggtccgccaggctccaggggaggggctggaatggatcgggatcatttatgctggtggtagcgcatactacgcgacctgggcaaacggccgattcaccatcgccaaaacctcgtcgaccacggtggatctgaagatgaccagtctgacaaccgaggacacggccacctatttctgtgccagagggacatttgatggttatgagttg

SEQ ID NO: 953 - DNA, 36 bp, Oryctolagus cuniculus
  anti-IL-6 antibody Ab33 light chain CDR 1
cagtccagtcagagtgtttataataactacttatcc

SEQ ID NO: 954 - DNA, 21 bp, Oryctolagus cuniculus
  anti-IL-6 antibody Ab33 light chain CDR 2
actgcatccagcctggcatct

SEQ ID NO: 955 - DNA, 30 bp, Oryctolagus cuniculus
  anti-IL-6 antibody Ab33 light chain CDR 3
caaggctattatagtggtcctataattact

SEQ ID NO: 956 - DNA, 15 bp, Oryctolagus cuniculus
  anti-IL-6 antibody Ab33 heavy chain CDR 1
aactactacatacaa

SEQ ID NO: 957 - DNA, 48 bp, Oryctolagus cuniculus
  anti-IL-6 antibody Ab33 heavy chain CDR 2
atcatttatgctggtggtagcgcatactacgcgacctgggcaaacggc

SEQ ID NO: 958 - DNA, 24 bp, Oryctolagus cuniculus
  anti-IL-6 antibody Ab33 heavy chain CDR 3
gggacatttgatggttatgagttg

SEQ ID NO: 959 - protein, 122 aa, Oryctolagus cuniculus
  anti-IL-6 antibody Ab34 light chain variable domain
MDTRAPTQLLGLLLLWLPGATFAQVLTQTPSPVSVPVGDTVTISCQSSESVYSNNLLSWYQQKPGQPPKLLIYRASNLASGVPSRFKGSGSGTQFTLTISGAQCDDAATYYCQGYYSGVINS

SEQ ID NO: 960 - protein, 124 aa, Oryctolagus cuniculus
  anti-IL-6 antibody Ab34 heavy chain variable domain
METGLRWLLLVAVLKGVQCQSVEESGGRLVTPGTPLTLTCTVSGFSLSSYFMSWVRQAPGEGLEYIGFINPGGSAYYASWASGRLTISKTSTTVDLKITSPTTEDTATYFCARILIVSYGAFTI

SEQ ID NO: 961 - protein, 13 aa, Oryctolagus cuniculus
  anti-IL-6 antibody Ab34 light chain CDR 1
QSSESVYSNNLLS

SEQ ID NO: 962 - protein, 7 aa, Oryctolagus cuniculus
  anti-IL-6 antibody Ab34 light chain CDR 2
RASNLAS

SEQ ID NO: 963 - protein, 10 aa, Oryctolagus cuniculus
  anti-IL-6 antibody Ab34 light chain CDR 3
QGYYSGVINS

SEQ ID NO: 964 - protein, 5 aa, Oryctolagus cuniculus
  anti-IL-6 antibody Ab34 heavy chain CDR 1
SYFMS

SEQ ID NO: 965 - protein, 16 aa, Oryctolagus cuniculus
  anti-IL-6 antibody Ab34 heavy chain CDR 2
FINPGGSAYYASWASG

SEQ ID NO: 966 - protein, 11 aa, Oryctolagus cuniculus
  anti-IL-6 antibody Ab34 heavy chain CDR 3
ILIVSYGAFTI

SEQ ID NO: 967 - DNA, 366 bp, Oryctolagus cuniculus
  anti-IL-6 antibody Ab34 light chain variable domain
atggacacgagggcccccactcagctgctggggctcctgctgctctggctcccaggtgccacatttgcccaagtgctgacccagactccatcccctgtgtctgtccctgtgggagacacagtcaccatcagttgccagtccagtgagagcgtttatagtaataacctcttatcctggtatcagcagaaaccagggcagcctcccaagctcctgatctacagggcatccaatctggcatctggtgtcccatcgcggttcaaaggcagtggatctgggacacagttcactctcaccatcagcggcgcacagtgtgacgatgctgccacttactactgtcaaggctattatagtggtgtcattaatagt

SEQ ID NO: 968 - DNA, 372 bp, Oryctolagus cuniculus
  anti-IL-6 antibody Ab34 heavy chain variable domain
atggagactgggctgcgctggcttctcctggtcgctgtgctcaaaggtgtccagtgtcagtcggtggaggagtccgggggtcgcctggtcacgcctgggacacccctgacactcacctgcacagtgtctggattctccctcagtagctacttcatgagctgggtccgccaggctccaggggaggggctggaatacatcggattcattaatcctggtggtagcgcatactacgcgagctgggcgagtggccgactcaccatctccaaaacctcgaccacggtagatctgaaaatcaccagtccgacaaccgaggacacggccacctatttctgtgccaggattcttattgtttcttatggagcctttaccatc

SEQ ID NO: 969 - DNA, 39 bp, Oryctolagus cuniculus
  anti-IL-6 antibody Ab34 light chain CDR 1
cagtccagtgagagcgtttatagtaataacctcttatcc

SEQ ID NO: 970 - DNA, 21 bp, Oryctolagus cuniculus
  anti-IL-6 antibody Ab34 light chain CDR 2
agggcatccaatctggcatct

SEQ ID NO: 971 - DNA, 30 bp, Oryctolagus cuniculus
  anti-IL-6 antibody Ab34 light chain CDR 3
caaggctattatagtggtgtcattaatagt

SEQ ID NO: 972 - DNA, 15 bp, Oryctolagus cuniculus
  anti-IL-6 antibody Ab34 heavy chain CDR 1
agctacttcatgagc

SEQ ID NO: 973 - DNA, 48 bp, Oryctolagus cuniculus
  anti-IL-6 antibody Ab34 heavy chain CDR 2
ttcattaatcctggtggtagcgcatactacgcgagctgggcgagtggc

SEQ ID NO: 974 - DNA, 33 bp, Oryctolagus cuniculus
  anti-IL-6 antibody Ab34 heavy chain CDR 3
attcttattgtttcttatggagcctttaccatc

SEQ ID NO: 975 - protein, 122 aa, Oryctolagus cuniculus
  anti-IL-6 antibody Ab35 light chain variable domain
MDTRAPTQLLGLLLLWLPGARCAYDMTQTPASVEVAVGGTVTIKCQATESIGNELSWYQQKPGQAPKLLIYSASTLASGVPSRFKGSGSGTQFTLTITGVECDDAATYYCQQGYSSANIDNA

SEQ ID NO: 976 - protein, 128 aa, Oryctolagus cuniculus
  anti-IL-6 antibody Ab35 heavy chain variable domain
METGLRWLLLVAVLKGVQCQSLEESGGRLVTPGTPLTLTCTVSGFSLSKYYMSWVRQAPEKGLKYIGYIDSTTVNTYYATWARGRFTISKTSTTVDLKITSPTSEDTATYFCARGSTYFTDGGHRLDL

SEQ ID NO: 977 - protein, 11 aa, Oryctolagus cuniculus
  anti-IL-6 antibody Ab35 light chain CDR 1
QATESIGNELS

SEQ ID NO: 978 - protein, 7 aa, Oryctolagus cuniculus
  anti-IL-6 antibody Ab35 light chain CDR 2
SASTLAS

SEQ ID NO: 979 - protein, 12 aa, Oryctolagus cuniculus
  anti-IL-6 antibody Ab35 light chain CDR 3
QQGYSSANIDNA

SEQ ID NO: 980 - protein, 5 aa, Oryctolagus cuniculus
  anti-IL-6 antibody Ab35 heavy chain CDR 1
KYYMS

SEQ ID NO: 981 - protein, 17 aa, Oryctolagus cuniculus
  anti-IL-6 antibody Ab35 heavy chain CDR 2
YIDSTTVNTYYATWARG

SEQ ID NO: 982 - protein, 14 aa, Oryctolagus cuniculus
  anti-IL-6 antibody Ab35 heavy chain CDR 3
GSTYFTDGGHRLDL

SEQ ID NO: 983 - DNA, 366 bp, Oryctolagus cuniculus
  anti-IL-6 antibody Ab35 light chain variable domain
atggacacgagggcccccactcagctgctggggctcctgctgctctggctcccaggtgccagatgtgcctatgatatgacccagactccagcctctgtggaggtagctgtgggaggcacagtcaccatcaagtgccaggccactgagagcattggcaatgagttatcctggtatcagcagaaaccagggcaggctcccaagctcctgatctattctgcatccactctggcatctggggtcccatcgcggttcaaaggcagtggatctgggacacagttcactctcaccatcaccggcgtggagtgtgatgatgctgccacttactactgtcaacagggttatagtagtgctaatattgataatgct

SEQ ID NO: 984 - DNA, 384 bp, Oryctolagus cuniculus
  anti-IL-6 antibody Ab35 heavy chain variable domain
atggagactgggctgcgctggcttctcctggtcgctgtgctcaaaggtgtccagtgtcagtcgctggaggagtccgggggtcgcctggtcacgcctgggacacccctgacactcacctgcaccgtctctggattctccctcagtaagtactacatgagctgggtccgccaggctccagagaaggggctgaaatacatcggatacattgatagtactactgttaatacatactacgcgacctgggcgagaggccgattcaccatctccaaaacctcgaccacggtggatctgaagatcaccagtccgacaagtgaggacacggccacctatttctgtgccagaggaagtacttattttactgatggaggccatcggttggatctc

SEQ ID NO: 985 - DNA, 33 bp, Oryctolagus cuniculus
  anti-IL-6 antibody Ab35 light chain CDR 1
caggccactgagagcattggcaatgagttatcc

SEQ ID NO: 986 - DNA, 21 bp, Oryctolagus cuniculus
  anti-IL-6 antibody Ab35 light chain CDR 2
tctgcatccactctggcatct

SEQ ID NO: 987 - DNA, 36 bp, Oryctolagus cuniculus
  anti-IL-6 antibody Ab35 light chain CDR 3
caacagggttatagtagtgctaatattgataatgct

SEQ ID NO: 988 - DNA, 15 bp, Oryctolagus cuniculus
  anti-IL-6 antibody Ab35 heavy chain CDR 1
aagtactacatgagc

SEQ ID NO: 989 - DNA, 51 bp, Oryctolagus cuniculus
  anti-IL-6 antibody Ab35 heavy chain CDR 2
tacattgatagtactactgttaatacatactacgcgacctgggcgagaggc

SEQ ID NO: 990 - DNA, 42 bp, Oryctolagus cuniculus
  anti-IL-6 antibody Ab35 heavy chain CDR 3
ggaagtacttattttactgatggaggccatcggttggatctc

SEQ ID NO: 991 - protein, 122 aa, Oryctolagus cuniculus; identical to SEQ ID NO: 975
  anti-IL-6 antibody Ab36 light chain variable domain

SEQ ID NO: 992 - protein, 124 aa, Oryctolagus cuniculus
  anti-IL-6 antibody Ab36 heavy chain variable domain
METGLRWLLLVAVLKGVQCQSLEESGGRLVTPGTPLTLTCTVSGFSLSTYNMGWVRQAPGKGLEWIGSITIDGRTYYASWAKGRFTVSKSSTTVDLKMTSLTTGDTATYFCARILIVSYGAFTI

SEQ ID NO: 993 - protein, 11 aa, Oryctolagus cuniculus
  anti-IL-6 antibody Ab36 light chain CDR 1
QATESIGNELS

SEQ ID NO: 994 - protein, 7 aa, Oryctolagus cuniculus
  anti-IL-6 antibody Ab36 light chain CDR 2
SASTLAS

SEQ ID NO: 995 - protein, 12 aa, Oryctolagus cuniculus
  anti-IL-6 antibody Ab36 light chain CDR 3
QQGYSSANIDNA

SEQ ID NO: 996 - protein, 5 aa, Oryctolagus cuniculus
  anti-IL-6 antibody Ab36 heavy chain CDR 1
TYNMG

SEQ ID NO: 997 - protein, 16 aa, Oryctolagus cuniculus
  anti-IL-6 antibody Ab36 heavy chain CDR 2
SITIDGRTYYASWAKG

SEQ ID NO: 998 - protein, 11 aa, Oryctolagus cuniculus
  anti-IL-6 antibody Ab36 heavy chain CDR 3
ILIVSYGAFTI

SEQ ID NO: 999 - DNA, 366 bp, Oryctolagus cuniculus; identical to SEQ ID NO: 983
  anti-IL-6 antibody Ab36 light chain variable domain

SEQ ID NO: 1000 - DNA, 372 bp, Oryctolagus cuniculus
  anti-IL-6 antibody Ab36 heavy chain variable domain
atggagactgggctgcgctggcttctcctggtcgctgtgctcaaaggtgtccagtgtcagtcgctggaggagtccgggggtcgcctggtaacgcctgggacacccctgacactcacctgcacagtctctggattctccctcagtacctacaacatgggctgggtccgccaggctccagggaaggggctggaatggatcggaagtattactattgatggtcgcacatactacgcgagctgggcgaaaggccgattcaccgtctccaaaagctcgaccacggtggatctgaaaatgaccagtctgacaaccggggacacggccacctatttctgtgccaggattcttattgtttcttatggggcctttaccatc

SEQ ID NO: 1001 - DNA, 33 bp, Oryctolagus cuniculus; identical to SEQ ID NO: 985
  anti-IL-6 antibody Ab36 light chain CDR 1

SEQ ID NO: 1002 - DNA, 21 bp, Oryctolagus cuniculus
  anti-IL-6 antibody Ab36 light chain CDR 2
tctgcatccactctggcatct

SEQ ID NO: 1003 - DNA, 36 bp, Oryctolagus cuniculus; identical to SEQ ID NO: 987
  anti-IL-6 antibody Ab36 light chain CDR 3

SEQ ID NO: 1004 - DNA, 15 bp, Oryctolagus cuniculus
  anti-IL-6 antibody Ab36 heavy chain CDR 1
acctacaacatgggc

SEQ ID NO: 1005 - DNA, 48 bp, Oryctolagus cuniculus
  anti-IL-6 antibody Ab36 heavy chain CDR 2
agtattactattgatggtcgcacatactacgcgagctgggcgaaaggc

SEQ ID NO: 1006 - DNA, 33 bp, Oryctolagus cuniculus
  anti-IL-6 antibody Ab36 heavy chain CDR 3
attcttattgtttcttatggggcctttaccatc

SEQ ID NO: 1007 - protein, 105 aa, Artificial Sequence [sequence omitted]

SEQ ID NO: 1008 - DNA, 315 bp, Artificial Sequence; identical to SEQ ID NO: 419
  Kappa constant domain of Ab1

SEQ ID NO: 1009 - protein, 330 aa, Artificial Sequence; identical to SEQ ID NO: 420
  Gamma-1 constant domain of Ab1

SEQ ID NO: 1010 - DNA, 990 bp, Artificial Sequence; identical to SEQ ID NO: 421
  Gamma-1 constant domain of Ab1

SEQ ID NO: 1011 - protein, 15 aa, Homo sapiens
VPPGEDSKDVAAPHR

SEQ ID NO: 1012 - protein, 15 aa, Homo sapiens
GEDSKDVAAPHRQPL

SEQ ID NO: 1013 - protein, 15 aa, Homo sapiens
SKDVAAPHRQPLTSS

SEQ ID NO: 1014 - protein, 15 aa, Homo sapiens
VAAPHRQPLTSSERI

SEQ ID NO: 1015 - protein, 15 aa, Homo sapiens
PHRQPLTSSERIDKQ

SEQ ID NO: 1016 - protein, 15 aa, Homo sapiens
QPLTSSERIDKQIRY

SEQ ID NO: 1017 - protein, 15 aa, Homo sapiens
TSSERIDKQIRYILD

SEQ ID NO: 1018 - protein, 15 aa, Homo sapiens
ERIDKQIRYILDGIS

SEQ ID NO: 1019 - protein, 15 aa, Homo sapiens
DKQIRYILDGISALR

SEQ ID NO: 1020 - protein, 15 aa, Homo sapiens
IRYILDGISALRKET

SEQ ID NO: 1021 - protein, 15 aa, Homo sapiens
ILDGISALRKETCNK

SEQ ID NO: 1022 - protein, 15 aa, Homo sapiens
GISALRKETCNKSNM

SEQ ID NO: 1023 - protein, 15 aa, Homo sapiens
ALRKETCNKSNMCES

SEQ ID NO: 1024 - protein, 15 aa, Homo sapiens
KETCNKSNMCESSKE

SEQ ID NO: 1025 - protein, 15 aa, Homo sapiens
CNKSNMCESSKEALA

SEQ ID NO: 1026 - protein, 15 aa, Homo sapiens
SNMCESSKEALAENN

SEQ ID NO: 1027 - protein, 15 aa, Homo sapiens
CESSKEALAENNLNL

SEQ ID NO: 1028 - protein, 15 aa, Homo sapiens
SKEALAENNLNLPKM

SEQ ID NO: 1029 - protein, 15 aa, Homo sapiens
ALAENNLNLPKMAEK

SEQ ID NO: 1030 - protein, 15 aa, Homo sapiens
ENNLNLPKMAEKDGC

SEQ ID NO: 1031 - protein, 15 aa, Homo sapiens
LNLPKMAEKDGCFQS

SEQ ID NO: 1032 - protein, 15 aa, Homo sapiens
PKMAEKDGCFQSGFN

SEQ ID NO: 1033 - protein, 15 aa, Homo sapiens
AEKDGCFQSGFNEET

SEQ ID NO: 1034 - protein, 15 aa, Homo sapiens
DGCFQSGFNEETCLV

SEQ ID NO: 1035 - protein, 15 aa, Homo sapiens
FQSGFNEETCLVKII

SEQ ID NO: 1036 - protein, 15 aa, Homo sapiens
GFNEETCLVKIITGL

SEQ ID NO: 1037 - protein, 15 aa, Homo sapiens
EETCLVKIITGLLEF

SEQ ID NO: 1038 - protein, 15 aa, Homo sapiens
CLVKIITGLLEFEVY

SEQ ID NO: 1039 - protein, 15 aa, Homo sapiens
KIITGLLEFEVYLEY

SEQ ID NO: 1040 - protein, 15 aa, Homo sapiens
TGLLEFEVYLEYLQN

SEQ ID NO: 1041 - protein, 15 aa, Homo sapiens
LEFEVYLEYLQNRFE

SEQ ID NO: 1042 - protein, 15 aa, Homo sapiens
EVYLEYLQNRFESSE

SEQ ID NO: 1043 - protein, 15 aa, Homo sapiens
LEYLQNRFESSEEQA

SEQ ID NO: 1044 - protein, 15 aa, Homo sapiens
LQNRFESSEEQARAV

SEQ ID NO: 1045 - protein, 15 aa, Homo sapiens
RFESSEEQARAVQMS

SEQ ID NO: 1046 - protein, 15 aa, Homo sapiens
SSEEQARAVQMSTKV

SEQ ID NO: 1047 - protein, 15 aa, Homo sapiens
EQARAVQMSTKVLIQ

SEQ ID NO: 1048 - protein, 15 aa, Homo sapiens
RAVQMSTKVLIQFLQ

SEQ ID NO: 1049 - protein, 15 aa, Homo sapiens
QMSTKVLIQFLQKKA

SEQ ID NO: 1050 - protein, 15 aa, Homo sapiens
TKVLIQFLQKKAKNL

SEQ ID NO: 1051 - protein, 15 aa, Homo sapiens
LIQFLQKKAKNLDAI

SEQ ID NO: 1052 - protein, 15 aa, Homo sapiens
FLQKKAKNLDAITTP

SEQ ID NO: 1053 - protein, 15 aa, Homo sapiens
KKAKNLDAITTPDPT

SEQ ID NO: 1054 - protein, 15 aa, Homo sapiens
KNLDAITTPDPTTNA

SEQ ID NO: 1055 - protein, 15 aa, Homo sapiens
DAITTPDPTTNASLL

SEQ ID NO: 1056 - protein, 15 aa, Homo sapiens
TTPDPTTNASLLTKL

SEQ ID NO: 1057 - protein, 15 aa, Homo sapiens
DPTTNASLLTKLQAQ

SEQ ID NO: 1058 - protein, 15 aa, Homo sapiens
TNASLLTKLQAQNQW

SEQ ID NO: 1059 - protein, 15 aa, Homo sapiens
SLLTKLQAQNQWLQD

SEQ ID NO: 1060 - protein, 15 aa, Homo sapiens
TKLQAQNQWLQDMTT

SEQ ID NO: 1061 - protein, 15 aa, Homo sapiens
QAQNQWLQDMTTHLI

SEQ ID NO: 1062 - protein, 15 aa, Homo sapiens
NQWLQDMTTHLILRS

SEQ ID NO: 1063 - protein, 15 aa, Homo sapiens
LQDMTTHLILRSFKE

SEQ ID NO: 1064 - protein, 15 aa, Homo sapiens
MTTHLILRSFKEFLQ

SEQ ID NO: 1065 - protein, 15 aa, Homo sapiens
HLILRSFKEFLQSSL

SEQ ID NO: 1066 - protein, 15 aa, Homo sapiens
LRSFKEFLQSSLRAL

SEQ ID NO: 1067 - protein, 15 aa, Homo sapiens
FKEFLQSSLRALRQM